(19) United States
(12) Patent Application Publication
(10) Pub. No.: US 2024/0368708 A1
(43) Pub. Date: Nov. 7, 2024

(54) METHOD FOR DETECTING AN ANALYTE IN A PATHOGEN-COMPRISING SAMPLE

(71) Applicant: Resolve BioSciences GmbH, Monheim am Rhein (DE)

(72) Inventors: Christian Korfhage, Langenfeld (DE); Frank Reinecke, Essen (DE); Andreas Geipel, Mettmann (DE)

(73) Assignee: Resolve BioSciences GmbH, Monheim am Rhein (DE)

(21) Appl. No.: 18/292,537

(22) PCT Filed: Aug. 4, 2022
(86) PCT No.: PCT/EP2022/071935

§ 371 Date: Jan. 26, 2024

Related U.S. Application Data
(60) Provisional application No. 63/230,247, filed on Aug. 6, 2021.

(51) Int. Cl. C12Q 1/6888; C12Q 1/6841

(52) U.S. Cl. CPC C12Q 1/6888; C12Q 1/6841

(57) ABSTRACT

The present disclosure pertains to novel multiplex methods and kits for detecting different analytes in a sample in parallel by sequential signal-encoding of said analytes. In particular, the present disclosure pertains to a method that results in inactivation of pathogens for in-situ spatial transcriptomics (e.g. Molecular Cartography) without the step DNA or RNA preparation prior to analysis.

### RELATED APPLICATIONS

The present disclosure claims the benefit of priority to U.S. Provisional Application Ser. No. 63/230,247, filed Aug. 6, 2021, and claims the benefit of priority to and is a US national phase entry of PCT Application Number PCT/EP2022/071935, filed Aug. 4, 2022, the contents of both of which are hereby incorporated by reference.

### SEQUENCE LISTING

The instant application contains a Sequence Listing which has been submitted electronically in XML file format and is hereby incorporated by reference in its entirety. Said XML copy, created on Feb. 20, 2024, is named Resolve_003US_SL.xml and is 1,820,014 bytes in size. It replaces a sequence listing, “Resolve.003pr Sequence Listing.txt” that accompanied the original filing, prepared Aug. 5, 2021, and is 275 kb in size. The contents of each listing are incorporated by reference in their entirety.

### FIELD OF THE DISCLOSURE

The technology provided herein relates to methods for detecting analytes in a pathogen-comprising sample, in particular by detecting the analytes in the pathogen-inactivated sample by a a multiplex spatial transcriptomics method, as well as in vitro methods for the diagnosis of a disease.

### BACKGROUND

Methods for detecting specific molecular, cellular and pathogen targets (such as viruses, bacteria, or other pathogens) are fundamental tools for medical and veterinary diagnostics, environmental testing, and industrial quality control. Examples of methods for detecting specific targets in clinical medicine include over-the-counter rapid pregnancy tests, microbiological culture tests for determining the resistance of infectious agents to specific antibiotics, and highly automated tests for cancer markers in blood samples. Detecting pathogen contaminants in food, high throughput screening of candidate compounds for drug discovery, and quantifying active ingredients in pharmaceuticals exemplify industrial manufacturing applications that depend on methods for determining the presence of specific targets. Environmental applications requiring testing for specific targets include detecting water supply contamination, airborne biothreat agents, and household fungal contaminants.

For example, the COVID-19 pandemic caused by SARS-COV-2 highlights clinical needs to detect infection, track strain evolution, and identify biomarkers of disease course. Analysis of gene expression depends on instruments that may not be placed with in labs of higher safety level used for detecting pathogens. Therefore, these analyses of samples with a higher safety level are not possible if the instruments are located in the same laboratory. An alternative method is to extract the biomolecules of interest which in turn reduce the safety level so that the analysis can be performed outside of higher saftety level lab. Typically, spatial transcriptomic analysis of biosafety samples are done after RNA or DNA is isolated. This is typical for a scenario using the Visium technology (10x genomics) or the GeoMx system (Nanostring; https://www.nature.com/articles/s41467-021-21361-7).

The analysis and detection of small quantities of analytes in biological and non-biological samples has become a routine practice in the clinical and analytical environment. Numerous analytical methods have been established for this purpose. Some of them use encoding techniques assigning a particular readable code to a specific first analyte which differs from a code assigned to a specific second analyte.

One of the prior art techniques in this field is the so-called ‘single molecule fluorescence in-situ hybridization’ (smFISH) essentially developed to detect mRNA molecules in a sample. In Lubeck et al. (2014), Single-cell in situ RNA profiling by sequential hybridization, Nat. Methods 11 (4), p. 360-361, the mRNAs of interest are detected via specific directly labeled probe sets. After one round of hybridization and detection, the set of mRNA specific probes is eluted from the mRNAs and the same set of probes with other (or the same) fluorescent labels is used in the next round of hybridization and imaging to generate gene specific color-code schemes over several rounds. The technology needs several differently tagged probe sets per transcript and needs to denature these probe sets after every detection round.

A further development of this technology does not use directly labeled probe sets. Instead, the oligonucleotides of the probe sets provide nucleic acid sequences that serve as initiator for hybridization chain reactions (HCR), a technology that enables signal amplification; see Shah et al. (2016), In situ transcription profiling of single cells reveals spatial organization of cells in the mouse hippocampus, Neuron 92 (2), p. 342-357.

Another technique referred to as ‘multiplexed error robust fluorescence in situ hybridization’ (merFISH) is described by Chen et al. (2015), RNA imaging. Spatially resolved, highly multiplexed RNA profiling in single cells, Science 348 (6233): aaa6090. There, the mRNAs of interest are detected via specific probe sets that provide additional sequence elements for the subsequent specific hybridization of fluorescently labeled oligonucleotides. Each probe set provides four different sequence elements out of a total of 16 sequence elements. After hybridization of the specific probe sets to the mRNAs of interest, the so-called readout hybridizations are performed. In each readout hybridization, one out of the 16 fluorescently labeled oligonucleotides complementary to one of the sequence elements is hybridized. All readout oligonucleotides use the same fluorescent color. After imaging, the fluorescent signals are destroyed via illumination and the next round of readout hybridization takes place without a denaturing step. As a result, a binary code is generated for each mRNA species. A unique signal signature of 4 signals in 16 rounds is created using only a single hybridization round for binding of specific probe sets to the mRNAs of interest, followed by 16 rounds of hybridization of readout oligonucleotides labeled by a single fluorescence color.

A further development of this technology improves the throughput by using two different fluorescent colors, eliminating the signals via disulfide cleavage between the readout-oligonucleotides and the fluorescent label and an alternative hybridization buffer; see Moffitt et al. (2016), High-throughput single-cell gene-expression profiling with multiplexed error-robust fluorescence in situ hybridization, Proc. Natl. Acad. Sci. U S A. 113 (39), p. 11046-11051.

A technology referred to as ‘intron seqFISH’ is described in Shah et al. (2018), Dynamics and spatial genomics of the nascent transcriptome by intron seqFISH, Cell 117 (2), p. 363-376. There, the mRNAs of interest are detected via specific probe sets that provide additional sequence elements for the subsequent specific hybridization of fluorescently labeled oligonucleotides. Each probe set provides one out of 12 possible sequence elements (representing the 12 ‘pseudo colors’ used) per color-coding round. Each color-coding round consists of four serial hybridizations. In each of these serial hybridizations, three readout probes, each labeled with a different fluorophore, are hybridized to the corresponding elements of the mRNA-specific probe sets. After imaging, the readout probes are stripped off by a 55% formamide buffer and the next hybridization follows. After 5 color-coding rounds with 4 serial hybridizations each, the color-codes are completed.

EP 0 611 828 discloses the use of a bridging element to recruit a signal generating element to probes that specifically bind to an analyte. A more specific statement describes the detection of nucleic acids via specific probes that recruit a bridging nucleic acid molecule. This bridging nucleic acids eventually recruit signal-generating nucleic acids. This document also describes the use of a bridging element with more than one binding site for the signal generating element for signal amplification like branched DNA.

Player et al. (2001), Single-copy gene detection using branched DNA (bDNA) in situ hybridization, J. Histochem. Cytochem. 49 (5), p. 603-611, describe a method where the nucleic acids of interest are detected via specific probe sets providing an additional sequence element. In a second step, a preamplifier oligonucleotide is hybridized to this sequence element. This preamplifier oligonucleotide comprises multiple binding sites for amplifier oligonucleotides that are hybridized in a subsequent step. These amplifier oligonucleotides provide multiple sequence elements for the labeled oligonucleotides. This way a branched oligonucleotide tree is build up that leads to an amplification of the signal.

A further development of this method referred to as is described by Wang et al. (2012), RNAscope: a novel in situ RNA analysis platform for formalin-fixed, paraffin-embedded tissues, J. Mol. Diagn. 14 (1), p.22-29, which uses another design of the mRNA-specific probes. Here two of the mRNA-specific oligonucleotides have to hybridize in close proximity to provide a sequence that can recruit the preamplifier oligonucleotide. This way the specificity of the method is increased by reducing the number of false positive signals.

Choi et al. (2010), Programmable in situ amplification for multiplexed imaging of mRNA expression, Nat. Biotechnol. 28 (11), p. 1208-1212, disclose a method known as ‘HCR-hybridization chain reaction’. The mRNAs of interest are detected via specific probe sets that provide an additional sequence element. The additional sequence element is an initiator sequence to start the hybridization chain reaction. Basically, the hybridization chain reaction is based on metastable oligonucleotide hairpins that self-assemble into polymers after a first hairpin is opened via the initiator sequence.

A further development of the technology uses so called split initiator probes that have to hybridize in close proximity to form the initiator sequence for HCR, similarly to the RNAscope technology, this reduces the number of false positive signals; see Choi et al. (2018), Third-generation in situ hybridization chain reaction: multiplexed, quantitative, sensitive, versatile, robust. Development 145 (12).

Mateo et al. (2019), Visualizing DNA folding and RNA in embryos at single-cell resolution, Nature Vol, 568, p. 49ff., disclose a method called ‘optical reconstruction of chromatin structure (ORCA). This method is intended to make the chromosome line visible.

EP 2 992 115 B1 describes a method of sequential single molecule hybridization and provides technologies for detecting and/or quantifying nucleic acids in cells, tissues, organs or organisms through sequential barcoding.

Against this background, it is an object underlying the present disclosure to provide a method for detecting an analyte in a pathogen-comprising sample by means of which the disadvantages of the prior art methods can be reduced or even avoided.

### SUMMARY OF THE DISCLOSURE

The present disclosure pertains to novel multiplex methods and kits for detecting different analytes in a sample in parallel by sequential signal-encoding of said analytes. In particular, the present disclosure pertains to a method that results in inactivation of pathogens for in-situ spatial transcriptomics (e.g. Molecular Cartography) without the step DNA or RNA preparation prior to analysis. One important advantage is that the analysis can be done outside of a BSL3 or BSL4 lab.

In an advantageous embodiment of the present disclosure is that the method for detecting an analyte in a pathogen-comprising sample comprises the steps of:
1) Taking a sample containing pathogens with a risk level>2.
2) Inactivation of the pathogen within the sample without isolation RNA or DNA from the pathogen or sample
3) Performing spatial*omics analysis.

In a first aspect, embodiments of the disclosure in particular pertains to a method for detecting an analyte in a pathogen-comprising sample comprising:
i) Inactivation of the pathogen within the sample without isolation of RNA and/or DNA from the pathogen or the sample;
ii) Detecting the analyte by spatial transcriptomics.

In particular, the spatial transcriptomics detecting comprises a multiplex method for detecting different analytes in a sample by sequential signal-encoding of said analytes, comprising:

(A) contacting the sample with at least twenty (20) different sets of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising
(aa) a binding element(S) that specifically interacts with one of the different analytes to be encoded, and
(bb) an identifier element (T) comprising a nucleotide sequence which is unique to the analyte to be encoded (unique identifier sequence),

wherein the analyte-specific probes of a particular set of analyte-specific probes differ from the analyte-specific probes of another set of analyte-specific probes in the nucleotide sequence of the identifier element (T),

wherein the analyte-specific probes in each set of analyte-specific probes binds to the same analyte and comprises the same nucleotide sequence of the identifier element (T) which is unique to said analyte; and

(B) contacting the sample with at least one set of decoding oligonucleotides per analyte, wherein in each set of decoding oligonucleotides for an individual analyte each decoding oligonucleotide comprises:
(aa) an identifier connector element (t) comprising a nucleotide sequence which is essentially complementary to at least a section of the unique identifier sequence of the identifier element (T) of the corresponding analyte-specific probe set, and
(bb) a translator element (c) comprising a nucleotide sequence allowing a specific hybridization of a signal oligonucleotide;

wherein the decoding oligonucleotides of a set for an individual analyte differ from the decoding oligonucleotides of another set for a different analyte in the first connect element (t); and

(C) contacting the sample with at least a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and
(bb) a signal element.

(D) Detecting the signal caused by the signal element;

(E) selectively removing the decoding oligonucleotides and signal oligonucleotides from the sample, thereby essentially maintaining the specific binding of the analyte-specific probes to the analytes to be encoded;

(F) Performing at least three (3) further cycles comprising steps B) to E) to generate an encoding scheme with a code word per analyte, wherein in particular the last cycle may stop with step (D).

In a second aspect, embodiments of this disclosure relate to in vitro methods for diagnosis of a disease due to a pathogen infection like a viral or bacterial infection comprising the use of a method according to the present disclosure.

In a third aspect, embodiments of this disclosure provide in vitro methods for diagnosis of a disease in plants caused by infectious and/or parasitic origin, said method comprising the use of a method according to the present disclosure.

Before the disclosure is described in detail, it is to be understood that this disclosure is not limited to the particular component parts of the steps of the methods described. It is also to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. It is moreover to be understood that, in case parameter ranges are given which are delimited by numeric values, the ranges are deemed to include these limitation values.

### BRIEF DESCRIPTION OF THE DRAWINGS

FIG. 1: Embodiment where the analyte is a nucleic acid and the probe set comprises oligonucleotides specifically binding to the analyte. The probes comprise a unique identifier sequence allowing hybridization of decoding oligonucleotides.

FIG. 2: Embodiment where the analyte is a protein and the probe set comprises proteins (here: antibodies) specifically binding to the analyte. The probes comprise a unique identifier sequence allowing hybridization of decoding oligonucleotides.

FIG. 3: Flowchart of the method according to the disclosure.

FIG. 4: Alternative options for the application of decoding and signal oligonucleotides.

FIG. 5: Example for signal encoding of three different nucleic acid sequences by two different signal types and three detection rounds; in this example, the encoding scheme includes error detection.

FIG. 6: Number of generated code words (logarithmic scale) against number of detection cycles.

FIG. 7: Calculated total efficiency of a 5-round encoding scheme based on single step efficiencies.

FIG. 8: Comparison of relative transcript abundances between different experiments.

FIG. 9: Correlation of relative transcript abundances between different experiments.

FIG. 10: Comparison of intercellular distribution of signals.

FIG. 11: Comparison of intracellular distribution of signals.

FIG. 12: Distribution pattern of different cell cycle dependent transcripts.

FIG. 13: Detection of multiple targets using a 8 round code with 2 labels (A and B) and no label (−). The targets 1, 2, 3, 4, 5, 20, and n are represented. The rounds 1, 2, 3, and 8 of the coding scheme are represented. Herein, the blank is part of the code.

FIG. 14: Detection of multiple targets can be performed by an encoding scheme using a detectable marker. The ending scheme may comprise also the “0” as a marker. That means that at a specific position the transcript is not detected. Consequently, the encoding scheme may be represented by the following constructs using only two gene specific probes:
1) With detectable label F: detectable during imaging
2) With detectable label F and quencher Q: not detectable during imaging
3) With quencher Q: not detectable during imaging
4) Without label F: not detectable during imaging
5) Without signaling oligonucleotide: not detectable during imaging
6) With a decoder oligonucleotide that cannot recruit a signaling oligonucleotide
7) Without decoder oligonucleotide: not detectable during imaging

FIG. 15: Possible structures of a multi-decoder. The numbers depict the examples. (A) is the unique identifier sequence, (a) is the corresponding sequence of the decoding oligonucleotide or multi-decoder and (c1) to (c3) are different sequence elements, that specifically bind to different signal oligonucleotides. Examples 2 to 5 show different versions of multi decoders. The order of the different sequence elements as well as the number of signal oligonucleotide binding elements is not fixed. Example 1 shows a normal decoding oligonucleotide since there is only one signal oligonucleotide binding element (c1).

FIG. 16: Example for signal encoding of three different nucleic acid sequences by using multi-decoders and two different signal oligonucleotides creating three different signal types and three detection rounds. In this example, the encoding scheme includes error detection and correction.

FIG. 17: Number of generated code words (logarithmic scale) against number of detection cycles. The number of code words for merFISH does not exponentially increase with the number of detection cycles but gets less effective with each added round. In contrast, the number of code words for intronSeqFISH, the method of the present disclosure without using multi-decoders, the method with multi-decoders increases exponentially. The slope of the curve for the method using multi-decoders is much higher than that of the prior invention, leading to more than 20 000 000 times more code words usable after 20 rounds of detection.

### DETAILED DESCRIPTION OF THE DISCLOSURE

Disclosed herein are novel multiplex methods and kits for detecting different analytes in a sample by sequential signal-encoding of said analytes.

The present disclosure describes the usage of a set of labeled and unlabeled nucleic acid sequences for specific quantitative and/or spatial detection of different analytes in parallel via specific hybridization. The technology allows the discrimination of more different analytes than different detection signals are available. The discrimination may be realized via sequential signal-coding of the analytes achieved by several cycles of specific hybridization, detection of signals and selective elution of the hybridized nucleic acid sequences.

In contrast to other state-of-the-art methods, the oligonucleotides providing the detectable signal are not directly interacting with sample-specific nucleic acid sequences but are mediated by so called “decoding-oligonucleotides”. This mechanism decouples the dependency between the analyte-specific oligonucleotides and the signal oligonucleotides. The use of decoding-oligonucleotides allows a much higher flexibility while dramatically decreasing the number of different signal oligonucleotides needed which in turn increases the coding capacity achieved with a certain number of detection rounds. The utilization of decoding-oligonucleotides leads to a sequential signal-coding technology that is more flexible, cheaper, simpler, faster and/or more accurate than other methods.

### A. Definitions

According to the present disclosure an “analyte” is the subject to be specifically detected as being present or absent in a sample and, in case of its presence, to encode it. It can be any kind of entity, including a protein, polypeptide, protein or a nucleic acid molecule (e.g. RNA, PNA or DNA) of interest. The analyte provides at least one site for specific binding with analyte-specific probes. Sometimes herein the term “analyte” is replaced by “target”. An “analyte” according to the disclosure incudes a complex of subjects, e.g. at least two individual nucleic acid, protein or peptides molecules. In an embodiment of the disclosure an “analyte” excludes a chromosome. In another embodiment of the disclosure an “analyte” excludes DNA.

In some embodiments, an analyte may be a “coding sequence”, “encoding sequence”, “structural nucleotide sequence” or “structural nucleic acid molecule” which refers to a nucleotide sequence that is translated into a polypeptide, usually via mRNA, when placed under the control of appropriate regulatory sequences. The boundaries of the coding sequence are determined by a translation start codon at the 5′-terminus and a translation stop codon at the 3′-terminus. A coding sequence can include, but is not limited to, genomic DNA, cDNA, EST and recombinant nucleotide sequences.

A “sample” as referred to herein is a composition in liquid or solid form suspected of comprising the analytes to be encoded. In particular, the sample is a biological sample, preferably comprising biological tissue, further preferably comprising biological cells and/or extracts and/or part of cells. For example, thee cell is a prokaryotic cells or a eukaryotic cell, in particular a mammalian cell, in particular a human cell. In some embodiments, the biological tissue, biological cells, extracts and/or part of cells are fixed. In particular, the analytes are fixed in a permeabilized sample, such as a cell-containing sample. In particular, the sample is a pathogen-comprising sample and may be any kind of substance, tissue, biological stuff, organism etc. that comtain a pathogen. The sample can be any kind of a solid, a fluidic sample, a smear, a surface that comprise pathogenic material. The sample may comprise eukaryotic, archeae, procaryotic organisms, or viruses. The sample may derive from an organism, an environmental sample, an excretion from an organism, or a surface.

A “pathogen” includes every type of biological entity (eukaryotic, archeae, procaryotic, virus, viroid, protein, pure RNA) that is defined to have a risk for the life humans, animals, plants, or microorganism communities (e.g. in gut, fermenter, lakes etc.) with a “risk level” higher than 2. The risk level is defined by local authorities and may differ locally (e.g. from country to country). E.g., the risk level in Germany is defined by the BioStoffV. In other countries the risk level is defined by other laws and regulations (e.g., CDC in USA). In any case, the risk level of a pathogen is defined in a process based on scientific experience.

“Inactivation of a pathogen” means in particular that a risk level of a pathogen higher than 2 is reduced to at least a risk level of 2 or lower. Ideally, the pathogen inactivating process do not avoid the detection of the pathogen within the detection process. In some embodiments, the pathogen inactivating process do not reduce the detection efficiency of the pathogen within the detection process. The inactivation includes any kind of physical, chemical, biochemical, or biological treatment of a sample to reduce the risk level from >2 to a risk level of 2 or lower. Inactivation includes also any kind of combination of physical, chemical, biochemical, or biological treatment. Therefore, pathogen inactivation comprises to stop the pathogen in a given sample from contaminating the desired sample by rendering them non-infectious, e.g. viral inactivation renders viruses unable to infect. Examples of methods for measuring the inactivation of pathogens are described in WO1993/015215A1.

A pathogen inactivation method may or may not achieve complete inactivation, it is useful to consider a specific example. A bacterial culture is said to be sterilized if an aliquot of the culture, when transferred to a fresh culture plate and permitted to grow, is undetectable after a certain time period. The time period and the growth conditions (e.g., temperature) define an “amplification factor”. This amplification factor along with the limitations of the detection method (e.g., visual inspection of the culture plate for the appearance of a bacterial colony) define the sensitivity of the inactivation method. A minimal number of viable bacteria must be applied to the plate for a signal to be detectable. With the optimum detection method, this minimal number is one bacterial cell. With a suboptimal detection method, the minimal number of bacterial cells applied so that a signal is observed may be much greater than 1. The detection method determines a “threshold” below which the method appears to be completely effective (and above which the method is, in fact, only partially effective).

Furthermore, inactivation of viruses may be demonstrated by a biological assay such as an enzyme assay (e.g., reduction in reverse transcriptase) or cell culture (e.g., virus-induced host cell death). A more sensitive method has been described by C. V. Hanson er. al. for quantifying HIV. See J. Clin. Microbiol. 23:2030 (1990). The method is a plaque assay employing HIV-sensitive cells in a monolayer. A fluorescent stain is used and detection is made by visualization.

For example, a spiking study is a study done in order to determine the possible methods of pathogen like viral inactivation. The results of these studies are numerical and, based on these numbers, researchers can determine whether or not the process on which the study was conducted will be suitable for the viruses they are trying to extract and the solution from which they are trying to extract them. For example, it may be shown through experimentation, that increasing the viral count (or level of activity) of a sample by a factor of 10⁴ or 10⁵ of the original will only change the virus inactivation ratios by one order of magnitude. From this knowledge, spiking studies have been created in which the virus number (or level of activation) is increased or “spiked” by a factor of 10⁴ or 10⁵ of the original sample. This new high number or level of activity is then run through the process stream and purified. The number or level of activity is taken at the beginning and at the end of the process stream and used in the calculation of Reduction Factor. Reduction factor (RF) for a virus removal or inactivation step is calculated using the following equation (Note for Guidance on Virus Validation Studies: The Design, Contribution and Interpretation of Studies Validating the Inactivation and Removal of Viruses, EMEA CPMP BWP, 268/95 1996): RFₛₜₑₚ=log₁₀ [(V1×T1)/(V2×T2)], wherein V1=volume of spiked feedstock prior to the clearance step; T1=virus concentration of spiked feedstock prior to the clearance step; V2=volume of material after the clearance step; and T2=virus concentration of material after the clearance step.

Furthermore, virucidal activity studies may be performed by using a quantitative suspension test with 30 s exposure time (as descriped in World Health Organization. WHO guidelines on hand hygiene in health care: first global patient safety challenge clean care is safer care. Geneva: the Organization; 2009. [cited 2020 Apr. 8]). One part virus suspension may be mixed with one part organic load (0.3% bovine serum albumin as an interfering substance) and 8 parts disinfectant solution of different concentrations. After a 30 s exposure, samples may be serially diluted and and the 50% tissue culture infectious dose (TCID50) per milliliter are determined by using crystal violet staining and subsequently scoring the number of wells displaying cytopathic effects. The TCID50 is calculated by the Spearman-Kärber algorithm, as described (George VG, Hierholzer JC, Ades EW. Cell culture. In: Virology methods manual. Mahy BWJ, Kangro HO, editors. Academic Press: London; 1996. p. 3-24). The cytotoxic effects of disinfectants can be monitored by using crystal violet staining and optical analysis for altered density and morphology of the cellular monolayer in the absence of virus. We quantified cytotoxic effects analogous to the TCID₅₀/mL of the virus infectivity. The dose-response curves can be determined as percent normalized virus inactivation versus percent log disinfectant concentration by nonlinear regression using the robust fitting method on the normalized TCID₅₀ data implemented in Prism version 8.0.3 (GraphPad, https://www.graphpad.com). The reduction factors (RFs) for each treatment condition can be calculated with the formular:

$$RF = treatment - control = {log}_{10} ( \frac{{\sum }_{i = 1}^{n} {x}_{i}}{n} ) - {log}_{10} ( \frac{{\sum }_{j = 1}^{m} {x}_{j}}{m} )$$

Physical inactivation treatments for pathogens according to the present disclosure are not limited to but may include any kind of changing temperature, electromagnetic waves, light (including also visible and invisible light), incubation the sample with radioactive substances, etc. that reduce the risk level of a pathogen higher than 2 to at least to a risk level of 2 or lower. Combinations thereof or other treatments are not excluded.

Some examples without limitations to those:

Treatment by dry temperature: >30° C., >40° C., >50° C., >60° C., >70° C., >80° C., >90° C., >100°° C.>110° C. etc.

Treatment by temperature in solution >30° C., >40° C., >50° C., >60° C., >70° C., >80° C., >90° C., >100°° C.>110° C. etc.

Treatment by X-Ray, radioactivity, UV Light, blue light, red lighty, infrared light etc.

Chemical inactivation treatments for pathogens according to the present disclosure are not limited to but may include any kind treating the sample by of changing the pathogen environment like pH, salts, fraction of polar to non-polar solvents, oxidative reagents, reductive reagents, reagents that perform covalent or non-covalent linkage to the pathogen or degradative reagents that cleave at least parts of pathogen. All reactions have in common that they reduce the risk level of a pathogen higher than 2 to at least a risk level of 2 or lower. Combinations thereof or other treatments are not excluded. Some examples without limitations to those:

Treatment by redox reactive reagents such as but not limited to Beta-Mercaptoethanol, Dithiothreitol (DTT), or Dithioerythrit, metal based such as sodium borohydride (NaBH₄) or lithium aluminium hydride (LiAlH₄). peroxides (e.g.H₂O₂), Ozone, potassium permanganate, organic peroxides (e.g. tert-butyl hydroperoxide), or sodium perborate, hypochlorites, and other redoxreactive reagents which may be chosen by a researcher from the galvanic series. Redox reagents are all agents and conditions that are able to oxidize or rduce other chemical compunds. The redox reactivity of such reagents can be measured as an electrode potential compared to the standard electrode potential. Redox reactive agents can change their redox-reactive capability by certain reaction conditions that change temperature, pH, concentration, or other parameters (see text book for redox reactions). From this view, the experienced user can change the agents and reactions parameters to find the right redox-reactive substance. For example, the pathogen containing sample is treated by the redox reactive substance in a way so that the pathogen sticks more tightly to the sample or surface or it inactivates the entry mechanism fro pathogens or any other reaction required by the pathogen to propagate in living organisms. Typical pathogen inactivating reagents that result in crosslinkiung of pathogens are aldehydes, formaldehyde, glutaraldehyde, Boinsche Solution, etc. . . . Typical pathogen inactivating reagents that result in a change of the lipophily of the solution are alcohols such as methanol, ethanol, propanol, etc. . . . Typical pathogen inactivating reagents that change of the water tension are detergents such as SDS, Triton, NP etc. . . .

For example, a composition for a chemical inactivation treatment may consist/comprise 80% (vol/vol) ethanol, 1.45% (vol/vol) glycerol, and 0.125% (vol/vol) hydrogen peroxide. A further composition may consist/comprise 75% (vol/vol) 2-propanol, 1.45% (vol/vol) glycerol, and 0.125% (vol/vol) hydrogen peroxide. A further composition may consist/comprise 80% (wt/wt) ethanol, 0.725% (vol/vol) glycerol, and 0.125% (vol/vol) hydrogen peroxide. A further composition may consist/comprise 75% (wt/wt) 2-propanol, 0.725% (vol/vol) glycerol, and 0.125% (vol/vol) hydrogen peroxide.

Biochemical inactivation treatments for pathogens according to the present disclosure are not limited to but may include any kind of enzymatic reactions, competing reactions or reversibel or irreversible reactions, adding biomolecules (e.g. proteins, fatty acids, carbohydrates, nucleic acids or mixture thereof) that reduce the risk level of a pathogen higher than 2 to at least to a risk level of 2 or lower. Combinations thereof or other treatments are not excluded. Typical pathogen inactivating reagents that icludes enzymes such as biomolecule degrading enzymes, protein degrading enzymes, enzymes that degrades lipohilic or amphiphilic biomolecules, enzymes that degrades polymers, antibodies etc..

Biological inactivation treatments for pathogens according to the present disclosure are not limited to but may include any kind treating the sample by a biological component that perform the reduction of the risk level of a pathogen higher than 2 to at least a risk level of 2 or lower. Biological inactivation treatments for pathogens according to the present disclosure are not limited to but may include any kind of treating the sample by counteracting phages, viruses, bacteria, or other organisms or parts of organisms that reduce the the risk level of a pathogen higher than 2 to at least to a risk level of 2 or lower. Combinations thereof or other treatments are not excluded.

Spatial transcriptomics (or Spatial *omics) according to the present disclosure means any kind of analysis where data from the sample are derived in a spatial manner from in-situ samples of tissues or whole organisms. The in situ sample may be a section of an organ or an organism. The in-situ sample may be not pretreated or pretreated in a way that is required for improving the result. Spatial*omics may included the detection of small molecules compunds of tissues or cells, proteins, DNA, and/or RNA. More preferentially, spatial*omics is restricted to proteins, DNA, and/or RNA. More preferentially, spatial*omics is restricted to DNA and/or RNA. Even more preferentially, spatial*omics is restricted to smFISH. Even more preferentially, spatial*omics is restricted to any kind of sequential smFISH.

In particular, the spatial transcriptomics detecting comprises a multiplex method for detecting different analytes in the pathogen-comprising sample by sequential signal-encoding of said analytes, comprising an inactivation of the pathogen within the sample without isolation of RNA and/or DNA from the pathogen or the sample, and
(A) contacting the sample with at least twenty (20) different sets of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising
   (aa) a binding element(S) that specifically interacts with one of the different analytes to be encoded, and
   (bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
(B) contacting the sample with at least one set of decoding oligonucleotides per analyte, wherein in each set of decoding oligonucleotides for an individual analyte each decoding oligonucleotide comprises:
   (aa) an identifier connector element (t) comprising a nucleotide sequence which is essentially complementary to at least a section of the unique identifier sequence of the identifier element (T) of the corresponding analyte-specific probe set, and
   (bb) a translator element (c) comprising a nucleotide sequence allowing a specific hybridization of a signal oligonucleotide;

wherein the decoding oligonucleotides of a set for an individual analyte differ from the decoding oligonucleotides of another set for a different analyte in the first connect element (t); and
(C) contacting the sample with at least a set of signal oligonucleotides, each signal oligonucleotide comprising:
   (aa) a translator connector element (C) comprising a nucleotide sequence which is essentially complementary to at least a section of the nucleotide sequence of a translator element (c) comprised in a decoding oligonucleotide, and (bb) a signal element.
(D) Detecting the signal caused by the signal element;
(E) Selectively removing the decoding oligonucleotides and signal oligonucleotides from the sample, thereby essentially maintaining the specific binding of the analyte-specific probes to the analytes to be encoded;
(F) Performing at least three (3) further cycles comprising steps B) to E) to generate an encoding scheme with a code word per analyte, wherein in particular the last cycle may stop with step (D).

As used in the present disclosure, “cell”, “cell line”, and “cell culture” can be used interchangeably and all such designations include progeny. Thus, the words “transformants” or “transformed cells” include the primary subject cell and cultures derived therefrom without regard for the number of transfers. It is also understood that all progeny may not be precisely identical in DNA content, due to deliberate or inadvertent mutations. Mutant progeny that have the same functionality as screened for in the originally transformed cell are included.

One important advantage of the methods according to the present disclosure is that the analysis can be done outside of a BSL3 or BSL4 lab. A biosafety level (BSL), or pathogen/protection level, is a set of biocontainment precautions required to isolate dangerous biological agents in an enclosed laboratory facility. Thee levels of containment range from the lowest biosafety level 1 (BSL-1) to the highest at level 4 (BSL-4). In the United States, the Centers for Disease Control and Prevention (CDC) have specified these levels. In the European Union, the same biosafety levels are defined in a directive. In Canada the four levels are known as Containment Levels. Facilities with these designations are also sometimes given as P1 through P4 (for pathogen or protection level), as in the term P3 laboratory. For example Biosafety level 3 (BSL-3) is appropriate for work involving pathogens which can cause serious and potentially lethal disease via the inhalation route. Biosafety level 4 (BSL-4) is the highest level of biosafety precautions, and is appropriate for work with agents that could easily be aerosol-transmitted within the laboratory and cause severe to fatal disease in humans for which there are no available vaccines or treatments.

An “encoding scheme” may describe a set of code words that are associated with the analytes to be detected. Each code word refers to one of the analytes and can be distinguished from all other code words. A code word hereby is a sequence of signs provided by the detection cycles of the method. A sign within a code word is a detectable signal or the absence of a signal. A code word does not need to comprise of all different signals used in the method. The number of signs in a code word is defined by the number of detection cycles.

An “oligonucleotide” as used herein, refers to s short nucleic acid molecule, such as DNA, PNA, LNA or RNA. The length of the oligonucleotides is within the range 4-200 nucleotides (nt), preferably 6-80 nt, more preferably 8-60 nt, more preferably 10-50 nt, more preferably 12 to 35 depending on the number of consecutive sequence elements. The nucleic acid molecule can be fully or partially single-stranded. The oligonucleotides may be linear or may comprise hairpin or loop structures. The oligonucleotides may comprise modifications such as biotin, labeling moieties, blocking moieties, or other modifications.

The “analyte-specific probe” consists of at least two elements, namely the so-called binding element(S) which specifically interacts with one of the analytes, and a so-called identifier element (T) comprising the ‘unique identifier sequence’. The binding element(S) may be a nucleic acid such as a hybridization sequence or an aptamer, or a peptidic structure such as an antibody.

Also a “probe” consists of at least two elements, namely the so-called binding element(S) which specifically interacts with one of the analytes, and a so-called identifier element (T) comprising the ‘unique identifier sequence’. The binding element(S) may be a nucleic acid such as a hybridization sequence or an aptamer, or a peptidic structure such as an antibody.

In particular, in some embodiments the binding element(S) comprises moieties which are affinity moieties from affinity substances or affinity substances in their entirety selected from the group consisting of antibodies, antibody fragments, receptor ligands, enzyme substrates, lectins, cytokines, lymphokines, interleukins, angiogenic or virulence factors, allergens, peptidic allergens, recombinant allergens, allergen-idiotypical antibodies, autoimmune-provoking structures, tissue-rejection-inducing structures, immunoglobulin constant regions and their derivatives, mutants or combinations thereof. In further advantageous embodiments, the antibody fragment is a Fab, an scFv; a single domain, or a fragment thereof, a bis scFv, Fab2, Fab3, minibody, maxibody, diabody, triabody, tetrabody or tandab, in particular a single-chain variable fragment (scFv).

The “unique identifier sequence” as comprised by the analyte-specific probe is unique in its sequence compared to other unique identifiers. “Unique” in this context means that it specifically identifies only one analyte, such as Cyclin A, Cyclin D, Cyclin E etc., or, alternatively, it specifically identifies only a group of ana-lytes, independently whether the group of analytes comprises a gene family or not.

Therefore, the analyte or a group of analytes to be encoded by this unique identifier can be distinguished from all other analytes or groups of analytes that are to be encoded based on the unique identifier sequence of the identifier element (T). Or, in other words, there is only one ‘unique identifier sequence’ for a particular analyte or a group of analytes, but not more than one, i.e. not even two. Due to the uniqueness of the unique identifier sequence the identifier element (T) hybridizes to exactly one type of decoding oligonucleotides. The length of the unique identifier sequence is within the range 8-60 nt, preferably 12-40 nt, more preferably 14-20 nt, depending on the number of analytes encoded in parallel and the stability of interaction needed. A unique identifier may be a sequence element of the analyte-specific probe, attached directly or by a linker, a covalent bond or high affinity binding modes, e.g. antibody-antigen interaction, streptavidin-biotin interaction etc. It is understood that the term “analyte specific probe” includes a plurality of probes which may differ in their binding elements (S) in a way that each probe binds to the same analyte but possibly to different parts thereof, for instance to different (e.g. neighboring) or overlapping sections of the nucleotide sequence comprised by the nucleic acid molecule to be encoded-ed. However, each of the plurality of the probes comprises the same identifier element (T).

A “bipartite labeling probe” comprises a binding sequence capable of hybridizing the analyte and a binding probe sequence capable of binding a detectable signal molecule like a fluorophore or a nucleic acid sequence comprising a fluorophore.

A “decoding oligonucleotide” or an “adapter” or a/adapter segment” consists of at least two sequence elements. One sequence element that can specifically bind to a unique identifier sequence, referred to as an “identifier connector element” (t) or “first connector element” (t), and a second sequence element specifically binding to a signal oligonucleotide, referred to as “translator element” (c). The length of the sequence elements is within the range 8-60 nt, preferably 12-40 nt, more preferably 14-20 nt, de-pending on the number of analytes to be encoded in parallel, the stability of interaction needed and the number of different signal oligonucleotides used. The length of the two sequence elements may or may not be the same.

In some advantageous embodiments, the decoding oligonucleotide in the kits and/or methods of the present disclosure may be a “multi-decoder”. A “multi-decoder” is a decoding oligonucleotide that consists of at least three sequence elements. One sequence element (the identifier connector element (t)) can specifically bind to a unique identifier sequence (identifier element (T)) and at least two other sequence elements (translator elements (c)) specifically bind different signal oligonucleotides (each of these sequence elements specifically binds a signal oligonucleotide that differs to all other signal oligonucleotides recruited by other elements of the multi-decoder). The length of the sequence elements is within the range 8-60 nt, preferably 12-40 nt, more preferably 14-20 nt, depending on the number of analytes detected in parallel, the stability needed and the number of different signal oligonucleotides used. The length of the sequence elements may or may not be the same.

Therefore, in some advantageous embodiments, the decoding oligonucleotide is a multi-decoder comprising
an identifier connector element (t) comprising a nucleotide sequence which is essentially complementary to at least a section of the unique identifier sequence of the identifier element (T) of the corresponding analyte-specific probe set, and
at least two translator elements (c) comprising each a nucleotide sequence allowing a specific hybridization of a different signal oligonucleotide.

Therefore, the first translator element binds a different signal oligonucleotide as the second translator element. In particular, the signal oligonucleotides differ in the signal element comprised in the signal oligonucleotide, e.g. in the kind of the fluorophore.

A “signal oligonucleotide” or a “reporter” as used herein comprises at least two elements, a so-called “translator connector element” (C) or “second connector element” (C) having a nucleotide sequence specifically hybridizable to at least a section of the nucleotide sequence of the translator element (c) of the decoding oligonucleotide, and a “signal element” which provides a detectable signal. This element can either actively generate a detectable signal or provide such a signal via manipulation, e.g. fluorescent excitation. Typical signal elements are, for example, enzymes that catalyze a detectable reaction, fluorophores, radioactive elements or dyes.

A “set” refers to a plurality of moieties or subjects, e.g. analyte-specific probes or decoding oligonucleotides, whether the individual members of said plurality are identical or different from each other. In an analyte specific probe set, the analyte specific probes are identical in the identifier element (T) but may comprise a different binding element(S) for specifically interacting with the same analyte but for specifically interacting with different sub-structures of the same analyte to be encoded.

“Selective denaturation” may be the process of eliminating bound decoding oligonucleotides and signal oligonucleotides with highest efficiency while at the same time the target specific probes have to stay hybridized with the highest efficiency. The total efficiency of these two combined events may to be at least 0.22 for two detection cycles, 0.37 for three detection cycles, 0.47 for four detection cycles, 0.55 for five detection cycles, 0.61 for six detection cycles, 0.65 for seven detection cycles, 0.69 for eight detection cycles, 0.72 for nine detection cycles and 0.74 for 10 detection cycles, 0.76 for 11 detection cycles and 0.78 for 12 detection cycles.

In an embodiment of the disclosure a single set refers to a plurality of oligonucleotides.

An “analyte specific probe set” refers to a plurality of moieties or sub-jects, e.g. analyte-specific probes that are different from each other and bind to independent regions of the analyte. A single analyte specific probe set is further characterized by the same unique identifier.

A “decoding oligonucleotide set” refers to a plurality of decoding oligonucleotides specific for a certain unique identifier needed to realize the encoding independent of the length of the code word. Each and all of the decoding oligonucleotides included in a “decoding oligonucleotide set” bind to the same unique identifier element (T) of the analyte-specific probe.

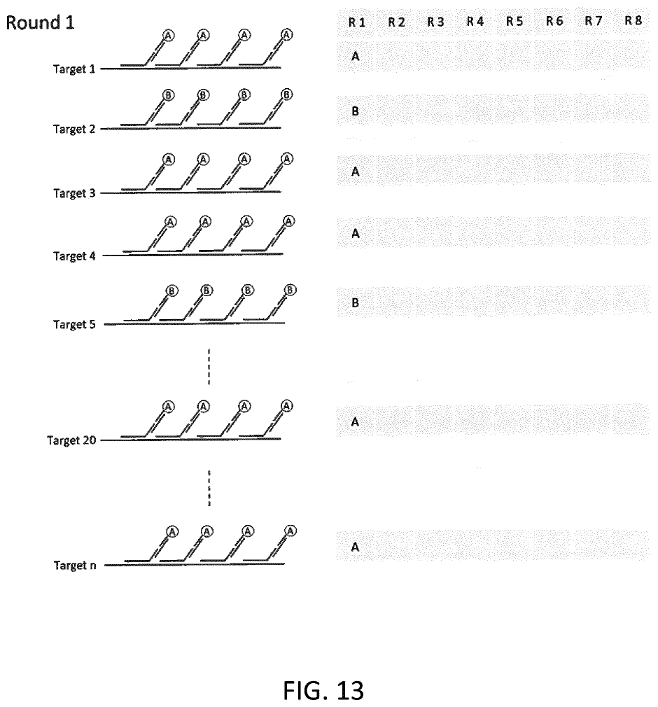

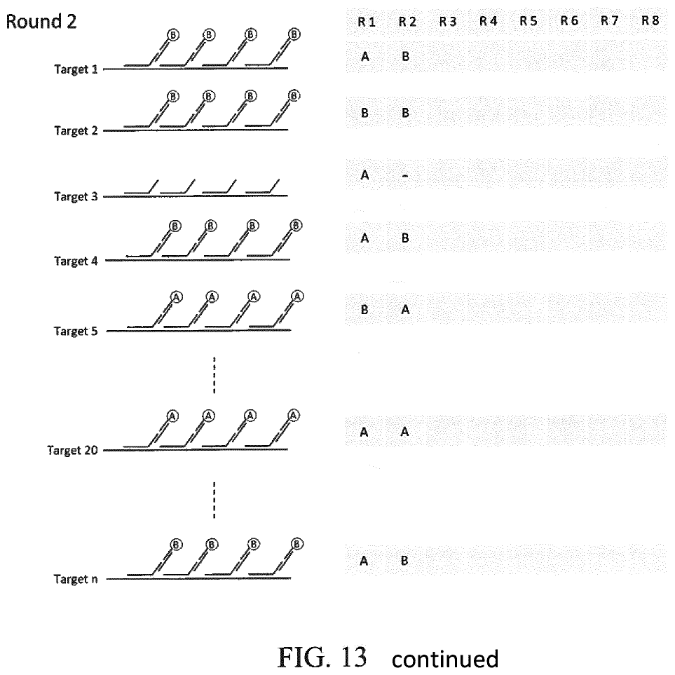

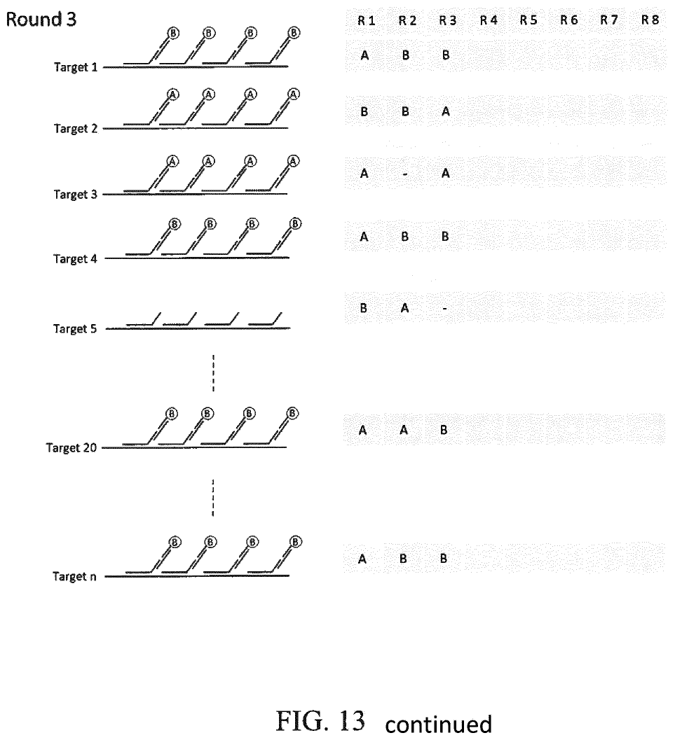

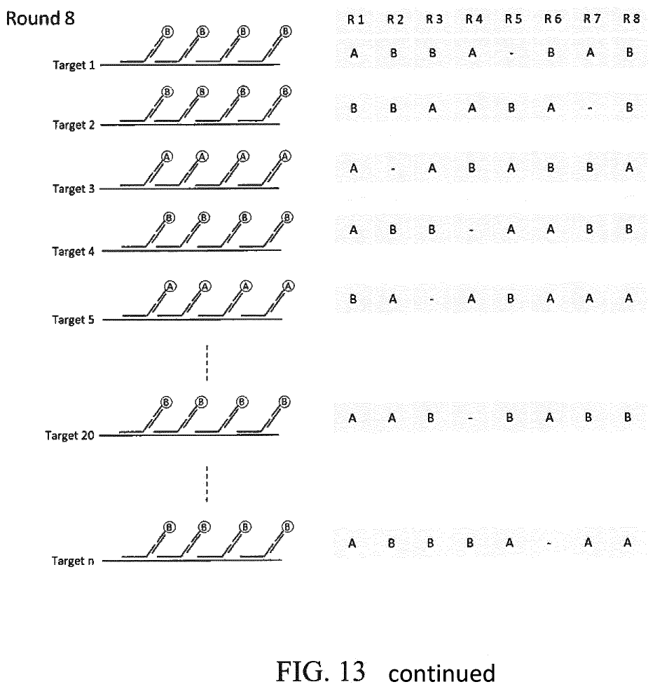

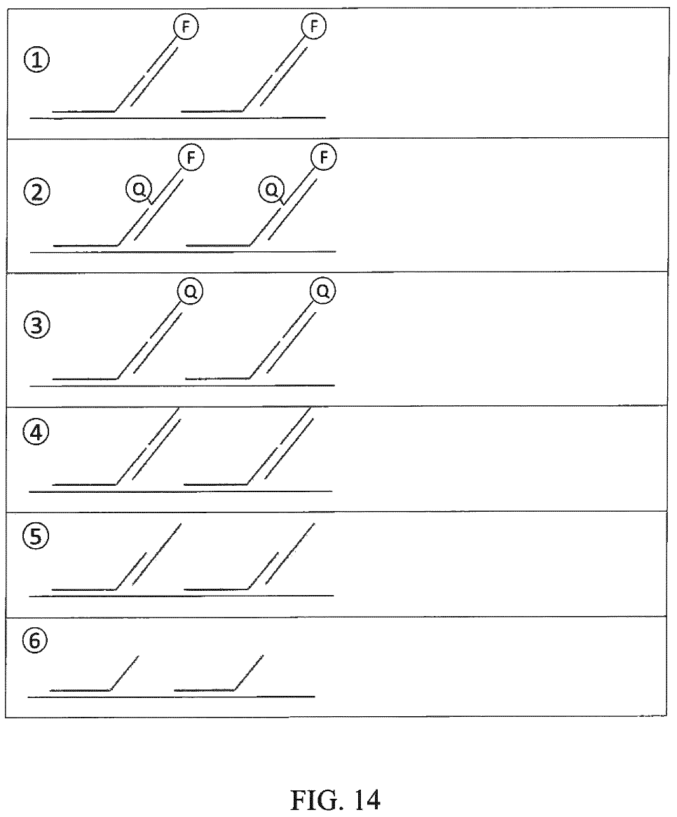

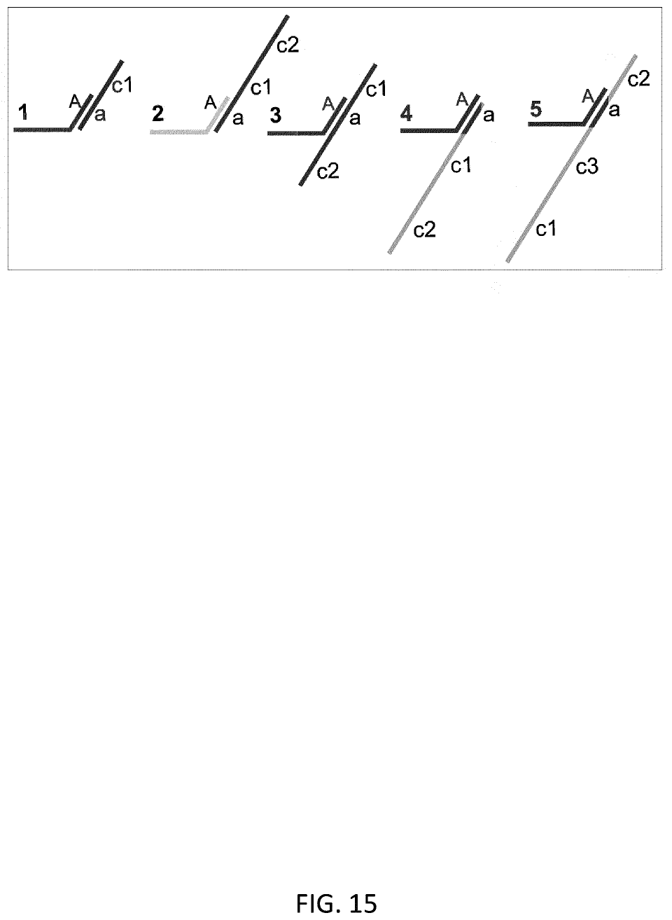

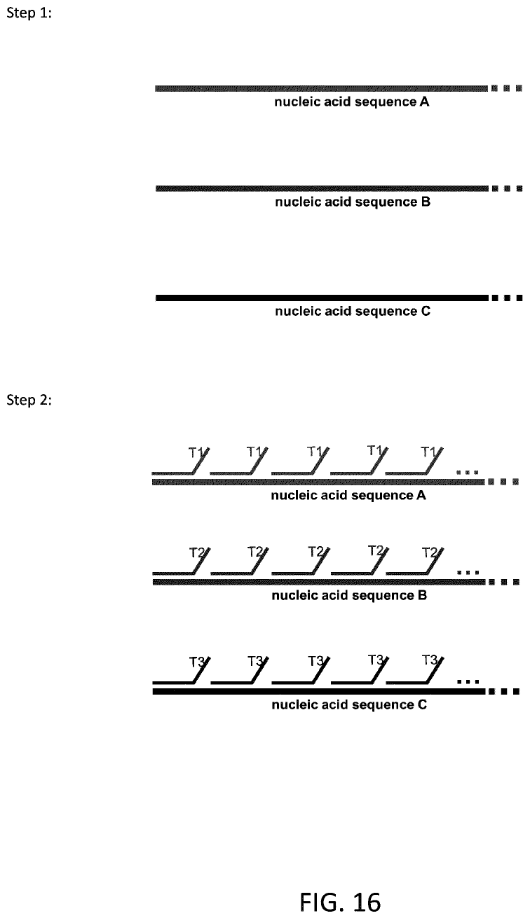

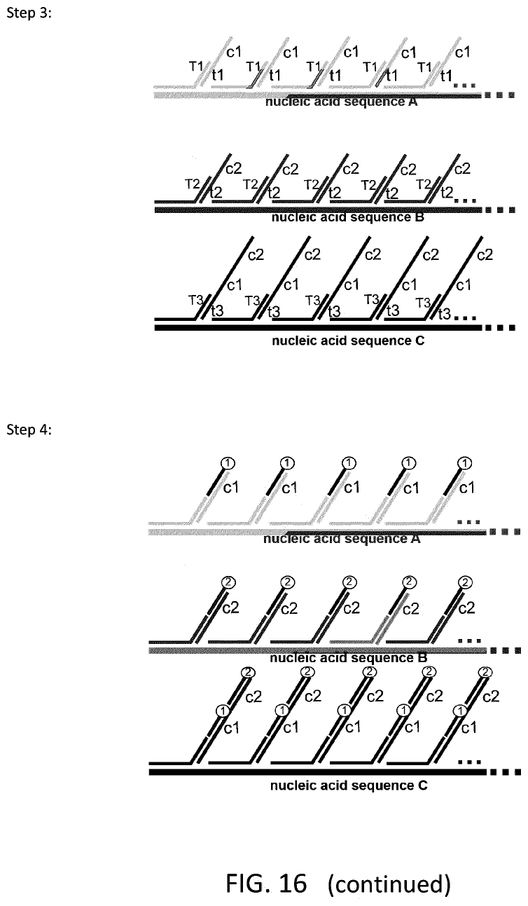

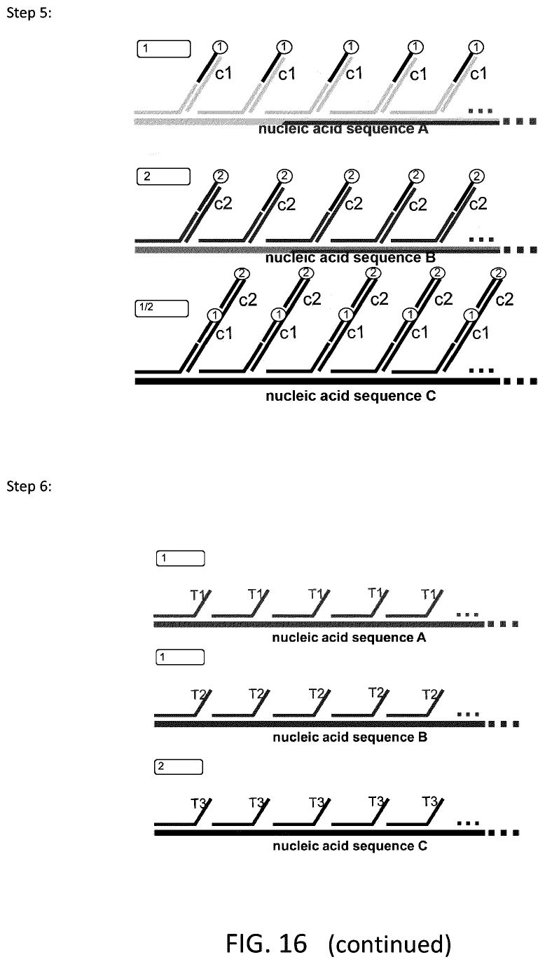

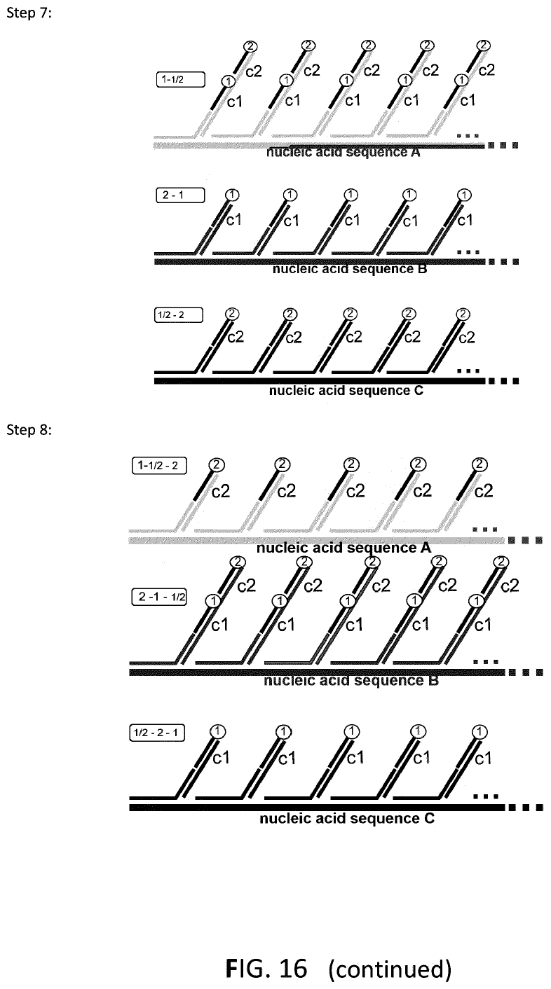

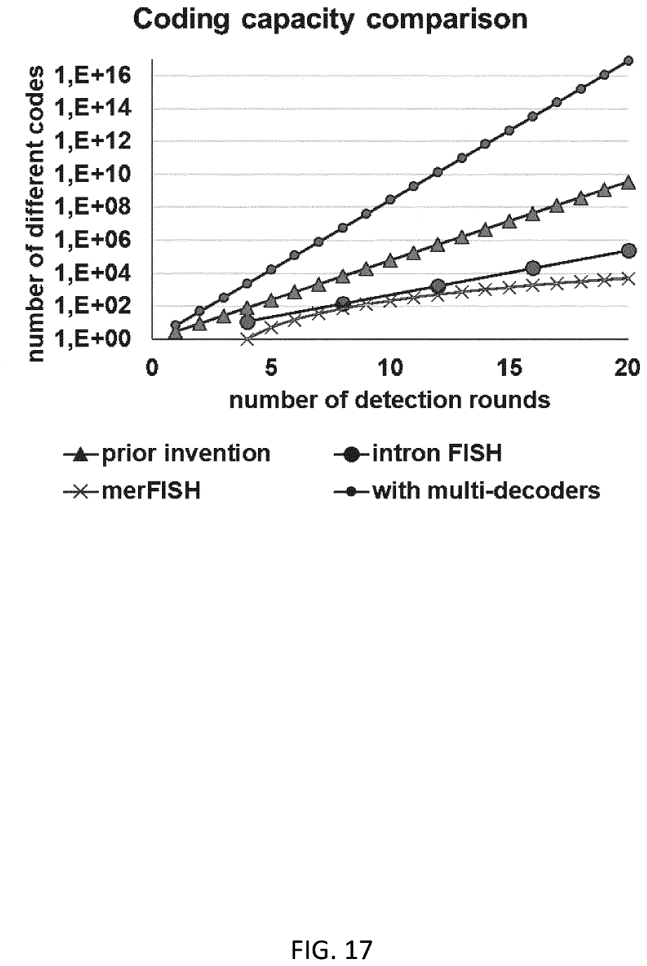

In certain embodiments, this pattern of binding or hybridization of the decoding oligonucleotides may be converted into a “code word.” For example, the code words could be also “10¹” and “1¹⁰” for an analyte, where a value of 1 represents binding and a value of 0 represents no binding. The code words may also have longer lengths in other embodiments (see FIG. 13). A code word can be directly related to a specific unique identifier sequence of a analyte-specific probe. Accordingly, different analyte-specific probe may match certain code words, which can then be used to identify the different analytes of the analyte-specific probe based on the binding patterns of the decoding oligonucleotide. However, if no binding is evident, then the code word would be “000” in this example.

The values in each code word can also be assigned in different fashions in some embodiments. For example, a value of 0 could represent binding while a value of 1 represents no binding. Similarly, a value of 1 could represent binding of a secondary nucleic acid probe with one type of signaling entity while a value of 0 could represent binding of a secondary nucleic acid probe with another type of distinguishable signaling entity. These signaling entities could be distinguished, for example, via different colors of fluorescence. In some cases, values in code words need not be confined to 0 and 1. The values could also be drawn from larger alphabets, such as ternary (e.g., 0, 1, and 2) or quaternary (e.g., 0, 1, 2, and 3) systems. Each different value could, for example, be represented by a different distinguishable signaling entity, including (in some cases) one value that may be represented by the absence of signal.

The code words for each analyte may be assigned sequentially, or may be assigned at random. For instance, a first analyte may be assigned to 101, while a second nucleic acid target may be assigned to 110. In addition, in some embodiments, the code words may be assigned using an error-detection system or an error-correcting system, such as a Hamming system, a Golay code, or an extended Hamming system (or a SECDED system, i.e., single error correction, double error detection). Generally speaking, such systems can be used to identify where errors have occurred, and in some cases, such systems can also be used to correct the errors and determine what the correct code word should have been. For example, a code word such as 001 may be detected as invalid and corrected using such a system to 101, e.g., if 001 is not previously assigned to a different target sequence. A variety of different error-correcting codes can be used, many of which have previously been developed for use within the computer industry; however, such error-correcting systems have not typically been used within biological systems. Additional examples of such error-correcting codes are discussed in more detail below.

“Essentially complementary” means, when referring to two nucleotide sequences, that both sequences can specifically hybridize to each other under stringent conditions, thereby forming a hybrid nucleic acid molecule with a sense and an antisense strand connected to each other via hydrogen bonds (Watson-and-Crick base pairs). “Essentially complementary” includes not only perfect base-pairing along the entire strands, i.e. perfect complementary sequences but also imperfect complementary sequences which, however, still have the capability to hybridize to each other under stringent conditions. Among experts it is well accepted that an “essentially complementary” sequence has at least 88% sequence identity to a fully or perfectly complementary sequence.

“Percent sequence identity” or “percent identity” in turn means that a sequence is compared to a claimed or described sequence after alignment of the sequence to be compared (the “Compared Sequence”) with the described or claimed sequence (the “Reference Sequence”). The percent identity is then determined according to the following formula: percent identity=100 [1-(C/R)] wherein C is the number of differences between the Reference Sequence and the Compared Sequence over the length of alignment between the Reference Sequence and the Compared Sequence, wherein
(i) each base or amino acid in the Reference Sequence that does not have a corresponding aligned base or amino acid in the Compared Sequence and
(ii) each gap in the Reference Sequence and
(iii) each aligned base or amino acid in the Reference Sequence that is different from an aligned base or amino acid in the Compared Sequence, constitutes a difference and (iiii) the alignment has to start at position 1 of the aligned sequences;
and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the herein above calculated percent identity is less than the specified percent identity.

In the “incubation” steps as understood herein the respective moieties or subjects such as probes or oligonucleotide, are brought into contact with each other under conditions well known to the skilled person allowing a specific binding or hybridization reaction, e.g. pH, temperature, salt conditions etc. Such steps may therefore, be preferably carried out in a liquid environment such as a buffer system which is well known in the art.

The “removing” steps according to the disclosure may include the washing away of the moieties or subjects to be removed such as the probes or oligonucleotides by certain conditions, e.g. pH, temperature, salt conditions etc., as known in the art.

It is understood that in an embodiment of the method according to the present disclosure a plurality of analytes can be encoded in parallel. This requires the use of different sets of analyte-specific probes in step (1). The analyte-specific probes of a particular set differ from the analyte-specific probes of another set. This means that the analyte-specific probes of set 1 bind to analyte 1, the analyte-specific probes of set 2 bind to analyte 2, the analyte-specific probes of set 3 bind to analyte 3, etc. In this embodiment also the use of different sets of decoding oligonucleotides is required in the methods according to the present disclosure.

The decoding oligonucleotides of a particular set differ from the decoding oligonucleotides of another set. This means, the decoding oligonucleotides of set 1 bind to the analyte-specific probes of above set 1 of analyte-specific probes, the decoding oligonucleotides of set 2 bind to the analyte-specific probes of above set 2 of analyte-specific probes, the decoding oligonucleotides of set 3 bind to the analyte-specific probes of above set 3 of analyte-specific probes, etc.

In this embodiment where a plurality of analytes is to be encoded in parallel the different sets of analyte-specific probes may be provided as a premixture of different sets of analyte-specific probes and/or the different sets of decoding oligonucleotides may be provided as a premixture of different sets of decoding oligonucleotides. Each mixture may be contained in a single vial. Alternatively, the different sets of analyte-specific probes and/or the different sets of decoding oligonucleotides may be provided in steps singularly.

A “kit” is a combination of individual elements useful for carrying out the use and/or method of the disclosure, wherein the elements are optimized for use together in the methods. The kits may also contain additional reagents, chemicals, buffers, reaction vials etc. which may be useful for carrying out the method according to the disclosure. Such kits unify all essential elements required to work the method according to the disclosure, thus minimizing the risk of errors. Therefore, such kits also allow semi-skilled laboratory staff to perform the method according to the present disclosure.

The term “quencher” or “quencher dye” or “quencher molecule” refers to a dye or an equivalent molecule, such as nucleoside guanosine (G) or 2′-deoxyguanosine (dG), which is capable of reducing the fluorescence of a fluorescent reporter dye or donor dye. A quencher dye may be a fluorescent dye or non-fluorescent dye. When the quencher is a fluorescent dye, its fluorescence wavelength is typically substantially different from that of the reporter dye and the quencher fluorescence is usually not monitored during an assay. Some embodiments of the present disclosure disclose signal oligonucleotides comprising a quencher and/or a quencher in combination with a signal element (see FIG. 14), and therefore the signal oligonucleotides is not detectable during imaging.

In an embodiment of the disclosure the sample is a biological sample, preferably comprising biological tissue, further preferably comprising biological cells. A biological sample may be derived from an organ, organoids, cell cultures, stem cells, cell suspensions, primary cells, samples infected by viruses, bacteria or fungi, eukaryotic or prokaryotic samples, smears, disease samples, a tissue section.

The method is particularly qualified to encode, identify, detect, count or quantify analytes or single analytes molecules in a biological sample, i.e. such as a sample which contains nucleic acids or proteins as said analytes. It is understood that the biological sample may be in a form as it is in its natural environment (i.e. liquid, semi-liquid, solid etc.), or processed, e.g. as a dried film on the surface of a device which may be re-liquefied before the method is carried out.

In another embodiment of the disclosure prior to step (2) the biological tissue and/or biological cells are fixed. For example, in some embodiments, the cell and/or the tissue is fixed prior to introducing the probes, e.g., to preserve the positions of the analytes like nucleic acids within the cell. Techniques for fixing cells are known to those of ordinary skill in the art. As non-limiting examples, a cell may be fixed using chemicals such as formaldehyde, paraformaldehyde, glutaraldehyde, ethanol, methanol, acetone, acetic acid, or the like. In one embodiment, a cell may be fixed using Hepes-glutamic acid buffer-mediated organic solvent (HOPE).

This measure has the advantage that the analytes to be encoded, e.g. the nuclei acids or proteins, are immobilized and cannot escape. In doing so, the analytes then prepared for a better detection or encoding by the method according to the disclosure.

In yet a further embodiment within the set of analyte-specific probes the individual analyte-specific probes comprise binding elements (S1, S2, S3, S4, S5) which specifically interact with different sub-structures of one of the analytes to be encoded.

By this measure the method becomes even more robust and reliable because the signal intensity obtained at the end of the method or a cycle, respectively, is increased. It is understood, that the individual probes of a set while binding to the same analyte differ in their binding position or binding site at or on the analyte. The binding elements S1, S2, S3, S4, S5 etc. of the first, second, third fourth, fifth etc. analyte-specific probes therefore bind to or at a different position which, however, may or may not overlap.

A multiplex method or assay allow the simultaneously measurement of multiple analytes. According to the present disclosure it may be used to determine the presence or absence of a plurality of predetermined (known) analytes like nucleic acid target sequences in a sample. An analyte may be “predetermined” in that its sequence is known to design a probe that binds to the that target.

In some advantageous embodiments according to the present disclosure at least 20, in particular at least 25, in particular at least 30 different analytes are detected and/or quantified in a sample in parallel. For example, there may be at least 5, at least 10, at least 20, at least 50, at least 75, at least 100, at least 300, at least 1,000, at least 3,000, at least 10,000, or at least 30,000 distinguishable analyte-specific probes that are applied to a sample, e.g., simultaneously or sequentially.

In some advantageous embodiments for the multiplexing twenty (20) or more different sets of analyte-specific probes for encoding of at least 20 different analytes or more are required, in particular more than 50, more than 100 or more than 200. In the multiplexing methods of the present disclosure, in particular at least 20 different groups of analytes (e.g. mRNA molecules) i.e. tags are targeted.

In some advantageous embodiments, at least 4 rounds to collect information for identification of the analyte are carried out, wherein multiple readout increases the accuracy of identification and avoids false positives. The unique tag can be identified by various techniques, including hybridization, e.g. with labeled probes, directly or indirectly or by sequencing (by synthesis, ligation). In particular, the identity of the tag can be encoded with one single signal (binary code), two or more signals, wherein the signal can be a fluorescent label (e.g. attached to an oligonucleotide).

In some advantageous embodiments according to the present disclosure, the kit does not comprise sets of analyte-specific probes as defined under item A).

Preferably, if the analyte in methods according to the present disclosure is a nucleic acid, each set of analyte-specific probes comprises at least five (10) analyte-specific probes, in particular at least fifteen (15) analyte-specific probes, in particular at least twenty (20) analyte-specific probes which specifically interact with different sub-structures of the same analyte. Nucleic acid analyte includes specific DNA molecules, e.g. genomic DNA, nuclear DNA, mitochondrial DNA, viral DNA, bacterial DNA, extra-or intracellular DNA etc., and specific mRNA molecules, e.g. hnRNA, miRNA, viral RNA, bacterial RNA, extra-or intracellular RNA, etc.

Preferably, if the analyte in the methods according to the present disclosure is a peptide, a polypeptide or a protein, each set of analyte-specific probes comprises at least two (2) analyte-specific probes, in particular at least three (3) analyte-specific probes, in particular at least four (4) analyte-specific probes which specifically interact with different sub-structures of the same analyte.

In some advantageous embodiments according to the present disclosure the method comprises the use of at least two different sets of signal oligonucleotides, wherein the signal oligonucleotides in each set comprise a different signal element and comprise a different connector element (C).

In particular, the method comprises the use of at least two different sets of decoding oligonucleotides per analyte, wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and wherein the decoding oligonucleotides of the different sets per analyte differ in the translator element (c) comprising a nucleotide sequence allowing a specific hybridization of a signal oligonucleotide.

In some embodiments the method comprises the use of least two different sets of decoding oligonucleotides per analyte, wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and wherein the decoding oligonucleotides of the different sets for at least one analyte differ in the translator element (c) comprising a nucleotide sequence allowing a specific hybridization of a signal oligonucleotide.

In some advantageous embodiments, the number of different sets of decoding oligonucleotides per analyte comprising different translator elements (c) corresponds to the number of different sets of signal oligonucleotides comprising different connector elements (C). However, the decoding oligonucleotides in a particular set of decoding oligonucleotides may interacts with identical identifier elements (T) which are unique to a particular analyte. In particular, all sets of decoding oligonucleotides for the different analytes may comprise the same type(s) of translator element(s) (c).

In particular, the present disclosure is directed to a method for detecting an analyte in a pathogen-comprising sample including acts of exposing the pathogen inactivated sample to a plurality of analyte-specific probes; for each of the analyte-specific probes, determining binding of the analyte-specific probes within the sample; creating code words based on the binding of the analyte-specific probes, the decoding oligonucleotides and the signal oligonucleotides; and for at least some of the code words, matching the code word to a valid code word. In certain embodiments, this pattern of binding or hybridization of the analyte-specific probes, the decoding oligonucleotides and the signal oligonucleotides may be converted into a “code word.” For example, for instance, the code words may be “10¹” and “1¹⁰” for a first analyte and a second analyte, respectively, where a value of 1 represents binding and a value of 0 represents no binding of decoding oligonucleotides and/or the binding of signal oligonucleotides without and/or quenched signal element. The analyte in the detection round/cycle is therefore not detectable during imaging.

To create such a zero (0) in a code word for an individual analyte the method may comprise the use of:

(D) at least a set of non-signal decoding oligonucleotides for binding to a particular identifier element (T) of analyte-specific probes, wherein the decoding oligonucleotides in the same set of non-signal decoding oligonucleotides interacting with the same different identifier element (T),
wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

To create such a zero (0) in a code word for an individual analyte the method may comprise the use of:

(D) at least a set of non-signal decoding oligonucleotides for binding to a particular identifier element (T) of analyte-specific probes, wherein the decoding oligonucleotides in the same set of non-signal decoding oligonucleotides interacting with the same different identifier element (T),
wherein each non-signal decoding oligonucleotide comprises an identifier connector element (t) comprising a nucleotide sequence which is essentially complementary to at least a section of a unique identifier sequence, and comprise a translator element that does not interact/bind to a signal oligonucleotide due to an instable binding sequence and/or due to the translator element is to short (c) comprising a nucleotide sequence allowing a specific hybridization of a signal oligonucleotide.

In some advantageous embodiments, the method may comprise the use of:

(D) at least two (2) different sets of non-signal decoding oligonucleotides for binding to at least two different identifier elements (T) of analyte-specific probes, each set of non-signal decoding oligonucleotides interacting with a different identifier element (T),
wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

Furthermore, in some advantageous embodiments the method may comprise the use of:

(E) a set of non-signal oligonucleotides, each non-signal oligonucleotide comprising:

(aa) a translator connector element (C) comprising a nucleotide sequence which is essentially complementary to at least a section of the nucleotide sequence of the translator element (c), and (bb) a quencher (Q), a signal element and a quencher (Q), or does not comprise a signal element.

In some advantageous embodiments, the method may comprise the use of:

(E) at least two sets of non-signal oligonucleotides, each non-signal oligonucleotide comprising:

(aa) a translator connector element (C) comprising a nucleotide sequence which is essentially complementary to at least a section of the nucleotide sequence of the translator element (c), and (bb) a quencher (Q), a signal element and a quencher (Q), or does not comprise a signal element.

In some advantageous embodiments, the different sets of non-signal oligonucleotides may be comprised in a pre-mixture of different sets of non-signal oligonucleotides or exist separately.

Further, in some embodiments the decoding oligonucleotides in a particular set of decoding oligonucleotides interacts with identical identifier elements (T) which are unique to a particular analyte.

In some advantageous embodiments, the different sets of decoding oligonucleotides may be comprised in a pre-mixture of different sets of decoding oligonucleotides or exist separately. In some advantageous embodiments, the different sets of analyte-specific probes may be comprised in a pre-mixture of different sets of analyte-specific probes or exist separately. In some advantageous embodiments, the different sets of signal oligonucleotides may be comprised in a pre-mixture of different sets of signal oligonucleotides or exist separately.

In some advantageous embodiments, a mixture of decoding oligonucleotides and/or multi-decoders is provided that specifically hybridize to the unique identifier sequences of the probe sets. In some embodiments, the decoding oligonucleotides comprise of at least two sequence elements, a first element that is complementary to the unique identifier sequences of the corresponding probe set and a second sequence element (translator element) that provides a sequence for the specific hybridization of a signal oligonucleotide, the translator element defines the type of signal that is recruited to the decoding oligonucleotide. In some embodiments multi-decoders comprising at least three sequence elements are used, a first element that is complementary to the unique identifier sequences of the corresponding probe set and at least to additional sequence elements (translator elements) that provide sequences for the specific hybridization of at least two different signal oligonucleotides. The translator elements define the type of signals that are recruited to the multi-decoder. Different possible structures of a multi-decoder can be seen in FIG. 15. Since a multi-decoder does recruit a full signal oligonucleotide per translator element, the brightness of the signals in each channel is not lower than the brightness of signals with decoding oligonucleotides.

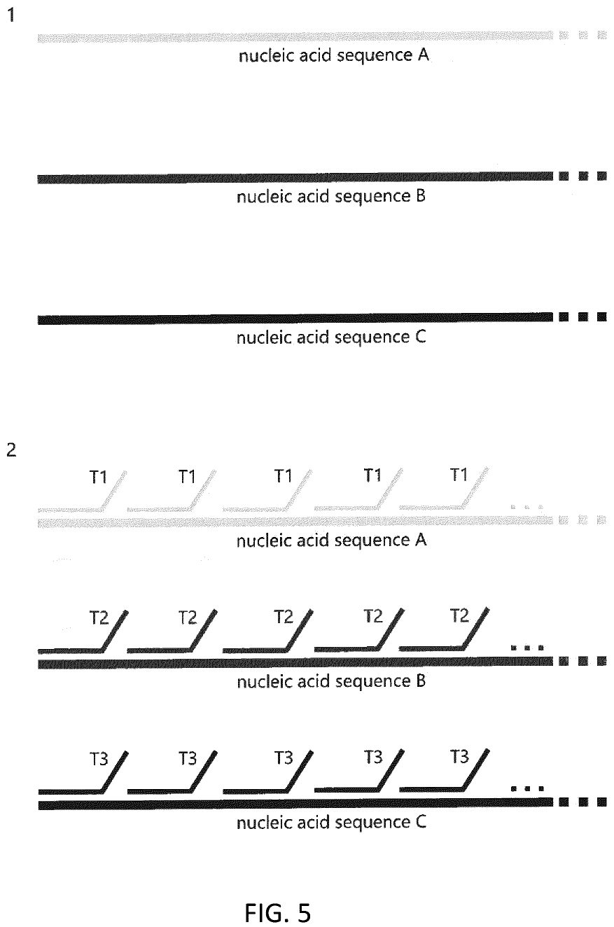

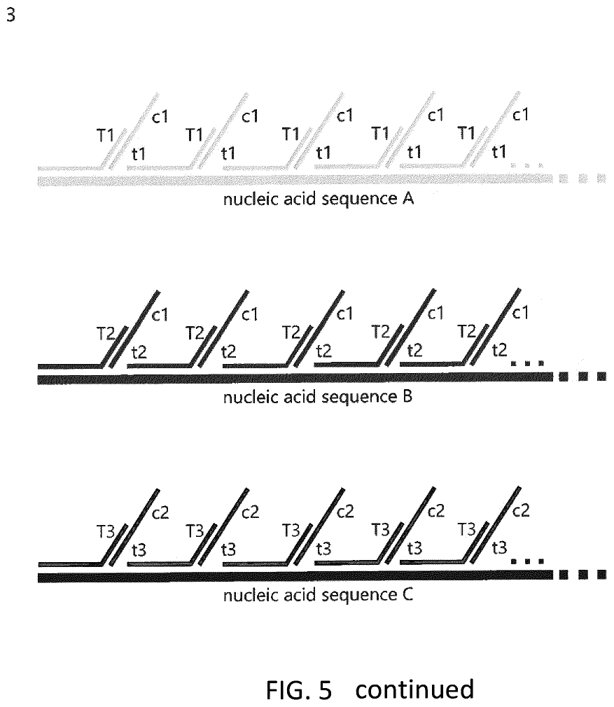

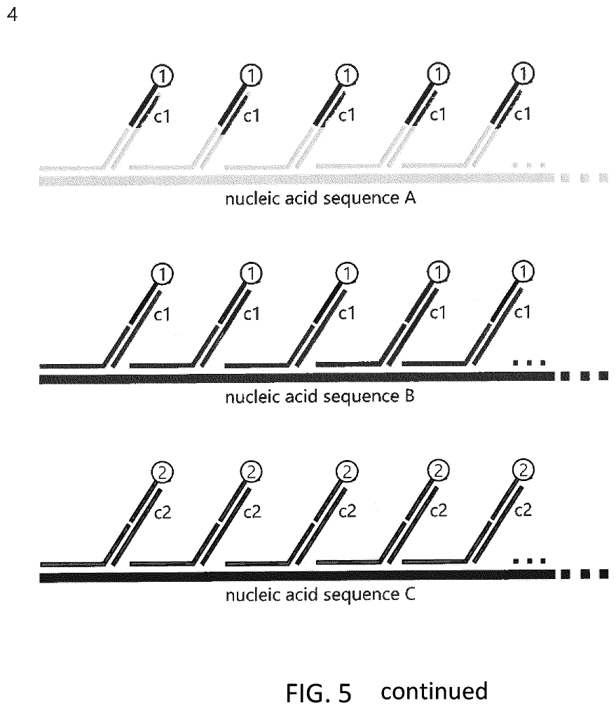

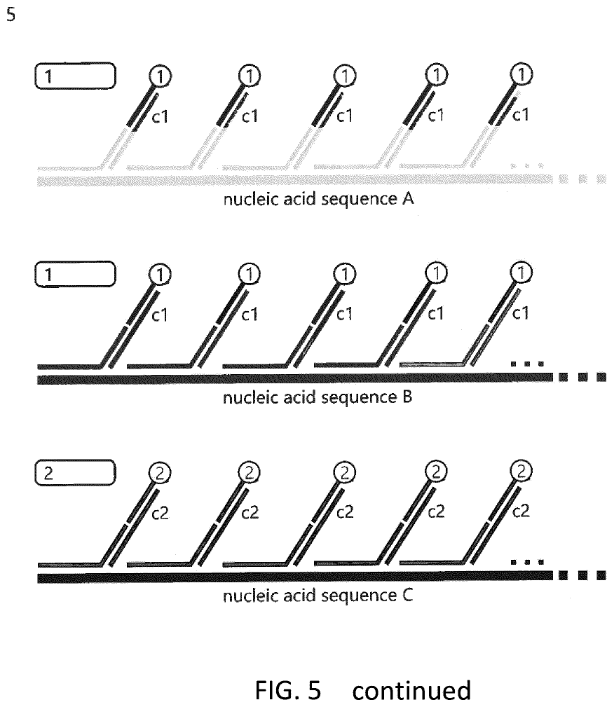

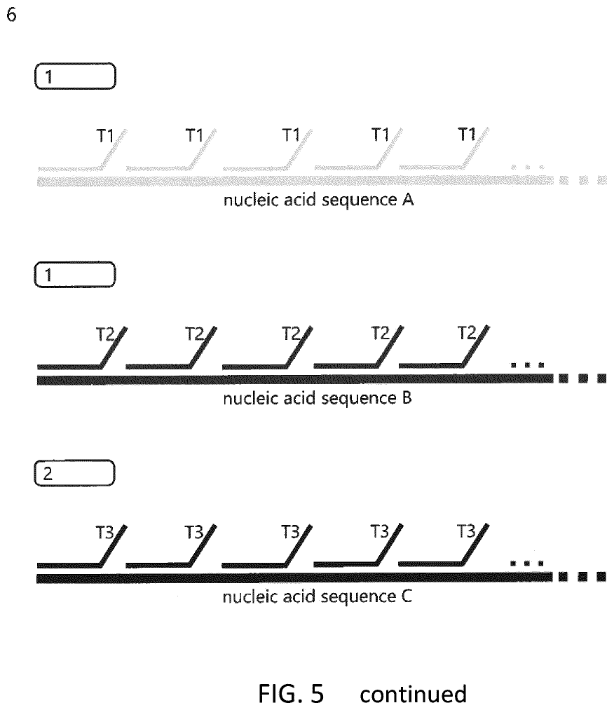

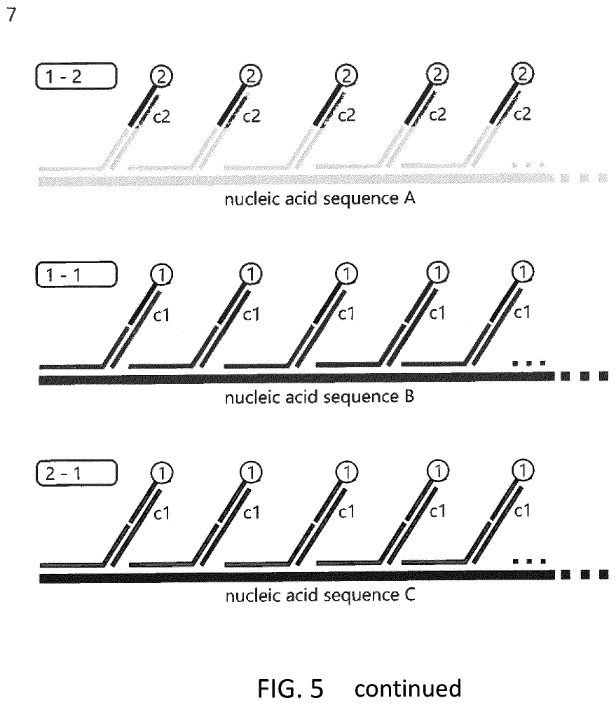

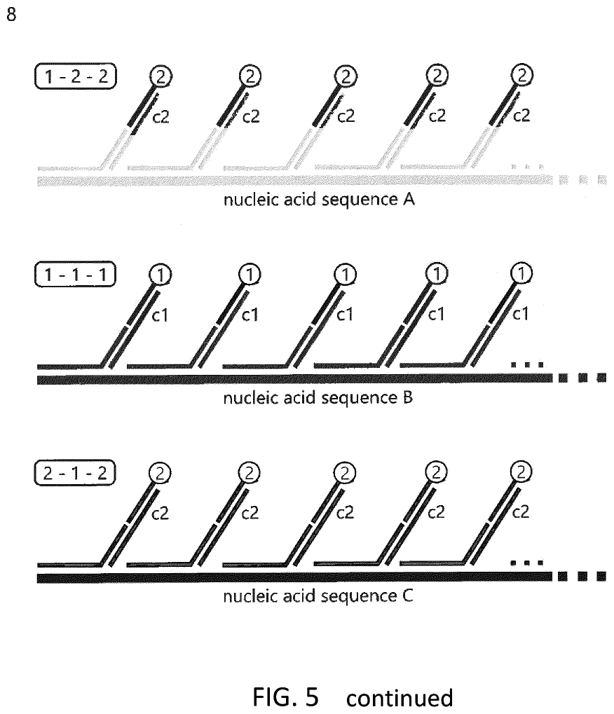

The usage of multi-decoders increases further the efficiency of the encoding scheme. FIG. 16 shows a possible encoding scheme using multi-decoders based upon the same conditions used for the examples with the decoding oligonucleotide with two sequence elements. One can clearly see that the multi-decoder based encoding scheme can create a higher hamming distance, with the same number of rounds and the same number of different signal oligonucleotides used in the example of FIG. 5.

As mentioned above the analyte to be encoded may be a nucleic acid, preferably DNA, PNA or RNA, in particular mRNA, a peptide, polypeptide, a protein, and/or mixtures thereof.

In some advantageous embodiments, the binding element(S) comprises an amino acid sequence allowing a specific binding to the analyte to be encoded. The binding element(S) may comprise moieties which are affinity moieties from affinity substances or affinity substances in their entirety selected from the group consisting of antibodies, antibody fragments, anticalin proteins, receptor ligands, enzyme substrates, lectins, cytokines, lymphokines, interleukins, angiogenic or virulence factors, allergens, peptidic allergens, recombinant allergens, allergen-idiotypical antibodies, autoimmune-provoking structures, tissue-rejection-inducing structures, immunoglobulin constant regions and combinations thereof.

In some advantageous embodiments, the binding element(S) may comprise or is an antibody or an antibody fragment selected from the group consisting of Fab, scFv; single domain, or a fragment thereof, bis scFv, F(ab)2, F(ab)3, minibody, diabody, triabody, tetrabody and tandab.

The present disclosure pertains in particular to a method for detecting an analyte in a pathogen-comprising sample comprising
i) Inactivation of the pathogen within the sample without isolation of RNA and/or DNA from the pathogen or the sample;
ii) Detecting the analyte by spatial transcriptomics, wherein the spatial transcriptomics detecting comprises or is a multiplex method for detecting different analytes in a sample by sequential signal-encoding of said analytes, comprising the steps of:

(A) contacting the sample with at least twenty (20) different sets of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising
(aa) a binding element(S) that specifically interacts with one of the different analytes to be encoded, and
(bb) an identifier element (T) comprising a nucleotide sequence which is unique to the analyte to be encoded (unique identifier sequence),

wherein the analyte-specific probes of a particular set of analyte-specific probes differ from the analyte-specific probes of another set of analyte-specific probes in the nucleotide sequence of the identifier element (T),

wherein the analyte-specific probes in each set of analyte-specific probes binds to the same analyte and comprises the same nucleotide sequence of the identifier element (T) which is unique to said analyte; and

(B) contacting the sample with at least one set of decoding oligonucleotides per analyte, wherein in each set of decoding oligonucleotides for an individual analyte each decoding oligonucleotide comprises:
(aa) an identifier connector element (t) comprising a nucleotide sequence which is essentially complementary to at least a section of the unique identifier sequence of the identifier element (T) of the corresponding analyte-specific probe set, and
(bb) a translator element (c) comprising a nucleotide sequence allowing a specific hybridization of a signal oligonucleotide;

wherein the decoding oligonucleotides of a set for an individual analyte differ from the decoding oligonucleotides of another set for a different analyte in the first connect element (t); and

(C) contacting the sample with at least a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and (bb) a signal element.

(D) Detecting the signal caused by the signal element;

(E) selectively removing the decoding oligonucleotides and signal oligonucleotides from the sample, thereby essentially maintaining the specific binding of the analyte-specific probes to the analytes to be encoded;

(F) Performing at least three (3) further cycles comprising steps B) to E) to generate an encoding scheme with a code word per analyte, wherein in particular the last cycle may stop with step (D).

As mentioned above, the method according to the present disclosure comprises selectively removing the decoding oligonucleotides and signal oligonucleotides from the sample, thereby essentially maintaining the specific binding of the analyte-specific probes to the analyte to be encoded. In particular all steps are performed sequentially. However some steps may be performed simultaneously, in particular the contacting steps A) to C), in particular B) and C).

By this measure the requirements for another round/cycle of binding further decoding oligonucleotides to the same analyte-specific probes are established, thus finally resulting in a code or encoding scheme comprising more than one signal. This step is realized by applying conditions and factors well known to the skilled person, e.g. pH, temperature, salt conditions, oligonucleotide concentration, polymers etc.

In another embodiment of the present disclosure, the method may comprise repeating steps (B)-(E) at least three times to generate an encoding scheme. With this measure a code of four signals in case of four cycles/rounds which are carried out by the user, where ‘n’ is an integer representing the number of rounds. The encoding capacity of the method according to the disclosure is herewith increased depending on the nature of the analyte and the needs of the operator. In an embodiment of the disclosure said encoding scheme is predetermined and allocated to the analyte to be encoded.

However, this measure enables a precise experimental set-up by providing the appropriate sequential order of the employed decoding and signal oligonucleotides and, therefore, allows the correct allocation of a specific analyte to a respective encoding scheme. The decoding oligonucleotides which are used in repeated steps (B)-(D2) may comprise a translator element (c2) which is identical with the translator element (c1) of the decoding oligonucleotides used in previous steps (B)-(E). In another embodiment of the disclosure decoding oligonucleotides are used in repeated steps (B)-(E) comprising a translator element (c2) which differs from the translator element (c1) of the decoding oligonucleotides used in previous steps (B)-(E). It is understood that the decoding elements may or may not be changed from round to round, i.e. in the second round (B)-(E) comprising the translator element c2, in the third round (B)-(E) comprising the translator element c3, in the fourth round (B)-(E) comprising the translator element c4 etc., wherein ‘n’ is an integer representing the number of rounds.

The signal oligonucleotides which are used in repeated steps (B)-(E) may comprise a signal element which is identical with the signal element of the decoding oligonucleotides used in previous steps (B)-(E). In a further embodiment of the disclosure signal oligonucleotides are used in repeated steps (B)-(E) comprising a signal element which differs from the signal element of the decoding oligonucleotides used in previous steps (B)-(E). In some embodiments no-signal oligonucleotides and/or no-signal decoding oligonucleotides for an individual analyte are used, resulting to the value 0 in the code word for this cycle/position. In some embodiments in a repeated cycle no decoding oligonucleotides for an individual analyte is contacted with the sample resulting also to the value 0 in the code word for this cycle/position.

By this measure each round the same or a different signal is provided resulting in an encoding scheme characterized by a signal sequence consisting of numerous different signals. This measure allows the creation of a unique code or code word which differs from all other code words of the encoding scheme. In another embodiment of the disclosure, the binding element(S) of the analyte-specific probe comprises a nucleic acid comprising a nucleotide sequence allowing a specific binding to the analyte to be encoded, preferably a specific hybridization to the analyte to be encoded.

In some advantageous embodiments, all steps are automated, in particular wherein steps B) to F) are automated, in particular by using a robotic system and/or an optical multiplexing system according to the present disclosure. In some examples, the steps may be performed in a fluidic system.

As mentioned above, with the methods according to the present disclosure an encoding scheme with a code word per analyte is generated. Therefore, each analyte may be associated with a specific code word, wherein said code word comprise a number of positions, and wherein each position corresponds to one cycle resulting in a plurality of distinguishable encoding schemes with the plurality of code words. In particular, said encoding scheme may be predetermined and allocated to the analyte to be encoded.

In some advantageous embodiments, the code words obtained for the individual analytes in the performed cycles comprise the detected signals and additionally at least one element corresponding to no detected signal like 0,1 or 0,1,2 etc. (see also FIG. 13 and FIG. 14). In particular, no signal is detected for at least one analyte within at least one cycle if using the a non-signal probe according to FIG. 14, No. 2 to 4, or a non-signal decoding oligonucleotide as shown in FIG. 14 No. 5, or if in one cycle no decoding oligonucleotide is contacted with the corresponding identifier sequence comprised on analyte-specific probe interacting with the corresponding analyte in the sample. In this cycle the position has the value zero (0).

In some advantageous embodiments, at least for one individual analyte a position of the code word is zero (0).In particular, the code word zero (0) is generated by using no decoding oligonucleotides having an identifier connector element (t) comprising a nucleotide sequence which is essentially complementary to at least a section of the unique identifier sequence of the identifier element (T) of a corresponding analyte-specific probe for an individual analyte. As mentioned above, in some embodiments, if at least for one individual analyte a position of the code word is zero (0) in this cycle no corresponding decoding oligonucleotides having an identifier connector element (t) comprising a nucleotide sequence which is essentially complementary to at least a section of the unique identifier sequence of the identifier element (T) of a corresponding analyte-specific probe for an individual analyte are used.

Furthermore, in some advantageous embodiments the sample is contacted with at least two different sets of signal oligonucleotides, wherein the signal oligonucleotides in each set comprise a different signal element and comprise a different connector element (C).

In more particular embodiments, the sample is contacted with at least two different sets of decoding oligonucleotides per analyte,
wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and
wherein the decoding oligonucleotides of the different sets per analyte differ in the translator element (c) comprising a nucleotide sequence allowing a specific hybridization of a signal oligonucleotide.

In more particular embodiments, the sample is contacted with at least two different sets of decoding oligonucleotides per analyte,
wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and
wherein the decoding oligonucleotides of the different sets per analyte differ in the translator element (c) comprising a nucleotide sequence allowing a specific hybridization of a signal oligonucleotide;
wherein only one set of decoding oligonucleotides per analyte is used per cycle, and/or wherein different sets of decoding oligonucleotides are used in different cycles in combination with the corresponding set of signal oligonucleotides in the same cycle.

In some advantageous embodiments, the number of different sets of decoding oligonucleotides per analyte comprising different translator elements (c) corresponds to the number of different sets of signal oligonucleotides comprising different connector elements (C). All sets of decoding oligonucleotides for the different analytes may comprise the same type(s) of translator element(s) (c). In some advantageous embodiments of the method according to the present disclosure, the sample is contacted with at least a set of non-signal decoding oligonucleotides for binding to a particular identifier element (T) of analyte-specific probes, wherein the decoding oligonucleotides in the same set of non-signal decoding oligonucleotides interacting with the same different identifier element (T), wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

As mentioned above, the sample may be contacted with at least two (2) different sets of non-signal decoding oligonucleotides for binding to at least two different identifier elements (T) of analyte-specific probes, each set of non-signal decoding oligonucleotides interacting with a different identifier element (T), wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

In some advantageous embodiments of the method according to the present disclosure, the different sets of non-signal decoding oligonucleotides may be comprised in a pre-mixture of different sets of non-signal decoding oligonucleotides or exist separately.

Furthermore, in some advantageous embodiments of the method according to the present disclosure, the sample is contacted with a set of non-signal oligonucleotides, each non-signal oligonucleotide comprising:

(aa) a translator connector element (C) comprising a nucleotide sequence which is essentially complementary to at least a section of the nucleotide sequence of the translator element (c), and (bb) a quencher (Q), a signal element and a quencher (Q), or does not comprise a signal element.

In further embodiments, the sample may be contacted with: at least two sets of non-signal oligonucleotides, each non-signal oligonucleotide comprising:

(aa) a translator connector element (C) comprising a nucleotide sequence which is essentially complementary to at least a section of the nucleotide sequence of the translator element (c), and (bb) a quencher (Q), a signal element and a quencher (Q), or does not comprise a signal element.

As mentioned above, the different sets of non-signal oligonucleotides may be comprised in a pre-mixture of different sets of non-signal oligonucleotides or exist separately.

In further embodiments, the decoding oligonucleotides in a particular set of decoding oligonucleotides interacts with identical identifier elements (T) which are unique to a particular analyte.

As mentioned above, the different sets of decoding oligonucleotides may be comprised in a pre-mixture of different sets of decoding oligonucleotides or exist separately as well as the different sets of analyte-specific probes may be comprised in a pre-mixture of different sets of analyte-specific probes or exist separately as well the different sets of signal oligonucleotides may be comprised in a pre-mixture of different sets of signal oligonucleotides or exist separately.

In some advantageous embodiments of the method according to the present disclosure, the binding element(S) comprise a nucleic acid comprising a nucleotide sequence allowing a specific binding to the analyte to be encoded, preferably a specific hybridization to the analyte to be encoded.

In some advantageous embodiments of the method according to the present disclosure, after step A) and before step B) the non-bound analyte-specific probes may be removed, in particular by washing, further after step B) and before step C) the non-bound decoding oligonucleotides may be removed, in particular by washing further, after step C) and before step D) the non-bound signal oligonucleotides may be removed, in particular by washing.

In some advantageous embodiments of the method according to the present disclosure, the analyte specific probes may be incubated with the sample, thereby allowing a specific binding of the analyte specific probes to the analytes to be encoded, further the decoding oligonucleotides may be incubated with the sample, thereby allowing a specific hybridization of the decoding oligonucleotides to identifier elements (T) of the respective analyte-specific probes, further the signal oligonucleotides may be incubated with the sample, thereby allowing a specific hybridization of the signal oligonucleotides to translator elements (T) of the respective decoding oligonucleotides.

As mentioned above, the analyte to be encoded may be a nucleic acid, preferably DNA, PNA, RNA, in particular mRNA, a peptide, polypeptide, a protein or combinations thereof. Therefore, the binding element(S) may comprise an amino acid sequence allowing a specific binding to the analyte to be encoded. Examples for a binding element(S) are moieties which are affinity moieties from affinity substances or affinity substances in their entirety selected from the group consisting of antibodies, antibody fragments, anticalin proteins, receptor ligands, enzyme substrates, lectins, cytokines, lymphokines, interleukins, angiogenic or virulence factors, allergens, peptidic allergens, recombinant allergens, allergen-idiotypical antibodies, autoimmune-provoking structures, tissue-rejection-inducing structures, immunoglobulin constant regions and combinations thereof. In particular, the binding element(S) is an antibody or an antibody fragment selected from the group consisting of Fab, scFv; single domain, or a fragment thereof, bis scFv, Fab 2, Fab 3, minibody, diabody, triabody, tetrabody and tandab.

By this measure the method is further developed to such an extent that the encoded analytes can be detected by any means which is adapted to visualize the signal element. Examples of detectable physical features include e.g. light, chemical reactions, molecular mass, radioactivity, etc.

In some advantageous embodiments, the signal caused by the signal element, therefore in particular the binding of the signal oligonucleotides to the decoding oligonucleotides, interacting with the corresponding analyte probes, bound to the respective analyte is determined by:
(a) Imaging at least a portion of the sample; and/or
(b) Using an optical imaging technique; and/or
(c) Using a fluorescence imaging technique; and/or
(d) Multi-color fluorescence imaging technique; and/or
(e) Super-resolution fluorescence imaging technique.

The method according to the present disclosure may be used ideally for in vitro methods for diagnosis of a disease selected from the group comprising cancer, neuronal diseases, cardiovascular diseases, inflammatory diseases, autoimmune diseases, diseases due to a viral or bacterial infection, skin diseases, skeletal muscle diseases, dental diseases and prenatal diseases.

Further, the method according to the present disclosure may be used also ideally for in vitro methods for diagnosis of a disease in plants selected from the group comprising: diseases caused by biotic stress, preferably by infectious and/or parasitic origin, or diseases caused by abiotic stress, preferably caused by nutritional deficiencies and/or unfavorable environment.

Further, the method according to the present disclosure may be used also ideally for in vitro methods for screening, identifying and/or testing a substance and/or drug comprising:
(a) contacting a test sample comprising a sample with a substance and/or drug
(b) detecting different analytes in a sample by sequential signal-encoding of said analytes with a method according to the present disclosure.

In some advantageous embodiments, the spatial transcriptomics method described herein is used for specific detection of many different analytes in parallel. The technology allows to distinguish a higher number of analytes than different signals are available. The process includes at least four consecutive rounds of specific binding, signal detection and selective denaturation (if a next round is required), eventually producing a signal code. To decouple the dependency between the analyte specific binding and the oligonucleotides providing the detectable signal, a so called “decoding”-oligonucleotide is introduced. The decoding oligonucleotide transcribes the information of the analyte specific probe set to the signal oligonucleotides.

In a specific embodiment, the spatial transcriptomics method method may comprise the steps of 1. providing one or more analyte specific probe sets, the set of analyte specific probes consist of one or more different probes, each differing in the binding moiety that specifically interacts with the analyte, all probes of a single probe set are tethered to a sequence element (unique identifier), that is unique to a single probe set and allows the specific hybridization of a decoding oligonucleotide, 2. specific binding of the probe sets to their target binding sites of the analyte, 3. eliminating non-bound probes (e.g. by a wash step), 4. providing a mixture of decoding oligonucleotides that specifically hybridize to the unique identifier sequences of the probe sets, the decoding oligonucleotides comprise of at least two sequence elements, a first element that is complementary to the unique identifier sequences of the corresponding probe set and a second sequence element (translator element) that provides a sequence for the specific hybridization of a signal oligonucleotide, the translator element defines the type of signal that is recruited to the decoding oligonucleotide, 5. specific hybridization of the decoding oligonucleotides to the unique identifier sequences provided by the bound probe sets, 6. eliminating non-bound decoding oligonucleotides (e.g. by washing step), 7. providing a mixture of signal oligonucleotides, comprising of a signal that can be detected and a nucleic acid sequence that specifically hybridizes to the translator element of one of the decoding oligonucleotides used in the former hybridization step, 8. specific hybridization of the signal oligonucleotides, 9. eliminating non-bound signal oligonucleotides, 10. detection of the signals, 11. selective release of decoding oligonucleotides and signal oligonucleotides while the binding of specific probe sets to the analyte is almost or completely unaffected, 12. eliminating released decoding oligonucleotide and signal oligonucleotides (e.g. by a washing step) while the binding of specific probes sets to the analytes is almost or completely unaffected, repeating the steps 4 to 12 at least three times until the detection of a sufficient number of signals to generate an encoding scheme for each different analyte of interest. It is to be understood that the before-mentioned features and those to be mentioned in the following cannot only be used in the combination indicated in the respective case, but also in other combinations or in an isolated manner without departing from the scope of the disclosure.

The disclosure is now further explained by means of embodiments resulting in additional features, characteristics and advantages of the disclosure. The embodiments are of pure illustrative nature and do not limit the scope or range of the disclosure. The features mentioned in the specific embodiments are general features of the disclosure which are not only applicable in the specific embodiment but also in an isolated manner in the context of any embodiment of the disclosure.

The spatial transcriptomics method method disclosed herein is used for specific detection of many different analytes in parallel. The technology allows distinguishing a higher number of analytes than different signals are available. The process preferably includes at least two consecutive rounds of specific binding, signal detection and selective denaturation (if a next round is required), eventually producing a signal code. To decouple the dependency between the analyte specific binding and the oligonucleotides providing the detectable signal, a so called “decoding” oligonucleotide is introduced. The decoding oligonucleotide transcribes the information of the analyte specific probe set to the signal oligonucleotides.

The present disclosure pertains further to methods for detecting an analyte in a pathogen-comprising sample, comprising an inactivation of the pathogen within the sample without isolation of RNA and/or DNA from the pathogen or the sample, and
attaching a plurality of analyte-specific probes to the analyte, wherein the analyte-specific probes independently attach to the analyte and wherein the analyte-specific probes share a common identifier segment (T);
annealing a plurality of first decoding oligonucleotides to the analyte-specific probes, wherein the first decoding oligonucleotides share a first common region that is reverse complementary to the common identifier segment and a second common region;
annealing a first signal oligonucleotide to at least one of the plurality of first decoding oligonucleotides such that an oligo tethered to the first signal oligonucleotide is reverse complementary to the second common region;
detecting the first signal oligonucleotide;
removing the plurality of first decoding oligonucleotides;
annealing a plurality of second decoding oligonucleotides to the analyte-specific probes, wherein the second decoding oligonucleotides share a first common region that is reverse complementary to the common identifier segment and a second decoding oligonucleotides second common region that differs from the second common region of the first decoding;
annealing a second signal oligonucleotide to at least one of the plurality of second decoding oligonucleotides such that an oligo tethered to the signal oligonucleotide is reverse complementary to the second decoding oligonucleotide second common region; and detecting the second signal oligonucleotide.

In particular, in the above mentioned embodiment a second aliquot of a plurality of first decoding oligonucleotides is annealed to the analyte-specific probes. Furthermore, a first aliquot of a plurality of first decoding oligonucleotides is annealed to the analyte-specific probes.

In some embodiments, no second signal oligonucleotide to the at least one of the plurality of first decoding oligonucleotides is annealed. In particular, no third signal oligonucleotide to the at least one of the plurality of first decoding oligonucleotides is annealed.

### Methods and Examples

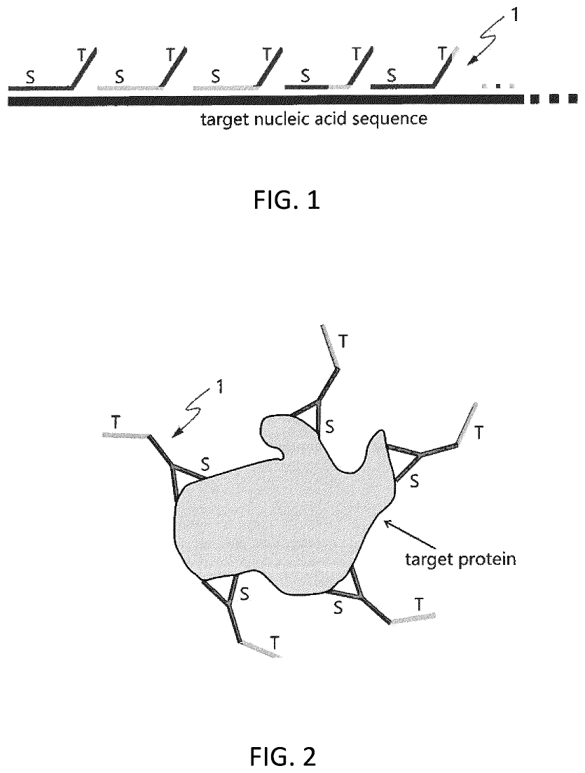

In an application variant, the analyte or target is nucleic acid, e.g. DNA or RNA, and the probe set comprises oligonucleotides that are partially or completely complementary to the whole sequence or a subsequence of the nucleic acid sequence to be detected (FIG. 1). The nucleic acid sequence specific oligonucleotide probe sets comprising analyte-specific probes (1) including a binding element(S) that specifically hybridizes to the target nucleic acid sequence to be detected, and an identifier element (T) comprising a nucleotide sequence which is unique to said set of analyte-specific probes (unique identifier sequence).

In a further application variant, the analyte or target is a protein and the probe set comprises one or more proteins, e.g. antibodies (FIG. 2). The protein specific probe set comprising analyte-specific probes (1) including a binding element (T) such as the (hyper-) variable region of an antibody, that specifically interacts with the target protein to be detected, and the identifier element (T).

In a further application variant, at least one analyte is a nucleic acid and at least a second analyte is a protein and at least the first probe set binds to the nucleic acid sequence and at least the second probe set binds specifically to the protein analyte. Other combinations are possible as well.

An Embodiments of the general method of the present disclosure may be:

Step 1: Applying the at least 20 analyte-or target-specific probe sets. The target nucleic acid sequence is incubated with a probe set consisting of oligonucleotides with sequences complementary to the target nucleic acid. In this example, a probe set of 5 different probes is shown, each comprising a sequence element complementary to an individual subsequence of the target nucleic acid sequence (S1 to S5). In this example, the regions do not overlap. Each of the oligonucleotides targeting the same nucleic acid sequence comprises the identifier element or unique identifier sequence (T), respectively.

Step 2: Hybridization of the probe set. The probe set is hybridized to the target nucleic acid sequence under conditions allowing a specific hybridization. After the incubation, the probes are hybridized to their corresponding target sequences and provide the identifier element (T) for the next steps.

Step 3: Eliminating non-bound probes. After hybridization, the unbound oligonucleotides are eliminated, e.g. by washing steps.

Step 4: Applying the decoding oligonucleotides. The decoding oligonucleotides consisting of at least two sequence elements (t) and (c) are applied. While sequence element (t) is complementary to the unique identifier sequence (T), the sequence element (c) provides a region for the subsequent hybridization of signal oligonucleotides (translator element).

Step 5: Hybridization of decoding oligonucleotides. The decoding oligonucleotides are hybridized with the unique identifier sequences of the probes (T) via their complementary first sequence elements (t). After incubation, the decoding oligonucleotides provide the translator sequence element (c) for a subsequent hybridization step.

Step 6: Eliminating the excess of decoding oligonucleotides. After hybridization, the unbound decoding oligonucleotides are eliminated, e.g. by washing steps.

Step 7: Applying the signal oligonucleotide. The signal oligonucleotides are applied. The signal oligonucleotides comprise at least one second connector element (C) that is essentially complementary to the translator sequence element (c) and at least one signal element that provides a detectable signal (F).

Step 8: Hybridization of the signal oligonucleotides. The signal oligonucleotides are hybridized via the complementary sequence connector element (C) to the translator element (c) of decoding oligonucleotide. After incubation, the signal oligonucleotides are hybridized to their corresponding decoding oligonucleotides and provide a signal (F) that can be detected.

Step 9: Eliminating the excess of signal oligonucleotides. After hybridization, the unbound signal oligonucleotides are eliminated, e.g. by washing steps.

Step 10: Signal detection. The signals provided by the signal oligonucleotides are detected.

The following steps (steps 11 and 12) are unnecessary for the last detection round.

Step 11: Selective denaturation. The hybridization between the unique identifier sequence (T) and the first sequence element (t) of the decoding oligonucleotides is dissolved. The destabilization can be achieved via different mechanisms well known to the trained person like for example: increased temperature, denaturing agents, etc. The target-or analyte-specific probes are not affected by this step.

Step 12: Eliminating the denatured decoding oligonucleotides. The denatured decoding oligonucleotides and signal oligonucleotides are eliminated (e.g. by washing steps) leaving the specific probe sets with free unique identifier sequences, reusable in a next round of hybridization and detection (steps 4 to 10). This detection cycle (steps 4 to 12) is repeated at least four times until the planed encoding scheme is completed.

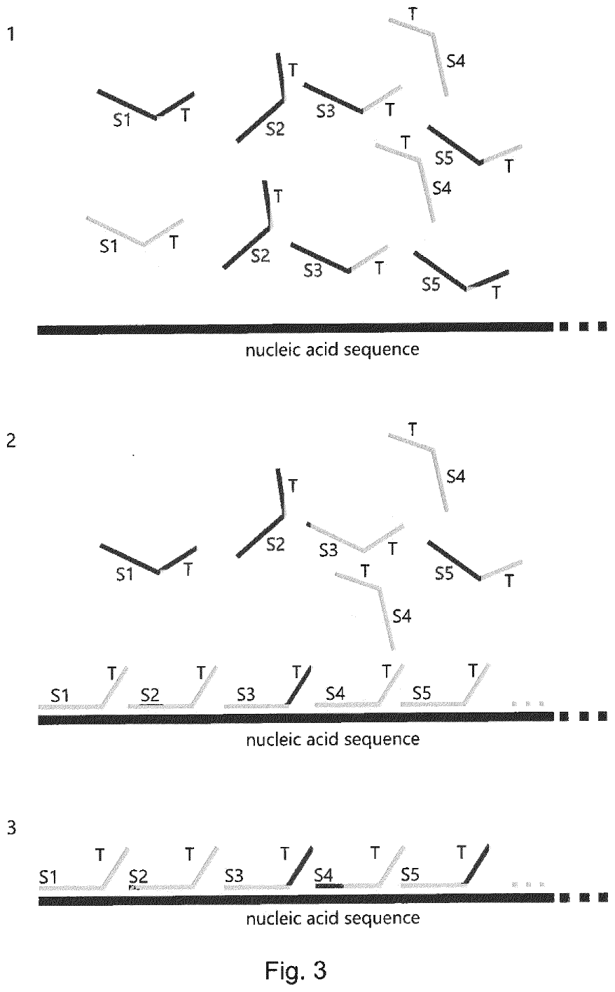

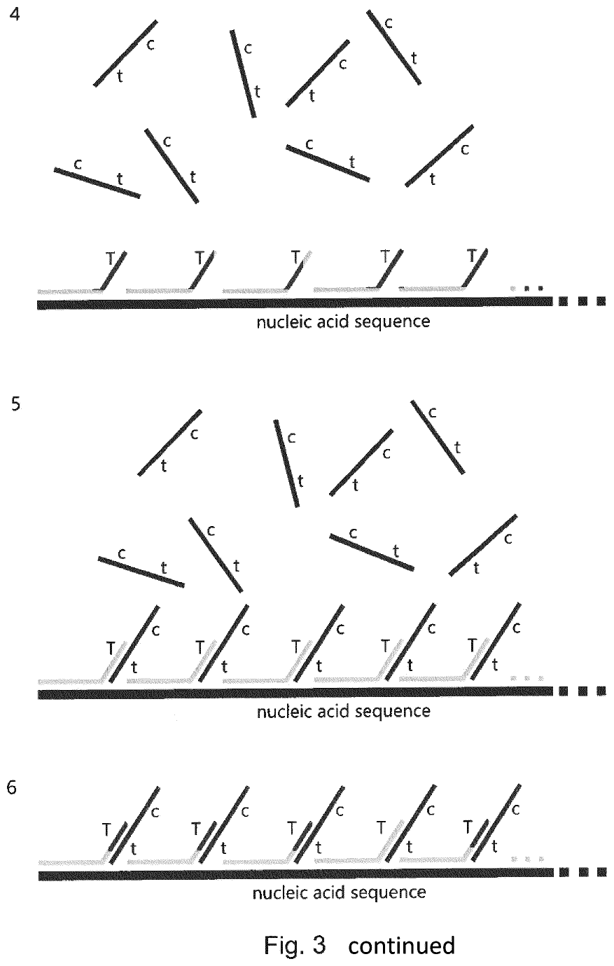

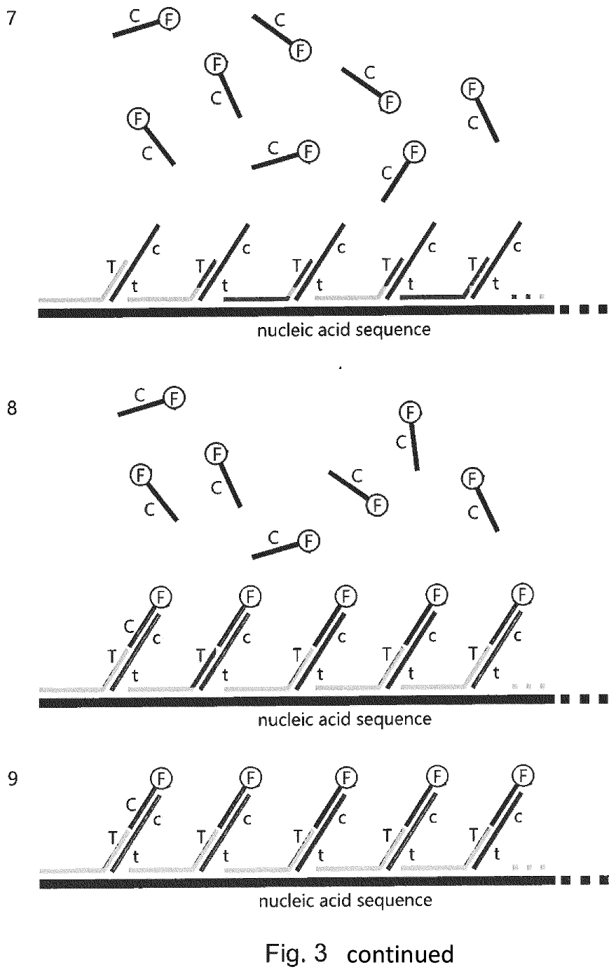

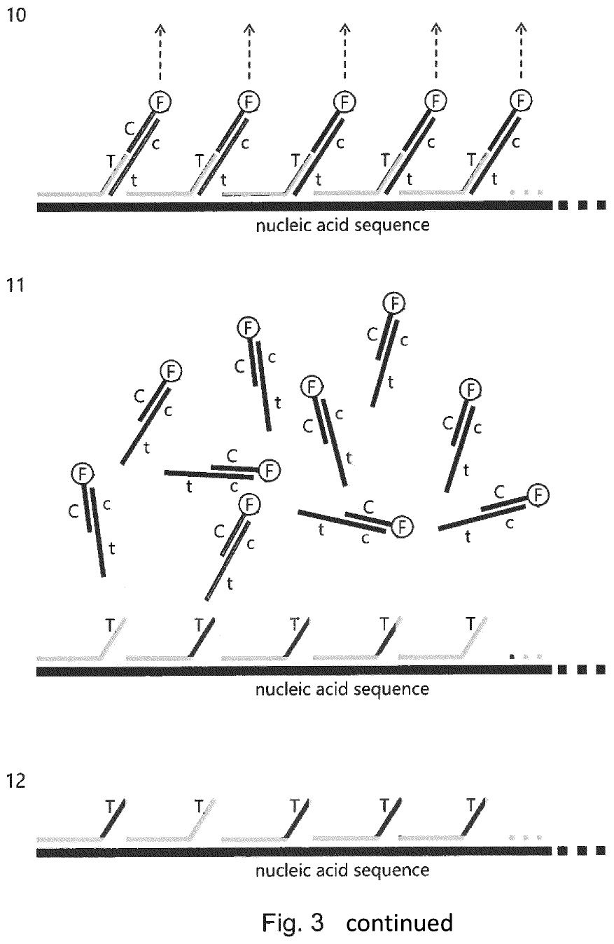

Another Embodiment of the general method of the present disclosure using multi-decoders may be (FIG. 16):
Step 1: Target nucleic acids: In this example three different target nucleic acids (A), (B) and (C) have to be detected and differentiated by using only two different types of signal oligonucleotides. Before starting the experiment, a certain encoding scheme is set. In this example, the three different nucleic acid sequences are encoded by three rounds of detection with three different signal types (1), (2) and (½) and a resulting hamming distance of 3 to allow for error detection. The planed code words are:
   sequence A: (1)-(1)-(2)
   sequence B: (2)-(2)-(1/2)
   sequence C: (1/2)-(½)-(1)
Step 2: Hybridization of the probe sets: For each target nucleic acid, an own probe set is applied, specifically hybridizing to the corresponding nucleic acid sequence of interest. Each probe set provides a unique identifier sequence (T1), (T2) or (T3). This way each different target nucleic acid is uniquely labeled. In this example sequence (A) is labeled with (T1), sequence (B) with (T2) and sequence (C) with (T3). The illustration in FIG. 16 summarizes Steps 1 to 3 of FIG. 3.
Step 3: Hybridization of the decoding oligonucleotides and multi-decoders: For each unique identifier present, a certain decoding oligonucleotide or multi-decoder is applied specifically hybridizing to the corresponding unique identifier sequence by its first sequence element (here (t1) to (T1), (t2) to (T2) and (t3) to (T3)). Each of the decoding oligonucleotides or multi-decoders provides a translator or two translator elements that define the signals that will be generated after hybridization of signal oligonucleotides. Here nucleic acid sequence (A) is labeled with (c1), (B) is labeled with (c2) and (C) is labeled with both translator elements (c1) and (c2) resulting in the signal (½). The illustration in FIG. 16 summarizes steps 4 to 6 of FIG. 3.
Step 4: Hybridization of signal oligonucleotides: For each type of translator element, a signal oligonucleotide with a certain signal, differentiable from signals of other signal oligonucleotides, is applied. This signal oligonucleotide can specifically hybridize to the corresponding translator element. The illustration in FIG. 16 summarizes steps 7 to 9 of FIG. 3.
Step 5: Signal detection for the encoding scheme: The different signals are detected. Note that in this example the nucleic acids (A), (B) and (C) can already be distinguished after the first round of detection. This is in contrast to the step 5 of FIG. 5 explained by the additional signal type (1/2) that can be realized due to multi-decoders. Although nucleic acid sequences can already be distinguished, the additional rounds contribute to the planned hamming distance of 3. The illustration in FIG. 16 corresponds to step 10 of FIG. 3.
Step 6: Selective denaturation: The decoding (and signal) oligonucleotides and/or multi-decoders of all nucleic acid sequences to be detected are selectively denatured and eliminated as described in steps 11 and 12 of FIG. 3. Afterwards the unique identifier sequences of the different probe sets can be used for the next round of hybridization and detection.
Step 7: Second round of detection: A next round of hybridization and detection is done as described in steps 3 to 5. Note that in this new round the mix of different decoding oligonucleotides and multi-decoders is changed. For example, decoding oligonucleotide of nucleic acid sequence (A) used in the first round comprised of sequence elements (t1) and (c1) while the new multi decoder of round 2 comprises of the sequence elements (t1), (c1) and (c2). Note that now a hamming distance of 2 is already given after 2 rounds, which is the final result of the example in FIG. 3 after 3 rounds.
Step 8: Third round of detection: Again, a new combination of decoding oligonucleotides and/or multi-decoders is used leading to new signal combinations. After signal detection, the resulting code words for the three different nucleic acid sequences are not only unique and therefore distinguishable but comprise a hamming distance of 3 to other code words. Due to the hamming distance, an error in the detection of the signals (signal exchange) would not result in a valid code word and therefore could be detected and because of hamming distance 3 also corrected, in contrast to the encoding scheme of FIG. 3. This way three different nucleic acids can be distinguished in three detection rounds with two different signals, allowing an error detection and correction.

Note that in every round of detection, the type of signal provided by a certain unique identifier is controlled by the use of a certain decoding oligonucleotide. As a result, the sequence of decoding oligonucleotides applied in the detection cycles transcribes the binding specificity of the probe set into a unique signal sequence.

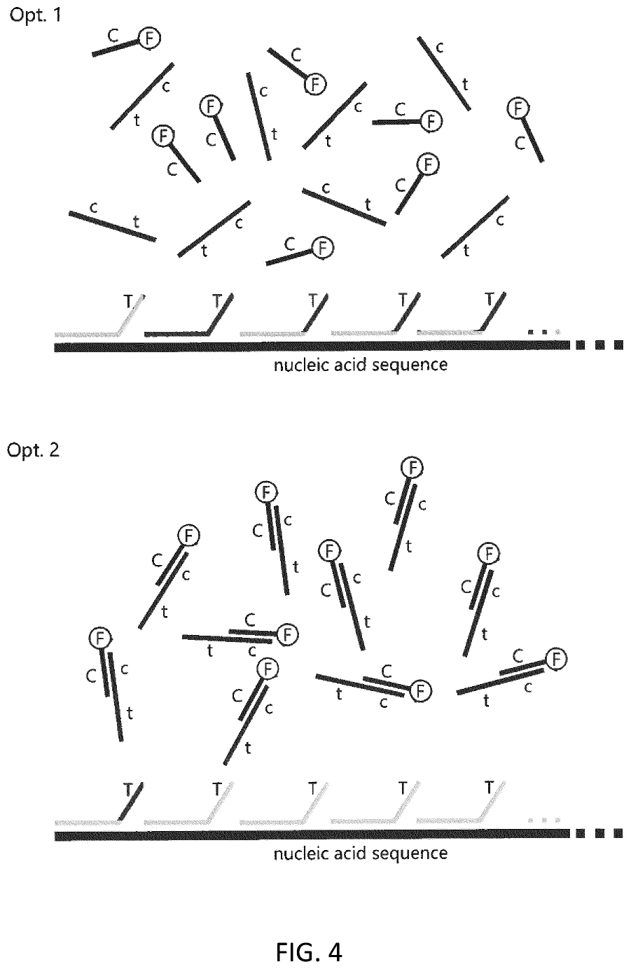

The steps of decoding oligonucleotide hybridization (steps 4 to 6) and signal oligonucleotide hybridization (steps 7 to 9) can also be combined in two alternative ways as shown in FIG. 4.

Opt. 1: Simultaneous hybridization. Instead of the steps 4 to 9 of FIG. 3, specific hybridization of decoding oligonucleotides and signal oligonucleotides can also be done simultaneously leading to the same result as shown in step 9 of FIG. 3, after eliminating the excess decoding-and signal oligonucleotides.

Opt. 2: Preincubation. Additionally to option 1 of FIG. 3, decoding-and signal oligonucleotides can be preincubated in a separate reaction before being applied to the target nucleic acid with the already bound specific probe set.

### 1. Example For Signal Encoding of Three Different Nucleic Acid Sequences by Two Different Signal Types and Three Detection Rounds

FIG. 3 shows the general concept of generation and detection of specific signals mediated by decoding oligonucleotides. It does not show the general concept of encoding that can be achieved by this procedure. To illustrate the use of the process shown in FIG. 3 for the generation of an encoding scheme, FIG. 5 shows a general example for a multiple round encoding experiment with three different nucleic acid sequences. In this example, the encoding scheme includes error detection.

Step 1: Target nucleic acids. In this example three different target nucleic acids (A), (B) and (C) have to be detected and differentiated by using only two different types of signal. Before starting the experiment, a certain encoding scheme is set. In this example, the three different nucleic acid sequences are encoded by three rounds of detection with two different signals (1) and (2) and a resulting hamming distance of 2 to allow for error detection. The planed code words are:
sequence A: (1)-(2)-(2);
sequence B: (1)-(1)-(1);
sequence C: (2)-(1)-(2).

Step 2: Hybridization of the probe sets. For each target nucleic acid, an own probe set is applied, specifically hybridizing to the corresponding nucleic acid sequence of interest. Each probe set provides a unique identifier sequence (T1), (T2) or (T3). This way each different target nucleic acid is uniquely labeled. In this example sequence (T) is labeled with (T1), sequence (B) with (T2) and sequence (C) with (T3). The illustration summarizes steps 1 to 3 of FIG. 3.

Step 3: Hybridization of the decoding oligonucleotides. For each unique identifier present, a certain decoding oligonucleotide is applied specifically hybridizing to the corresponding unique identifier sequence by its first sequence element (here (t1) to (T1), (t2) to (T2) and (t3) to (T3)). Each of the decoding oligonucleotides provides a translator element that defines the signal that will be generated after hybridization of signal oligonucleotides. Here nucleic acid sequences (A) and (B) are labeled with the translator element (c1) and sequence (C) is labeled with (c2). The illustration summarizes steps 4 to 6 of FIG. 3.

Step 4: Hybridization of signal oligonucleotides. For each type of translator element, a signal oligonucleotide with a certain signal (2), differentiable from signals of other signal oligonucleotides, is applied. This signal oligonucleotide can specifically hybridize to the corresponding translator element. The illustration summarizes steps 7 to 9 of FIG. 3.

Step 5: Signal detection for the encoding scheme. The different signals are detected. Note that in this example the nucleic acid sequence (C) can be distinguished from the other sequences by the unique signal (2) it provides, while sequences (A) and (B) provide the same kind of signal (1) and cannot be distinguished after the first cycle of detection. This is due to the fact, that the number of different nucleic acid sequences to be detected exceeds the number of different signals available. The illustration corresponds to step 10 of FIG. 3.

Step 6: Selective denaturation. The decoding (and signal) oligonucleotides of all nucleic acid sequences to be detected are selectively denatured and eliminated as described in steps 11 and 12 of FIG. 3. Afterwards the unique identifier sequences of the different probe sets can be used for the next round of hybridization and detection.

Step 7: Second round of detection. A next round of hybridization and detection is done as described in steps 3 to 5. Note that in this new round the mix of different decoding oligonucleotides is changed. For example, decoding oligonucleotide of nucleic acid sequence (A) used in the first round comprised of sequence elements (t1) and (c1) while the new decoding oligonucleotide comprises of the sequence elements (t1) and (c2). Note that now all three sequences can clearly be distinguished due to the unique combination of first and second round signals.

Step 8: Third round of detection. Again, a new combination of decoding oligonucleotides is used leading to new signal combinations. After signal detection, the resulting code words for the three different nucleic acid sequences are not only unique and therefore distinguishable but comprise a hamming distance of 2 to other code words. Due to the hamming distance, an error in the detection of the signals (signal exchange) would not result in a valid code word and therefore could be detected. By this way three different nucleic acids can be distinguished in three detection rounds with two different signals, allowing error detection.

### 2. Advantages Over Prior Art Technologies

### Coding Strategy

Compared to state-of-the-art methods, one particular advantage of the method according to the disclosure is the use of decoding oligonucleotides breaking the dependencies between the target specific probes and the signal oligonucleotides.

Without decoupling target specific probes and signal generation, two different signals can only be generated for a certain target if using two different molecular tags. Each of these molecular tags can only be used once. Multiple readouts of the same molecular tag do not increase the information about the target. In order to create an encoding scheme, a change of the target specific probe set after each round is required (SeqFISH) or multiple molecular tags must be present on the same probe set (like merFISH, intronSeqFISH).

Following the method according to the disclosure, different signals are achieved by using different decoding oligonucleotides reusing the same unique identifier (molecular tag) and a small number of different, mostly cost-intensive signal oligonucleotides. This leads to several advantages in contrast to the other methods.
(1) The encoding scheme is not defined by the target specific probe set as it is the case for all other methods of prior art. Here the encoding scheme is transcribed by the decoding oligonucleotides. This leads to a much higher flexibility concerning the number of rounds and the freedom in signal choice for the code words. Looking on the methods of prior art (e.g. merFISH or intronSeqFISH), the encoding scheme (number, type and sequence of detectable signals) for all target sequences is predefined by the presence of the different tag sequences on the specific probe sets (4 of 16 different tags per probe set in the case of merFISH and 5 of 60 different tags in the case of intron FISH). In order to produce a sufficient number of different tags per probe set, the methods use rather complex oligonucleotide designs with several tags present on one target specific oligonucleotide. In order to change the encoding scheme for a certain target nucleic acid, the specific probe set has to be replaced. The method according to the disclosure describes the use of a single unique tag sequence (unique identifier) per analyte, because it can be reused in every detection round to produce a new information. The encoding scheme is defined by the order of decoding oligonucleotides that are used in the detection rounds. Therefore, the encoding scheme is not predefined by the specific probes (or the unique tag sequence) but can be adjusted to different needs, even during the experiment. This is achieved by simply changing the decoding oligonucleotides used in the detection rounds or adding additional detection rounds.
(2) The number of different signal oligonucleotides must match the number of different tag sequences with methods of prior art (16 in the case of merFISH and 60 in the case of intronSeqFISH). Using the method according to the disclosure, the number of different signal oligonucleotides matches the number of different signals used. Due to this, the number of signal oligonucleotides stays constant for the method described here and never exceeds the number of different signals but increases with the complexity of the encoding scheme in the methods of prior art (more detection rounds more different signal oligonucleotides needed). As a result, the method described here leads to a much lower complexity (unintended interactions of signal oligonucleotides with environment or with each other) and dramatically reduces the cost of the assay since the major cost factor are the signal oligonucleotides.
(3) In the methods of prior art, the number of different signals generated by a target specific probe set is restricted by the number of different tag sequences the probe set can provide. Since each additional tag sequence increases the total size of the target specific probe, there is a limitation to the number of different tags a single probe can provide. This limitation is given by the size dependent increase of several problems (unintended inter- and intramolecular interactions, costs, diffusion rate, stability, errors during synthesis etc.). Additionally, there is a limitation of the total number of target specific probes that can be applied to a certain analyte. In case of nucleic acids, this limitation is given by the length of the target sequence and the proportion of suitable binding sites. These factors lead to severe limitations in the number of different signals a probe set can provide (4 signals in the case of merFISH and 5 signals in the case of intronSeqFISH). This limitation substantially affects the number of different code words that can be produced with a certain number of detection rounds. In the approach of the disclosure only one tag is needed and can be freely reused in every detection round. This leads to a low oligonucleotide complexity/length and at the same time to the maximum encoding efficiency possible (number of colorsnumber of rounds). The vast differences of coding capacity of our method compared to the other methods is shown in FIGS. 1 and 5. Due to this in approach of the disclosure a much lower number of detection rounds is needed to produce the same amount of information. A lower number of detection rounds is connected to lower cost, lower experimental time, lower complexity, higher stability and success rate, lower amount of data to be collected and analyzed and a higher accuracy of the results.

### Coding Ccapacity

All three methods compared in the Table 1 below use specific probe sets that are not denatured between different rounds of detection. For intronSeqFISH there are four detection rounds needed to produce the pseudo colors of one coding round, therefore data is only given for rounds 4, 8,12,16 and 20. The merFISH-method uses a constant number of 4 signals, therefore the data starts with the smallest number of rounds possible. After 8 detection rounds our method exceeds the maximum coding capacity reached with 20 rounds of merFISH (depicted with one asterisk) and after 12 rounds of detection the maximum coding capacity of intron FISH is exceeded (depicted with two asterisks). For the method according to the disclosure usage of 3 different signals is assumed (as is with intronSeqFISH).

TABLE 1
Comparison of coding capacity

| | CODING CAPACITY | | |
|---|---|---|---|
| | Method of the | | |
| NUMBER OF | present | intron | |
| ROUNDS: | disclosure | FISH | merFISH |
| 1 | 3 | — | — |
| 2 | 9 | — | — |
| 3 | 27 | — | — |
| 4 | 81 | 12 | 1 |
| 5 | 243 | — | 5 |
| 6 | 729 | — | 15 |
| 7 | 2187 | — | 35 |
| 8* | 6561 | 144 | 70 |
| 9 | 19683 | — | 126 |
| 10 | 59049 | — | 210 |
| 11 | 177147 | — | 330 |
| 12** | 531441 | 1728 | 495 |
| 13 | 1594323 | — | 715 |
| 14 | 4782969 | — | 1001 |
| 15 | 14348907 | — | 1365 |
| 16 | 43046721 | 20736 | 1820 |
| 17 | 129140163 | — | 2380 |
| 18 | 387420489 | — | 3060 |
| 19 | 1162261467 | — | 3876 |
| 20 | 3486784401 | 248832 | 4845 |

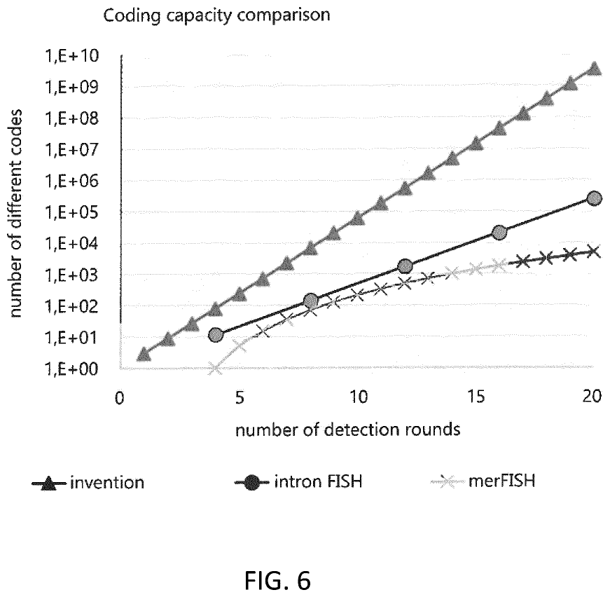

As shown in FIG. 6 the number of code words for merFISH does not exponentially increase with the number of detection cycles but gets less effective with each added round. In contrast, the number of code words for intronSeqFISH in the method according to the disclosure increases exponentially. The slope of the curve for the proposed method is much higher than that of intron FISH, leading to more than 10,000 times more code words usable after 20 rounds of detection.

Note that this maximum efficiency of coding capacity is also reached in case of seqFISH, where specific probes are denatured after every detection round and a new probe set is specifically hybridized to the target sequence for each detection round. However, this method has major downsides to technologies using only one specific hybridization for their encoding scheme (all other methods):
(1) For the efficient denaturation of the specific probes, rather crude conditions must be used (high temperatures, high concentrations of denaturing agent, long incubation times) leading to much higher probability for the loss or the damage of the analyte.
(2) For each round of detection an own probe set has to be used for every target nucleic acid sequence. Therefore, the number of specific probes needed for the experiment scales with the number of different signals needed for the encoding scheme. This dramatically increases the complexity and the cost of the assay.
(3) Because the hybridization efficiency of every target nucleic acid molecule is subject to some probabilistic effects, the fluctuations of signal intensity between the different detection rounds is much higher than in methods using only one specific hybridization event, reducing the proportion of complete codes.
(4) The time needed for the specific hybridization is much longer than for the hybridization of signal oligonucleotides or decoding oligonucleotides (as can be seen in the method parts of the intronSeqFISH, merFISH and seqFISH publications), which dramatically increases the time needed to complete an experiment.

Due to these reasons all other methods use a single specific hybridization event and accept the major downside of lower code complexity and therefore the need of more detection rounds and a higher oligonucleotide design complexity.

The method according to the disclosure combines the advantages of seqFISH (mainly complete freedom concerning the encoding scheme) with all advantages of methods using only one specific hybridization event while eliminating the major problems of such methods.

Note that the high numbers of code words produced after 20 rounds can also be used to introduce higher hamming distances (differences) between different code words, allowing error detection of 1, 2 or even more errors and even error corrections. Therefore, even very high coding capacities are still practically relevant.

As mentioned above, the usage of multi-decoders further increases the coding capacity of the encoding scheme. Instead of being limited to the having exactly the same number of different signal types as different signal oligonucleotides and corresponding translator elements, the use of multi-decoders increases the signal types that can be used to: (N×(N+1))/2 (with N being the number of different signal oligonucleotides used). For the code used in table 1 with 3 different signal oligonucleotides this would mean the following 7 different signal types could be used: (S1), (S2), (S3), (S1/S2), (S1/S3), (S2/S3), (S1/S2/S3). The effect to the coding efficiency can be seen in Table 1b and FIG. 17.

| | CODING CAPACITY | | | |
|---|---|---|---|---|
| | | Method of the | | |
| | Method of the | present disclosure | | |
| NUMBER OF | present | with multi- | intron | |
| ROUNDS: | disclosure | decoders | FISH | merFISH |
| 1 | 3 | 7 | — | — |
| 2 | 9 | 49 | — | — |
| 3 | 27 | 343 | — | — |
| 4 | 81 | 2401 | 12 | 1 |
| 5* | 243 | 16807 | — | 5 |
| 6 | 729 | 117649 | — | 15 |
| 7** | 2187 | 823543 | — | 35 |
| 8 | 6561 | 5764801 | 144 | 70 |
| 9 | 19683 | 40353607 | — | 126 |
| 10 | 59049 | 282475249 | — | 210 |
| 11 | 177147 | 1977326743 | — | 330 |
| 12*** | 531441 | 1.3841 × 10\hat{ }10 | 1728 | 495 |
| 13 | 1594323 | 9.6889 × 10\hat{ }10 | — | 715 |
| 14 | 4782969 | 6.7822 × 10\hat{ }11 | — | 1001 |
| 15 | 14348907 | 4.7476 × 10\hat{ }12 | — | 1365 |
| 16 | 43046721 | 3.3233 × 10\hat{ }13 | 20736 | 1820 |
| 17 | 129140163 | 2.3263 × 10\hat{ }14 | — | 2380 |
| 18 | 387420489 | 1.6284 × 10\hat{ }15 | — | 3060 |
| 19 | 1162261467 | 1.1399 × 10\hat{ }16 | — | 3876 |
| 20 | 3486784401 | 7.9792 × 10\hat{ }16 | 248832 | 4845 |

Table 1b shows the coding capacity of the four methods. All four methods compared in the table use specific probe sets that are not denatured between different rounds of detection. For intronSeqFISH there are four detection rounds needed to produce the pseudo colors of one coding round, therefore data is only given for rounds 4, 8,12,16 and 20. The merFISH-method uses a constant number of 4 signals, therefore the data starts with the smallest number of rounds possible. After 4 detection rounds the method with multi-decoders as described here exceeds the maximum coding capacity reached with 20 rounds of merFISH (depicted with one asterisk), after 7 rounds of detection the maximum coding capacity of intron FISH is exceeded (depicted with two asterisks) and after 12 rounds of detection the maximum coding capacity of the method of the present disclosure is exceeded (depicted with three asterisks). The usage of 3 different signal oligonucleotides is assumed (as is with intronSeqFISH).

### 3. Selective Denaturation, Oligonucleotide Assembly and Reuse of Unique Identifiers are Surprisingly Efficient

A key factor of the method according to the disclosure is the consecutive process of decoding oligonucleotide binding, signal oligonucleotide binding, signal detection and selective denaturation. In order to generate an encoding scheme, this process has to be repeated several times (depending on the length of the code word). Because the same unique identifier is reused in every detection cycle, all events from the first to the last detection cycle are depending on each other. Additionally, the selective denaturation depends on two different events: While the decoding oligonucleotide has to be dissolved from the unique identifier with highest efficiency, specific probes have to stay hybridized with highest efficiency.

Due to this the efficiency E of the whole encoding process can be described by the following equation:

$$E = {B}_{sp} \times {( {B}_{de} \times {B}_{si} \times {E}_{de} \times {S}_{sp} )}^{n}$$

E=total efficiency
Bₛₚ=binding of specific probes
B_{de}=binding of decoding oligonucleotides
Bₛᵢ=binding of signal oligonucleotides
E_{de}=elimination of decoding oligonucleotides
Sₛₚ=stability of specific probes during elimination process
n=number of detection cycles

Based on this equation the efficiency of each single step can be estimated for a given total efficiency of the method. The calculation is hereby based on the assumption, that each process has the same efficiency. The total efficiency describes the portion of successfully decodable signals of the total signals present.

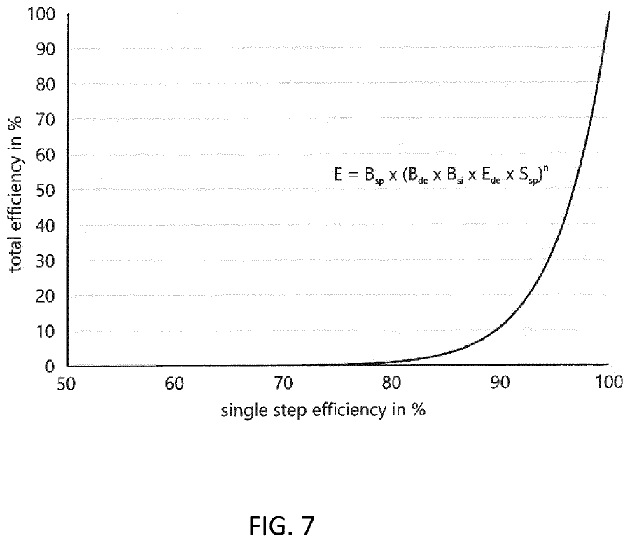

The total efficiency of the method is dependent on the efficiency of each single step of the different factors described by the equation. Under the assumption of an equally distributed efficiency the total efficiency can be plotted against the single step efficiency as shown in FIG. 7. As can be seen, a practically relevant total efficiency for an encoding scheme with 5 detection cycles can only be achieved with single step efficiencies clearly above 90%. For example, to achieve a total efficiency of 50% an average efficiency within each single step of 97.8% is needed. These calculations are even based on the assumption of a 100% signal detection and analysis efficiency. Due to broad DNA melting curves of oligonucleotides of a variety of sequences, the inventors assumed prior to experiments that the selective denaturation would work less efficient for denaturation of decoding oligonucleotides and that sequence specific binding probes are not stable enough. In contrast to this assumption, we found a surprising effectiveness of all steps and a high stability of sequence specific probes during selective denaturation.

Experimentally, the inventors achieved a total decoding efficiency of about 30% to 65% based on 5 detection cycles. A calculation of the efficiency of each single step (Bsp, Bde, Bsi, Ede, Ssp) by the formula given above revealed an average efficiency of about 94.4% to 98%. These high efficiencies are very surprising and cannot easily be anticipated by a well-trained person in this field.

4. Experimental data

### Background

The experiment shows the specific detection of 10 to 50 different mRNAs species in parallel with single molecule resolution. It is based on 5 detection cycles, 3 different fluorescent signals and an encoding scheme without signal gaps and a hamming distance of 2 (error detection). The experiment proofs the enablement and functionality of the method according to the disclosure.

Oligonucleotides and their sequences

All oligonucleotide sequences used in the experiment (target specific probes, decoding oligonucleotides, signal oligonucleotides) are listed in the sequence listing of the appendix. The signal oligonucleotide R: ST05*O_Atto594 was ordered from biomers.net GmbH. All other oligonucleotides were ordered from Integrated DNA Technologies. Oligonucleotides were dissolved in water. The stock solutions (100 μM) were stored at −20° C.

### Experimental Overview

The 50 different target specific probe sets are divided into 5 groups. The name of the transcript to be detected and the name of the target specific probe set are the same (transcript variant names of www.ensemble.org). The term “new” indicates a revised probe design. All oligonucleotide sequences of the probe sets can be found in the sequence listing. The table lists the unique identifier name of the probe set as well as the names of the decoding oligonucleotides used in the different detection cycles. The resulting code shows the sequence of fluorescent signals generated during the 5 detection cycles (G (reen) =Alexa Fluor 488, O (range) =Atto 594, Y (ellow) =Alexa Fluor 546).

TABLE 2
Experimental overview

| target | unique | Decoding oligonucleotides in detection cycle: | | | | | resulting |
|---|---|---|---|---|---|---|---|
| transcript | identifier | 1 | 2 | 3 | 4 | 5 | code |

Group 1

| | | | | | | | |
|---|---|---|---|---|---|---|---|
| DDX5-201 | ST21 | ST21-ST07 | ST21-ST05 | ST21-ST07 | ST21-ST05 | ST21-ST06 | GOGOY |
| RAD17-208 | ST02 | ST02-ST06 | ST02-ST07 | ST02-ST06 | ST02-ST06 | ST02-ST07 | YGYYG |
| SPOCK1-202 | ST03 | ST03-ST06 | ST03-ST06 | ST03-ST07 | ST03-ST05 | ST03-ST05 | YYGOO |
| FBXO32-203 | ST04 | ST04-ST07 | ST04-ST06 | ST04-ST06 | ST04-ST06 | ST04-ST05 | GYYYO |
| THRAP3-203 | ST14 | ST14-ST07 | ST14-ST05 | ST14-ST05 | ST14-ST07 | ST14-ST05 | GOOGO |
| GART-203 | ST11 | ST11-ST06 | ST11-ST07 | ST11-ST06 | ST11-ST05 | ST11-ST05 | YGYOO |
| KAT2A-201 | ST13 | ST13-ST06 | ST13-ST06 | ST13-ST07 | ST13-ST06 | ST13-ST07 | YYGYG |
| HPRT1-201 | ST12 | ST12-ST06 | ST12-ST07 | ST12-ST07 | ST12-ST06 | ST12-ST06 | YGGYY |
| CCNA2-201 | ST22 | ST22-ST05 | ST22-ST07 | ST22-ST06 | ST22-ST06 | ST22-ST05 | OGYYO |
| NKRF-201 | ST23 | ST23-ST05 | ST23-ST06 | ST23-ST07 | ST23-ST06 | ST23-ST05 | OYGYO |

Group 2

| | | | | | | | |
|---|---|---|---|---|---|---|---|
| CCNE1-201-new | NT01 | NT01-ST07 | NT01-ST07 | NT01-ST06 | NT01-ST06 | NT01-ST06 | GGYYY |
| COG5-201 | NT03 | NT03-ST06 | NT03-ST06 | NT03-ST05 | NT03-ST05 | NT03-ST06 | YYOOY |
| FBN1-201 | NT04 | NT04-ST05 | NT04-ST07 | NT04-ST06 | NT04-ST07 | NT04-ST07 | OGYGG |
| DYNC1H1-201 | NT05 | NT05-ST07 | NT05-ST06 | NT05-ST07 | NT05-ST06 | NT05-ST06 | GYGYY |
| CKAP5-202 | NT06 | NT06-ST05 | NT06-ST06 | NT06-ST07 | NT06-ST05 | NT06-ST06 | OYGOY |
| KRAS-202 | NT07 | NT07-ST06 | NT07-ST05 | NT07-ST06 | NT07-ST06 | NT07-ST05 | YOYYO |
| EGFR-207 | NT08 | NT08-ST07 | NT08-ST06 | NT08-ST05 | NT08-ST05 | NT08-ST05 | GYOOO |
| TP53-205 | NT09 | NT09-ST06 | NT09-ST05 | NT09-ST06 | NT09-ST07 | NT09-ST07 | YOYGG |
| NF1-204 | XT01 | XT01-ST06 | XT01-ST06 | XT01-ST07 | XT01-ST07 | XT01-ST06 | YYGGY |
| NF2-204 | XT02 | XT02-ST07 | XT02-ST06 | XT02-ST05 | XT02-ST06 | XT02-ST07 | GYOYG |

Group 3

| | | | | | | | |
|---|---|---|---|---|---|---|---|
| ACO2-201 | XT03 | XT03-ST06 | XT03-ST06 | XT03-ST06 | XT03-ST07 | XT03-ST05 | YYYGO |
| AKT1-211 | XT04 | XT04-ST07 | XT04-ST06 | XT04-ST07 | XT04-ST07 | XT04-ST05 | GYGGO |
| LYPLAL1-202 | XT05 | XT05-ST07 | XT05-ST05 | XT05-ST06 | XT05-ST05 | XT05-ST05 | GOYOO |
| PKD2-201 | XT06 | XT06-ST06 | XT06-ST07 | XT06-ST05 | XT06-ST05 | XT06-ST07 | YGOOG |
| ENG-204 | XT09 | XT09-ST05 | XT09-ST05 | XT09-ST06 | XT09-ST06 | XT09-ST06 | OOYYY |
| FANCE-201 | XT10 | XT10-ST05 | XT10-ST07 | XT10-ST06 | XT10-ST05 | XT10-ST06 | OGYOY |
| MET-201 | XT12 | XT12-ST05 | XT12-ST06 | XT12-ST05 | XT12-ST06 | XT12-ST06 | OYOYY |
| NOTCH2-201 | XT13 | XT13-ST05 | XT13-ST05 | XT13-ST06 | XT13-ST07 | XT13-ST05 | OOYGO |
| SPOP-206 | XT14 | XT14-ST05 | XT14-ST07 | XT14-ST05 | XT14-ST07 | XT14-ST06 | OGOGY |
| ABL1-202 | XT16 | XT16-ST05 | XT16-ST06 | XT16-ST06 | XT16-ST05 | XT16-ST05 | OYYOO |

Group 4

| | | | | | | | |
|---|---|---|---|---|---|---|---|
| ATP11C-202 | XT17 | XT17-ST07 | XT17-ST06 | XT17-ST06 | XT17-ST05 | XT17-ST06 | GYYOY |
| BCR-202 | XT18 | XT18-ST05 | XT18-ST06 | XT18-ST06 | XT18-ST07 | XT18-ST06 | OYYGY |
| CAV1-205 | XT19 | XT19-ST07 | XT19-ST05 | XT19-ST06 | XT19-ST07 | XT19-ST06 | GOYGY |
| CDK2-201 | XT20 | XT20-ST05 | XT20-ST05 | XT20-ST07 | XT20-ST07 | XT20-ST06 | OOGGY |
| DCAF1-202 | XT201 | XT201-ST06 | XT201-ST06 | XT201-ST05 | XT201-ST07 | XT201-ST07 | YYOGG |
| FHOD1-201 | XT202 | XT202-ST05 | XT202-ST07 | XT202-ST07 | XT202-ST05 | XT202-ST07 | OGGOG |
| GMDS-202 | XT203 | XT203-ST07 | XT203-ST05 | XT203-ST07 | XT203-ST06 | XT203-ST05 | GOGYO |
| IFNAR1-201 | XT204 | XT204-ST06 | XT204-ST07 | XT204-ST05 | XT204-ST07 | XT204-ST05 | YGOGO |
| NSMF-203 | XT206 | XT206-ST07 | XT206-ST06 | XT206-ST07 | XT206-ST05 | XT206-ST07 | GYGOG |
| POLA2-201 | XT208 | XT208-ST06 | XT208-ST06 | XT208-ST06 | XT208-ST05 | XT208-ST07 | YYYOG |

Group 5

| | | | | | | | |
|---|---|---|---|---|---|---|---|
| BRCA1-210new | NT10 | NT10-ST07 | NT10-ST05 | NT10-ST06 | NT10-ST06 | NT10-ST07 | GOYYG |
| JAK1-201new | XT11 | XT11-ST05 | XT11-ST05 | XT11-ST07 | XT11-ST06 | XT11-ST07 | OOGYG |
| STRAP-202 | XT207 | XT207-ST07 | XT207-ST07 | XT207-ST06 | XT207-ST05 | XT207-ST07 | GGYOG |
| SERPINB5-201 | XT209 | XT209-ST07 | XT209-ST05 | XT209-ST05 | XT209-ST05 | XT209-ST07 | GOOOG |
| SETX-201 | XT210 | XT210-ST06 | XT210-ST07 | XT210-ST06 | XT210-ST07 | XT210-ST06 | YGYGY |
| WDFY1-201 | XT212 | XT212-ST05 | XT212-ST07 | XT212-ST05 | XT212-ST06 | XT212-ST07 | OGOYG |
| TACC1-201 | XT213 | XT213-ST05 | XT213-ST07 | XT213-ST07 | XT213-ST07 | XT213-ST05 | OGGGO |
| KIF2A-203 | XT214 | XT214-ST07 | XT214-ST07 | XT214-ST06 | XT214-ST07 | XT214-ST05 | GGYGO |
| CDT1-201 | XT215 | XT215-ST07 | XT215-ST07 | XT215-ST07 | XT215-ST05 | XT215-ST05 | GGGOO |
| CENPE-202 | XT216 | XT216-ST07 | XT216-ST06 | XT216-ST05 | XT216-ST07 | XT216-ST06 | GYOGY |

### Variations of the Experiment

Some variations of the experiment have been performed. Experiments 1 to 4 mainly differ in the number of transcripts detected in parallel. The groups listed as target specific probe sets refer to table 6. Experiments 5 to 8 are single round, single target controls for comparison with the decoded signals.

TABLE 3
Variations of the experiment

| | | Nr. of | Imaging |
|---|---|---|---|
| | Target specific probe | detection | with |
| Experiment | sets used | cycles | trolox |
| 1. 50 transcripts_T+ | Groups 1 to 5 of table 6 | 5 | + |
| 2. 50 transcripts_T− | Groups 1 to 5 of table 6 | 5 | − |
| 3. 30 transcripts_T+ | Groups 2 to 4 of table 6 | 5 | + |
| 4. 10 transcripts_T+ | Group 1 of table 6 | 5 | + |
| 5. DDX5 | DDX5-ST21 | 1 | − |
| 6. RAD17 | RAD17-ST02 | 1 | − |
| 7. SPOCK1 | SPOCK1-ST03 | 1 | − |
| 8. THRAP3 | THRAP3-ST14 | 1 | − |

### Experimental Details

A. Seeding and Cultivation of Cells

Hela cells were grown in Hela cell culture medium to nearly 100% confluency. The Hela cell culture medium comprises DMEM (Thermo Fisher, Cat.: 31885) with 10% FCS (Biochrom, Cat.: S0415), 1% Penicillin-Streptomycin (Sigma-Adrich, Cat.: P0781) and 1% MEM Non-Essential Amino Acids Solution (Thermo Fisher, Cat.: 11140035). After aspiration of cell culture medium, cells were trypsinized by incubation with trypsin EDTA solution (Sigma-Aldrich, Cat.: T3924) for 5 min at 37° C. after a washing step with PBS (1,424 g/l Na₂HPO₄*2H₂O, 0,276 g/l, NaH₂PO₄*2H₂O, 8,19 g/l NaCl in water, pH 7,4). Cells were then seeded on the wells of a u-Slide 8 Well ibidiTreat (Ibidi, Cat.: 80826). The number of cells per well was adjusted to reach about 50% confluency after adhesion of the cells. Cells were incubated over night with 200 μl HeLa cell culture medium per well.

### B. Fixation of Cells

After aspiration of cell culture medium and two washing steps with 200 μl 37° C. warm PBS per well, cells were fixed with 200 μl precooled methanol (−20° C., Roth, Cat.: 0082.1) for 10 min at −20° C.

C. Counterstaining with Sudan Black

Methanol was aspirated and 150 μl of 0.2% Sudan Black-solution diluted in 70% ethanol were added to each well. Wells were incubated for 5 min in the dark at room temperature. After incubation cells were washed three times with 400 μl 70% ethanol per well to eliminate the excess of Sudan Black-solution.

D. Hybridization of Analyte/Target-Specific Probes

Before hybridization, cells were equilibrated with 200 μl sm-wash-buffer. The sm-wash-buffer comprises 30 mM Na3Citrate, 300 mM NaCl, pH7, 10% formamide (Roth, Cat.: P040.1) and 5 mM Ribonucleoside Vanadyl Complex (NEB, Cat.: S1402S). For each target-specific probe set 1 ul of a 100 μM oligonucleotide stock solution was added to the mixture. The oligonucleotide stock solution comprises equimolar amounts of all target specific oligonucleotides of the corresponding target specific probe set. The total volume of the mixture was adjusted to 100 μl with water and mixed with 100 μl of a 2× concentrated hybridization buffer solution. The 2× concentrated hybridization buffer comprises 120 mM Na₃Citrate, 1200 mM NaCl, pH7, 20% formamide and 20 mM Ribonucleoside Vanadyl Complex. The resulting 200 μl hybridization mixture was added to the corresponding well and incubated at 37° C. for 2 h. Afterwards cells were washed three times with 200 μl per well for 10 min with target probe wash buffer at 37° C. The target probe wash buffer comprises 30 mM Na₃Citrate, 300 mM NaCl, pH7, 20% formamide and 5 mM Ribonucleoside Vanadyl Complex.

### E. Hybridization of Decoding Oligonucleotides

Before hybridization, cells were equilibrated with 200 μl sm-wash-buffer. For each decoding oligonucleotide 1,5 μl of a 5 μM stock solution were added to the mixture. The total volume of the mixture was adjusted to 75 μl with water and mixed with 75 μl of a 2× concentrated hybridization buffer solution. The resulting 150 μl decoding oligonucleotide hybridization mixture was added to the corresponding well and incubated at room temperature for 45 min.

Afterwards cells were washed three times with 200 μl per well for 2 min with sm-wash-buffer at room temperature.

### F. Hybridization of Signal Oligonucleotides

Before hybridization, cells were equilibrated with 200 μl sm-wash-buffer. The signal oligonucleotide hybridization mixture was the same for all rounds of experiments 1 to 4 and comprised 0,3 μM of each signal oligonucleotide (see table A3) in 1× concentrated hybridization buffer solution. In each round 150 μl of this solution were added per well and incubated at room temperature for 45 min. The procedure was the same for experiments 5 to 8 with the exception that the final concentration of each signal oligonucleotide was 0,15 μM. Afterwards cells were washed three times with 200 μl per well for 2 min with sm-wash-buffer at room temperature.

### G. Fluorescence and White Light Imaging

Cells were washed once with 200 μl of imaging buffer per well at room temperature. In experiments without Trolox (see table 7, last column) imaging buffer comprises 30 mM Na3Citrate, 300 mM NaCl, pH7 and 5 mM Ribonucleoside Vanadyl Complex. In experiments with Trolox, imaging buffer additionally contains 10% VectaCell Trolox Antifade Reagent (Vector laboratories, Cat.: CB-1000), resulting in a final Trolox concentration of 10 mM.

A Zeiss Axiovert 200M microscope with a 63× immersion oil objective (Zeiss, apochromat) with numerical aperture of 1.4, a pco.edge 4.2 CMOS camera (PCO AG) and an LED-light source (Zeiss, colibri 7) was used for imaging of the regions. Filter sets and LED-wavelengths were adjusted to the different optima of the fluorophores used. Illumination times per image were 1000 ms for Alexa Fluor 546 and Atto 594 and 400 ms for Alexa Fluor 488.

In each experiment, three regions were randomly chosen for imaging. For each region, a z-stack of 32 images was detected with a z-step size of 350 nm. Additionally, one white light image was taken from the regions. In experiments with more than one detection cycle, the regions of the first detection round were found back and imaged in every subsequent round.

### H. Selective Denaturation

For selective denaturation, every well was incubated with 200 μl of sm-wash-buffer at 42° C. for 6 min. This procedure was repeated six times.

Steps (E) to (H) were repeated 5 times in experiments 1 to 4. Step (H) was omitted for the 5^{th} detection cycle.

### 1. Analysis

Based on custom ImageJ-plugins a semi-automated analysis of the raw data was performed to distinguish the specific fluorescent signals from the background. The resulting 3D-point clouds of all three fluorescent channels were combined in silico with a custom VBA script. The resulting combined 3D-point clouds of the 5 detection cycles were aligned to each other on the basis of a VBA script. The resulting alignments revealed the code words for each unique signal detected. Successfully decoded signals were used for quantitative and spatial analysis of the experiments based on custom VBA-scripts and ImageJ-plugins.

### Results

### 1. Absolute Numbers of Decoded Signals

The absolute numbers of successfully decoded signals for all transcripts are listed for each region of each experiment in the following Table 4. In summary, the sum of correct codes depicts the total number of decoded signals that were assigned to transcripts detectable in the corresponding experiment, while the sum of incorrect codes it the total number of decoded signals not detectable in the corresponding experiment. The total number of signals comprises successfully decoded as well as unsuccessfully decoded signals.

TABLE 4
Absolute numbers of decoded signals

| | Experiment 1: | | | Experiment 2: | | | Experiment 3: | | | Experiment 4: | | |
|---|---|---|---|---|---|---|---|---|---|---|---|---|
| transcript | region: | | | region: | | | region: | | | region: | | |
| name: | 1 | 2 | 3 | 1 | 2 | 3 | 1 | 2 | 3 | 1 | 2 | 3 |

Group 1

| | | | | | | | | | | | | |
|---|---|---|---|---|---|---|---|---|---|---|---|---|
| DDX5-201 | 1214 | 1136 | 927 | 1144 | 1509 | 1176 | 2964 | 2034 | 2141 | 8 | 2 | 2 |
| RAD17-208 | 40 | 126 | 50 | 26 | 55 | 62 | 22 | 30 | 33 | 7 | 9 | 10 |
| SPOCK1-202 | 581 | 655 | 301 | 483 | 875 | 149 | 1349 | 621 | 539 | 2 | 10 | 8 |
| FBXO32-203 | 153 | 175 | 68 | 113 | 106 | 78 | 301 | 160 | 269 | 5 | 13 | 9 |
| THRAP3-203 | 1079 | 2180 | 1035 | 810 | 1179 | 1047 | 2318 | 1609 | 1609 | 6 | 8 | 16 |
| GART-203 | 422 | 397 | 346 | 350 | 333 | 202 | 766 | 658 | 569 | 5 | 3 | 2 |
| KAT2A-201 | 141 | 310 | 166 | 174 | 307 | 186 | 382 | 315 | 340 | 5 | 1 | 3 |
| HPRT1-201 | 63 | 79 | 34 | 44 | 116 | 71 | 85 | 88 | 112 | 1 | 0 | 6 |
| CCNA2-201 | 91 | 248 | 205 | 95 | 134 | 138 | 241 | 151 | 238 | 10 | 20 | 9 |
| NKRF-201 | 162 | 318 | 153 | 101 | 99 | 135 | 313 | 254 | 209 | 7 | 5 | 12 |

Group 2

| | | | | | | | | | | | | |
|---|---|---|---|---|---|---|---|---|---|---|---|---|
| CCNE1-201-new | 75 | 180 | 38 | 57 | 110 | 97 | 0 | 0 | 1 | 122 | 104 | 120 |
| COG5-201 | 57 | 21 | 38 | 26 | 45 | 23 | 0 | 0 | 1 | 56 | 86 | 60 |
| FBN1-201 | 202 | 1338 | 499 | 456 | 513 | 571 | 0 | 1 | 3 | 450 | 778 | 895 |
| DYNC1H1-201 | 554 | 892 | 398 | 664 | 1026 | 666 | 4 | 3 | 7 | 823 | 1148 | 1248 |
| CKAP5-202 | 43 | 23 | 77 | 51 | 98 | 74 | 1 | 2 | 1 | 94 | 190 | 212 |
| KRAS-202 | 331 | 417 | 355 | 302 | 333 | 230 | 0 | 0 | 1 | 279 | 637 | 371 |
| EGFR-207 | 527 | 252 | 372 | 293 | 519 | 322 | 0 | 1 | 0 | 411 | 719 | 818 |
| TP53-205 | 116 | 324 | 194 | 138 | 198 | 169 | 0 | 1 | 4 | 182 | 280 | 181 |
| NF1-204 | 381 | 676 | 320 | 347 | 522 | 416 | 3 | 2 | 0 | 507 | 642 | 659 |
| NF2-204 | 434 | 638 | 361 | 336 | 468 | 401 | 0 | 0 | 2 | 508 | 523 | 636 |

Group 3

| | | | | | | | | | | | | |
|---|---|---|---|---|---|---|---|---|---|---|---|---|
| ACO2-201 | 456 | 759 | 444 | 333 | 367 | 345 | 0 | 0 | 0 | 556 | 681 | 681 |
| AKT1-211 | 351 | 710 | 301 | 230 | 437 | 297 | 0 | 0 | 3 | 558 | 614 | 690 |
| LYPLAL1-202 | 65 | 62 | 51 | 33 | 58 | 51 | 7 | 6 | 8 | 28 | 34 | 42 |
| PKD2-201 | 72 | 194 | 124 | 95 | 129 | 69 | 1 | 0 | 0 | 122 | 164 | 169 |
| ENG-204 | 472 | 890 | 458 | 446 | 494 | 558 | 0 | 2 | 0 | 1145 | 799 | 1119 |
| FANCE-201 | 24 | 82 | 61 | 56 | 67 | 56 | 0 | 0 | 2 | 119 | 131 | 153 |
| MET-201 | 268 | 744 | 333 | 426 | 275 | 462 | 0 | 0 | 0 | 790 | 662 | 782 |
| NOTCH2-201 | 344 | 823 | 404 | 172 | 256 | 208 | 4 | 0 | 3 | 402 | 779 | 772 |
| SPOP-206 | 43 | 377 | 139 | 68 | 117 | 100 | 0 | 0 | 0 | 215 | 289 | 300 |
| ABL1-202 | 224 | 72 | 218 | 107 | 122 | 153 | 4 | 1 | 1 | 302 | 393 | 480 |

Group 4

| | | | | | | | | | | | | |
|---|---|---|---|---|---|---|---|---|---|---|---|---|
| ATP11C-202 | 170 | 116 | 121 | 86 | 165 | 128 | 2 | 1 | 1 | 130 | 206 | 234 |
| BCR-202 | 180 | 401 | 185 | 177 | 149 | 169 | 1 | 0 | 0 | 321 | 372 | 485 |
| CAV1-205 | 728 | 777 | 644 | 328 | 653 | 567 | 2 | 0 | 5 | 613 | 997 | 852 |
| CDK2-201 | 306 | 937 | 367 | 297 | 385 | 358 | 4 | 1 | 3 | 568 | 742 | 888 |
| DCAF1-202 | 119 | 292 | 187 | 59 | 67 | 65 | 0 | 1 | 0 | 108 | 171 | 131 |
| FHOD1-201 | 60 | 233 | 143 | 132 | 185 | 157 | 1 | 0 | 1 | 194 | 294 | 300 |
| GMDS-202 | 67 | 124 | 49 | 56 | 80 | 63 | 7 | 2 | 3 | 59 | 73 | 114 |
| IFNAR1-201 | 81 | 221 | 135 | 99 | 104 | 55 | 1 | 0 | 1 | 159 | 266 | 238 |
| NSMF-203 | 448 | 583 | 386 | 293 | 453 | 331 | 8 | 4 | 11 | 525 | 608 | 616 |
| POLA2-201 | 74 | 111 | 62 | 45 | 72 | 67 | 2 | 4 | 2 | 41 | 75 | 57 |

Group 5

| | | | | | | | | | | | | |
|---|---|---|---|---|---|---|---|---|---|---|---|---|
| BRCA1-210new | 230 | 704 | 248 | 324 | 439 | 374 | 2 | 1 | 0 | 2 | 3 | 3 |
| JAK1-201new | 157 | 554 | 223 | 185 | 259 | 263 | 0 | 1 | 0 | 5 | 4 | 2 |
| STRAP-202 | 42 | 75 | 31 | 18 | 52 | 40 | 3 | 1 | 0 | 6 | 4 | 4 |
| SERPINB5-201 | 324 | 598 | 343 | 286 | 344 | 364 | 6 | 13 | 9 | 0 | 1 | 2 |
| SETX-201 | 212 | 540 | 254 | 291 | 439 | 336 | 2 | 2 | 2 | 12 | 9 | 5 |
| WDFY1-201 | 43 | 516 | 218 | 176 | 206 | 147 | 0 | 0 | 0 | 4 | 2 | 8 |
| TACC1-201 | 69 | 373 | 131 | 80 | 156 | 118 | 1 | 0 | 0 | 21 | 29 | 32 |
| KIF2A-203 | 442 | 879 | 445 | 298 | 519 | 430 | 5 | 1 | 2 | 6 | 11 | 9 |
| CDT1-201 | 31 | 27 | 33 | 19 | 61 | 28 | 15 | 8 | 9 | 0 | 8 | 1 |
| CENPE-202 | 192 | 246 | 215 | 94 | 262 | 225 | 0 | 1 | 0 | 8 | 9 | 9 |

Summary

| | | | | | | | | | | | | |
|---|---|---|---|---|---|---|---|---|---|---|---|---|
| sum of | 12960 | 23405 | 12890 | 11319 | 15917 | 12797 | 8741 | 5920 | 6059 | 10387 | 13457 | 14303 |
| correct codes: | | | | | | | | | | | | |
| sum of | 0 | 0 | 0 | 0 | 0 | 0 | 86 | 60 | 86 | 120 | 151 | 152 |
| incorrect codes: | | | | | | | | | | | | |
| total number | 42959 | 72157 | 32185 | 32037 | 58793 | 35470 | 13549 | 9182 | 10109 | 26701 | 32966 | 35451 |
| of signals: | | | | | | | | | | | | |
| % successfully | 30.2 | 32.4 | 40.0 | 35.3 | 27.1 | 36.1 | 64.5 | 64.5 | 59.9 | 38.9 | 40.8 | 40.3 |
| decoded: | | | | | | | | | | | | |

Table 4 shows a very low number of incorrectly decoded signals compared to the number of correctly decoded signals. The absolute values for decoded signals of a certain transcript are very similar between different regions of one experiment. The fraction of the total number of signals that can be successfully decoded is between 27.1% and 64.5%. This fraction depends on the number of transcripts and/or the total number of signals present in the respective region/experiment.

### Conclusion

The method according to the disclosure produces a low amount of incorrectly assigned code words and can therefore be considered specific. The fraction of successfully decodable signals is very high, even with very high numbers of signals per region and very high numbers of transcripts detected in parallel. The high fraction of assignable signals and the high specificity make the method practically useful.

### Comparison of Relative Transcript Abundancies Between Different Experiments

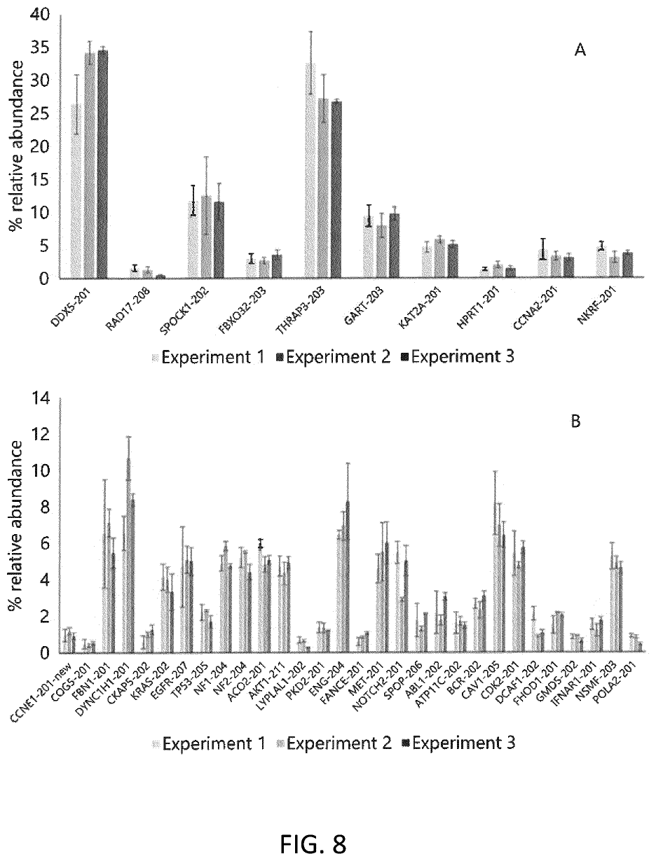

As shown in FIG. 8 for both comparisons (A and B) the overlap of detected transcripts between the experiments is used for the analysis. Each bar represents the mean abundance of all three regions of an experiment. The standard deviation between these regions is also indicated. Correlation of relative transcript abundancies between different experiments

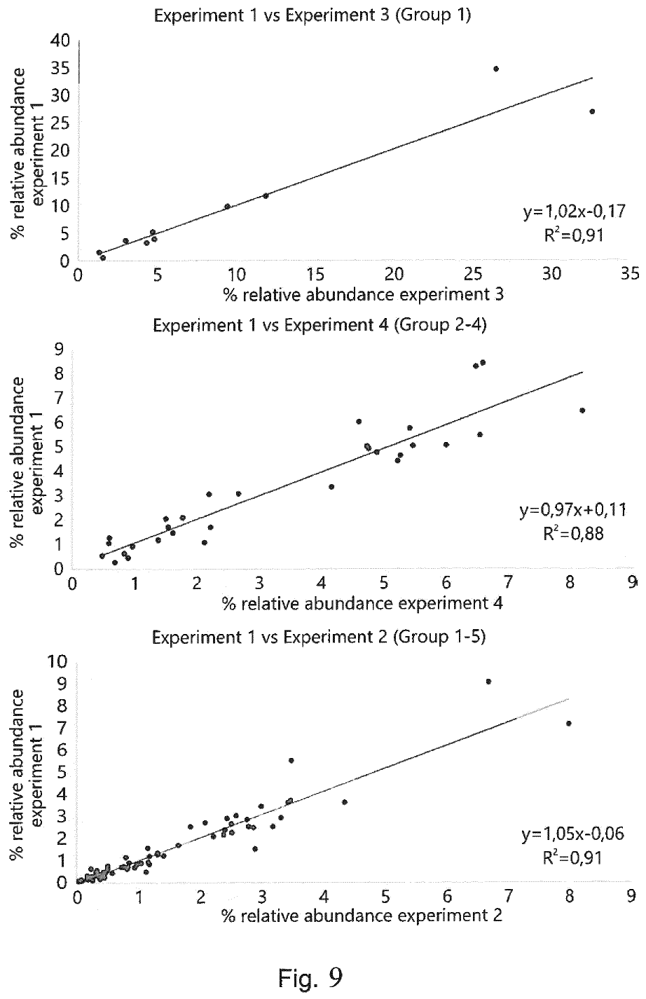

As can be seen in FIG. 9 the mean relative abundances of transcripts from experiment 1 are correlated to the abundances of the overlapping transcripts of experiment 3, 4 and 2. The correlation coefficient as well as the formula for the linear regression are indicated for each correlation.

FIG. 8 shows low standard deviations, indicating low variations of relative abundances between different regions of one experiment. The differences of relative abundances between transcripts from different experiments are also very low. This is the case for the comparison of transcripts from group 1 (FIG. 8A), that were detected in experiments 1, 2 and 3. It is also the case for the comparison of the transcripts from groups 2, 3 and 4 that were overlapping between experiments 1, 2 and 4. The very high correlation of these abundances can also be seen in FIG. 9. The abundances of transcripts from experiment 1 correlate very well with the abundances of the other multi round experiments. The correlation factors are between 0.88 and 0.91, while the slope of the linear regressions is between 0.97 and 1.05.

### Conclusion

The relative abundancies of transcripts correlate very well between different regions of one experiment but also between different experiments. This can be clearly seen by the comparisons of FIGS. 3 and 4. The main difference between the experiments is the number of different targets and hence the total number of signals detected. Therefore, the number of transcripts detected as well as the number and density of signals does not interfere with the ability of the method to accurately quantify the number of transcripts. The very good correlations further support the specificity and stability of the method, even with very high numbers of signals.

### Comparison of Intercellular Distribution of Signals

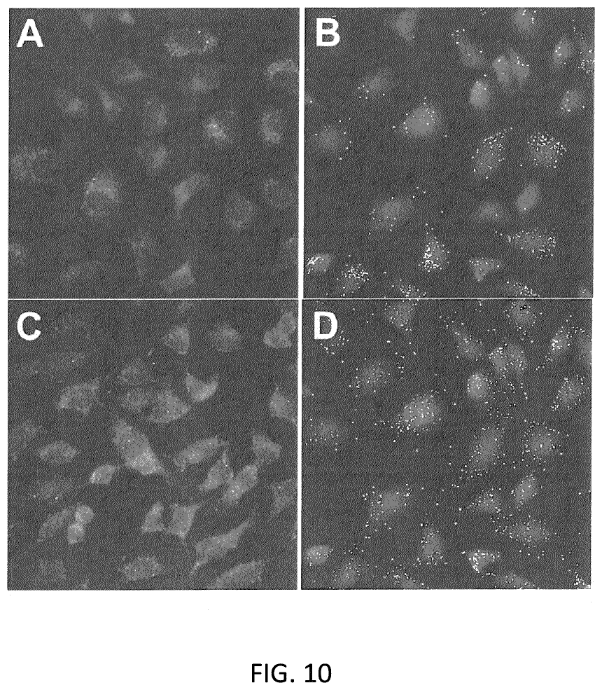

In FIG. 10 the maximum projections of image stacks are shown. A: region 1 of experiment 7 (single round, single transcript experiment detecting SPOCK1), B: 2D-projection of all selected signals from experiment 1, region 1 assigned to SPOCK1, C: region 1 of experiment 8 (single round, single transcript experiment detecting THRAP3), D: 2D-projection of all selected signals from experiment 1, region 1 assigned to THRAP3.

### Comparison of Intracellular Distribution of Signals

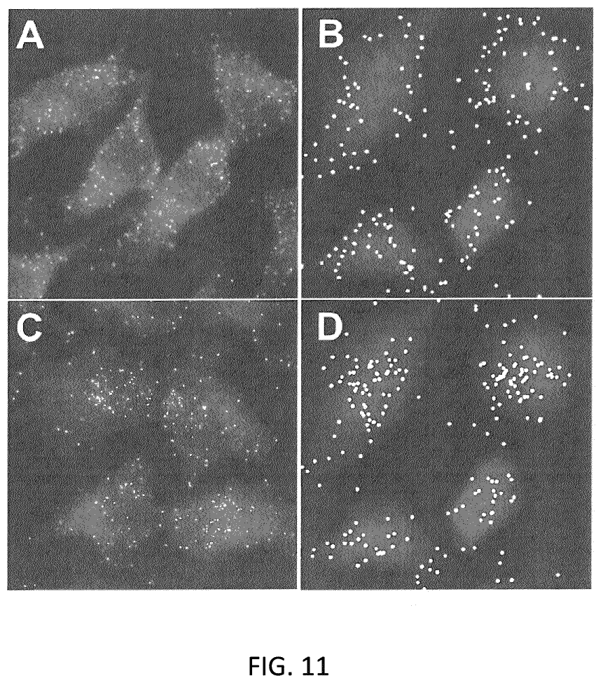

In FIG. 11 the maximum projections of image stacks are shown. Magnified sub regions of the corresponding regions are shown. A: region 1 of experiment 8 (single round, single transcript experiment detecting THRAP3), B: 2D-projection of selected signals from experiment 1, region 1 assigned to THRAP3, C: region 1 of experiment 5 (single round, single transcript experiment detecting DDX5), D: 2D-projection of all selected signals from experiment 1, region 1 assigned to DDX5.

FIG. 10 shows huge differences of intercellular distributions between different transcripts. SPOCK1 seems to be highly abundant in some cells but nearly absent in other cells (FIG. 10A). THRAP3 shows a more uniform distribution over all cells of a region (FIG. 10 C). These spatial distribution patterns can also clearly be observed with the point clouds assigned to the corresponding transcripts from experiment 1 (FIG. 10 B and D).

FIG. 11 shows huge differences of intracellular distributions between different transcripts. THRAP3 can be mainly observed in the periphery (cytoplasm) of the cells (FIG. 11A), while DDX5 shows a higher abundance in the center (nucleus) of the cells (FIG. 11 C). These intracellular distributions can also be observed with the point clouds of experiment 1 assigned to THRAP3 and DDX5 (FIG. 11 B and D).

### Conclusion

Next to the reliability of quantification, the point clouds of multi round experiments also show the same intracellular and intercellular distribution patterns of transcripts. This is clearly proven by the direct comparison of the assigned point clouds with signals from single round experiments detecting only one characteristic mRNA-species.

### Distribution Pattern of Different Cell Cycle Dependent Transcripts

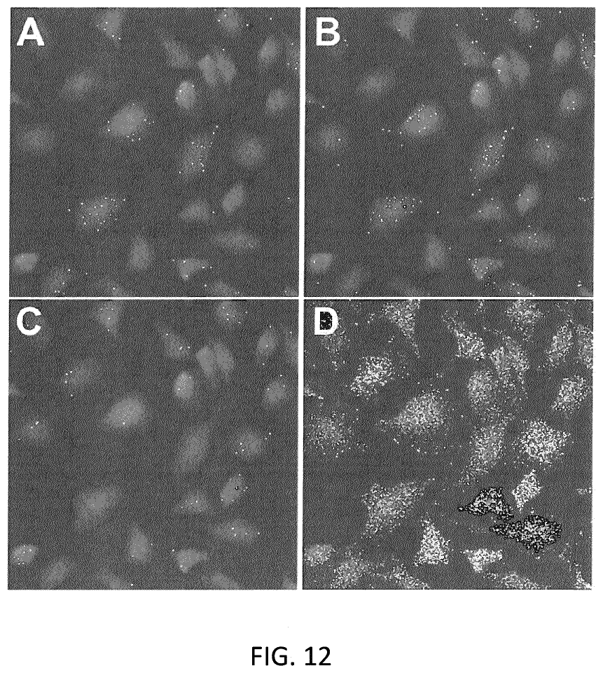

All images of FIG. 12 show region 1 of experiment 1. In each image, a point cloud is shown, that is assigned to a certain transcript, A: CCNA2, B: CENPE, C: CCNE1, D: all transcripts. FIG. 12 shows the transcripts of three different cell cycle dependent proteins. CENPE (FIG. 12 B) is also known as

Centromere protein E and accumulates during G2 phase. It is proposed to be responsible for spindle elongation and for chromosome movement. It is not present during interphase. CCNA2 (FIG. 12A) is also known as Cyclin A2. It regulates the cell cycle progression by interacting with CDK1 during transition from G2 to M-phase. Interestingly there is an obvious colocalization of both mRNA-species.

They are mainly present in the three central cells of region 1. CCNE1 (FIG. 12 C) is also known as Cyclin E1. This cyclin interacts with CDK2 and is responsible for the transition from G1 to S-phase. FIG. 12 shows clearly, that the transcripts of this gene are not present in the three central cells, but quite equally distributed over the other cells. It therefore shows an anti-localization to the other two transcripts. The data for the corresponding point-clouds are derived from a point cloud with a very high number of points and a very high point density (FIG. 12 D gives an impression).

### Conclusion

The three decoded point clouds of cell cycle dependent proteins shown in FIG. 12, show distribution patterns that can be explained by their corresponding function. These data strongly suggest, that our method reliably produces biological relevant data, even with a low number of signals per cell (FIG. 12 C) and with very high signal densities (FIG. 12 D).

### SEQUENCE LISTING

In the accompanying sequence listing SEQ ID Nos. 1-1247 refer to nucleotide sequences of exemplary target-specific oligonucleotides. The oligonucleotides listed consist of a target specific binding site (5′-end) a spacer/linker sequence (gtaac or tagac) and the unique identifier sequence, which is the same for all oligonucleotides of one probe set.

In the accompanying sequence listing SEQ ID Nos. 1248-1397 refer to nucleotide sequences of exemplary decoding oligonucleotides.

In the accompanying sequence listing SEQ ID Nos. 1398-1400 refer to the nucleotide sequences of exemplary signal oligonucleotides. For each signal oligonucleotide the corresponding fluorophore is present twice. One fluorophore is covalently linked to the 5′-end and one fluorophore is covalently linked to the 3′-end. SEQ ID No. 1398 comprises at its 5′ terminus “5Alex488N”, and at its 3′ terminus “3AlexF488N”. SEQ ID No. 1399 comprises at its 5′ terminus “5Alex546”, and at its 3′ terminus 3Alex546N. SEQ ID No. 1400 comprises at its 5′ terminus and at its 3′ terminus “Atto594”.

We claim:

1-65. (canceled)

66. A method for detecting an analyte in a pathogen-comprising sample comprising: i) inactivation of the pathogen within the sample without isolation of nucleic acids from the pathogen or the sample; and ii) detecting the analyte using spatial transcriptomics.

67. The method of claim 66, wherein the pathogen-comprising sample is selected from the group consisting of a solid, a fluidic sample, a smear and a surface comprising pathogenic material.

68. The method of claim 66, wherein the pathogen-comprising sample is a biological sample.

69. The method of claim 66, wherein the pathogen-comprising sample comprises one or more eukaryotic, archaeal, or eubacterial, viral or viroidal organisms.

70. The method of claim 66, wherein the pathogen-comprising sample comprises formalin-fixed, paraffin-embedded tissues containing a pathogen and/or wherein the pathogen-comprising sample is frozen or alcoholic stabilized.

71. The method of claim 66, wherein the pathogen is inactivated by a treatment selected from the list consisting of a physical, chemical, biochemical and/or biological treatment.

72. The method of claim 71, wherein the treatment is a physical treatment selected from the list consisting of temperature change, electromagnetic waves and light including visible and invisible light like UV irradiation, temperature treatment with a temperature higher than 30° C., 40°° C., 50° C., 60° C., 70° C., 80°° C., 90° C., 100° C. or 110° C., treatment by X-ray, radioactivity, UV-light, blue light red light and/or infrared light, or combinations thereof.

73. The method of claim 71, wherein the treatment is a chemical treatment selected from the list consisting of pH change, salt treatment, treatment with a fraction of a polar or non-polar solvent, treatment with an oxidative or reductive reagent, treatment with a reagent that perform covalent or non-covalent linkage to the pathogen and treatment with a degenerative reagent that cleaves at least parts of the pathogen, or combinations thereof.

74. The method of claim 71, wherein the treatment is a biological treatment selected from the list consisting of treatment with an enzyme and treatment with a biomolecule.

75. The method of claim 66, wherein the spatial transcriptomics detecting comprises a multiplex method for detecting different analytes in the pathogen-comprising sample by sequential signal-encoding of said analytes, comprising an inactivation of the pathogen within the sample without isolation of RNA and/or DNA from the pathogen or the sample, and (A) contacting the sample with at least twenty (20) different sets of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising (aa) a binding element(S) that specifically interacts with one of the different analytes to be encoded, and (bb) an identifier element (T) comprising a nucleotide sequence which is unique to the analyte to be encoded (unique identifier sequence), wherein the analyte-specific probes of a particular set of analyte-specific probes differ from the analyte-specific probes of another set of analyte-specific probes in the nucleotide sequence of the identifier element (T), wherein the analyte-specific probes in each set of analyte-specific probes binds to the same analyte and comprises the same nucleotide sequence of the identifier element (T) which is unique to said analyte; and (B) contacting the sample with at least one set of decoding oligonucleotides per analyte, wherein in each set of decoding oligonucleotides for an individual analyte each decoding oligonucleotide comprises: (aa) an identifier connector element (t) comprising a nucleotide sequence which is essentially complementary to at least a section of the unique identifier sequence of the identifier element (T) of the corresponding analyte-specific probe set, and (bb) a translator element (c) comprising a nucleotide sequence allowing a specific hybridization of a signal oligonucleotide; wherein the decoding oligonucleotides of a set for an individual analyte differ from the decoding oligonucleotides of another set for a different analyte in the first connect element (t); and (C) contacting the sample with at least a set of signal oligonucleotides, each signal oligonucleotide comprising: (aa) a translator connector element (C) comprising a nucleotide sequence which is essentially complementary to at least a section of the nucleotide sequence of a translator element (c) comprised in a decoding oligonucleotide, and (bb) a signal element; (D) detecting the signal caused by the signal element; (E) selectively removing the decoding oligonucleotides and signal oligonucleotides from the sample, thereby essentially maintaining the specific binding of the analyte-specific probes to the analytes to be encoded; (F) performing at least three (3) further cycles comprising steps B) to E) to generate an encoding scheme with a code word per analyte, wherein in particular the last cycle may stop with step (D).

76. The method of claim 75, wherein steps (B) through (D) are automated.

77. The method of claim 75, wherein all steps are automated.

78. The method of claim 75, wherein the code word per analyte obtained for the individual analytes in the performed cycles comprise the signals detected and additionally at least one element corresponding to no detected signal.

79. The method of claim 75, wherein no signal is detected for at least one analyte within at least one cycle.

80. The method of claim 75, wherein for at least for one individual analyte a position of the code word is null, no signal or zero (0).

81. The method of claim 80, wherein the code word zero (0) is generated by using no decoding oligonucleotides having an identifier connector element (t) comprising a nucleotide sequence which is essentially complementary to at least a section of the unique identifier sequence of the identifier element (T) of a corresponding analyte-specific probe for an individual analyte.

82. The method of claim 80, wherein if at least for one individual analyte a position of the code word is zero (0) in this cycle no corresponding decoding oligonucleotides having an identifier connector element (t) comprising a nucleotide sequence which is essentially complementary to at least a section of the unique identifier sequence of the identifier element (T) of a corresponding analyte-specific probe for an individual analyte are used.

83. The method of claim 80, wherein between step (C) and step (D) non bound signal oligonucleotides are removed.

84. The method of claim 80, wherein between step (B) and step (C) non bound signal oligonucleotides are removed.

85. The method of claim 66, wherein the analytes are fixed in a permeabilized sample.


SEQUENCE LISTING (1400 sequences)

SEQ ID NO: 1 - DNA, 45 bp, synthetic construct
  misc_feature 1..45: note = Target Specific Oligonucleotide
  source 1..45: mol_type = other DNA
actactagagaccggtagaaatgagtaacgattaccgacttatcc

SEQ ID NO: 2 - DNA, 42 bp, synthetic construct
  misc_feature 1..42: note = Target Specific Oligonucleotide
  source 1..42: mol_type = other DNA
tggcggggaacgaagtatatagtaacgattaccgacttatcc

SEQ ID NO: 3 - DNA, 40 bp, synthetic construct
  misc_feature 1..40: note = Target Specific Oligonucleotide
  source 1..40: mol_type = other DNA
tggcgtcaatggttgcggtgtaacgattaccgacttatcc

SEQ ID NO: 4 - DNA, 41 bp, synthetic construct
  misc_feature 1..41: note = Target Specific Oligonucleotide
  source 1..41: mol_type = other DNA
tcggtcactcgaataacccggtaacgattaccgacttatcc

SEQ ID NO: 5 - DNA, 41 bp, synthetic construct
  misc_feature 1..41: note = Target Specific Oligonucleotide
  source 1..41: mol_type = other DNA
ctccaaatcgaggtgcaccagtaacgattaccgacttatcc

SEQ ID NO: 6 - DNA, 40 bp, synthetic construct
  misc_feature 1..40: note = Target Specific Oligonucleotide
  source 1..40: mol_type = other DNA
ggccagttcccgagttggtgtaacgattaccgacttatcc

SEQ ID NO: 7 - DNA, 42 bp, synthetic construct
  misc_feature 1..42: note = Target Specific Oligonucleotide
  source 1..42: mol_type = other DNA
agacttcaagcgacatgctctgtaacgattaccgacttatcc

SEQ ID NO: 8 - DNA, 42 bp, synthetic construct
  misc_feature 1..42: note = Target Specific Oligonucleotide
  source 1..42: mol_type = other DNA
ggagcaccaccgtagatacaagtaacgattaccgacttatcc

SEQ ID NO: 9 - DNA, 44 bp, synthetic construct
  misc_feature 1..44: note = Target Specific Oligonucleotide
  source 1..44: mol_type = other DNA
ctctccaaatcacgtatttgtgggtaacgattaccgacttatcc

SEQ ID NO: 10 - DNA, 45 bp, synthetic construct
  misc_feature 1..45: note = Target Specific Oligonucleotide
  source 1..45: mol_type = other DNA
agagtttgcctatcaggtcttattgtaacgattaccgacttatcc

SEQ ID NO: 11 - DNA, 40 bp, synthetic construct
  misc_feature 1..40: note = Target Specific Oligonucleotide
  source 1..40: mol_type = other DNA
ggccaagtcgcactccacagtaacgattaccgacttatcc

SEQ ID NO: 12 - DNA, 43 bp, synthetic construct
  misc_feature 1..43: note = Target Specific Oligonucleotide
  source 1..43: mol_type = other DNA
tccttttctacgtcatgacacagtaacgattaccgacttatcc

SEQ ID NO: 13 - DNA, 46 bp, synthetic construct
  misc_feature 1..46: note = Target Specific Oligonucleotide
  source 1..46: mol_type = other DNA
ttccattagacgaataagtttttcagtaacgattaccgacttatcc

SEQ ID NO: 14 - DNA, 44 bp, synthetic construct
  misc_feature 1..44: note = Target Specific Oligonucleotide
  source 1..44: mol_type = other DNA
tttctggtaagctcatcacatctgtaacgattaccgacttatcc

SEQ ID NO: 15 - DNA, 46 bp, synthetic construct
  misc_feature 1..46: note = Target Specific Oligonucleotide
  source 1..46: mol_type = other DNA
gagttagggtagtcataattgatgagtaacgattaccgacttatcc

SEQ ID NO: 16 - DNA, 47 bp, synthetic construct
  misc_feature 1..47: note = Target Specific Oligonucleotide
  source 1..47: mol_type = other DNA
ttcttccaattcgatgaatataatccgtaacgattaccgacttatcc

SEQ ID NO: 17 - DNA, 48 bp, synthetic construct
  misc_feature 1..48: note = Target Specific Oligonucleotide
  source 1..48: mol_type = other DNA
tgttattaggtgtaaagaaagtgtatggtaacgattaccgacttatcc

SEQ ID NO: 18 - DNA, 42 bp, synthetic construct
  misc_feature 1..42: note = Target Specific Oligonucleotide
  source 1..42: mol_type = other DNA
gagataaggtcgctcacttgcgtaacgattaccgacttatcc

SEQ ID NO: 19 - DNA, 42 bp, synthetic construct
  misc_feature 1..42: note = Target Specific Oligonucleotide
  source 1..42: mol_type = other DNA
acctctgtcttcgaccaactggtaacgattaccgacttatcc

SEQ ID NO: 20 - DNA, 41 bp, synthetic construct
  misc_feature 1..41: note = Target Specific Oligonucleotide
  source 1..41: mol_type = other DNA
acccctggaacgacctgaacgtaacgattaccgacttatcc

SEQ ID NO: 21 - DNA, 41 bp, synthetic construct
  misc_feature 1..41: note = Target Specific Oligonucleotide
  source 1..41: mol_type = other DNA
agtatctgtcccgacggtcagtaacgattaccgacttatcc

SEQ ID NO: 22 - DNA, 40 bp, synthetic construct
  misc_feature 1..40: note = Target Specific Oligonucleotide
  source 1..40: mol_type = other DNA
accccttttgcccgcagaggtaacgattaccgacttatcc

SEQ ID NO: 23 - DNA, 44 bp, synthetic construct
  misc_feature 1..44: note = Target Specific Oligonucleotide
  source 1..44: mol_type = other DNA
tttaagcaggctagagtaacctcgtaacgattaccgacttatcc

SEQ ID NO: 24 - DNA, 45 bp, synthetic construct
  misc_feature 1..45: note = Target Specific Oligonucleotide
  source 1..45: mol_type = other DNA
gcattggataaccaatcataggtggtaacgattaccgacttatcc

SEQ ID NO: 25 - DNA, 41 bp, synthetic construct
  misc_feature 1..41: note = Target Specific Oligonucleotide
  source 1..41: mol_type = other DNA
tcccctagtccgagttgctcgtaaccctaattatgggaatc

SEQ ID NO: 26 - DNA, 43 bp, synthetic construct
  misc_feature 1..43: note = Target Specific Oligonucleotide
  source 1..43: mol_type = other DNA
ccaatttgcacttggatgtgtagtaaccctaattatgggaatc

SEQ ID NO: 27 - DNA, 43 bp, synthetic construct
  misc_feature 1..43: note = Target Specific Oligonucleotide
  source 1..43: mol_type = other DNA
ttttgccagtagaacaagcatggtaaccctaattatgggaatc

SEQ ID NO: 28 - DNA, 40 bp, synthetic construct
  misc_feature 1..40: note = Target Specific Oligonucleotide
  source 1..40: mol_type = other DNA
ggaggcaaggttccgcaacgtaaccctaattatgggaatc

SEQ ID NO: 29 - DNA, 44 bp, synthetic construct
  misc_feature 1..44: note = Target Specific Oligonucleotide
  source 1..44: mol_type = other DNA
agaattccacgtaaatcacatgcgtaaccctaattatgggaatc

SEQ ID NO: 30 - DNA, 44 bp, synthetic construct
  misc_feature 1..44: note = Target Specific Oligonucleotide
  source 1..44: mol_type = other DNA
tatgcatgtacagggaagtatccgtaaccctaattatgggaatc

SEQ ID NO: 31 - DNA, 43 bp, synthetic construct
  misc_feature 1..43: note = Target Specific Oligonucleotide
  source 1..43: mol_type = other DNA
gtgtgcatgccttagaaaaagcgtaaccctaattatgggaatc

SEQ ID NO: 32 - DNA, 43 bp, synthetic construct
  misc_feature 1..43: note = Target Specific Oligonucleotide
  source 1..43: mol_type = other DNA
agcttttgtgcccttatcatgagtaaccctaattatgggaatc

SEQ ID NO: 33 - DNA, 46 bp, synthetic construct
  misc_feature 1..46: note = Target Specific Oligonucleotide
  source 1..46: mol_type = other DNA
cttcattagtatcttgtcacttggggtaaccctaattatgggaatc

SEQ ID NO: 34 - DNA, 45 bp, synthetic construct
  misc_feature 1..45: note = Target Specific Oligonucleotide
  source 1..45: mol_type = other DNA
agatgatgaccctaccaaatttgggtaaccctaattatgggaatc

SEQ ID NO: 35 - DNA, 44 bp, synthetic construct
  misc_feature 1..44: note = Target Specific Oligonucleotide
  source 1..44: mol_type = other DNA
aggcaagagatagatatgtgggcgtaaccctaattatgggaatc

SEQ ID NO: 36 - DNA, 44 bp, synthetic construct
  misc_feature 1..44: note = Target Specific Oligonucleotide
  source 1..44: mol_type = other DNA
cagctgccactataatcatgtttgtaaccctaattatgggaatc

SEQ ID NO: 37 - DNA, 45 bp, synthetic construct
  misc_feature 1..45: note = Target Specific Oligonucleotide
  source 1..45: mol_type = other DNA
ggaacatgctaatttaaggtgagtgtaaccctaattatgggaatc

SEQ ID NO: 38 - DNA, 46 bp, synthetic construct
  misc_feature 1..46: note = Target Specific Oligonucleotide
  source 1..46: mol_type = other DNA
aggactttgctatatcaagtagttcgtaaccctaattatgggaatc

SEQ ID NO: 39 - DNA, 46 bp, synthetic construct
  misc_feature 1..46: note = Target Specific Oligonucleotide
  source 1..46: mol_type = other DNA
aaaggagatagtaacaatggttttcgtaaccctaattatgggaatc

SEQ ID NO: 40 - DNA, 43 bp, synthetic construct
  misc_feature 1..43: note = Target Specific Oligonucleotide
  source 1..43: mol_type = other DNA
aaatcagtggttcaccctgttcgtaaccctaattatgggaatc

SEQ ID NO: 41 - DNA, 48 bp, synthetic construct
  misc_feature 1..48: note = Target Specific Oligonucleotide
  source 1..48: mol_type = other DNA
cttgtcataagataattaggcaaattagtaaccctaattatgggaatc

SEQ ID NO: 42 - DNA, 47 bp, synthetic construct
  misc_feature 1..47: note = Target Specific Oligonucleotide
  source 1..47: mol_type = other DNA
tcattaagttaatgctaaggatctttgtaaccctaattatgggaatc

SEQ ID NO: 43 - DNA, 43 bp, synthetic construct
  misc_feature 1..43: note = Target Specific Oligonucleotide
  source 1..43: mol_type = other DNA
tgtcactggtacaagtggacttgtaaccctaattatgggaatc

SEQ ID NO: 44 - DNA, 45 bp, synthetic construct
  misc_feature 1..45: note = Target Specific Oligonucleotide
  source 1..45: mol_type = other DNA
ttggtcactatacaagtgacttctgtaaccctaattatgggaatc

SEQ ID NO: 45 - DNA, 43 bp, synthetic construct
  misc_feature 1..43: note = Target Specific Oligonucleotide
  source 1..43: mol_type = other DNA
aggaaaatgggcgtaaaggagggtaaccctaattatgggaatc

SEQ ID NO: 46 - DNA, 46 bp, synthetic construct
  misc_feature 1..46: note = Target Specific Oligonucleotide
  source 1..46: mol_type = other DNA
ttcatctacaatagggacaacaaacgtaaccctaattatgggaatc

SEQ ID NO: 47 - DNA, 44 bp, synthetic construct
  misc_feature 1..44: note = Target Specific Oligonucleotide
  source 1..44: mol_type = other DNA
gataagtctacgtggaaaagcatgtaaccctaattatgggaatc

SEQ ID NO: 48 - DNA, 45 bp, synthetic construct
  misc_feature 1..45: note = Target Specific Oligonucleotide
  source 1..45: mol_type = other DNA
caggactacagttaagcatttactgtaaccctaattatgggaatc

SEQ ID NO: 49 - DNA, 43 bp, synthetic construct
  misc_feature 1..43: note = Target Specific Oligonucleotide
  source 1..43: mol_type = other DNA
gtctaggaaattgccgtggttggtaaccctaattatgggaatc

SEQ ID NO: 50 - DNA, 42 bp, synthetic construct
  misc_feature 1..42: note = Target Specific Oligonucleotide
  source 1..42: mol_type = other DNA
aatcatcgtctcgaaagcggtgtaaccctaattatgggaatc

SEQ ID NO: 51 - DNA, 43 bp, synthetic construct
  misc_feature 1..43: note = Target Specific Oligonucleotide
  source 1..43: mol_type = other DNA
cttgaccaaattcgaaggtccagtaaccctaattatgggaatc

SEQ ID NO: 52 - DNA, 43 bp, synthetic construct
  misc_feature 1..43: note = Target Specific Oligonucleotide
  source 1..43: mol_type = other DNA
gatgactctgttcgcatcctcggtaaccctaattatgggaatc

SEQ ID NO: 53 - DNA, 42 bp, synthetic construct
  misc_feature 1..42: note = Target Specific Oligonucleotide
  source 1..42: mol_type = other DNA
gatgctagtgtcaaacctgccgtaaccctaattatgggaatc

SEQ ID NO: 54 - DNA, 44 bp, synthetic construct
  misc_feature 1..44: note = Target Specific Oligonucleotide
  source 1..44: mol_type = other DNA
tgatacagggctcgtacttatccgtaaccctaattatgggaatc

SEQ ID NO: 55 - DNA, 43 bp, synthetic construct
  misc_feature 1..43: note = Target Specific Oligonucleotide
  source 1..43: mol_type = other DNA
aggagtcacacgagttgaaaaggtaaccctaattatgggaatc

SEQ ID NO: 56 - DNA, 44 bp, synthetic construct
  misc_feature 1..44: note = Target Specific Oligonucleotide
  source 1..44: mol_type = other DNA
ctcattacaccgaggtatgaagggtaaccctaattatgggaatc

SEQ ID NO: 57 - DNA, 40 bp, synthetic construct
  misc_feature 1..40: note = Target Specific Oligonucleotide
  source 1..40: mol_type = other DNA
ctctgtcacggctcgggtggtaaccctaattatgggaatc

SEQ ID NO: 58 - DNA, 43 bp, synthetic construct
  misc_feature 1..43: note = Target Specific Oligonucleotide
  source 1..43: mol_type = other DNA
ctaccatatgtacccgacctcagtaaccctaattatgggaatc

SEQ ID NO: 59 - DNA, 44 bp, synthetic construct
  misc_feature 1..44: note = Target Specific Oligonucleotide
  source 1..44: mol_type = other DNA
aaccttaggtcgggtatagagaggtaaccctaattatgggaatc

SEQ ID NO: 60 - DNA, 44 bp, synthetic construct
  misc_feature 1..44: note = Target Specific Oligonucleotide
  source 1..44: mol_type = other DNA
gaagcacttagacactgtaaggcgtaaccctaattatgggaatc

SEQ ID NO: 61 - DNA, 42 bp, synthetic construct
  misc_feature 1..42: note = Target Specific Oligonucleotide
  source 1..42: mol_type = other DNA
aataatcaccggcagtaacgggtaaccctaattatgggaatc

SEQ ID NO: 62 - DNA, 43 bp, synthetic construct
  misc_feature 1..43: note = Target Specific Oligonucleotide
  source 1..43: mol_type = other DNA
aggaagaaacctatggcagacagtaaccctaattatgggaatc

SEQ ID NO: 63 - DNA, 46 bp, synthetic construct
  misc_feature 1..46: note = Target Specific Oligonucleotide
  source 1..46: mol_type = other DNA
catgtgttattacgatgttctttgtgtaaccctaattatgggaatc

SEQ ID NO: 64 - DNA, 45 bp, synthetic construct
  misc_feature 1..45: note = Target Specific Oligonucleotide
  source 1..45: mol_type = other DNA
actactgtttggtacttgtatctggtaaccctaattatgggaatc

SEQ ID NO: 65 - DNA, 42 bp, synthetic construct
  misc_feature 1..42: note = Target Specific Oligonucleotide
  source 1..42: mol_type = other DNA
aaatcagtggcggacagtagcgtaaccctaattatgggaatc

SEQ ID NO: 66 - DNA, 41 bp, synthetic construct
  misc_feature 1..41: note = Target Specific Oligonucleotide
  source 1..41: mol_type = other DNA
caggcaatgccgactggattgtaaccctaattatgggaatc

SEQ ID NO: 67 - DNA, 48 bp, synthetic construct
  misc_feature 1..48: note = Target Specific Oligonucleotide
  source 1..48: mol_type = other DNA
tcttatctaagaccaactataggtatggtaaccctaattatgggaatc

SEQ ID NO: 68 - DNA, 43 bp, synthetic construct
  misc_feature 1..43: note = Target Specific Oligonucleotide
  source 1..43: mol_type = other DNA
gggaactgtctattgagcactcgtaaccctaattatgggaatc

SEQ ID NO: 69 - DNA, 47 bp, synthetic construct
  misc_feature 1..47: note = Target Specific Oligonucleotide
  source 1..47: mol_type = other DNA
attacaatctttagtactcatggaaagtaaccctaattatgggaatc

SEQ ID NO: 70 - DNA, 46 bp, synthetic construct
  misc_feature 1..46: note = Target Specific Oligonucleotide
  source 1..46: mol_type = other DNA
aaggtattctcatgcctagaatattgtaaccctaattatgggaatc

SEQ ID NO: 71 - DNA, 43 bp, synthetic construct
  misc_feature 1..43: note = Target Specific Oligonucleotide
  source 1..43: mol_type = other DNA
tggacagacacgttgtcatttggtaaccctaattatgggaatc

SEQ ID NO: 72 - DNA, 48 bp, synthetic construct
  misc_feature 1..48: note = Target Specific Oligonucleotide
  source 1..48: mol_type = other DNA
ctctcattacaatgctatacatttaacgtaaccctaattatgggaatc

SEQ ID NO: 73 - DNA, 41 bp, synthetic construct
  misc_feature 1..41: note = Target Specific Oligonucleotide
  source 1..41: mol_type = other DNA
tgcagctcgcaacggaacaagtaacggattttacaacttta

SEQ ID NO: 74 - DNA, 43 bp, synthetic construct
  misc_feature 1..43: note = Target Specific Oligonucleotide
  source 1..43: mol_type = other DNA
gagtccatccggacattgacctgtaacggattttacaacttta

SEQ ID NO: 75 - DNA, 41 bp, synthetic construct
  misc_feature 1..41: note = Target Specific Oligonucleotide
  source 1..41: mol_type = other DNA
cttgaacggcctcgacgagggtaacggattttacaacttta

SEQ ID NO: 76 - DNA, 43 bp, synthetic construct
  misc_feature 1..43: note = Target Specific Oligonucleotide
  source 1..43: mol_type = other DNA
cctcctcggttatattggccccgtaacggattttacaacttta

SEQ ID NO: 77 - DNA, 44 bp, synthetic construct
  misc_feature 1..44: note = Target Specific Oligonucleotide
  source 1..44: mol_type = other DNA
accgagacttcgaaaatgaacgagtaacggattttacaacttta

SEQ ID NO: 78 - DNA, 43 bp, synthetic construct
  misc_feature 1..43: note = Target Specific Oligonucleotide
  source 1..43: mol_type = other DNA
cctctggctcgaaactgaaagggtaacggattttacaacttta

SEQ ID NO: 79 - DNA, 44 bp, synthetic construct
  misc_feature 1..44: note = Target Specific Oligonucleotide
  source 1..44: mol_type = other DNA
tgctatggactagatctcggcaagtaacggattttacaacttta

SEQ ID NO: 80 - DNA, 42 bp, synthetic construct
  misc_feature 1..42: note = Target Specific Oligonucleotide
  source 1..42: mol_type = other DNA
gccttgaccggtcagaagacggtaacggattttacaacttta

SEQ ID NO: 81 - DNA, 42 bp, synthetic construct
  misc_feature 1..42: note = Target Specific Oligonucleotide
  source 1..42: mol_type = other DNA
taccacagagatcgcagctgcgtaacggattttacaacttta

SEQ ID NO: 82 - DNA, 40 bp, synthetic construct
  misc_feature 1..40: note = Target Specific Oligonucleotide
  source 1..40: mol_type = other DNA
accagcccgaacctcgcccgtaacggattttacaacttta

SEQ ID NO: 83 - DNA, 44 bp, synthetic construct
  misc_feature 1..44: note = Target Specific Oligonucleotide
  source 1..44: mol_type = other DNA
actgtatgcttgccggtaattctgtaacggattttacaacttta

SEQ ID NO: 84 - DNA, 41 bp, synthetic construct
  misc_feature 1..41: note = Target Specific Oligonucleotide
  source 1..41: mol_type = other DNA
gaacatgaaccggccccgacgtaacggattttacaacttta

SEQ ID NO: 85 - DNA, 42 bp, synthetic construct
  misc_feature 1..42: note = Target Specific Oligonucleotide
  source 1..42: mol_type = other DNA
caggaatcccgtcaagggtgggtaacggattttacaacttta

SEQ ID NO: 86 - DNA, 47 bp, synthetic construct
  misc_feature 1..47: note = Target Specific Oligonucleotide
  source 1..47: mol_type = other DNA
tctcgatttacctcgtttaagatctcgtaacggattttacaacttta

SEQ ID NO: 87 - DNA, 43 bp, synthetic construct
  misc_feature 1..43: note = Target Specific Oligonucleotide
  source 1..43: mol_type = other DNA
agaaccatggtcgaactgacctgtaacggattttacaacttta

SEQ ID NO: 88 - DNA, 41 bp, synthetic construct
  misc_feature 1..41: note = Target Specific Oligonucleotide
  source 1..41: mol_type = other DNA
tctgaggggctacgctgagagtaacggattttacaacttta

SEQ ID NO: 89 - DNA, 41 bp, synthetic construct
  misc_feature 1..41: note = Target Specific Oligonucleotide
  source 1..41: mol_type = other DNA
aagcctctgttacgcccacggtaacggattttacaacttta

SEQ ID NO: 90 - DNA, 46 bp, synthetic construct
  misc_feature 1..46: note = Target Specific Oligonucleotide
  source 1..46: mol_type = other DNA
aatgattcaactcgtactggatcccgtaacggattttacaacttta

SEQ ID NO: 91 - DNA, 44 bp, synthetic construct
  misc_feature 1..44: note = Target Specific Oligonucleotide
  source 1..44: mol_type = other DNA
cctcggaaatcccgattctgatagtaacggattttacaacttta

SEQ ID NO: 92 - DNA, 41 bp, synthetic construct
  misc_feature 1..41: note = Target Specific Oligonucleotide
  source 1..41: mol_type = other DNA
ggcccctactcggttgtggggtaacggattttacaacttta

SEQ ID NO: 93 - DNA, 45 bp, synthetic construct
  misc_feature 1..45: note = Target Specific Oligonucleotide
  source 1..45: mol_type = other DNA
ggaaaatggaacgaaactcctgttgtaacggattttacaacttta

SEQ ID NO: 94 - DNA, 40 bp, synthetic construct
  misc_feature 1..40: note = Target Specific Oligonucleotide
  source 1..40: mol_type = other DNA
ctgggtcgtgcgaggtcctgtaacggattttacaacttta

SEQ ID NO: 95 - DNA, 40 bp, synthetic construct
  misc_feature 1..40: note = Target Specific Oligonucleotide
  source 1..40: mol_type = other DNA
cacggggctaggacggggtgtaacggattttacaacttta

SEQ ID NO: 96 - DNA, 39 bp, synthetic construct
  misc_feature 1..39: note = Target Specific Oligonucleotide
  source 1..39: mol_type = other DNA
ccgatggcgtactcgtcgggtaacccttattcggtacta

SEQ ID NO: 97 - DNA, 42 bp, synthetic construct
  misc_feature 1..42: note = Target Specific Oligonucleotide
  source 1..42: mol_type = other DNA
gatcagatcagaatccgaggtggtaacccttattcggtacta

SEQ ID NO: 98 - DNA, 40 bp, synthetic construct
  misc_feature 1..40: note = Target Specific Oligonucleotide
  source 1..40: mol_type = other DNA
ggttgtactcgcgacagttggtaacccttattcggtacta

SEQ ID NO: 99 - DNA, 40 bp, synthetic construct
  misc_feature 1..40: note = Target Specific Oligonucleotide
  source 1..40: mol_type = other DNA
gacctgctcattcgaggtgagtaacccttattcggtacta

SEQ ID NO: 100 - DNA, 39 bp, synthetic construct
  misc_feature 1..39: note = Target Specific Oligonucleotide
  source 1..39: mol_type = other DNA
tcacgtagtagcggcttcggtaacccttattcggtacta

SEQ ID NO: 101 - DNA, 42 bp, synthetic construct
  misc_feature 1..42: note = Target Specific Oligonucleotide
  source 1..42: mol_type = other DNA
ccaaagatgggcctacttgtcagtaacccttattcggtacta

SEQ ID NO: 102 - DNA, 39 bp, synthetic construct
  misc_feature 1..39: note = Target Specific Oligonucleotide
  source 1..39: mol_type = other DNA
gacagagctgcacgcttgggtaacccttattcggtacta

SEQ ID NO: 103 - DNA, 39 bp, synthetic construct
  misc_feature 1..39: note = Target Specific Oligonucleotide
  source 1..39: mol_type = other DNA
ctgggcctgtttgcgctcagtaacccttattcggtacta

SEQ ID NO: 104 - DNA, 38 bp, synthetic construct
  misc_feature 1..38: note = Target Specific Oligonucleotide
  source 1..38: mol_type = other DNA
tccttaggcatgcgcggcgtaacccttattcggtacta

SEQ ID NO: 105 - DNA, 39 bp, synthetic construct
  misc_feature 1..39: note = Target Specific Oligonucleotide
  source 1..39: mol_type = other DNA
cagcggcagtactcgctgtgtaacccttattcggtacta

SEQ ID NO: 106 - DNA, 38 bp, synthetic construct
  misc_feature 1..38: note = Target Specific Oligonucleotide
  source 1..38: mol_type = other DNA
atgccaccgatgacccgcgtaacccttattcggtacta

SEQ ID NO: 107 - DNA, 38 bp, synthetic construct
  misc_feature 1..38: note = Target Specific Oligonucleotide
  source 1..38: mol_type = other DNA
atcagcgtcgctccctcggtaacccttattcggtacta

SEQ ID NO: 108 - DNA, 41 bp, synthetic construct
  misc_feature 1..41: note = Target Specific Oligonucleotide
  source 1..41: mol_type = other DNA
ctttgagagctcgaacatcgtgtaacccttattcggtacta

SEQ ID NO: 109 - DNA, 42 bp, synthetic construct
  misc_feature 1..42: note = Target Specific Oligonucleotide
  source 1..42: mol_type = other DNA
ttccagtagttaaggcagagcagtaacccttattcggtacta

SEQ ID NO: 110 - DNA, 39 bp, synthetic construct
  misc_feature 1..39: note = Target Specific Oligonucleotide
  source 1..39: mol_type = other DNA
gagctgcaggatcgggtccgtaacccttattcggtacta

SEQ ID NO: 111 - DNA, 41 bp, synthetic construct
  misc_feature 1..41: note = Target Specific Oligonucleotide
  source 1..41: mol_type = other DNA
acatgagtggtttcgtagcgggtaacccttattcggtacta

SEQ ID NO: 112 - DNA, 40 bp, synthetic construct
  misc_feature 1..40: note = Target Specific Oligonucleotide
  source 1..40: mol_type = other DNA
tgccgatgacatggaactcggtaacccttattcggtacta

SEQ ID NO: 113 - DNA, 43 bp, synthetic construct
  misc_feature 1..43: note = Target Specific Oligonucleotide
  source 1..43: mol_type = other DNA
tggtgtaattgaccttgtaggtagtaacccttattcggtacta

SEQ ID NO: 114 - DNA, 39 bp, synthetic construct
  misc_feature 1..39: note = Target Specific Oligonucleotide
  source 1..39: mol_type = other DNA
cattggccgaaagcaggctgtaacccttattcggtacta

SEQ ID NO: 115 - DNA, 40 bp, synthetic construct
  misc_feature 1..40: note = Target Specific Oligonucleotide
  source 1..40: mol_type = other DNA
gagacctctgccgaaactgggtaacccttattcggtacta

SEQ ID NO: 116 - DNA, 41 bp, synthetic construct
  misc_feature 1..41: note = Target Specific Oligonucleotide
  source 1..41: mol_type = other DNA
agtcttgtgcttcgggtcaaagtaacccttattcggtacta

SEQ ID NO: 117 - DNA, 41 bp, synthetic construct
  misc_feature 1..41: note = Target Specific Oligonucleotide
  source 1..41: mol_type = other DNA
cctcttctcgcctggcatagggtaacccttattcggtacta

SEQ ID NO: 118 - DNA, 41 bp, synthetic construct
  misc_feature 1..41: note = Target Specific Oligonucleotide
  source 1..41: mol_type = other DNA
gtaacggtgaaaatggaccgggtaacccttattcggtacta

SEQ ID NO: 119 - DNA, 42 bp, synthetic construct
  misc_feature 1..42: note = Target Specific Oligonucleotide
  source 1..42: mol_type = other DNA
tccctctaagggattaatgccagtaacccttattcggtacta

SEQ ID NO: 120 - DNA, 43 bp, synthetic construct
  misc_feature 1..43: note = Target Specific Oligonucleotide
  source 1..43: mol_type = other DNA
agttcaagtatcccgcgactagtaacaattagagtgaaatacc

SEQ ID NO: 121 - DNA, 43 bp, synthetic construct
  misc_feature 1..43: note = Target Specific Oligonucleotide
  source 1..43: mol_type = other DNA
gaacgcaggctgtttactgttgtaacaattagagtgaaatacc

SEQ ID NO: 122 - DNA, 44 bp, synthetic construct
  misc_feature 1..44: note = Target Specific Oligonucleotide
  source 1..44: mol_type = other DNA
ggtccaggtaaactaatggctggtaacaattagagtgaaatacc

SEQ ID NO: 123 - DNA, 44 bp, synthetic construct
  misc_feature 1..44: note = Target Specific Oligonucleotide
  source 1..44: mol_type = other DNA
ctctgttgagtttacctcgcaagtaacaattagagtgaaatacc

SEQ ID NO: 124 - DNA, 44 bp, synthetic construct
  misc_feature 1..44: note = Target Specific Oligonucleotide
  source 1..44: mol_type = other DNA
tacttcaactaaccagtccacggtaacaattagagtgaaatacc

SEQ ID NO: 125 - DNA, 44 bp, synthetic construct
  misc_feature 1..44: note = Target Specific Oligonucleotide
  source 1..44: mol_type = other DNA
ccatgagacaaggcttaagactgtaacaattagagtgaaatacc

SEQ ID NO: 126 - DNA, 43 bp, synthetic construct
  misc_feature 1..43: note = Target Specific Oligonucleotide
  source 1..43: mol_type = other DNA
cggccaaagaatagtcgtagcgtaacaattagagtgaaatacc

SEQ ID NO: 127 - DNA, 45 bp, synthetic construct
  misc_feature 1..45: note = Target Specific Oligonucleotide
  source 1..45: mol_type = other DNA
ccatttccctaaggtatgtgtgagtaacaattagagtgaaatacc

SEQ ID NO: 128 - DNA, 46 bp, synthetic construct
  misc_feature 1..46: note = Target Specific Oligonucleotide
  source 1..46: mol_type = other DNA
ctctcatactgttagtgatgtctggtaacaattagagtgaaatacc

SEQ ID NO: 129 - DNA, 42 bp, synthetic construct
  misc_feature 1..42: note = Target Specific Oligonucleotide
  source 1..42: mol_type = other DNA
cagctttgtcccgtgactgtgtaacaattagagtgaaatacc

SEQ ID NO: 130 - DNA, 41 bp, synthetic construct
  misc_feature 1..41: note = Target Specific Oligonucleotide
  source 1..41: mol_type = other DNA
tgctgcaatgctagcagcggtaacaattagagtgaaatacc

SEQ ID NO: 131 - DNA, 46 bp, synthetic construct
  misc_feature 1..46: note = Target Specific Oligonucleotide
  source 1..46: mol_type = other DNA
aagacaggaacctatcaatgtagtgtaacaattagagtgaaatacc

SEQ ID NO: 132 - DNA, 46 bp, synthetic construct
  misc_feature 1..46: note = Target Specific Oligonucleotide
  source 1..46: mol_type = other DNA
tagattagattatgcccaagtcaggtaacaattagagtgaaatacc

SEQ ID NO: 133 - DNA, 46 bp, synthetic construct
  misc_feature 1..46: note = Target Specific Oligonucleotide
  source 1..46: mol_type = other DNA
catgtaacccactttaggtttacagtaacaattagagtgaaatacc

SEQ ID NO: 134 - DNA, 47 bp, synthetic construct
  misc_feature 1..47: note = Target Specific Oligonucleotide
  source 1..47: mol_type = other DNA
ccagtctttcgtattaatgattcaggtaacaattagagtgaaatacc

SEQ ID NO: 135 - DNA, 42 bp, synthetic construct
  misc_feature 1..42: note = Target Specific Oligonucleotide
  source 1..42: mol_type = other DNA
tgcaacccgtctcgtcttcggtaacaattagagtgaaatacc

SEQ ID NO: 136 - DNA, 47 bp, synthetic construct
  misc_feature 1..47: note = Target Specific Oligonucleotide
  source 1..47: mol_type = other DNA
gtgtaggtatcatctgtaatgtacagtaacaattagagtgaaatacc

SEQ ID NO: 137 - DNA, 41 bp, synthetic construct
  misc_feature 1..41: note = Target Specific Oligonucleotide
  source 1..41: mol_type = other DNA
tcccggacttcagtaccgcgtaacaattagagtgaaatacc

SEQ ID NO: 138 - DNA, 41 bp, synthetic construct
  misc_feature 1..41: note = Target Specific Oligonucleotide
  source 1..41: mol_type = other DNA
cgaggaggttgcgaaaggcgtaacaattagagtgaaatacc

SEQ ID NO: 139 - DNA, 49 bp, synthetic construct
  misc_feature 1..49: note = Target Specific Oligonucleotide
  source 1..49: mol_type = other DNA
tatacatatactcaacacttatagagggtaacaattagagtgaaatacc

SEQ ID NO: 140 - DNA, 49 bp, synthetic construct
  misc_feature 1..49: note = Target Specific Oligonucleotide
  source 1..49: mol_type = other DNA
gtaatagataccataatttgtacttgggtaacaattagagtgaaatacc

SEQ ID NO: 141 - DNA, 40 bp, synthetic construct
  misc_feature 1..40: note = Target Specific Oligonucleotide
  source 1..40: mol_type = other DNA
tgctgctgcgctagacccgtaacaattagagtgaaatacc

SEQ ID NO: 142 - DNA, 40 bp, synthetic construct
  misc_feature 1..40: note = Target Specific Oligonucleotide
  source 1..40: mol_type = other DNA
gcgtggcgagccaaagacgtaacaattagagtgaaatacc

SEQ ID NO: 143 - DNA, 40 bp, synthetic construct
  misc_feature 1..40: note = Target Specific Oligonucleotide
  source 1..40: mol_type = other DNA
tccgcggttgttggacgggtaacaattagagtgaaatacc

SEQ ID NO: 144 - DNA, 41 bp, synthetic construct
  misc_feature 1..41: note = Target Specific Oligonucleotide
  source 1..41: mol_type = other DNA
ctgtcacatacgcaaactggtgtaacatcgttatagctaga

SEQ ID NO: 145 - DNA, 41 bp, synthetic construct
  misc_feature 1..41: note = Target Specific Oligonucleotide
  source 1..41: mol_type = other DNA
tcgccatataccggtcaaagagtaacatcgttatagctaga

SEQ ID NO: 146 - DNA, 42 bp, synthetic construct
  misc_feature 1..42: note = Target Specific Oligonucleotide
  source 1..42: mol_type = other DNA
tggatggtgcaataatccgagggtaacatcgttatagctaga

SEQ ID NO: 147 - DNA, 43 bp, synthetic construct
  misc_feature 1..43: note = Target Specific Oligonucleotide
  source 1..43: mol_type = other DNA
agtgctgatcccttaagtatgtcgtaacatcgttatagctaga

SEQ ID NO: 148 - DNA, 43 bp, synthetic construct
  misc_feature 1..43: note = Target Specific Oligonucleotide
  source 1..43: mol_type = other DNA
acctccattaaccaatccagaaggtaacatcgttatagctaga

SEQ ID NO: 149 - DNA, 43 bp, synthetic construct
  misc_feature 1..43: note = Target Specific Oligonucleotide
  source 1..43: mol_type = other DNA
tggtgtacttgacccacttatttgtaacatcgttatagctaga

SEQ ID NO: 150 - DNA, 41 bp, synthetic construct
  misc_feature 1..41: note = Target Specific Oligonucleotide
  source 1..41: mol_type = other DNA
cagaagagaacgtggagcagggtaacatcgttatagctaga

SEQ ID NO: 151 - DNA, 44 bp, synthetic construct
  misc_feature 1..44: note = Target Specific Oligonucleotide
  source 1..44: mol_type = other DNA
tccctgtgaagtttatagacttcagtaacatcgttatagctaga

SEQ ID NO: 152 - DNA, 40 bp, synthetic construct
  misc_feature 1..40: note = Target Specific Oligonucleotide
  source 1..40: mol_type = other DNA
tgtaaaagcaaacgcacgccgtaacatcgttatagctaga

SEQ ID NO: 153 - DNA, 44 bp, synthetic construct
  misc_feature 1..44: note = Target Specific Oligonucleotide
  source 1..44: mol_type = other DNA
attcagatgacgagaaatgatacagtaacatcgttatagctaga

SEQ ID NO: 154 - DNA, 39 bp, synthetic construct
  misc_feature 1..39: note = Target Specific Oligonucleotide
  source 1..39: mol_type = other DNA
tggtcacgccatttccggcgtaacatcgttatagctaga

SEQ ID NO: 155 - DNA, 45 bp, synthetic construct
  misc_feature 1..45: note = Target Specific Oligonucleotide
  source 1..45: mol_type = other DNA
agcaagtataccataaggaaattcagtaacatcgttatagctaga

SEQ ID NO: 156 - DNA, 40 bp, synthetic construct
  misc_feature 1..40: note = Target Specific Oligonucleotide
  source 1..40: mol_type = other DNA
ctcgccgtcctgtcgattttgtaacatcgttatagctaga

SEQ ID NO: 157 - DNA, 38 bp, synthetic construct
  misc_feature 1..38: note = Target Specific Oligonucleotide
  source 1..38: mol_type = other DNA
agcgagccgagaactccggtaacatcgttatagctaga

SEQ ID NO: 158 - DNA, 43 bp, synthetic construct
  misc_feature 1..43: note = Target Specific Oligonucleotide
  source 1..43: mol_type = other DNA
tattttgttcagacaacatggctgtaacatcgttatagctaga

SEQ ID NO: 159 - DNA, 40 bp, synthetic construct
  misc_feature 1..40: note = Target Specific Oligonucleotide
  source 1..40: mol_type = other DNA
ccacttggtacaacggagccgtaacatcgttatagctaga

SEQ ID NO: 160 - DNA, 40 bp, synthetic construct
  misc_feature 1..40: note = Target Specific Oligonucleotide
  source 1..40: mol_type = other DNA
cagaacacctgcgaggagaggtaacatcgttatagctaga

SEQ ID NO: 161 - DNA, 39 bp, synthetic construct
  misc_feature 1..39: note = Target Specific Oligonucleotide
  source 1..39: mol_type = other DNA
ttcatcagcgacgcccctggtaacatcgttatagctaga

SEQ ID NO: 162 - DNA, 41 bp, synthetic construct
  misc_feature 1..41: note = Target Specific Oligonucleotide
  source 1..41: mol_type = other DNA
cctgcaaaaaaacggtcacgtgtaacatcgttatagctaga

SEQ ID NO: 163 - DNA, 41 bp, synthetic construct
  misc_feature 1..41: note = Target Specific Oligonucleotide
  source 1..41: mol_type = other DNA
tgtgggagtcccttaggtcaagtaacatcgttatagctaga

SEQ ID NO: 164 - DNA, 38 bp, synthetic construct
  misc_feature 1..38: note = Target Specific Oligonucleotide
  source 1..38: mol_type = other DNA
gcatcgggagcacgcactgtaacatcgttatagctaga

SEQ ID NO: 165 - DNA, 39 bp, synthetic construct
  misc_feature 1..39: note = Target Specific Oligonucleotide
  source 1..39: mol_type = other DNA
ggctctgcacaacgcttgcgtaacatcgttatagctaga

SEQ ID NO: 166 - DNA, 41 bp, synthetic construct
  misc_feature 1..41: note = Target Specific Oligonucleotide
  source 1..41: mol_type = other DNA
agccaggacacaatagtcagggtaacatcgttatagctaga

SEQ ID NO: 167 - DNA, 41 bp, synthetic construct; identical to SEQ ID NO: 166
  misc_feature 1..41: note = Target Specific Oligonucleotide
  source 1..41: mol_type = other DNA

SEQ ID NO: 168 - DNA, 42 bp, synthetic construct
  misc_feature 1..42: note = Target Specific Oligonucleotide
  source 1..42: mol_type = other DNA
ttgcacatcctacggtcttctgtaacttactacggagttaac

SEQ ID NO: 169 - DNA, 43 bp, synthetic construct
  misc_feature 1..43: note = Target Specific Oligonucleotide
  source 1..43: mol_type = other DNA
gtagctcattcgcaaatcttgggtaacttactacggagttaac

SEQ ID NO: 170 - DNA, 41 bp, synthetic construct
  misc_feature 1..41: note = Target Specific Oligonucleotide
  source 1..41: mol_type = other DNA
cctttaagcgcgtcgtgtccgtaacttactacggagttaac

SEQ ID NO: 171 - DNA, 39 bp, synthetic construct
  misc_feature 1..39: note = Target Specific Oligonucleotide
  source 1..39: mol_type = other DNA
cctcgacgcatgatgccggtaacttactacggagttaac

SEQ ID NO: 172 - DNA, 40 bp, synthetic construct
  misc_feature 1..40: note = Target Specific Oligonucleotide
  source 1..40: mol_type = other DNA
gccccatggctcgtgtagggtaacttactacggagttaac

SEQ ID NO: 173 - DNA, 42 bp, synthetic construct
  misc_feature 1..42: note = Target Specific Oligonucleotide
  source 1..42: mol_type = other DNA
ccaattgtgttccggcaagttgtaacttactacggagttaac

SEQ ID NO: 174 - DNA, 43 bp, synthetic construct
  misc_feature 1..43: note = Target Specific Oligonucleotide
  source 1..43: mol_type = other DNA
gataggatttggtcggaaacctgtaacttactacggagttaac

SEQ ID NO: 175 - DNA, 41 bp, synthetic construct
  misc_feature 1..41: note = Target Specific Oligonucleotide
  source 1..41: mol_type = other DNA
tagttgccaacggtgttgtagtaacttactacggagttaac

SEQ ID NO: 176 - DNA, 42 bp, synthetic construct
  misc_feature 1..42: note = Target Specific Oligonucleotide
  source 1..42: mol_type = other DNA
cttggcctatgcggaagtaacgtaacttactacggagttaac

SEQ ID NO: 177 - DNA, 41 bp, synthetic construct
  misc_feature 1..41: note = Target Specific Oligonucleotide
  source 1..41: mol_type = other DNA
ctcgcagcgataggaaccatgtaacttactacggagttaac

SEQ ID NO: 178 - DNA, 44 bp, synthetic construct
  misc_feature 1..44: note = Target Specific Oligonucleotide
  source 1..44: mol_type = other DNA
tttgcattcgtccatatcaactggtaacttactacggagttaac

SEQ ID NO: 179 - DNA, 43 bp, synthetic construct
  misc_feature 1..43: note = Target Specific Oligonucleotide
  source 1..43: mol_type = other DNA
tcaatatcaacgggtaaaccgggtaacttactacggagttaac

SEQ ID NO: 180 - DNA, 45 bp, synthetic construct
  misc_feature 1..45: note = Target Specific Oligonucleotide
  source 1..45: mol_type = other DNA
ttgttactgacgtgggaaatattggtaacttactacggagttaac

SEQ ID NO: 181 - DNA, 40 bp, synthetic construct
  misc_feature 1..40: note = Target Specific Oligonucleotide
  source 1..40: mol_type = other DNA
tctccgctgatacccgggtgtaacttactacggagttaac

SEQ ID NO: 182 - DNA, 42 bp, synthetic construct
  misc_feature 1..42: note = Target Specific Oligonucleotide
  source 1..42: mol_type = other DNA
ggccagtcaccgaaatttcatgtaacttactacggagttaac

SEQ ID NO: 183 - DNA, 43 bp, synthetic construct
  misc_feature 1..43: note = Target Specific Oligonucleotide
  source 1..43: mol_type = other DNA
agttcgtagcctatctcacactgtaacttactacggagttaac

SEQ ID NO: 184 - DNA, 42 bp, synthetic construct
  misc_feature 1..42: note = Target Specific Oligonucleotide
  source 1..42: mol_type = other DNA
cacattcccgtacgtttgctggtaacttactacggagttaac

SEQ ID NO: 185 - DNA, 45 bp, synthetic construct
  misc_feature 1..45: note = Target Specific Oligonucleotide
  source 1..45: mol_type = other DNA
agtgagtccctatgtatcctttctgtaacttactacggagttaac

SEQ ID NO: 186 - DNA, 44 bp, synthetic construct
  misc_feature 1..44: note = Target Specific Oligonucleotide
  source 1..44: mol_type = other DNA
aagcattataacgtgatccacaggtaacttactacggagttaac

SEQ ID NO: 187 - DNA, 38 bp, synthetic construct
  misc_feature 1..38: note = Target Specific Oligonucleotide
  source 1..38: mol_type = other DNA
tgcgccctgaagcgcacgtaacttactacggagttaac

SEQ ID NO: 188 - DNA, 47 bp, synthetic construct
  misc_feature 1..47: note = Target Specific Oligonucleotide
  source 1..47: mol_type = other DNA
taaaaaatagcacgattacagtatacgtaacttactacggagttaac

SEQ ID NO: 189 - DNA, 44 bp, synthetic construct
  misc_feature 1..44: note = Target Specific Oligonucleotide
  source 1..44: mol_type = other DNA
catatcgacggattgagcctaacgtaacttactacggagttaac

SEQ ID NO: 190 - DNA, 42 bp, synthetic construct
  misc_feature 1..42: note = Target Specific Oligonucleotide
  source 1..42: mol_type = other DNA
cggtttctcgcgagagaaatagtaacttactacggagttaac

SEQ ID NO: 191 - DNA, 43 bp, synthetic construct
  misc_feature 1..43: note = Target Specific Oligonucleotide
  source 1..43: mol_type = other DNA
cttcccatctcgtgtaacatgagtaacttactacggagttaac

SEQ ID NO: 192 - DNA, 41 bp, synthetic construct
  misc_feature 1..41: note = Target Specific Oligonucleotide
  source 1..41: mol_type = other DNA
gcatttcacatcggactgtaccgtaacttcgaaacggaaac

SEQ ID NO: 193 - DNA, 38 bp, synthetic construct
  misc_feature 1..38: note = Target Specific Oligonucleotide
  source 1..38: mol_type = other DNA
tggaattctcggcggaccagtaacttcgaaacggaaac

SEQ ID NO: 194 - DNA, 41 bp, synthetic construct
  misc_feature 1..41: note = Target Specific Oligonucleotide
  source 1..41: mol_type = other DNA
gaagtggaaatacgacctttgcgtaacttcgaaacggaaac

SEQ ID NO: 195 - DNA, 39 bp, synthetic construct
  misc_feature 1..39: note = Target Specific Oligonucleotide
  source 1..39: mol_type = other DNA
gctggtcccttgcgtaacatgtaacttcgaaacggaaac

SEQ ID NO: 196 - DNA, 43 bp, synthetic construct
  misc_feature 1..43: note = Target Specific Oligonucleotide
  source 1..43: mol_type = other DNA
ttttcatctattcgagatgctcccgtaacttcgaaacggaaac

SEQ ID NO: 197 - DNA, 40 bp, synthetic construct
  misc_feature 1..40: note = Target Specific Oligonucleotide
  source 1..40: mol_type = other DNA
tattcatcccgtggggtagtagtaacttcgaaacggaaac

SEQ ID NO: 198 - DNA, 42 bp, synthetic construct
  misc_feature 1..42: note = Target Specific Oligonucleotide
  source 1..42: mol_type = other DNA
taaaaataggcccggatttgtctgtaacttcgaaacggaaac

SEQ ID NO: 199 - DNA, 42 bp, synthetic construct
  misc_feature 1..42: note = Target Specific Oligonucleotide
  source 1..42: mol_type = other DNA
tccaaccttgacgacaagatagggtaacttcgaaacggaaac

SEQ ID NO: 200 - DNA, 40 bp, synthetic construct
  misc_feature 1..40: note = Target Specific Oligonucleotide
  source 1..40: mol_type = other DNA
aggcaacagtcgaaccattctgtaacttcgaaacggaaac

SEQ ID NO: 201 - DNA, 40 bp, synthetic construct
  misc_feature 1..40: note = Target Specific Oligonucleotide
  source 1..40: mol_type = other DNA
gcttcatttgcgactgctcttgtaacttcgaaacggaaac

SEQ ID NO: 202 - DNA, 41 bp, synthetic construct
  misc_feature 1..41: note = Target Specific Oligonucleotide
  source 1..41: mol_type = other DNA
catcaggacgcttgtattggtggtaacttcgaaacggaaac

SEQ ID NO: 203 - DNA, 41 bp, synthetic construct
  misc_feature 1..41: note = Target Specific Oligonucleotide
  source 1..41: mol_type = other DNA
atcttcaatccgagaatccagcgtaacttcgaaacggaaac

SEQ ID NO: 204 - DNA, 42 bp, synthetic construct
  misc_feature 1..42: note = Target Specific Oligonucleotide
  source 1..42: mol_type = other DNA
ctgtagtgtggtagtaaggaagagtaacttcgaaacggaaac

SEQ ID NO: 205 - DNA, 42 bp, synthetic construct
  misc_feature 1..42: note = Target Specific Oligonucleotide
  source 1..42: mol_type = other DNA
gaacaccttacttacaacacctggtaacttcgaaacggaaac

SEQ ID NO: 206 - DNA, 43 bp, synthetic construct
  misc_feature 1..43: note = Target Specific Oligonucleotide
  source 1..43: mol_type = other DNA
gtacattgtacaccgttacaatgggtaacttcgaaacggaaac

SEQ ID NO: 207 - DNA, 42 bp, synthetic construct
  misc_feature 1..42: note = Target Specific Oligonucleotide
  source 1..42: mol_type = other DNA
tttcaagtcgtctatgttagctggtaacttcgaaacggaaac

SEQ ID NO: 208 - DNA, 44 bp, synthetic construct
  misc_feature 1..44: note = Target Specific Oligonucleotide
  source 1..44: mol_type = other DNA
caaagcaactatatgaagcttcattgtaacttcgaaacggaaac

SEQ ID NO: 209 - DNA, 42 bp, synthetic construct
  misc_feature 1..42: note = Target Specific Oligonucleotide
  source 1..42: mol_type = other DNA
catacttctatcgagagctcagggtaacttcgaaacggaaac

SEQ ID NO: 210 - DNA, 43 bp, synthetic construct
  misc_feature 1..43: note = Target Specific Oligonucleotide
  source 1..43: mol_type = other DNA
acgaagaaatcgagtaggtctagggtaacttcgaaacggaaac

SEQ ID NO: 211 - DNA, 41 bp, synthetic construct
  misc_feature 1..41: note = Target Specific Oligonucleotide
  source 1..41: mol_type = other DNA
gaatcattgagtcgacccttcagtaacttcgaaacggaaac

SEQ ID NO: 212 - DNA, 41 bp, synthetic construct
  misc_feature 1..41: note = Target Specific Oligonucleotide
  source 1..41: mol_type = other DNA
tgatgtgcttaagtagtgcagcgtaacttcgaaacggaaac

SEQ ID NO: 213 - DNA, 41 bp, synthetic construct
  misc_feature 1..41: note = Target Specific Oligonucleotide
  source 1..41: mol_type = other DNA
tccacggcatgatacatacaacgtaacttcgaaacggaaac

SEQ ID NO: 214 - DNA, 40 bp, synthetic construct
  misc_feature 1..40: note = Target Specific Oligonucleotide
  source 1..40: mol_type = other DNA
tctcagagcatcccgaatccagtaacttcgaaacggaaac

SEQ ID NO: 215 - DNA, 42 bp, synthetic construct
  misc_feature 1..42: note = Target Specific Oligonucleotide
  source 1..42: mol_type = other DNA
gtggaacgaagagtaggtagtttgtaacttcgaaacggaaac

SEQ ID NO: 216 - DNA, 39 bp, synthetic construct
  misc_feature 1..39: note = Target Specific Oligonucleotide
  source 1..39: mol_type = other DNA
gatcaatactggacggagtcaggtaacctcgtcggatca

SEQ ID NO: 217 - DNA, 38 bp, synthetic construct
  misc_feature 1..38: note = Target Specific Oligonucleotide
  source 1..38: mol_type = other DNA
gagcttgttactcgtgccttggtaacctcgtcggatca

SEQ ID NO: 218 - DNA, 37 bp, synthetic construct
  misc_feature 1..37: note = Target Specific Oligonucleotide
  source 1..37: mol_type = other DNA
cttcttacacttgcggacgcgtaacctcgtcggatca

SEQ ID NO: 219 - DNA, 41 bp, synthetic construct
  misc_feature 1..41: note = Target Specific Oligonucleotide
  source 1..41: mol_type = other DNA
caatacctattccgttacacacttgtaacctcgtcggatca

SEQ ID NO: 220 - DNA, 39 bp, synthetic construct
  misc_feature 1..39: note = Target Specific Oligonucleotide
  source 1..39: mol_type = other DNA
gctccttcagtccggttttattgtaacctcgtcggatca

SEQ ID NO: 221 - DNA, 36 bp, synthetic construct
  misc_feature 1..36: note = Target Specific Oligonucleotide
  source 1..36: mol_type = other DNA
gacgcacgagccgtgatctgtaacctcgtcggatca

SEQ ID NO: 222 - DNA, 41 bp, synthetic construct
  misc_feature 1..41: note = Target Specific Oligonucleotide
  source 1..41: mol_type = other DNA
gactgctaaggcataggaattttcgtaacctcgtcggatca

SEQ ID NO: 223 - DNA, 39 bp, synthetic construct
  misc_feature 1..39: note = Target Specific Oligonucleotide
  source 1..39: mol_type = other DNA
caccacataattacggggacacgtaacctcgtcggatca

SEQ ID NO: 224 - DNA, 34 bp, synthetic construct
  misc_feature 1..34: note = Target Specific Oligonucleotide
  source 1..34: mol_type = other DNA
cggcaaggcccttcgcagtaacctcgtcggatca

SEQ ID NO: 225 - DNA, 37 bp, synthetic construct
  misc_feature 1..37: note = Target Specific Oligonucleotide
  source 1..37: mol_type = other DNA
tccatctggtacgtggtggggtaacctcgtcggatca

SEQ ID NO: 226 - DNA, 38 bp, synthetic construct
  misc_feature 1..38: note = Target Specific Oligonucleotide
  source 1..38: mol_type = other DNA
gtcctgccgcgtatgatttctgtaacctcgtcggatca

SEQ ID NO: 227 - DNA, 37 bp, synthetic construct
  misc_feature 1..37: note = Target Specific Oligonucleotide
  source 1..37: mol_type = other DNA
tgttcaggctgacgactgcagtaacctcgtcggatca

SEQ ID NO: 228 - DNA, 36 bp, synthetic construct
  misc_feature 1..36: note = Target Specific Oligonucleotide
  source 1..36: mol_type = other DNA
gcatggaggtccgtcctgtgtaacctcgtcggatca

SEQ ID NO: 229 - DNA, 38 bp, synthetic construct
  misc_feature 1..38: note = Target Specific Oligonucleotide
  source 1..38: mol_type = other DNA
ttgagggagcgtaatcccaaggtaacctcgtcggatca

SEQ ID NO: 230 - DNA, 36 bp, synthetic construct
  misc_feature 1..36: note = Target Specific Oligonucleotide
  source 1..36: mol_type = other DNA
ccggcgtctgcgtacttccgtaacctcgtcggatca

SEQ ID NO: 231 - DNA, 39 bp, synthetic construct
  misc_feature 1..39: note = Target Specific Oligonucleotide
  source 1..39: mol_type = other DNA
cgactatctgcgtctatcatccgtaacctcgtcggatca

SEQ ID NO: 232 - DNA, 40 bp, synthetic construct
  misc_feature 1..40: note = Target Specific Oligonucleotide
  source 1..40: mol_type = other DNA
gagaattcgatgatcaactcacggtaacctcgtcggatca

SEQ ID NO: 233 - DNA, 35 bp, synthetic construct
  misc_feature 1..35: note = Target Specific Oligonucleotide
  source 1..35: mol_type = other DNA
tggcctgtcgtccggtctgtaacctcgtcggatca

SEQ ID NO: 234 - DNA, 38 bp, synthetic construct
  misc_feature 1..38: note = Target Specific Oligonucleotide
  source 1..38: mol_type = other DNA
cagggattccgtcatatggctgtaacctcgtcggatca

SEQ ID NO: 235 - DNA, 40 bp, synthetic construct
  misc_feature 1..40: note = Target Specific Oligonucleotide
  source 1..40: mol_type = other DNA
agacatcgatggtacatatgggtgtaacctcgtcggatca

SEQ ID NO: 236 - DNA, 37 bp, synthetic construct
  misc_feature 1..37: note = Target Specific Oligonucleotide
  source 1..37: mol_type = other DNA
tctgagctgtatcgctgcaagtaacctcgtcggatca

SEQ ID NO: 237 - DNA, 37 bp, synthetic construct
  misc_feature 1..37: note = Target Specific Oligonucleotide
  source 1..37: mol_type = other DNA
tcttaggcccattcgttggagtaacctcgtcggatca

SEQ ID NO: 238 - DNA, 37 bp, synthetic construct
  misc_feature 1..37: note = Target Specific Oligonucleotide
  source 1..37: mol_type = other DNA
ttatacaccgtgccgaacgcgtaacctcgtcggatca

SEQ ID NO: 239 - DNA, 37 bp, synthetic construct
  misc_feature 1..37: note = Target Specific Oligonucleotide
  source 1..37: mol_type = other DNA
atgccctttgcgatctgcacgtaacctcgtcggatca

SEQ ID NO: 240 - DNA, 42 bp, synthetic construct
  misc_feature 1..42: note = Target Specific Oligonucleotide
  source 1..42: mol_type = other DNA
ggcagaattccgaagttcagcgtaacataatcgtagtttcgg

SEQ ID NO: 241 - DNA, 42 bp, synthetic construct
  misc_feature 1..42: note = Target Specific Oligonucleotide
  source 1..42: mol_type = other DNA
gtttccttcacgacaggtgtggtaacataatcgtagtttcgg

SEQ ID NO: 242 - DNA, 44 bp, synthetic construct
  misc_feature 1..44: note = Target Specific Oligonucleotide
  source 1..44: mol_type = other DNA
ctggtagaaatgcgactaaagacgtaacataatcgtagtttcgg

SEQ ID NO: 243 - DNA, 44 bp, synthetic construct
  misc_feature 1..44: note = Target Specific Oligonucleotide
  source 1..44: mol_type = other DNA
tgaccagcatcgtttcatctaatgtaacataatcgtagtttcgg

SEQ ID NO: 244 - DNA, 42 bp, synthetic construct
  misc_feature 1..42: note = Target Specific Oligonucleotide
  source 1..42: mol_type = other DNA
ttcgaagttcaaccgagtgacgtaacataatcgtagtttcgg

SEQ ID NO: 245 - DNA, 42 bp, synthetic construct
  misc_feature 1..42: note = Target Specific Oligonucleotide
  source 1..42: mol_type = other DNA
ccaccaatccaatgcggaattgtaacataatcgtagtttcgg

SEQ ID NO: 246 - DNA, 42 bp, synthetic construct
  misc_feature 1..42: note = Target Specific Oligonucleotide
  source 1..42: mol_type = other DNA
agactcggtgccattcgtattgtaacataatcgtagtttcgg

SEQ ID NO: 247 - DNA, 42 bp, synthetic construct
  misc_feature 1..42: note = Target Specific Oligonucleotide
  source 1..42: mol_type = other DNA
ataactgctaactgcgcaaccgtaacataatcgtagtttcgg

SEQ ID NO: 248 - DNA, 43 bp, synthetic construct
  misc_feature 1..43: note = Target Specific Oligonucleotide
  source 1..43: mol_type = other DNA
gtagtgaggccgcttataaccagtaacataatcgtagtttcgg

SEQ ID NO: 249 - DNA, 43 bp, synthetic construct
  misc_feature 1..43: note = Target Specific Oligonucleotide
  source 1..43: mol_type = other DNA
ggtgatgattcgatggagtgaagtaacataatcgtagtttcgg

SEQ ID NO: 250 - DNA, 42 bp, synthetic construct
  misc_feature 1..42: note = Target Specific Oligonucleotide
  source 1..42: mol_type = other DNA
ctgctgctttacgtttggtgcgtaacataatcgtagtttcgg

SEQ ID NO: 251 - DNA, 43 bp, synthetic construct
  misc_feature 1..43: note = Target Specific Oligonucleotide
  source 1..43: mol_type = other DNA
tcatcaaccacgtctttggatagtaacataatcgtagtttcgg

SEQ ID NO: 252 - DNA, 44 bp, synthetic construct
  misc_feature 1..44: note = Target Specific Oligonucleotide
  source 1..44: mol_type = other DNA
aggcagatcttaaagtgttggttgtaacataatcgtagtttcgg

SEQ ID NO: 253 - DNA, 43 bp, synthetic construct
  misc_feature 1..43: note = Target Specific Oligonucleotide
  source 1..43: mol_type = other DNA
tgagggcttatacgaaagcaaggtaacataatcgtagtttcgg

SEQ ID NO: 254 - DNA, 44 bp, synthetic construct
  misc_feature 1..44: note = Target Specific Oligonucleotide
  source 1..44: mol_type = other DNA
gctcagtactgactttggtatgtgtaacataatcgtagtttcgg

SEQ ID NO: 255 - DNA, 45 bp, synthetic construct
  misc_feature 1..45: note = Target Specific Oligonucleotide
  source 1..45: mol_type = other DNA
cagtcaatcattagatccacatctgtaacataatcgtagtttcgg

SEQ ID NO: 256 - DNA, 40 bp, synthetic construct
  misc_feature 1..40: note = Target Specific Oligonucleotide
  source 1..40: mol_type = other DNA
ggaagctgctcgtcgaagcgtaacataatcgtagtttcgg

SEQ ID NO: 257 - DNA, 47 bp, synthetic construct
  misc_feature 1..47: note = Target Specific Oligonucleotide
  source 1..47: mol_type = other DNA
gagaagatacttatagcttcttgtctgtaacataatcgtagtttcgg

SEQ ID NO: 258 - DNA, 45 bp, synthetic construct
  misc_feature 1..45: note = Target Specific Oligonucleotide
  source 1..45: mol_type = other DNA
tcaatgctatctaactgatgaagagtaacataatcgtagtttcgg

SEQ ID NO: 259 - DNA, 46 bp, synthetic construct
  misc_feature 1..46: note = Target Specific Oligonucleotide
  source 1..46: mol_type = other DNA
atcttcccaattgatgtaagtacttgtaacataatcgtagtttcgg

SEQ ID NO: 260 - DNA, 40 bp, synthetic construct
  misc_feature 1..40: note = Target Specific Oligonucleotide
  source 1..40: mol_type = other DNA
catcacagtccgagacgccgtaacataatcgtagtttcgg

SEQ ID NO: 261 - DNA, 40 bp, synthetic construct
  misc_feature 1..40: note = Target Specific Oligonucleotide
  source 1..40: mol_type = other DNA
cattccaccggcctgtgcggtaacataatcgtagtttcgg

SEQ ID NO: 262 - DNA, 44 bp, synthetic construct
  misc_feature 1..44: note = Target Specific Oligonucleotide
  source 1..44: mol_type = other DNA
ggacagcactactctagagtaaggtaacataatcgtagtttcgg

SEQ ID NO: 263 - DNA, 43 bp, synthetic construct
  misc_feature 1..43: note = Target Specific Oligonucleotide
  source 1..43: mol_type = other DNA
tgccaagtaacttagcacacccgtaacataatcgtagtttcgg

SEQ ID NO: 264 - DNA, 38 bp, synthetic construct
  misc_feature 1..38: note = Target Specific Oligonucleotide
  source 1..38: mol_type = other DNA
gtgccacctttcaccgtgagtaaccggtagaattacgg

SEQ ID NO: 265 - DNA, 38 bp, synthetic construct
  misc_feature 1..38: note = Target Specific Oligonucleotide
  source 1..38: mol_type = other DNA
ccgcaggtacgacttgcctgtaaccggtagaattacgg

SEQ ID NO: 266 - DNA, 40 bp, synthetic construct
  misc_feature 1..40: note = Target Specific Oligonucleotide
  source 1..40: mol_type = other DNA
tgggcttcaatcagatggtcagtaaccggtagaattacgg

SEQ ID NO: 267 - DNA, 41 bp, synthetic construct
  misc_feature 1..41: note = Target Specific Oligonucleotide
  source 1..41: mol_type = other DNA
agaacaccagggtacgcatagtgtaaccggtagaattacgg

SEQ ID NO: 268 - DNA, 38 bp, synthetic construct
  misc_feature 1..38: note = Target Specific Oligonucleotide
  source 1..38: mol_type = other DNA
tctccgagagtgtcagcgggtaaccggtagaattacgg

SEQ ID NO: 269 - DNA, 38 bp, synthetic construct
  misc_feature 1..38: note = Target Specific Oligonucleotide
  source 1..38: mol_type = other DNA
gtcttcccggccggtcttggtaaccggtagaattacgg

SEQ ID NO: 270 - DNA, 39 bp, synthetic construct
  misc_feature 1..39: note = Target Specific Oligonucleotide
  source 1..39: mol_type = other DNA
catgttgcagattgtcgccagtaaccggtagaattacgg

SEQ ID NO: 271 - DNA, 39 bp, synthetic construct
  misc_feature 1..39: note = Target Specific Oligonucleotide
  source 1..39: mol_type = other DNA
ggttcagtcgtttgcgaacagtaaccggtagaattacgg

SEQ ID NO: 272 - DNA, 41 bp, synthetic construct
  misc_feature 1..41: note = Target Specific Oligonucleotide
  source 1..41: mol_type = other DNA
agctaccaattagacccactcggtaaccggtagaattacgg

SEQ ID NO: 273 - DNA, 40 bp, synthetic construct
  misc_feature 1..40: note = Target Specific Oligonucleotide
  source 1..40: mol_type = other DNA
cgtagttctcgtctccgatcagtaaccggtagaattacgg

SEQ ID NO: 274 - DNA, 39 bp, synthetic construct
  misc_feature 1..39: note = Target Specific Oligonucleotide
  source 1..39: mol_type = other DNA
ggagctgaacttgacgccaggtaaccggtagaattacgg

SEQ ID NO: 275 - DNA, 40 bp, synthetic construct
  misc_feature 1..40: note = Target Specific Oligonucleotide
  source 1..40: mol_type = other DNA
taatggatgtactcgttgggcgtaaccggtagaattacgg

SEQ ID NO: 276 - DNA, 38 bp, synthetic construct
  misc_feature 1..38: note = Target Specific Oligonucleotide
  source 1..38: mol_type = other DNA
tctgtgcatagccgtcccggtaaccggtagaattacgg

SEQ ID NO: 277 - DNA, 38 bp, synthetic construct
  misc_feature 1..38: note = Target Specific Oligonucleotide
  source 1..38: mol_type = other DNA
tggctggacggatcggatcgtaaccggtagaattacgg

SEQ ID NO: 278 - DNA, 37 bp, synthetic construct
  misc_feature 1..37: note = Target Specific Oligonucleotide
  source 1..37: mol_type = other DNA
ggtttgcgtcgttgcggcgtaaccggtagaattacgg

SEQ ID NO: 279 - DNA, 39 bp, synthetic construct
  misc_feature 1..39: note = Target Specific Oligonucleotide
  source 1..39: mol_type = other DNA
tctctggggacgtgacaaaggtaaccggtagaattacgg

SEQ ID NO: 280 - DNA, 42 bp, synthetic construct
  misc_feature 1..42: note = Target Specific Oligonucleotide
  source 1..42: mol_type = other DNA
gaattcatcagctagattggcaagtaaccggtagaattacgg

SEQ ID NO: 281 - DNA, 38 bp, synthetic construct
  misc_feature 1..38: note = Target Specific Oligonucleotide
  source 1..38: mol_type = other DNA
tcactccgtgggctaagcggtaaccggtagaattacgg

SEQ ID NO: 282 - DNA, 40 bp, synthetic construct
  misc_feature 1..40: note = Target Specific Oligonucleotide
  source 1..40: mol_type = other DNA
tgtaagggaacacggaagtgggtaaccggtagaattacgg

SEQ ID NO: 283 - DNA, 38 bp, synthetic construct
  misc_feature 1..38: note = Target Specific Oligonucleotide
  source 1..38: mol_type = other DNA
ctcaatggtggcgcggatcgtaaccggtagaattacgg

SEQ ID NO: 284 - DNA, 40 bp, synthetic construct
  misc_feature 1..40: note = Target Specific Oligonucleotide
  source 1..40: mol_type = other DNA
aaccactcaatctgcgtctcggtaaccggtagaattacgg

SEQ ID NO: 285 - DNA, 39 bp, synthetic construct
  misc_feature 1..39: note = Target Specific Oligonucleotide
  source 1..39: mol_type = other DNA
gctgtagggcgccattttgtgtaaccggtagaattacgg

SEQ ID NO: 286 - DNA, 38 bp, synthetic construct
  misc_feature 1..38: note = Target Specific Oligonucleotide
  source 1..38: mol_type = other DNA
tgagtgacggcattgcgcagtaaccggtagaattacgg

SEQ ID NO: 287 - DNA, 39 bp, synthetic construct
  misc_feature 1..39: note = Target Specific Oligonucleotide
  source 1..39: mol_type = other DNA
caaagtggctcatcgccaccgtaaccggtagaattacgg

SEQ ID NO: 288 - DNA, 45 bp, synthetic construct
  misc_feature 1..45: note = Target Specific Oligonucleotide
  source 1..45: mol_type = other DNA
tctatatgctaaatgtattgccatggtaacgcttctcataacact

SEQ ID NO: 289 - DNA, 43 bp, synthetic construct
  misc_feature 1..43: note = Target Specific Oligonucleotide
  source 1..43: mol_type = other DNA
ctatgaaacttcgtggtcactccgtaacgcttctcataacact

SEQ ID NO: 290 - DNA, 43 bp, synthetic construct
  misc_feature 1..43: note = Target Specific Oligonucleotide
  source 1..43: mol_type = other DNA
aactaactcatctgcagtaccatgtaacgcttctcataacact

SEQ ID NO: 291 - DNA, 43 bp, synthetic construct
  misc_feature 1..43: note = Target Specific Oligonucleotide
  source 1..43: mol_type = other DNA
aaaacctgcttgatccacattctgtaacgcttctcataacact

SEQ ID NO: 292 - DNA, 45 bp, synthetic construct
  misc_feature 1..45: note = Target Specific Oligonucleotide
  source 1..45: mol_type = other DNA
acattaacaaatcactcttgattcagtaacgcttctcataacact

SEQ ID NO: 293 - DNA, 43 bp, synthetic construct
  misc_feature 1..43: note = Target Specific Oligonucleotide
  source 1..43: mol_type = other DNA
ttttgcttagctcatggtaaacagtaacgcttctcataacact

SEQ ID NO: 294 - DNA, 45 bp, synthetic construct
  misc_feature 1..45: note = Target Specific Oligonucleotide
  source 1..45: mol_type = other DNA
gaggagctgttggataaataattttgtaacgcttctcataacact

SEQ ID NO: 295 - DNA, 43 bp, synthetic construct
  misc_feature 1..43: note = Target Specific Oligonucleotide
  source 1..43: mol_type = other DNA
aagcaggcaggtattgtatgattgtaacgcttctcataacact

SEQ ID NO: 296 - DNA, 45 bp, synthetic construct
  misc_feature 1..45: note = Target Specific Oligonucleotide
  source 1..45: mol_type = other DNA
gcaacttatatatcagtggtgaatggtaacgcttctcataacact

SEQ ID NO: 297 - DNA, 45 bp, synthetic construct
  misc_feature 1..45: note = Target Specific Oligonucleotide
  source 1..45: mol_type = other DNA
agattatgtatgcatgagaaccaacgtaacgcttctcataacact

SEQ ID NO: 298 - DNA, 45 bp, synthetic construct
  misc_feature 1..45: note = Target Specific Oligonucleotide
  source 1..45: mol_type = other DNA
gatcatgactctccttatggttaatgtaacgcttctcataacact

SEQ ID NO: 299 - DNA, 43 bp, synthetic construct
  misc_feature 1..43: note = Target Specific Oligonucleotide
  source 1..43: mol_type = other DNA
tgtgaaaccggtttataaacctagtaacgcttctcataacact

SEQ ID NO: 300 - DNA, 46 bp, synthetic construct
  misc_feature 1..46: note = Target Specific Oligonucleotide
  source 1..46: mol_type = other DNA
aataagattagctactgtctacagtggtaacgcttctcataacact

SEQ ID NO: 301 - DNA, 45 bp, synthetic construct
  misc_feature 1..45: note = Target Specific Oligonucleotide
  source 1..45: mol_type = other DNA
taaacaaatagtgatacatccacacgtaacgcttctcataacact

SEQ ID NO: 302 - DNA, 45 bp, synthetic construct
  misc_feature 1..45: note = Target Specific Oligonucleotide
  source 1..45: mol_type = other DNA
ttcagtcatctaacaatgtattcctgtaacgcttctcataacact

SEQ ID NO: 303 - DNA, 43 bp, synthetic construct
  misc_feature 1..43: note = Target Specific Oligonucleotide
  source 1..43: mol_type = other DNA
agctgtgggaaagtctttaactcgtaacgcttctcataacact

SEQ ID NO: 304 - DNA, 46 bp, synthetic construct
  misc_feature 1..46: note = Target Specific Oligonucleotide
  source 1..46: mol_type = other DNA
atagatgaactattgatgtacacaacgtaacgcttctcataacact

SEQ ID NO: 305 - DNA, 45 bp, synthetic construct
  misc_feature 1..45: note = Target Specific Oligonucleotide
  source 1..45: mol_type = other DNA
tacagatgaaattgagacctaaaacgtaacgcttctcataacact

SEQ ID NO: 306 - DNA, 45 bp, synthetic construct
  misc_feature 1..45: note = Target Specific Oligonucleotide
  source 1..45: mol_type = other DNA
agcaatttgggttaacagaaatagagtaacgcttctcataacact

SEQ ID NO: 307 - DNA, 46 bp, synthetic construct
  misc_feature 1..46: note = Target Specific Oligonucleotide
  source 1..46: mol_type = other DNA
aatccaatgggataagtactattagtgtaacgcttctcataacact

SEQ ID NO: 308 - DNA, 45 bp, synthetic construct
  misc_feature 1..45: note = Target Specific Oligonucleotide
  source 1..45: mol_type = other DNA
aatgttattccgtggaaaattacatgtaacgcttctcataacact

SEQ ID NO: 309 - DNA, 47 bp, synthetic construct
  misc_feature 1..47: note = Target Specific Oligonucleotide
  source 1..47: mol_type = other DNA
ttacagaattgtgtctgaaaaattatggtaacgcttctcataacact

SEQ ID NO: 310 - DNA, 40 bp, synthetic construct
  misc_feature 1..40: note = Target Specific Oligonucleotide
  source 1..40: mol_type = other DNA
atcagagaggcgctatgcctgtaacgcttctcataacact

SEQ ID NO: 311 - DNA, 38 bp, synthetic construct
  misc_feature 1..38: note = Target Specific Oligonucleotide
  source 1..38: mol_type = other DNA
cggcgacacgatacagcggtaacgcttctcataacact

SEQ ID NO: 312 - DNA, 40 bp, synthetic construct
  misc_feature 1..40: note = Target Specific Oligonucleotide
  source 1..40: mol_type = other DNA
cacttcaaatgcgcaacaagcgtaacgtcgctgaaaaatc

SEQ ID NO: 313 - DNA, 39 bp, synthetic construct
  misc_feature 1..39: note = Target Specific Oligonucleotide
  source 1..39: mol_type = other DNA
gtaagcactgcgcaagacaagtaacgtcgctgaaaaatc

SEQ ID NO: 314 - DNA, 41 bp, synthetic construct
  misc_feature 1..41: note = Target Specific Oligonucleotide
  source 1..41: mol_type = other DNA
gtgatgagctcgacaggatattgtaacgtcgctgaaaaatc

SEQ ID NO: 315 - DNA, 37 bp, synthetic construct
  misc_feature 1..37: note = Target Specific Oligonucleotide
  source 1..37: mol_type = other DNA
atggcgtcgtcggcacacgtaacgtcgctgaaaaatc

SEQ ID NO: 316 - DNA, 40 bp, synthetic construct
  misc_feature 1..40: note = Target Specific Oligonucleotide
  source 1..40: mol_type = other DNA
tttacaacagcgtggcaagtggtaacgtcgctgaaaaatc

SEQ ID NO: 317 - DNA, 40 bp, synthetic construct
  misc_feature 1..40: note = Target Specific Oligonucleotide
  source 1..40: mol_type = other DNA
aggcatgaagtgagacaatgcgtaacgtcgctgaaaaatc

SEQ ID NO: 318 - DNA, 41 bp, synthetic construct
  misc_feature 1..41: note = Target Specific Oligonucleotide
  source 1..41: mol_type = other DNA
cctggctagtggtatatgtcacgtaacgtcgctgaaaaatc

SEQ ID NO: 319 - DNA, 39 bp, synthetic construct
  misc_feature 1..39: note = Target Specific Oligonucleotide
  source 1..39: mol_type = other DNA
gctgatgatgttcaagcgcagtaacgtcgctgaaaaatc

SEQ ID NO: 320 - DNA, 41 bp, synthetic construct
  misc_feature 1..41: note = Target Specific Oligonucleotide
  source 1..41: mol_type = other DNA
ctgccagttagttaggcaagttgtaacgtcgctgaaaaatc

SEQ ID NO: 321 - DNA, 38 bp, synthetic construct
  misc_feature 1..38: note = Target Specific Oligonucleotide
  source 1..38: mol_type = other DNA
gcagctccgggctacaagtgtaacgtcgctgaaaaatc

SEQ ID NO: 322 - DNA, 40 bp, synthetic construct
  misc_feature 1..40: note = Target Specific Oligonucleotide
  source 1..40: mol_type = other DNA
tggcagctgtctaactggagcgtaacgtcgctgaaaaatc

SEQ ID NO: 323 - DNA, 38 bp, synthetic construct
  misc_feature 1..38: note = Target Specific Oligonucleotide
  source 1..38: mol_type = other DNA
acgcgtgtgcgagtagatggtaacgtcgctgaaaaatc

SEQ ID NO: 324 - DNA, 38 bp, synthetic construct
  misc_feature 1..38: note = Target Specific Oligonucleotide
  source 1..38: mol_type = other DNA
agctccacgaaggatgccagtaacgtcgctgaaaaatc

SEQ ID NO: 325 - DNA, 39 bp, synthetic construct
  misc_feature 1..39: note = Target Specific Oligonucleotide
  source 1..39: mol_type = other DNA
tgaggacggcatcgagatccgtaacgtcgctgaaaaatc

SEQ ID NO: 326 - DNA, 39 bp, synthetic construct
  misc_feature 1..39: note = Target Specific Oligonucleotide
  source 1..39: mol_type = other DNA
gtcttgagacccggtcttgggtaacgtcgctgaaaaatc

SEQ ID NO: 327 - DNA, 38 bp, synthetic construct
  misc_feature 1..38: note = Target Specific Oligonucleotide
  source 1..38: mol_type = other DNA
agtggtctggatcggtgcggtaacgtcgctgaaaaatc

SEQ ID NO: 328 - DNA, 39 bp, synthetic construct
  misc_feature 1..39: note = Target Specific Oligonucleotide
  source 1..39: mol_type = other DNA
tccattctgggtcgagtggagtaacgtcgctgaaaaatc

SEQ ID NO: 329 - DNA, 39 bp, synthetic construct
  misc_feature 1..39: note = Target Specific Oligonucleotide
  source 1..39: mol_type = other DNA
ggaccccaaggtgttccaaggtaacgtcgctgaaaaatc

SEQ ID NO: 330 - DNA, 38 bp, synthetic construct
  misc_feature 1..38: note = Target Specific Oligonucleotide
  source 1..38: mol_type = other DNA
agcagcttggctactccccgtaacgtcgctgaaaaatc

SEQ ID NO: 331 - DNA, 42 bp, synthetic construct
  misc_feature 1..42: note = Target Specific Oligonucleotide
  source 1..42: mol_type = other DNA
tttgggtatgggtactgtgtagagtaacgtcgctgaaaaatc

SEQ ID NO: 332 - DNA, 39 bp, synthetic construct
  misc_feature 1..39: note = Target Specific Oligonucleotide
  source 1..39: mol_type = other DNA
gccgcagggatagatccagggtaacgtcgctgaaaaatc

SEQ ID NO: 333 - DNA, 42 bp, synthetic construct
  misc_feature 1..42: note = Target Specific Oligonucleotide
  source 1..42: mol_type = other DNA
tgaagacagtcctatggacttccgtaacgtcgctgaaaaatc

SEQ ID NO: 334 - DNA, 37 bp, synthetic construct
  misc_feature 1..37: note = Target Specific Oligonucleotide
  source 1..37: mol_type = other DNA
aagctgaagcgcgggtcagtaacgtcgctgaaaaatc

SEQ ID NO: 335 - DNA, 37 bp, synthetic construct
  misc_feature 1..37: note = Target Specific Oligonucleotide
  source 1..37: mol_type = other DNA
ccacgcagcccttcgagagtaacgtcgctgaaaaatc

SEQ ID NO: 336 - DNA, 45 bp, synthetic construct
  misc_feature 1..45: note = Target Specific Oligonucleotide
  source 1..45: mol_type = other DNA
tagtaggtgtcgacaactagagcgtaactcttcaagattaatacc

SEQ ID NO: 337 - DNA, 44 bp, synthetic construct
  misc_feature 1..44: note = Target Specific Oligonucleotide
  source 1..44: mol_type = other DNA
gatggtcctgatcgagaaaccagtaactcttcaagattaatacc

SEQ ID NO: 338 - DNA, 44 bp, synthetic construct
  misc_feature 1..44: note = Target Specific Oligonucleotide
  source 1..44: mol_type = other DNA
aagaggacttcgctgaattgacgtaactcttcaagattaatacc

SEQ ID NO: 339 - DNA, 44 bp, synthetic construct
  misc_feature 1..44: note = Target Specific Oligonucleotide
  source 1..44: mol_type = other DNA
gaacctccgactgtatgtcagcgtaactcttcaagattaatacc

SEQ ID NO: 340 - DNA, 45 bp, synthetic construct
  misc_feature 1..45: note = Target Specific Oligonucleotide
  source 1..45: mol_type = other DNA
tctgaattagagcgatgttgacagtaactcttcaagattaatacc

SEQ ID NO: 341 - DNA, 41 bp, synthetic construct
  misc_feature 1..41: note = Target Specific Oligonucleotide
  source 1..41: mol_type = other DNA
gcattcctccgatcgcacagtaactcttcaagattaatacc

SEQ ID NO: 342 - DNA, 46 bp, synthetic construct
  misc_feature 1..46: note = Target Specific Oligonucleotide
  source 1..46: mol_type = other DNA
ctctatatttagctcgctgttcaagtaactcttcaagattaatacc

SEQ ID NO: 343 - DNA, 41 bp, synthetic construct
  misc_feature 1..41: note = Target Specific Oligonucleotide
  source 1..41: mol_type = other DNA
tattcatcacggcgcgcttgtaactcttcaagattaatacc

SEQ ID NO: 344 - DNA, 43 bp, synthetic construct
  misc_feature 1..43: note = Target Specific Oligonucleotide
  source 1..43: mol_type = other DNA
cattggggaccgtgcataaaagtaactcttcaagattaatacc

SEQ ID NO: 345 - DNA, 43 bp, synthetic construct
  misc_feature 1..43: note = Target Specific Oligonucleotide
  source 1..43: mol_type = other DNA
caaggggtgcactatttgggagtaactcttcaagattaatacc

SEQ ID NO: 346 - DNA, 43 bp, synthetic construct
  misc_feature 1..43: note = Target Specific Oligonucleotide
  source 1..43: mol_type = other DNA
ggaccgtatttcggcgaaatagtaactcttcaagattaatacc

SEQ ID NO: 347 - DNA, 43 bp, synthetic construct
  misc_feature 1..43: note = Target Specific Oligonucleotide
  source 1..43: mol_type = other DNA
atactgcacttgtcggcatgagtaactcttcaagattaatacc

SEQ ID NO: 348 - DNA, 43 bp, synthetic construct
  misc_feature 1..43: note = Target Specific Oligonucleotide
  source 1..43: mol_type = other DNA
atggcagatcgatccattggtgtaactcttcaagattaatacc

SEQ ID NO: 349 - DNA, 43 bp, synthetic construct
  misc_feature 1..43: note = Target Specific Oligonucleotide
  source 1..43: mol_type = other DNA
tccctttggaccgtcaagaaggtaactcttcaagattaatacc

SEQ ID NO: 350 - DNA, 42 bp, synthetic construct
  misc_feature 1..42: note = Target Specific Oligonucleotide
  source 1..42: mol_type = other DNA
aggcttgctgacatacgcaggtaactcttcaagattaatacc

SEQ ID NO: 351 - DNA, 42 bp, synthetic construct
  misc_feature 1..42: note = Target Specific Oligonucleotide
  source 1..42: mol_type = other DNA
cttgctttgtgcgaacacccgtaactcttcaagattaatacc

SEQ ID NO: 352 - DNA, 45 bp, synthetic construct
  misc_feature 1..45: note = Target Specific Oligonucleotide
  source 1..45: mol_type = other DNA
ctgatatcgaatgcaatggatgagtaactcttcaagattaatacc

SEQ ID NO: 353 - DNA, 43 bp, synthetic construct
  misc_feature 1..43: note = Target Specific Oligonucleotide
  source 1..43: mol_type = other DNA
taggactggtccgtcaaaaacgtaactcttcaagattaatacc

SEQ ID NO: 354 - DNA, 44 bp, synthetic construct
  misc_feature 1..44: note = Target Specific Oligonucleotide
  source 1..44: mol_type = other DNA
tgaccattctcgggacactaacgtaactcttcaagattaatacc

SEQ ID NO: 355 - DNA, 44 bp, synthetic construct
  misc_feature 1..44: note = Target Specific Oligonucleotide
  source 1..44: mol_type = other DNA
atgattgggtccgtaaaaatgcgtaactcttcaagattaatacc

SEQ ID NO: 356 - DNA, 45 bp, synthetic construct
  misc_feature 1..45: note = Target Specific Oligonucleotide
  source 1..45: mol_type = other DNA
gtttcatgtatggtaggaccaccgtaactcttcaagattaatacc

SEQ ID NO: 357 - DNA, 43 bp, synthetic construct
  misc_feature 1..43: note = Target Specific Oligonucleotide
  source 1..43: mol_type = other DNA
ctcttgcatcgtagcgaactagtaactcttcaagattaatacc

SEQ ID NO: 358 - DNA, 43 bp, synthetic construct
  misc_feature 1..43: note = Target Specific Oligonucleotide
  source 1..43: mol_type = other DNA
atgatgattccctcggtcagagtaactcttcaagattaatacc

SEQ ID NO: 359 - DNA, 46 bp, synthetic construct
  misc_feature 1..46: note = Target Specific Oligonucleotide
  source 1..46: mol_type = other DNA
agtagagaagatcgctgatatccggtaactcttcaagattaatacc

SEQ ID NO: 360 - DNA, 42 bp, synthetic construct
  misc_feature 1..42: note = Target Specific Oligonucleotide
  source 1..42: mol_type = other DNA
aacccttgacatcgccagtttgtaacgtaccgtttgtatatg

SEQ ID NO: 361 - DNA, 45 bp, synthetic construct
  misc_feature 1..45: note = Target Specific Oligonucleotide
  source 1..45: mol_type = other DNA
gaaggttaaacgagatttccaaaggtaacgtaccgtttgtatatg

SEQ ID NO: 362 - DNA, 41 bp, synthetic construct
  misc_feature 1..41: note = Target Specific Oligonucleotide
  source 1..41: mol_type = other DNA
gccactcgacatttctgccggtaacgtaccgtttgtatatg

SEQ ID NO: 363 - DNA, 42 bp, synthetic construct
  misc_feature 1..42: note = Target Specific Oligonucleotide
  source 1..42: mol_type = other DNA
tcatactgtccgcaacatccggtaacgtaccgtttgtatatg

SEQ ID NO: 364 - DNA, 41 bp, synthetic construct; identical to SEQ ID NO: 362
  misc_feature 1..41: note = Target Specific Oligonucleotide
  source 1..41: mol_type = other DNA

SEQ ID NO: 365 - DNA, 42 bp, synthetic construct; identical to SEQ ID NO: 363
  misc_feature 1..42: note = Target Specific Oligonucleotide
  source 1..42: mol_type = other DNA

SEQ ID NO: 366 - DNA, 43 bp, synthetic construct
  misc_feature 1..43: note = Target Specific Oligonucleotide
  source 1..43: mol_type = other DNA
tgaattttgcccgaacttcactgtaacgtaccgtttgtatatg

SEQ ID NO: 367 - DNA, 38 bp, synthetic construct
  misc_feature 1..38: note = Target Specific Oligonucleotide
  source 1..38: mol_type = other DNA
acacactcggagcgcgcgtaacgtaccgtttgtatatg

SEQ ID NO: 368 - DNA, 43 bp, synthetic construct
  misc_feature 1..43: note = Target Specific Oligonucleotide
  source 1..43: mol_type = other DNA
caaacctatatgcccgttgactgtaacgtaccgtttgtatatg

SEQ ID NO: 369 - DNA, 44 bp, synthetic construct
  misc_feature 1..44: note = Target Specific Oligonucleotide
  source 1..44: mol_type = other DNA
caggaacctttaccatgttcatggtaacgtaccgtttgtatatg

SEQ ID NO: 370 - DNA, 46 bp, synthetic construct
  misc_feature 1..46: note = Target Specific Oligonucleotide
  source 1..46: mol_type = other DNA
gaaaatctctacggatgaatttcttgtaacgtaccgtttgtatatg

SEQ ID NO: 371 - DNA, 43 bp, synthetic construct
  misc_feature 1..43: note = Target Specific Oligonucleotide
  source 1..43: mol_type = other DNA
gctgctaagatagccttgtgaggtaacgtaccgtttgtatatg

SEQ ID NO: 372 - DNA, 42 bp, synthetic construct
  misc_feature 1..42: note = Target Specific Oligonucleotide
  source 1..42: mol_type = other DNA
tggcactaaaaaccggagaacgtaacgtaccgtttgtatatg

SEQ ID NO: 373 - DNA, 39 bp, synthetic construct
  misc_feature 1..39: note = Target Specific Oligonucleotide
  source 1..39: mol_type = other DNA
tgcgggaccgccgatacagtaacgtaccgtttgtatatg

SEQ ID NO: 374 - DNA, 45 bp, synthetic construct
  misc_feature 1..45: note = Target Specific Oligonucleotide
  source 1..45: mol_type = other DNA
ggaaattaaacggagtcttacaacgtaacgtaccgtttgtatatg

SEQ ID NO: 375 - DNA, 40 bp, synthetic construct
  misc_feature 1..40: note = Target Specific Oligonucleotide
  source 1..40: mol_type = other DNA
agccagagagcggtatgccgtaacgtaccgtttgtatatg

SEQ ID NO: 376 - DNA, 41 bp, synthetic construct
  misc_feature 1..41: note = Target Specific Oligonucleotide
  source 1..41: mol_type = other DNA
ccaccaagtggggatgtgacgtaacgtaccgtttgtatatg

SEQ ID NO: 377 - DNA, 42 bp, synthetic construct
  misc_feature 1..42: note = Target Specific Oligonucleotide
  source 1..42: mol_type = other DNA
cacacatgacctttaagcgctgtaacgtaccgtttgtatatg

SEQ ID NO: 378 - DNA, 41 bp, synthetic construct
  misc_feature 1..41: note = Target Specific Oligonucleotide
  source 1..41: mol_type = other DNA
tccacagattgcgctgtctagtaacgtaccgtttgtatatg

SEQ ID NO: 379 - DNA, 41 bp, synthetic construct
  misc_feature 1..41: note = Target Specific Oligonucleotide
  source 1..41: mol_type = other DNA
ctccacggacaggttactgcgtaacgtaccgtttgtatatg

SEQ ID NO: 380 - DNA, 39 bp, synthetic construct
  misc_feature 1..39: note = Target Specific Oligonucleotide
  source 1..39: mol_type = other DNA
gcgtcatggcgtcagcacgtaacgtaccgtttgtatatg

SEQ ID NO: 381 - DNA, 39 bp, synthetic construct
  misc_feature 1..39: note = Target Specific Oligonucleotide
  source 1..39: mol_type = other DNA
tcctggccggcaacacacgtaacgtaccgtttgtatatg

SEQ ID NO: 382 - DNA, 42 bp, synthetic construct
  misc_feature 1..42: note = Target Specific Oligonucleotide
  source 1..42: mol_type = other DNA
agccattttgtcgaggtttgggtaacgtaccgtttgtatatg

SEQ ID NO: 383 - DNA, 44 bp, synthetic construct
  misc_feature 1..44: note = Target Specific Oligonucleotide
  source 1..44: mol_type = other DNA
catccgaagcatgatagttgatggtaacgtaccgtttgtatatg

SEQ ID NO: 384 - DNA, 43 bp, synthetic construct
  misc_feature 1..43: note = Target Specific Oligonucleotide
  source 1..43: mol_type = other DNA
tagtgtgttgcgtatcaagtatctagacgatgcgaattaacac

SEQ ID NO: 385 - DNA, 45 bp, synthetic construct
  misc_feature 1..45: note = Target Specific Oligonucleotide
  source 1..45: mol_type = other DNA
ttattgtgaactctagacatgagagtagacgatgcgaattaacac

SEQ ID NO: 386 - DNA, 46 bp, synthetic construct
  misc_feature 1..46: note = Target Specific Oligonucleotide
  source 1..46: mol_type = other DNA
acagtgaatactaagactgtaaaaactagacgatgcgaattaacac

SEQ ID NO: 387 - DNA, 41 bp, synthetic construct
  misc_feature 1..41: note = Target Specific Oligonucleotide
  source 1..41: mol_type = other DNA
acacaaggcacgtagaaacagtagacgatgcgaattaacac

SEQ ID NO: 388 - DNA, 41 bp, synthetic construct
  misc_feature 1..41: note = Target Specific Oligonucleotide
  source 1..41: mol_type = other DNA
caatggacagcggttgtgaaatagacgatgcgaattaacac

SEQ ID NO: 389 - DNA, 42 bp, synthetic construct
  misc_feature 1..42: note = Target Specific Oligonucleotide
  source 1..42: mol_type = other DNA
caccatcacctatcgacagagttagacgatgcgaattaacac

SEQ ID NO: 390 - DNA, 42 bp, synthetic construct
  misc_feature 1..42: note = Target Specific Oligonucleotide
  source 1..42: mol_type = other DNA
cattgcattacgttccacatggtagacgatgcgaattaacac

SEQ ID NO: 391 - DNA, 41 bp, synthetic construct
  misc_feature 1..41: note = Target Specific Oligonucleotide
  source 1..41: mol_type = other DNA
acacagagtgtccgatacccatagacgatgcgaattaacac

SEQ ID NO: 392 - DNA, 43 bp, synthetic construct
  misc_feature 1..43: note = Target Specific Oligonucleotide
  source 1..43: mol_type = other DNA
ggtgattacattgagtgctaggatagacgatgcgaattaacac

SEQ ID NO: 393 - DNA, 41 bp, synthetic construct
  misc_feature 1..41: note = Target Specific Oligonucleotide
  source 1..41: mol_type = other DNA
tccacataagcgttcaaggtatagacgatgcgaattaacac

SEQ ID NO: 394 - DNA, 44 bp, synthetic construct
  misc_feature 1..44: note = Target Specific Oligonucleotide
  source 1..44: mol_type = other DNA
cagaataatcgctattcctagctgtagacgatgcgaattaacac

SEQ ID NO: 395 - DNA, 40 bp, synthetic construct
  misc_feature 1..40: note = Target Specific Oligonucleotide
  source 1..40: mol_type = other DNA
ttacactcatacgtcgccggtagacgatgcgaattaacac

SEQ ID NO: 396 - DNA, 43 bp, synthetic construct
  misc_feature 1..43: note = Target Specific Oligonucleotide
  source 1..43: mol_type = other DNA
tggccataaaggctacttacaattagacgatgcgaattaacac

SEQ ID NO: 397 - DNA, 45 bp, synthetic construct
  misc_feature 1..45: note = Target Specific Oligonucleotide
  source 1..45: mol_type = other DNA
tttcaagttattcgatctgctaacctagacgatgcgaattaacac

SEQ ID NO: 398 - DNA, 45 bp, synthetic construct
  misc_feature 1..45: note = Target Specific Oligonucleotide
  source 1..45: mol_type = other DNA
agttagtcaaggactttactaaggttagacgatgcgaattaacac

SEQ ID NO: 399 - DNA, 42 bp, synthetic construct
  misc_feature 1..42: note = Target Specific Oligonucleotide
  source 1..42: mol_type = other DNA
ttacctcaaatacgggctaccatagacgatgcgaattaacac

SEQ ID NO: 400 - DNA, 47 bp, synthetic construct
  misc_feature 1..47: note = Target Specific Oligonucleotide
  source 1..47: mol_type = other DNA
gagttatattactctaactaaagccagtagacgatgcgaattaacac

SEQ ID NO: 401 - DNA, 43 bp, synthetic construct
  misc_feature 1..43: note = Target Specific Oligonucleotide
  source 1..43: mol_type = other DNA
ttgtcaacacgcataaaatctgctagacgatgcgaattaacac

SEQ ID NO: 402 - DNA, 43 bp, synthetic construct
  misc_feature 1..43: note = Target Specific Oligonucleotide
  source 1..43: mol_type = other DNA
tcatatacaatcggggatctgagtagacgatgcgaattaacac

SEQ ID NO: 403 - DNA, 43 bp, synthetic construct
  misc_feature 1..43: note = Target Specific Oligonucleotide
  source 1..43: mol_type = other DNA
cccacaaaaatacccgtaagttatagacgatgcgaattaacac

SEQ ID NO: 404 - DNA, 47 bp, synthetic construct
  misc_feature 1..47: note = Target Specific Oligonucleotide
  source 1..47: mol_type = other DNA
cactaaattattacgaattttgcaaagtagacgatgcgaattaacac

SEQ ID NO: 405 - DNA, 45 bp, synthetic construct
  misc_feature 1..45: note = Target Specific Oligonucleotide
  source 1..45: mol_type = other DNA
tcaaataaaaaccgtcaaaagtttatagacgatgcgaattaacac

SEQ ID NO: 406 - DNA, 42 bp, synthetic construct
  misc_feature 1..42: note = Target Specific Oligonucleotide
  source 1..42: mol_type = other DNA
agtgcaatggtatcacgtactgtagacgatgcgaattaacac

SEQ ID NO: 407 - DNA, 43 bp, synthetic construct
  misc_feature 1..43: note = Target Specific Oligonucleotide
  source 1..43: mol_type = other DNA
atgtgtcttgcaattggattccctagacgatgcgaattaacac

SEQ ID NO: 408 - DNA, 38 bp, synthetic construct
  misc_feature 1..38: note = Target Specific Oligonucleotide
  source 1..38: mol_type = other DNA
ggtgtgcgcgtcgtacactagacccgttataagtgttg

SEQ ID NO: 409 - DNA, 40 bp, synthetic construct
  misc_feature 1..40: note = Target Specific Oligonucleotide
  source 1..40: mol_type = other DNA
gtgtttagggtcgcggttgatagacccgttataagtgttg

SEQ ID NO: 410 - DNA, 42 bp, synthetic construct
  misc_feature 1..42: note = Target Specific Oligonucleotide
  source 1..42: mol_type = other DNA
gagaatggcgaagtaaatgccctagacccgttataagtgttg

SEQ ID NO: 411 - DNA, 42 bp, synthetic construct
  misc_feature 1..42: note = Target Specific Oligonucleotide
  source 1..42: mol_type = other DNA
gtagatggaatagacacggctgtagacccgttataagtgttg

SEQ ID NO: 412 - DNA, 43 bp, synthetic construct
  misc_feature 1..43: note = Target Specific Oligonucleotide
  source 1..43: mol_type = other DNA
gctcttaatgcatggtacaactgtagacccgttataagtgttg

SEQ ID NO: 413 - DNA, 43 bp, synthetic construct
  misc_feature 1..43: note = Target Specific Oligonucleotide
  source 1..43: mol_type = other DNA
aaaccagtatttcgtcacagtgatagacccgttataagtgttg

SEQ ID NO: 414 - DNA, 42 bp, synthetic construct
  misc_feature 1..42: note = Target Specific Oligonucleotide
  source 1..42: mol_type = other DNA
gatgggaacggtgtagagatgttagacccgttataagtgttg

SEQ ID NO: 415 - DNA, 42 bp, synthetic construct
  misc_feature 1..42: note = Target Specific Oligonucleotide
  source 1..42: mol_type = other DNA
ttccaaatgccgtcaaaactgttagacccgttataagtgttg

SEQ ID NO: 416 - DNA, 39 bp, synthetic construct
  misc_feature 1..39: note = Target Specific Oligonucleotide
  source 1..39: mol_type = other DNA
tgggtcacagacggtgtggtagacccgttataagtgttg

SEQ ID NO: 417 - DNA, 46 bp, synthetic construct
  misc_feature 1..46: note = Target Specific Oligonucleotide
  source 1..46: mol_type = other DNA
aatcaggtatacttctatccttgaaatagacccgttataagtgttg

SEQ ID NO: 418 - DNA, 43 bp, synthetic construct
  misc_feature 1..43: note = Target Specific Oligonucleotide
  source 1..43: mol_type = other DNA
tgctgtattagcaacttggaacttagacccgttataagtgttg

SEQ ID NO: 419 - DNA, 42 bp, synthetic construct
  misc_feature 1..42: note = Target Specific Oligonucleotide
  source 1..42: mol_type = other DNA
gccaataaagcgatggttgatctagacccgttataagtgttg

SEQ ID NO: 420 - DNA, 43 bp, synthetic construct
  misc_feature 1..43: note = Target Specific Oligonucleotide
  source 1..43: mol_type = other DNA
gtaaggatcactggatcctactgtagacccgttataagtgttg

SEQ ID NO: 421 - DNA, 43 bp, synthetic construct
  misc_feature 1..43: note = Target Specific Oligonucleotide
  source 1..43: mol_type = other DNA
gacctcattcagttgatgagaggtagacccgttataagtgttg

SEQ ID NO: 422 - DNA, 41 bp, synthetic construct
  misc_feature 1..41: note = Target Specific Oligonucleotide
  source 1..41: mol_type = other DNA
gcataacagacggttgcaagttagacccgttataagtgttg

SEQ ID NO: 423 - DNA, 41 bp, synthetic construct
  misc_feature 1..41: note = Target Specific Oligonucleotide
  source 1..41: mol_type = other DNA
acctaaagcgagttgctgagttagacccgttataagtgttg

SEQ ID NO: 424 - DNA, 41 bp, synthetic construct
  misc_feature 1..41: note = Target Specific Oligonucleotide
  source 1..41: mol_type = other DNA
gagtcagaaacacgcatggaatagacccgttataagtgttg

SEQ ID NO: 425 - DNA, 42 bp, synthetic construct
  misc_feature 1..42: note = Target Specific Oligonucleotide
  source 1..42: mol_type = other DNA
agcaactctgataggctcacactagacccgttataagtgttg

SEQ ID NO: 426 - DNA, 44 bp, synthetic construct
  misc_feature 1..44: note = Target Specific Oligonucleotide
  source 1..44: mol_type = other DNA
ccatacccactacggataaagatgtagacccgttataagtgttg

SEQ ID NO: 427 - DNA, 42 bp, synthetic construct
  misc_feature 1..42: note = Target Specific Oligonucleotide
  source 1..42: mol_type = other DNA
gctttctgtacgactcaggttttagacccgttataagtgttg

SEQ ID NO: 428 - DNA, 42 bp, synthetic construct
  misc_feature 1..42: note = Target Specific Oligonucleotide
  source 1..42: mol_type = other DNA
caatgttgagccactaaaccactagacccgttataagtgttg

SEQ ID NO: 429 - DNA, 42 bp, synthetic construct
  misc_feature 1..42: note = Target Specific Oligonucleotide
  source 1..42: mol_type = other DNA
atgcttggattaggtccaaagctagacccgttataagtgttg

SEQ ID NO: 430 - DNA, 41 bp, synthetic construct
  misc_feature 1..41: note = Target Specific Oligonucleotide
  source 1..41: mol_type = other DNA
cccggtgatggattagtttggtagacccgttataagtgttg

SEQ ID NO: 431 - DNA, 41 bp, synthetic construct
  misc_feature 1..41: note = Target Specific Oligonucleotide
  source 1..41: mol_type = other DNA
ccatggccataccctggaatttagacccgttataagtgttg

SEQ ID NO: 432 - DNA, 41 bp, synthetic construct
  misc_feature 1..41: note = Target Specific Oligonucleotide
  source 1..41: mol_type = other DNA
attatactgtccagcgtaggtggtagacaaccgtcgttaag

SEQ ID NO: 433 - DNA, 41 bp, synthetic construct
  misc_feature 1..41: note = Target Specific Oligonucleotide
  source 1..41: mol_type = other DNA
tcctggtaaggataggtaccatgtagacaaccgtcgttaag

SEQ ID NO: 434 - DNA, 41 bp, synthetic construct
  misc_feature 1..41: note = Target Specific Oligonucleotide
  source 1..41: mol_type = other DNA
cactgaaataggacggaatctgctagacaaccgtcgttaag

SEQ ID NO: 435 - DNA, 39 bp, synthetic construct
  misc_feature 1..39: note = Target Specific Oligonucleotide
  source 1..39: mol_type = other DNA
aaagtttattggcgcttgccgtagacaaccgtcgttaag

SEQ ID NO: 436 - DNA, 39 bp, synthetic construct
  misc_feature 1..39: note = Target Specific Oligonucleotide
  source 1..39: mol_type = other DNA
gcaatgccattagcgatacgatagacaaccgtcgttaag

SEQ ID NO: 437 - DNA, 38 bp, synthetic construct
  misc_feature 1..38: note = Target Specific Oligonucleotide
  source 1..38: mol_type = other DNA
tgattgaacaccacgcgacatagacaaccgtcgttaag

SEQ ID NO: 438 - DNA, 40 bp, synthetic construct
  misc_feature 1..40: note = Target Specific Oligonucleotide
  source 1..40: mol_type = other DNA
agtcttcgaagcactattgccatagacaaccgtcgttaag

SEQ ID NO: 439 - DNA, 39 bp, synthetic construct
  misc_feature 1..39: note = Target Specific Oligonucleotide
  source 1..39: mol_type = other DNA
tcaatggttgatcggcctctctagacaaccgtcgttaag

SEQ ID NO: 440 - DNA, 41 bp, synthetic construct
  misc_feature 1..41: note = Target Specific Oligonucleotide
  source 1..41: mol_type = other DNA
tggcatgatgtctaataggagtctagacaaccgtcgttaag

SEQ ID NO: 441 - DNA, 38 bp, synthetic construct
  misc_feature 1..38: note = Target Specific Oligonucleotide
  source 1..38: mol_type = other DNA
ctgcggtttagccttgacgttagacaaccgtcgttaag

SEQ ID NO: 442 - DNA, 38 bp, synthetic construct
  misc_feature 1..38: note = Target Specific Oligonucleotide
  source 1..38: mol_type = other DNA
cccagaggacgccatcattttagacaaccgtcgttaag

SEQ ID NO: 443 - DNA, 40 bp, synthetic construct
  misc_feature 1..40: note = Target Specific Oligonucleotide
  source 1..40: mol_type = other DNA
tttcttgaaagacgacagcagttagacaaccgtcgttaag

SEQ ID NO: 444 - DNA, 41 bp, synthetic construct
  misc_feature 1..41: note = Target Specific Oligonucleotide
  source 1..41: mol_type = other DNA
attaaatgttcggaaggatgacctagacaaccgtcgttaag

SEQ ID NO: 445 - DNA, 38 bp, synthetic construct
  misc_feature 1..38: note = Target Specific Oligonucleotide
  source 1..38: mol_type = other DNA
tcttttgtcagcacggttgctagacaaccgtcgttaag

SEQ ID NO: 446 - DNA, 38 bp, synthetic construct
  misc_feature 1..38: note = Target Specific Oligonucleotide
  source 1..38: mol_type = other DNA
ctggatcagctcgaccaggatagacaaccgtcgttaag

SEQ ID NO: 447 - DNA, 38 bp, synthetic construct
  misc_feature 1..38: note = Target Specific Oligonucleotide
  source 1..38: mol_type = other DNA
accatggtccgacttctgcctagacaaccgtcgttaag

SEQ ID NO: 448 - DNA, 40 bp, synthetic construct
  misc_feature 1..40: note = Target Specific Oligonucleotide
  source 1..40: mol_type = other DNA
ctggatattcggttatctgggctagacaaccgtcgttaag

SEQ ID NO: 449 - DNA, 41 bp, synthetic construct
  misc_feature 1..41: note = Target Specific Oligonucleotide
  source 1..41: mol_type = other DNA
atcccctttacgatactcttcagtagacaaccgtcgttaag

SEQ ID NO: 450 - DNA, 38 bp, synthetic construct
  misc_feature 1..38: note = Target Specific Oligonucleotide
  source 1..38: mol_type = other DNA
tccagagcgggaacagtatgtagacaaccgtcgttaag

SEQ ID NO: 451 - DNA, 38 bp, synthetic construct
  misc_feature 1..38: note = Target Specific Oligonucleotide
  source 1..38: mol_type = other DNA
gttgcctgcataagatgggctagacaaccgtcgttaag

SEQ ID NO: 452 - DNA, 38 bp, synthetic construct
  misc_feature 1..38: note = Target Specific Oligonucleotide
  source 1..38: mol_type = other DNA
ccttttgtgccgatgacccgtagacaaccgtcgttaag

SEQ ID NO: 453 - DNA, 37 bp, synthetic construct
  misc_feature 1..37: note = Target Specific Oligonucleotide
  source 1..37: mol_type = other DNA
gcttccgtggactgggacgtagacaaccgtcgttaag

SEQ ID NO: 454 - DNA, 41 bp, synthetic construct
  misc_feature 1..41: note = Target Specific Oligonucleotide
  source 1..41: mol_type = other DNA
ccatagaaggacgaggtatttcctagacaaccgtcgttaag

SEQ ID NO: 455 - DNA, 39 bp, synthetic construct
  misc_feature 1..39: note = Target Specific Oligonucleotide
  source 1..39: mol_type = other DNA
agaatgagtcgctgctcgatatagacaaccgtcgttaag

SEQ ID NO: 456 - DNA, 41 bp, synthetic construct
  misc_feature 1..41: note = Target Specific Oligonucleotide
  source 1..41: mol_type = other DNA
gcttcgacaaacgcaaagcgtagactcaatgatgataaaga

SEQ ID NO: 457 - DNA, 44 bp, synthetic construct
  misc_feature 1..44: note = Target Specific Oligonucleotide
  source 1..44: mol_type = other DNA
gctagttcacagaaagaccattttagactcaatgatgataaaga

SEQ ID NO: 458 - DNA, 41 bp, synthetic construct
  misc_feature 1..41: note = Target Specific Oligonucleotide
  source 1..41: mol_type = other DNA
cagctcatcgaaagcgaccctagactcaatgatgataaaga

SEQ ID NO: 459 - DNA, 40 bp, synthetic construct
  misc_feature 1..40: note = Target Specific Oligonucleotide
  source 1..40: mol_type = other DNA
cggctgctacaaacctcggtagactcaatgatgataaaga

SEQ ID NO: 460 - DNA, 42 bp, synthetic construct
  misc_feature 1..42: note = Target Specific Oligonucleotide
  source 1..42: mol_type = other DNA
aacaatttgaaggccccacaatagactcaatgatgataaaga

SEQ ID NO: 461 - DNA, 39 bp, synthetic construct
  misc_feature 1..39: note = Target Specific Oligonucleotide
  source 1..39: mol_type = other DNA
gcgtctgcaccggagacttagactcaatgatgataaaga

SEQ ID NO: 462 - DNA, 39 bp, synthetic construct
  misc_feature 1..39: note = Target Specific Oligonucleotide
  source 1..39: mol_type = other DNA
gcgctgctcaggcattggtagactcaatgatgataaaga

SEQ ID NO: 463 - DNA, 47 bp, synthetic construct
  misc_feature 1..47: note = Target Specific Oligonucleotide
  source 1..47: mol_type = other DNA
ttgttgtgtaacaacataatttcaagtagactcaatgatgataaaga

SEQ ID NO: 464 - DNA, 38 bp, synthetic construct
  misc_feature 1..38: note = Target Specific Oligonucleotide
  source 1..38: mol_type = other DNA
ctccacgtcggcgtgcatagactcaatgatgataaaga

SEQ ID NO: 465 - DNA, 40 bp, synthetic construct
  misc_feature 1..40: note = Target Specific Oligonucleotide
  source 1..40: mol_type = other DNA
ctgcggggattgtagccggtagactcaatgatgataaaga

SEQ ID NO: 466 - DNA, 42 bp, synthetic construct
  misc_feature 1..42: note = Target Specific Oligonucleotide
  source 1..42: mol_type = other DNA
atcttagctactgaccggctatagactcaatgatgataaaga

SEQ ID NO: 467 - DNA, 43 bp, synthetic construct
  misc_feature 1..43: note = Target Specific Oligonucleotide
  source 1..43: mol_type = other DNA
aagagattatacgcctcacggatagactcaatgatgataaaga

SEQ ID NO: 468 - DNA, 44 bp, synthetic construct
  misc_feature 1..44: note = Target Specific Oligonucleotide
  source 1..44: mol_type = other DNA
tttcgagtaacgaaattagctcctagactcaatgatgataaaga

SEQ ID NO: 469 - DNA, 39 bp, synthetic construct
  misc_feature 1..39: note = Target Specific Oligonucleotide
  source 1..39: mol_type = other DNA
gacccgcagattggcacgtagactcaatgatgataaaga

SEQ ID NO: 470 - DNA, 41 bp, synthetic construct
  misc_feature 1..41: note = Target Specific Oligonucleotide
  source 1..41: mol_type = other DNA
atggccccaatctcgtttggtagactcaatgatgataaaga

SEQ ID NO: 471 - DNA, 44 bp, synthetic construct
  misc_feature 1..44: note = Target Specific Oligonucleotide
  source 1..44: mol_type = other DNA
ccagtagctataacgaagtcctctagactcaatgatgataaaga

SEQ ID NO: 472 - DNA, 43 bp, synthetic construct
  misc_feature 1..43: note = Target Specific Oligonucleotide
  source 1..43: mol_type = other DNA
acaaattcccggacactatggatagactcaatgatgataaaga

SEQ ID NO: 473 - DNA, 43 bp, synthetic construct
  misc_feature 1..43: note = Target Specific Oligonucleotide
  source 1..43: mol_type = other DNA
ttcacagagttgataaggccactagactcaatgatgataaaga

SEQ ID NO: 474 - DNA, 43 bp, synthetic construct
  misc_feature 1..43: note = Target Specific Oligonucleotide
  source 1..43: mol_type = other DNA
aactttgccggtctctttacattagactcaatgatgataaaga

SEQ ID NO: 475 - DNA, 45 bp, synthetic construct
  misc_feature 1..45: note = Target Specific Oligonucleotide
  source 1..45: mol_type = other DNA
ttccaatgtgcaagaatgatttcttagactcaatgatgataaaga

SEQ ID NO: 476 - DNA, 43 bp, synthetic construct
  misc_feature 1..43: note = Target Specific Oligonucleotide
  source 1..43: mol_type = other DNA
ctccttctggggtatttcctgctagactcaatgatgataaaga

SEQ ID NO: 477 - DNA, 40 bp, synthetic construct
  misc_feature 1..40: note = Target Specific Oligonucleotide
  source 1..40: mol_type = other DNA
ccacttcagttggccggtatagactcaatgatgataaaga

SEQ ID NO: 478 - DNA, 39 bp, synthetic construct
  misc_feature 1..39: note = Target Specific Oligonucleotide
  source 1..39: mol_type = other DNA
tccgtcaacgtccgcagttagactcaatgatgataaaga

SEQ ID NO: 479 - DNA, 42 bp, synthetic construct
  misc_feature 1..42: note = Target Specific Oligonucleotide
  source 1..42: mol_type = other DNA
agtgagatcgccatagtgcaatagactcaatgatgataaaga

SEQ ID NO: 480 - DNA, 43 bp, synthetic construct
  misc_feature 1..43: note = Target Specific Oligonucleotide
  source 1..43: mol_type = other DNA
gaaggtgaccctataaggagtcatagactacaacaaaagatcg

SEQ ID NO: 481 - DNA, 41 bp, synthetic construct
  misc_feature 1..41: note = Target Specific Oligonucleotide
  source 1..41: mol_type = other DNA
aacaccgcttgcatagttgtgtagactacaacaaaagatcg

SEQ ID NO: 482 - DNA, 41 bp, synthetic construct
  misc_feature 1..41: note = Target Specific Oligonucleotide
  source 1..41: mol_type = other DNA
tacagacgcggaatcattctctagactacaacaaaagatcg

SEQ ID NO: 483 - DNA, 41 bp, synthetic construct
  misc_feature 1..41: note = Target Specific Oligonucleotide
  source 1..41: mol_type = other DNA
tggaggaaatgagcatgaccttagactacaacaaaagatcg

SEQ ID NO: 484 - DNA, 41 bp, synthetic construct
  misc_feature 1..41: note = Target Specific Oligonucleotide
  source 1..41: mol_type = other DNA
atggtgtacactcgaggctgatagactacaacaaaagatcg

SEQ ID NO: 485 - DNA, 41 bp, synthetic construct
  misc_feature 1..41: note = Target Specific Oligonucleotide
  source 1..41: mol_type = other DNA
ccttgtggtagacgttcagcttagactacaacaaaagatcg

SEQ ID NO: 486 - DNA, 44 bp, synthetic construct
  misc_feature 1..44: note = Target Specific Oligonucleotide
  source 1..44: mol_type = other DNA
ggatcaggtttaatggtcactatgtagactacaacaaaagatcg

SEQ ID NO: 487 - DNA, 40 bp, synthetic construct
  misc_feature 1..40: note = Target Specific Oligonucleotide
  source 1..40: mol_type = other DNA
cacacggtcaacgctgtacatagactacaacaaaagatcg

SEQ ID NO: 488 - DNA, 40 bp, synthetic construct
  misc_feature 1..40: note = Target Specific Oligonucleotide
  source 1..40: mol_type = other DNA
caggtgtttgcggaagttcctagactacaacaaaagatcg

SEQ ID NO: 489 - DNA, 40 bp, synthetic construct
  misc_feature 1..40: note = Target Specific Oligonucleotide
  source 1..40: mol_type = other DNA
gtgctcatggtcgtagaggatagactacaacaaaagatcg

SEQ ID NO: 490 - DNA, 43 bp, synthetic construct
  misc_feature 1..43: note = Target Specific Oligonucleotide
  source 1..43: mol_type = other DNA
aagtcaaagtacgtctcgatcattagactacaacaaaagatcg

SEQ ID NO: 491 - DNA, 39 bp, synthetic construct
  misc_feature 1..39: note = Target Specific Oligonucleotide
  source 1..39: mol_type = other DNA
tcgtagcagccgttggagatagactacaacaaaagatcg

SEQ ID NO: 492 - DNA, 39 bp, synthetic construct
  misc_feature 1..39: note = Target Specific Oligonucleotide
  source 1..39: mol_type = other DNA
tcgtggccagagtaggcattagactacaacaaaagatcg

SEQ ID NO: 493 - DNA, 40 bp, synthetic construct
  misc_feature 1..40: note = Target Specific Oligonucleotide
  source 1..40: mol_type = other DNA
ggtactctccaaacgctcggtagactacaacaaaagatcg

SEQ ID NO: 494 - DNA, 42 bp, synthetic construct
  misc_feature 1..42: note = Target Specific Oligonucleotide
  source 1..42: mol_type = other DNA
aggtacgaatagggatgtcgtctagactacaacaaaagatcg

SEQ ID NO: 495 - DNA, 42 bp, synthetic construct
  misc_feature 1..42: note = Target Specific Oligonucleotide
  source 1..42: mol_type = other DNA
agggtatcgtacatcgttccaatagactacaacaaaagatcg

SEQ ID NO: 496 - DNA, 39 bp, synthetic construct
  misc_feature 1..39: note = Target Specific Oligonucleotide
  source 1..39: mol_type = other DNA
caggtgatctgcgccgttgtagactacaacaaaagatcg

SEQ ID NO: 497 - DNA, 39 bp, synthetic construct
  misc_feature 1..39: note = Target Specific Oligonucleotide
  source 1..39: mol_type = other DNA
ccacaggcgatacaaccggtagactacaacaaaagatcg

SEQ ID NO: 498 - DNA, 39 bp, synthetic construct
  misc_feature 1..39: note = Target Specific Oligonucleotide
  source 1..39: mol_type = other DNA
cagcagcttcgagtgctggtagactacaacaaaagatcg

SEQ ID NO: 499 - DNA, 39 bp, synthetic construct
  misc_feature 1..39: note = Target Specific Oligonucleotide
  source 1..39: mol_type = other DNA
ccctcacaggacgtcgtcatagactacaacaaaagatcg

SEQ ID NO: 500 - DNA, 39 bp, synthetic construct
  misc_feature 1..39: note = Target Specific Oligonucleotide
  source 1..39: mol_type = other DNA
gcacgaagccttcggtgtctagactacaacaaaagatcg

SEQ ID NO: 501 - DNA, 41 bp, synthetic construct
  misc_feature 1..41: note = Target Specific Oligonucleotide
  source 1..41: mol_type = other DNA
tgggaagtgtcggctttcatgtagactacaacaaaagatcg

SEQ ID NO: 502 - DNA, 41 bp, synthetic construct; identical to SEQ ID NO: 501
  misc_feature 1..41: note = Target Specific Oligonucleotide
  source 1..41: mol_type = other DNA

SEQ ID NO: 503 - DNA, 38 bp, synthetic construct
  misc_feature 1..38: note = Target Specific Oligonucleotide
  source 1..38: mol_type = other DNA
tgctcgcactttggccgctagactacaacaaaagatcg

SEQ ID NO: 504 - DNA, 40 bp, synthetic construct
  misc_feature 1..40: note = Target Specific Oligonucleotide
  source 1..40: mol_type = other DNA
gtcttttggcacggtttctgttagactgtattcaacgtcc

SEQ ID NO: 505 - DNA, 40 bp, synthetic construct
  misc_feature 1..40: note = Target Specific Oligonucleotide
  source 1..40: mol_type = other DNA
tacttcttcaacgcgaagagctagactgtattcaacgtcc

SEQ ID NO: 506 - DNA, 40 bp, synthetic construct
  misc_feature 1..40: note = Target Specific Oligonucleotide
  source 1..40: mol_type = other DNA
tttgaggttgtatccgctgcttagactgtattcaacgtcc

SEQ ID NO: 507 - DNA, 44 bp, synthetic construct
  misc_feature 1..44: note = Target Specific Oligonucleotide
  source 1..44: mol_type = other DNA
caagttgactaaatctcgtactttctagactgtattcaacgtcc

SEQ ID NO: 508 - DNA, 43 bp, synthetic construct
  misc_feature 1..43: note = Target Specific Oligonucleotide
  source 1..43: mol_type = other DNA
gccttattaacggtatcttcagaatagactgtattcaacgtcc

SEQ ID NO: 509 - DNA, 40 bp, synthetic construct
  misc_feature 1..40: note = Target Specific Oligonucleotide
  source 1..40: mol_type = other DNA
tgttctggatttcgcaggtcctagactgtattcaacgtcc

SEQ ID NO: 510 - DNA, 42 bp, synthetic construct
  misc_feature 1..42: note = Target Specific Oligonucleotide
  source 1..42: mol_type = other DNA
gctttatcaggttatgttgcatgtagactgtattcaacgtcc

SEQ ID NO: 511 - DNA, 43 bp, synthetic construct
  misc_feature 1..43: note = Target Specific Oligonucleotide
  source 1..43: mol_type = other DNA
gattctggcttatagggtattcactagactgtattcaacgtcc

SEQ ID NO: 512 - DNA, 41 bp, synthetic construct
  misc_feature 1..41: note = Target Specific Oligonucleotide
  source 1..41: mol_type = other DNA
ttttcctcccgcaattcctagatagactgtattcaacgtcc

SEQ ID NO: 513 - DNA, 40 bp, synthetic construct
  misc_feature 1..40: note = Target Specific Oligonucleotide
  source 1..40: mol_type = other DNA
tgtttccgtcaaatcgtgtggtagactgtattcaacgtcc

SEQ ID NO: 514 - DNA, 41 bp, synthetic construct
  misc_feature 1..41: note = Target Specific Oligonucleotide
  source 1..41: mol_type = other DNA
ggttccctctagatcttgcctttagactgtattcaacgtcc

SEQ ID NO: 515 - DNA, 43 bp, synthetic construct
  misc_feature 1..43: note = Target Specific Oligonucleotide
  source 1..43: mol_type = other DNA
gcatgtaccacctatcatctaatgtagactgtattcaacgtcc

SEQ ID NO: 516 - DNA, 39 bp, synthetic construct
  misc_feature 1..39: note = Target Specific Oligonucleotide
  source 1..39: mol_type = other DNA
gttgccaacacgagctgacttagactgtattcaacgtcc

SEQ ID NO: 517 - DNA, 41 bp, synthetic construct
  misc_feature 1..41: note = Target Specific Oligonucleotide
  source 1..41: mol_type = other DNA
actgactactagttcaagcgcatagactgtattcaacgtcc

SEQ ID NO: 518 - DNA, 39 bp, synthetic construct
  misc_feature 1..39: note = Target Specific Oligonucleotide
  source 1..39: mol_type = other DNA
tctcttgctcgctttggacctagactgtattcaacgtcc

SEQ ID NO: 519 - DNA, 44 bp, synthetic construct
  misc_feature 1..44: note = Target Specific Oligonucleotide
  source 1..44: mol_type = other DNA
cactgctagaacaactatcaatttgtagactgtattcaacgtcc

SEQ ID NO: 520 - DNA, 41 bp, synthetic construct
  misc_feature 1..41: note = Target Specific Oligonucleotide
  source 1..41: mol_type = other DNA
ttacatggcttaagttggggagtagactgtattcaacgtcc

SEQ ID NO: 521 - DNA, 40 bp, synthetic construct
  misc_feature 1..40: note = Target Specific Oligonucleotide
  source 1..40: mol_type = other DNA
gtgaggggacgctcttgtatttagactgtattcaacgtcc

SEQ ID NO: 522 - DNA, 40 bp, synthetic construct
  misc_feature 1..40: note = Target Specific Oligonucleotide
  source 1..40: mol_type = other DNA
aacggtgctatgcctagtagatagactgtattcaacgtcc

SEQ ID NO: 523 - DNA, 40 bp, synthetic construct
  misc_feature 1..40: note = Target Specific Oligonucleotide
  source 1..40: mol_type = other DNA
tttgccttccctagagtgctatagactgtattcaacgtcc

SEQ ID NO: 524 - DNA, 43 bp, synthetic construct
  misc_feature 1..43: note = Target Specific Oligonucleotide
  source 1..43: mol_type = other DNA
atgtcccaatggatacttaaagcctagactgtattcaacgtcc

SEQ ID NO: 525 - DNA, 43 bp, synthetic construct
  misc_feature 1..43: note = Target Specific Oligonucleotide
  source 1..43: mol_type = other DNA
cttccagtaacgagatactttccttagactgtattcaacgtcc

SEQ ID NO: 526 - DNA, 42 bp, synthetic construct
  misc_feature 1..42: note = Target Specific Oligonucleotide
  source 1..42: mol_type = other DNA
caaccatgaattagtcccttgggtagactgtattcaacgtcc

SEQ ID NO: 527 - DNA, 44 bp, synthetic construct
  misc_feature 1..44: note = Target Specific Oligonucleotide
  source 1..44: mol_type = other DNA
cagcattattagacactttaactgttagactgtattcaacgtcc

SEQ ID NO: 528 - DNA, 43 bp, synthetic construct
  misc_feature 1..43: note = Target Specific Oligonucleotide
  source 1..43: mol_type = other DNA
tgcacatatgattgacgctcagtagactcttatattgagtggt

SEQ ID NO: 529 - DNA, 40 bp, synthetic construct
  misc_feature 1..40: note = Target Specific Oligonucleotide
  source 1..40: mol_type = other DNA
ttcaccgcctgcaacaagatagactcttatattgagtggt

SEQ ID NO: 530 - DNA, 44 bp, synthetic construct
  misc_feature 1..44: note = Target Specific Oligonucleotide
  source 1..44: mol_type = other DNA
ttcacttacaggtaacaccaagttagactcttatattgagtggt

SEQ ID NO: 531 - DNA, 45 bp, synthetic construct
  misc_feature 1..45: note = Target Specific Oligonucleotide
  source 1..45: mol_type = other DNA
aagcaatagatcgtccataagttatagactcttatattgagtggt

SEQ ID NO: 532 - DNA, 48 bp, synthetic construct
  misc_feature 1..48: note = Target Specific Oligonucleotide
  source 1..48: mol_type = other DNA
tacaagaaccctaattgtaataatagatagactcttatattgagtggt

SEQ ID NO: 533 - DNA, 44 bp, synthetic construct
  misc_feature 1..44: note = Target Specific Oligonucleotide
  source 1..44: mol_type = other DNA
gaataggaccaaagtgtcccttgtagactcttatattgagtggt

SEQ ID NO: 534 - DNA, 44 bp, synthetic construct
  misc_feature 1..44: note = Target Specific Oligonucleotide
  source 1..44: mol_type = other DNA
tccatcaggactaaatctcacactagactcttatattgagtggt

SEQ ID NO: 535 - DNA, 44 bp, synthetic construct
  misc_feature 1..44: note = Target Specific Oligonucleotide
  source 1..44: mol_type = other DNA
aaaggccatacgtttttcctacctagactcttatattgagtggt

SEQ ID NO: 536 - DNA, 44 bp, synthetic construct
  misc_feature 1..44: note = Target Specific Oligonucleotide
  source 1..44: mol_type = other DNA
gcagtgtacaatgaacgagaaattagactcttatattgagtggt

SEQ ID NO: 537 - DNA, 44 bp, synthetic construct
  misc_feature 1..44: note = Target Specific Oligonucleotide
  source 1..44: mol_type = other DNA
ggcattctgctttaagatcagaatagactcttatattgagtggt

SEQ ID NO: 538 - DNA, 40 bp, synthetic construct
  misc_feature 1..40: note = Target Specific Oligonucleotide
  source 1..40: mol_type = other DNA
ggccagccacgattcacagtagactcttatattgagtggt

SEQ ID NO: 539 - DNA, 39 bp, synthetic construct
  misc_feature 1..39: note = Target Specific Oligonucleotide
  source 1..39: mol_type = other DNA
gaagccagcgaccggacttagactcttatattgagtggt

SEQ ID NO: 540 - DNA, 41 bp, synthetic construct
  misc_feature 1..41: note = Target Specific Oligonucleotide
  source 1..41: mol_type = other DNA
ctgagggtcgtcgttgtctttagactcttatattgagtggt

SEQ ID NO: 541 - DNA, 43 bp, synthetic construct
  misc_feature 1..43: note = Target Specific Oligonucleotide
  source 1..43: mol_type = other DNA
ctgaaccactggcatagagttctagactcttatattgagtggt

SEQ ID NO: 542 - DNA, 40 bp, synthetic construct
  misc_feature 1..40: note = Target Specific Oligonucleotide
  source 1..40: mol_type = other DNA
aaatcaaccacgggtcgcgtagactcttatattgagtggt

SEQ ID NO: 543 - DNA, 40 bp, synthetic construct
  misc_feature 1..40: note = Target Specific Oligonucleotide
  source 1..40: mol_type = other DNA
agcaacagtcgacgagggatagactcttatattgagtggt

SEQ ID NO: 544 - DNA, 48 bp, synthetic construct
  misc_feature 1..48: note = Target Specific Oligonucleotide
  source 1..48: mol_type = other DNA
atatattccatactactaacagacatatagactcttatattgagtggt

SEQ ID NO: 545 - DNA, 38 bp, synthetic construct
  misc_feature 1..38: note = Target Specific Oligonucleotide
  source 1..38: mol_type = other DNA
tccacaccggcatcggctagactcttatattgagtggt

SEQ ID NO: 546 - DNA, 40 bp, synthetic construct
  misc_feature 1..40: note = Target Specific Oligonucleotide
  source 1..40: mol_type = other DNA
agaagggcaatccggtggctagactcttatattgagtggt

SEQ ID NO: 547 - DNA, 40 bp, synthetic construct
  misc_feature 1..40: note = Target Specific Oligonucleotide
  source 1..40: mol_type = other DNA
tcagggagtctgcgaccagtagactcttatattgagtggt

SEQ ID NO: 548 - DNA, 40 bp, synthetic construct
  misc_feature 1..40: note = Target Specific Oligonucleotide
  source 1..40: mol_type = other DNA
gctttgccagctcaccacttagactcttatattgagtggt

SEQ ID NO: 549 - DNA, 43 bp, synthetic construct
  misc_feature 1..43: note = Target Specific Oligonucleotide
  source 1..43: mol_type = other DNA
aaatacccataaggcgtgatgctagactcttatattgagtggt

SEQ ID NO: 550 - DNA, 44 bp, synthetic construct
  misc_feature 1..44: note = Target Specific Oligonucleotide
  source 1..44: mol_type = other DNA
attactatcctgcgtgaaatccatagactcttatattgagtggt

SEQ ID NO: 551 - DNA, 43 bp, synthetic construct
  misc_feature 1..43: note = Target Specific Oligonucleotide
  source 1..43: mol_type = other DNA
cccaaagtcgaacagttttgtctagactcttatattgagtggt

SEQ ID NO: 552 - DNA, 41 bp, synthetic construct
  misc_feature 1..41: note = Target Specific Oligonucleotide
  source 1..41: mol_type = other DNA
tcctcaaatgtccgaggatgatagacctattctatgcttcg

SEQ ID NO: 553 - DNA, 42 bp, synthetic construct
  misc_feature 1..42: note = Target Specific Oligonucleotide
  source 1..42: mol_type = other DNA
gtgaacacaacgtaagaacaggtagacctattctatgcttcg

SEQ ID NO: 554 - DNA, 42 bp, synthetic construct
  misc_feature 1..42: note = Target Specific Oligonucleotide
  source 1..42: mol_type = other DNA
actcctctggttacgcttcatttagacctattctatgcttcg

SEQ ID NO: 555 - DNA, 41 bp, synthetic construct
  misc_feature 1..41: note = Target Specific Oligonucleotide
  source 1..41: mol_type = other DNA
acctaacataaccgtgctgcctagacctattctatgcttcg

SEQ ID NO: 556 - DNA, 41 bp, synthetic construct
  misc_feature 1..41: note = Target Specific Oligonucleotide
  source 1..41: mol_type = other DNA
ttcagttgaccgtctggacactagacctattctatgcttcg

SEQ ID NO: 557 - DNA, 43 bp, synthetic construct
  misc_feature 1..43: note = Target Specific Oligonucleotide
  source 1..43: mol_type = other DNA
ctctgacacacgtaacaataacatagacctattctatgcttcg

SEQ ID NO: 558 - DNA, 43 bp, synthetic construct
  misc_feature 1..43: note = Target Specific Oligonucleotide
  source 1..43: mol_type = other DNA
agtaatgtgaggtacaactgcattagacctattctatgcttcg

SEQ ID NO: 559 - DNA, 41 bp, synthetic construct
  misc_feature 1..41: note = Target Specific Oligonucleotide
  source 1..41: mol_type = other DNA
gcttctttcccgcccaaaatatagacctattctatgcttcg

SEQ ID NO: 560 - DNA, 44 bp, synthetic construct
  misc_feature 1..44: note = Target Specific Oligonucleotide
  source 1..44: mol_type = other DNA
aaagaaaagcggtctactagattatagacctattctatgcttcg

SEQ ID NO: 561 - DNA, 41 bp, synthetic construct
  misc_feature 1..41: note = Target Specific Oligonucleotide
  source 1..41: mol_type = other DNA
tgacgtgcggcgaaatttatgtagacctattctatgcttcg

SEQ ID NO: 562 - DNA, 41 bp, synthetic construct
  misc_feature 1..41: note = Target Specific Oligonucleotide
  source 1..41: mol_type = other DNA
gcttattgcagcgatggatgatagacctattctatgcttcg

SEQ ID NO: 563 - DNA, 42 bp, synthetic construct
  misc_feature 1..42: note = Target Specific Oligonucleotide
  source 1..42: mol_type = other DNA
tgtcagtagacgatagaggaggtagacctattctatgcttcg

SEQ ID NO: 564 - DNA, 41 bp, synthetic construct
  misc_feature 1..41: note = Target Specific Oligonucleotide
  source 1..41: mol_type = other DNA
ttggttccgactacccaacagtagacctattctatgcttcg

SEQ ID NO: 565 - DNA, 45 bp, synthetic construct
  misc_feature 1..45: note = Target Specific Oligonucleotide
  source 1..45: mol_type = other DNA
ctcttaagtacgcaataattctctctagacctattctatgcttcg

SEQ ID NO: 566 - DNA, 43 bp, synthetic construct
  misc_feature 1..43: note = Target Specific Oligonucleotide
  source 1..43: mol_type = other DNA
gttccgtatatgtcggatctctctagacctattctatgcttcg

SEQ ID NO: 567 - DNA, 41 bp, synthetic construct
  misc_feature 1..41: note = Target Specific Oligonucleotide
  source 1..41: mol_type = other DNA
aaattcaccggacggagtgtttagacctattctatgcttcg

SEQ ID NO: 568 - DNA, 42 bp, synthetic construct
  misc_feature 1..42: note = Target Specific Oligonucleotide
  source 1..42: mol_type = other DNA
tttgtgaaaccgtagtggctcttagacctattctatgcttcg

SEQ ID NO: 569 - DNA, 42 bp, synthetic construct
  misc_feature 1..42: note = Target Specific Oligonucleotide
  source 1..42: mol_type = other DNA
acctctttaacgaaggtgtcagtagacctattctatgcttcg

SEQ ID NO: 570 - DNA, 41 bp, synthetic construct
  misc_feature 1..41: note = Target Specific Oligonucleotide
  source 1..41: mol_type = other DNA
ggactccggttctaacttggttagacctattctatgcttcg

SEQ ID NO: 571 - DNA, 43 bp, synthetic construct
  misc_feature 1..43: note = Target Specific Oligonucleotide
  source 1..43: mol_type = other DNA
atctctatatgacgtgctgttggtagacctattctatgcttcg

SEQ ID NO: 572 - DNA, 43 bp, synthetic construct
  misc_feature 1..43: note = Target Specific Oligonucleotide
  source 1..43: mol_type = other DNA
gttggaattattcggagactgagtagacctattctatgcttcg

SEQ ID NO: 573 - DNA, 40 bp, synthetic construct
  misc_feature 1..40: note = Target Specific Oligonucleotide
  source 1..40: mol_type = other DNA
gcacatttgacgacggcttctagacctattctatgcttcg

SEQ ID NO: 574 - DNA, 40 bp, synthetic construct
  misc_feature 1..40: note = Target Specific Oligonucleotide
  source 1..40: mol_type = other DNA
actccgtgtaaacgcagtggtagacctattctatgcttcg

SEQ ID NO: 575 - DNA, 43 bp, synthetic construct
  misc_feature 1..43: note = Target Specific Oligonucleotide
  source 1..43: mol_type = other DNA
acacataactcgttaacacgttctagacctattctatgcttcg

SEQ ID NO: 576 - DNA, 42 bp, synthetic construct
  misc_feature 1..42: note = Target Specific Oligonucleotide
  source 1..42: mol_type = other DNA
cagatctgttgagctaacagggtagacatgttataccatgcg

SEQ ID NO: 577 - DNA, 44 bp, synthetic construct
  misc_feature 1..44: note = Target Specific Oligonucleotide
  source 1..44: mol_type = other DNA
ttctagactatgcaaccctctaggtagacatgttataccatgcg

SEQ ID NO: 578 - DNA, 43 bp, synthetic construct
  misc_feature 1..43: note = Target Specific Oligonucleotide
  source 1..43: mol_type = other DNA
cacccatgtgagtaatacactgctagacatgttataccatgcg

SEQ ID NO: 579 - DNA, 42 bp, synthetic construct
  misc_feature 1..42: note = Target Specific Oligonucleotide
  source 1..42: mol_type = other DNA
agtcccatattgccgttcatgctagacatgttataccatgcg

SEQ ID NO: 580 - DNA, 42 bp, synthetic construct
  misc_feature 1..42: note = Target Specific Oligonucleotide
  source 1..42: mol_type = other DNA
atttcctcataacggtcatggctagacatgttataccatgcg

SEQ ID NO: 581 - DNA, 42 bp, synthetic construct
  misc_feature 1..42: note = Target Specific Oligonucleotide
  source 1..42: mol_type = other DNA
ttactgcctagctagcaggttatagacatgttataccatgcg

SEQ ID NO: 582 - DNA, 41 bp, synthetic construct
  misc_feature 1..41: note = Target Specific Oligonucleotide
  source 1..41: mol_type = other DNA
acgtgcccttggtactatgactagacatgttataccatgcg

SEQ ID NO: 583 - DNA, 41 bp, synthetic construct
  misc_feature 1..41: note = Target Specific Oligonucleotide
  source 1..41: mol_type = other DNA
cttgccaaaccctagcttggatagacatgttataccatgcg

SEQ ID NO: 584 - DNA, 45 bp, synthetic construct
  misc_feature 1..45: note = Target Specific Oligonucleotide
  source 1..45: mol_type = other DNA
attatcacttaacgaaggtcctttgtagacatgttataccatgcg

SEQ ID NO: 585 - DNA, 45 bp, synthetic construct
  misc_feature 1..45: note = Target Specific Oligonucleotide
  source 1..45: mol_type = other DNA
atatacagcctaagcaccaattatgtagacatgttataccatgcg

SEQ ID NO: 586 - DNA, 40 bp, synthetic construct
  misc_feature 1..40: note = Target Specific Oligonucleotide
  source 1..40: mol_type = other DNA
ggcaacaaaacgtcatggcatagacatgttataccatgcg

SEQ ID NO: 587 - DNA, 43 bp, synthetic construct
  misc_feature 1..43: note = Target Specific Oligonucleotide
  source 1..43: mol_type = other DNA
gtctgaggactaaccctaaagggtagacatgttataccatgcg

SEQ ID NO: 588 - DNA, 46 bp, synthetic construct
  misc_feature 1..46: note = Target Specific Oligonucleotide
  source 1..46: mol_type = other DNA
cttaaaatagatcgaaactctgtctctagacatgttataccatgcg

SEQ ID NO: 589 - DNA, 44 bp, synthetic construct
  misc_feature 1..44: note = Target Specific Oligonucleotide
  source 1..44: mol_type = other DNA
tgatctgtacaagctacgattttatagacatgttataccatgcg

SEQ ID NO: 590 - DNA, 43 bp, synthetic construct
  misc_feature 1..43: note = Target Specific Oligonucleotide
  source 1..43: mol_type = other DNA
aaatcagttataccaaggggagatagacatgttataccatgcg

SEQ ID NO: 591 - DNA, 45 bp, synthetic construct
  misc_feature 1..45: note = Target Specific Oligonucleotide
  source 1..45: mol_type = other DNA
taggaaaacatacgtatactgaatatagacatgttataccatgcg

SEQ ID NO: 592 - DNA, 40 bp, synthetic construct
  misc_feature 1..40: note = Target Specific Oligonucleotide
  source 1..40: mol_type = other DNA
agttggcctcacgttgcatttagacatgttataccatgcg

SEQ ID NO: 593 - DNA, 40 bp, synthetic construct
  misc_feature 1..40: note = Target Specific Oligonucleotide
  source 1..40: mol_type = other DNA
tttggtctcggcttgcgaattagacatgttataccatgcg

SEQ ID NO: 594 - DNA, 41 bp, synthetic construct
  misc_feature 1..41: note = Target Specific Oligonucleotide
  source 1..41: mol_type = other DNA
cagtagtcaacgcagcactcatagacatgttataccatgcg

SEQ ID NO: 595 - DNA, 44 bp, synthetic construct
  misc_feature 1..44: note = Target Specific Oligonucleotide
  source 1..44: mol_type = other DNA
caacaggatcacctaatattcccatagacatgttataccatgcg

SEQ ID NO: 596 - DNA, 43 bp, synthetic construct
  misc_feature 1..43: note = Target Specific Oligonucleotide
  source 1..43: mol_type = other DNA
tttggacagatatctcctcgaaatagacatgttataccatgcg

SEQ ID NO: 597 - DNA, 43 bp, synthetic construct
  misc_feature 1..43: note = Target Specific Oligonucleotide
  source 1..43: mol_type = other DNA
aaaggctatctacactttggcaatagacatgttataccatgcg

SEQ ID NO: 598 - DNA, 40 bp, synthetic construct
  misc_feature 1..40: note = Target Specific Oligonucleotide
  source 1..40: mol_type = other DNA
tgcctggatcctatcccgactagacatgttataccatgcg

SEQ ID NO: 599 - DNA, 40 bp, synthetic construct
  misc_feature 1..40: note = Target Specific Oligonucleotide
  source 1..40: mol_type = other DNA
tgtggtcacctacacgctgctagacatgttataccatgcg

SEQ ID NO: 600 - DNA, 43 bp, synthetic construct
  misc_feature 1..43: note = Target Specific Oligonucleotide
  source 1..43: mol_type = other DNA
ggtgagctggtaatctgaccttagacaatctatttaccctacg

SEQ ID NO: 601 - DNA, 41 bp, synthetic construct
  misc_feature 1..41: note = Target Specific Oligonucleotide
  source 1..41: mol_type = other DNA
gttgcaggcatgacgcaagtagacaatctatttaccctacg

SEQ ID NO: 602 - DNA, 43 bp, synthetic construct
  misc_feature 1..43: note = Target Specific Oligonucleotide
  source 1..43: mol_type = other DNA
aaaacgcctacgcatcatgtctagacaatctatttaccctacg

SEQ ID NO: 603 - DNA, 40 bp, synthetic construct
  misc_feature 1..40: note = Target Specific Oligonucleotide
  source 1..40: mol_type = other DNA
tggtcctgcgcggatgtctagacaatctatttaccctacg

SEQ ID NO: 604 - DNA, 41 bp, synthetic construct
  misc_feature 1..41: note = Target Specific Oligonucleotide
  source 1..41: mol_type = other DNA
ggtgagcttctccgtggcgtagacaatctatttaccctacg

SEQ ID NO: 605 - DNA, 41 bp, synthetic construct
  misc_feature 1..41: note = Target Specific Oligonucleotide
  source 1..41: mol_type = other DNA
agcgcagggccaattgacttagacaatctatttaccctacg

SEQ ID NO: 606 - DNA, 43 bp, synthetic construct
  misc_feature 1..43: note = Target Specific Oligonucleotide
  source 1..43: mol_type = other DNA
ccgggtacacggttttgatcttagacaatctatttaccctacg

SEQ ID NO: 607 - DNA, 40 bp, synthetic construct
  misc_feature 1..40: note = Target Specific Oligonucleotide
  source 1..40: mol_type = other DNA
ccggctcgatgtcgggtatagacaatctatttaccctacg

SEQ ID NO: 608 - DNA, 40 bp, synthetic construct
  misc_feature 1..40: note = Target Specific Oligonucleotide
  source 1..40: mol_type = other DNA
ggcgccttctcgccacattagacaatctatttaccctacg

SEQ ID NO: 609 - DNA, 40 bp, synthetic construct
  misc_feature 1..40: note = Target Specific Oligonucleotide
  source 1..40: mol_type = other DNA
cacgttgaagcgcgggtgtagacaatctatttaccctacg

SEQ ID NO: 610 - DNA, 44 bp, synthetic construct
  misc_feature 1..44: note = Target Specific Oligonucleotide
  source 1..44: mol_type = other DNA
gcccacagaaaccggtttaatgtagacaatctatttaccctacg

SEQ ID NO: 611 - DNA, 43 bp, synthetic construct
  misc_feature 1..43: note = Target Specific Oligonucleotide
  source 1..43: mol_type = other DNA
tcagaatgagcgcaatccaggtagacaatctatttaccctacg

SEQ ID NO: 612 - DNA, 42 bp, synthetic construct
  misc_feature 1..42: note = Target Specific Oligonucleotide
  source 1..42: mol_type = other DNA
ttcggacacaaagacgctcctagacaatctatttaccctacg

SEQ ID NO: 613 - DNA, 42 bp, synthetic construct
  misc_feature 1..42: note = Target Specific Oligonucleotide
  source 1..42: mol_type = other DNA
agcttgacgtaggtgtcggttagacaatctatttaccctacg

SEQ ID NO: 614 - DNA, 42 bp, synthetic construct
  misc_feature 1..42: note = Target Specific Oligonucleotide
  source 1..42: mol_type = other DNA
ggttcccgccaaatttcccgtagacaatctatttaccctacg

SEQ ID NO: 615 - DNA, 45 bp, synthetic construct
  misc_feature 1..45: note = Target Specific Oligonucleotide
  source 1..45: mol_type = other DNA
cagggctcatgatagtacaacagtagacaatctatttaccctacg

SEQ ID NO: 616 - DNA, 41 bp, synthetic construct
  misc_feature 1..41: note = Target Specific Oligonucleotide
  source 1..41: mol_type = other DNA
ggactttagcgcggtctcctagacaatctatttaccctacg

SEQ ID NO: 617 - DNA, 41 bp, synthetic construct
  misc_feature 1..41: note = Target Specific Oligonucleotide
  source 1..41: mol_type = other DNA
gcagccgttctaagcgctgtagacaatctatttaccctacg

SEQ ID NO: 618 - DNA, 41 bp, synthetic construct
  misc_feature 1..41: note = Target Specific Oligonucleotide
  source 1..41: mol_type = other DNA
gctggtaaaggcgcttccctagacaatctatttaccctacg

SEQ ID NO: 619 - DNA, 41 bp, synthetic construct
  misc_feature 1..41: note = Target Specific Oligonucleotide
  source 1..41: mol_type = other DNA
gaagaagtcggtgacgcggtagacaatctatttaccctacg

SEQ ID NO: 620 - DNA, 41 bp, synthetic construct
  misc_feature 1..41: note = Target Specific Oligonucleotide
  source 1..41: mol_type = other DNA
ggctggaaacctcgtccactagacaatctatttaccctacg

SEQ ID NO: 621 - DNA, 48 bp, synthetic construct
  misc_feature 1..48: note = Target Specific Oligonucleotide
  source 1..48: mol_type = other DNA
tttattagagtttaggaggtcagaaatagacaatctatttaccctacg

SEQ ID NO: 622 - DNA, 44 bp, synthetic construct
  misc_feature 1..44: note = Target Specific Oligonucleotide
  source 1..44: mol_type = other DNA
tgggaggtcccatattcagaggtagacaatctatttaccctacg

SEQ ID NO: 623 - DNA, 44 bp, synthetic construct
  misc_feature 1..44: note = Target Specific Oligonucleotide
  source 1..44: mol_type = other DNA
tccagggatgtcctacttgtcctagacaatctatttaccctacg

SEQ ID NO: 624 - DNA, 40 bp, synthetic construct
  misc_feature 1..40: note = Target Specific Oligonucleotide
  source 1..40: mol_type = other DNA
ccaggtttccggcacagtgggtaactgaattgtcacttta

SEQ ID NO: 625 - DNA, 46 bp, synthetic construct
  misc_feature 1..46: note = Target Specific Oligonucleotide
  source 1..46: mol_type = other DNA
tgttatactttgtcgctcttagtagagtaactgaattgtcacttta

SEQ ID NO: 626 - DNA, 44 bp, synthetic construct
  misc_feature 1..44: note = Target Specific Oligonucleotide
  source 1..44: mol_type = other DNA
agcttttaaccaggtttcgacttcgtaactgaattgtcacttta

SEQ ID NO: 627 - DNA, 45 bp, synthetic construct
  misc_feature 1..45: note = Target Specific Oligonucleotide
  source 1..45: mol_type = other DNA
gtatgagaatcccgataaaactggtgtaactgaattgtcacttta

SEQ ID NO: 628 - DNA, 43 bp, synthetic construct
  misc_feature 1..43: note = Target Specific Oligonucleotide
  source 1..43: mol_type = other DNA
aagaggacatcgaccaatcctcagtaactgaattgtcacttta

SEQ ID NO: 629 - DNA, 42 bp, synthetic construct
  misc_feature 1..42: note = Target Specific Oligonucleotide
  source 1..42: mol_type = other DNA
ggtttgtgcaagggtcgaaaacgtaactgaattgtcacttta

SEQ ID NO: 630 - DNA, 44 bp, synthetic construct
  misc_feature 1..44: note = Target Specific Oligonucleotide
  source 1..44: mol_type = other DNA
acacctctcgtagctatagatgtggtaactgaattgtcacttta

SEQ ID NO: 631 - DNA, 41 bp, synthetic construct
  misc_feature 1..41: note = Target Specific Oligonucleotide
  source 1..41: mol_type = other DNA
atgagagggcaaccgaggtgagtaactgaattgtcacttta

SEQ ID NO: 632 - DNA, 43 bp, synthetic construct
  misc_feature 1..43: note = Target Specific Oligonucleotide
  source 1..43: mol_type = other DNA
ccgctgtcagggtctatcattccgtaactgaattgtcacttta

SEQ ID NO: 633 - DNA, 41 bp, synthetic construct
  misc_feature 1..41: note = Target Specific Oligonucleotide
  source 1..41: mol_type = other DNA
gcaatgagcatatcctcgggcgtaactgaattgtcacttta

SEQ ID NO: 634 - DNA, 41 bp, synthetic construct
  misc_feature 1..41: note = Target Specific Oligonucleotide
  source 1..41: mol_type = other DNA
ggcttgagtgggttcacccaggtaactgaattgtcacttta

SEQ ID NO: 635 - DNA, 43 bp, synthetic construct
  misc_feature 1..43: note = Target Specific Oligonucleotide
  source 1..43: mol_type = other DNA
gcaaggtatggcaatagctgagtgtaactgaattgtcacttta

SEQ ID NO: 636 - DNA, 45 bp, synthetic construct
  misc_feature 1..45: note = Target Specific Oligonucleotide
  source 1..45: mol_type = other DNA
cagtaatagtagagacgccactacagtaactgaattgtcacttta

SEQ ID NO: 637 - DNA, 43 bp, synthetic construct
  misc_feature 1..43: note = Target Specific Oligonucleotide
  source 1..43: mol_type = other DNA
cttccaaagtgtcgcttcagagggtaactgaattgtcacttta

SEQ ID NO: 638 - DNA, 46 bp, synthetic construct
  misc_feature 1..46: note = Target Specific Oligonucleotide
  source 1..46: mol_type = other DNA
aggcaattttcccgatactttttattgtaactgaattgtcacttta

SEQ ID NO: 639 - DNA, 46 bp, synthetic construct
  misc_feature 1..46: note = Target Specific Oligonucleotide
  source 1..46: mol_type = other DNA
tgttagcttctatagtcactattcgagtaactgaattgtcacttta

SEQ ID NO: 640 - DNA, 45 bp, synthetic construct
  misc_feature 1..45: note = Target Specific Oligonucleotide
  source 1..45: mol_type = other DNA
tcaacaagtaatgtatcccgttcatgtaactgaattgtcacttta

SEQ ID NO: 641 - DNA, 44 bp, synthetic construct
  misc_feature 1..44: note = Target Specific Oligonucleotide
  source 1..44: mol_type = other DNA
tttccttggccgtaagttgttttcgtaactgaattgtcacttta

SEQ ID NO: 642 - DNA, 42 bp, synthetic construct
  misc_feature 1..42: note = Target Specific Oligonucleotide
  source 1..42: mol_type = other DNA
gggtcaacccagtctgttacctgtaactgaattgtcacttta

SEQ ID NO: 643 - DNA, 46 bp, synthetic construct
  misc_feature 1..46: note = Target Specific Oligonucleotide
  source 1..46: mol_type = other DNA
tctgagactgatagggaaacatatgggtaactgaattgtcacttta

SEQ ID NO: 644 - DNA, 42 bp, synthetic construct
  misc_feature 1..42: note = Target Specific Oligonucleotide
  source 1..42: mol_type = other DNA
tctttgccaccaatagcttggagtaactgaattgtcacttta

SEQ ID NO: 645 - DNA, 45 bp, synthetic construct
  misc_feature 1..45: note = Target Specific Oligonucleotide
  source 1..45: mol_type = other DNA
ctcattggaatggttagtagagcaagtaactgaattgtcacttta

SEQ ID NO: 646 - DNA, 44 bp, synthetic construct
  misc_feature 1..44: note = Target Specific Oligonucleotide
  source 1..44: mol_type = other DNA
ctgagtaaagagcgtgtattccaggtaactgaattgtcacttta

SEQ ID NO: 647 - DNA, 42 bp, synthetic construct
  misc_feature 1..42: note = Target Specific Oligonucleotide
  source 1..42: mol_type = other DNA
tggaggcataaagctggtaggcgtaactgaattgtcacttta

SEQ ID NO: 648 - DNA, 42 bp, synthetic construct
  misc_feature 1..42: note = Target Specific Oligonucleotide
  source 1..42: mol_type = other DNA
ggatctcaagcgttgcaggcgtaacgatagatttatacgacg

SEQ ID NO: 649 - DNA, 39 bp, synthetic construct
  misc_feature 1..39: note = Target Specific Oligonucleotide
  source 1..39: mol_type = other DNA
gcaccgcgagcggactagtaacgatagatttatacgacg

SEQ ID NO: 650 - DNA, 41 bp, synthetic construct
  misc_feature 1..41: note = Target Specific Oligonucleotide
  source 1..41: mol_type = other DNA
tgctgaggtcgctcacgaagtaacgatagatttatacgacg

SEQ ID NO: 651 - DNA, 44 bp, synthetic construct
  misc_feature 1..44: note = Target Specific Oligonucleotide
  source 1..44: mol_type = other DNA
ccggaccacgtagttaaatcttgtaacgatagatttatacgacg

SEQ ID NO: 652 - DNA, 43 bp, synthetic construct
  misc_feature 1..43: note = Target Specific Oligonucleotide
  source 1..43: mol_type = other DNA
aagttcttttgggcgatgccagtaacgatagatttatacgacg

SEQ ID NO: 653 - DNA, 48 bp, synthetic construct
  misc_feature 1..48: note = Target Specific Oligonucleotide
  source 1..48: mol_type = other DNA
gttcccttattagtctaatgttttgcgtaacgatagatttatacgacg

SEQ ID NO: 654 - DNA, 45 bp, synthetic construct
  misc_feature 1..45: note = Target Specific Oligonucleotide
  source 1..45: mol_type = other DNA
ttggaccagtgtacataaggatggtaacgatagatttatacgacg

SEQ ID NO: 655 - DNA, 41 bp, synthetic construct
  misc_feature 1..41: note = Target Specific Oligonucleotide
  source 1..41: mol_type = other DNA
tggcagtgcacggatggtcgtaacgatagatttatacgacg

SEQ ID NO: 656 - DNA, 47 bp, synthetic construct
  misc_feature 1..47: note = Target Specific Oligonucleotide
  source 1..47: mol_type = other DNA
ataccaattctagtgagaagttcacgtaacgatagatttatacgacg

SEQ ID NO: 657 - DNA, 44 bp, synthetic construct
  misc_feature 1..44: note = Target Specific Oligonucleotide
  source 1..44: mol_type = other DNA
tctctgtagggtaaggctctgtgtaacgatagatttatacgacg

SEQ ID NO: 658 - DNA, 45 bp, synthetic construct
  misc_feature 1..45: note = Target Specific Oligonucleotide
  source 1..45: mol_type = other DNA
tttagaggtagaccattgtgtgggtaacgatagatttatacgacg

SEQ ID NO: 659 - DNA, 42 bp, synthetic construct
  misc_feature 1..42: note = Target Specific Oligonucleotide
  source 1..42: mol_type = other DNA
agagagggcactctacgcttgtaacgatagatttatacgacg

SEQ ID NO: 660 - DNA, 44 bp, synthetic construct
  misc_feature 1..44: note = Target Specific Oligonucleotide
  source 1..44: mol_type = other DNA
caaaaagccctagctcagatgggtaacgatagatttatacgacg

SEQ ID NO: 661 - DNA, 43 bp, synthetic construct
  misc_feature 1..43: note = Target Specific Oligonucleotide
  source 1..43: mol_type = other DNA
cctctcctccctatttggctggtaacgatagatttatacgacg

SEQ ID NO: 662 - DNA, 46 bp, synthetic construct
  misc_feature 1..46: note = Target Specific Oligonucleotide
  source 1..46: mol_type = other DNA
ttttacaacgaactcattaggtccgtaacgatagatttatacgacg

SEQ ID NO: 663 - DNA, 45 bp, synthetic construct
  misc_feature 1..45: note = Target Specific Oligonucleotide
  source 1..45: mol_type = other DNA
ggagtagatcccccatctattccgtaacgatagatttatacgacg

SEQ ID NO: 664 - DNA, 44 bp, synthetic construct
  misc_feature 1..44: note = Target Specific Oligonucleotide
  source 1..44: mol_type = other DNA
gtctcttgcctcgtttctctccgtaacgatagatttatacgacg

SEQ ID NO: 665 - DNA, 43 bp, synthetic construct
  misc_feature 1..43: note = Target Specific Oligonucleotide
  source 1..43: mol_type = other DNA
aattctggcgtagcacaaacagtaacgatagatttatacgacg

SEQ ID NO: 666 - DNA, 45 bp, synthetic construct
  misc_feature 1..45: note = Target Specific Oligonucleotide
  source 1..45: mol_type = other DNA
ggccatgaacccctaaatctaaagtaacgatagatttatacgacg

SEQ ID NO: 667 - DNA, 42 bp, synthetic construct
  misc_feature 1..42: note = Target Specific Oligonucleotide
  source 1..42: mol_type = other DNA
gcccactggtaccatcttgggtaacgatagatttatacgacg

SEQ ID NO: 668 - DNA, 42 bp, synthetic construct
  misc_feature 1..42: note = Target Specific Oligonucleotide
  source 1..42: mol_type = other DNA
taccacgtggcactaacacagtaacgatagatttatacgacg

SEQ ID NO: 669 - DNA, 45 bp, synthetic construct
  misc_feature 1..45: note = Target Specific Oligonucleotide
  source 1..45: mol_type = other DNA
ttctagcttttagaccagcagttgtaacgatagatttatacgacg

SEQ ID NO: 670 - DNA, 44 bp, synthetic construct
  misc_feature 1..44: note = Target Specific Oligonucleotide
  source 1..44: mol_type = other DNA
cccaagtgctagtagaacctgcgtaacgatagatttatacgacg

SEQ ID NO: 671 - DNA, 47 bp, synthetic construct
  misc_feature 1..47: note = Target Specific Oligonucleotide
  source 1..47: mol_type = other DNA
aaacacatctatactcaaagacagagtaacgatagatttatacgacg

SEQ ID NO: 672 - DNA, 43 bp, synthetic construct
  misc_feature 1..43: note = Target Specific Oligonucleotide
  source 1..43: mol_type = other DNA
cacagacttgccgactctttggtaacgatagatttatacgacg

SEQ ID NO: 673 - DNA, 44 bp, synthetic construct
  misc_feature 1..44: note = Target Specific Oligonucleotide
  source 1..44: mol_type = other DNA
cacatgttaatgttcccgaccagtaacgatagatttatacgacg

SEQ ID NO: 674 - DNA, 44 bp, synthetic construct
  misc_feature 1..44: note = Target Specific Oligonucleotide
  source 1..44: mol_type = other DNA
attaatcgtttgcggatctgccgtaacgatagatttatacgacg

SEQ ID NO: 675 - DNA, 44 bp, synthetic construct
  misc_feature 1..44: note = Target Specific Oligonucleotide
  source 1..44: mol_type = other DNA
tgggtaacatcggacaagtttggtaacgatagatttatacgacg

SEQ ID NO: 676 - DNA, 44 bp, synthetic construct
  misc_feature 1..44: note = Target Specific Oligonucleotide
  source 1..44: mol_type = other DNA
tctgaagtttcgagccaatgttgtaacgatagatttatacgacg

SEQ ID NO: 677 - DNA, 44 bp, synthetic construct
  misc_feature 1..44: note = Target Specific Oligonucleotide
  source 1..44: mol_type = other DNA
ccattttcaccgctgaaatagagtaacgatagatttatacgacg

SEQ ID NO: 678 - DNA, 43 bp, synthetic construct
  misc_feature 1..43: note = Target Specific Oligonucleotide
  source 1..43: mol_type = other DNA
ctggatcacatgcgttgtcaggtaacgatagatttatacgacg

SEQ ID NO: 679 - DNA, 45 bp, synthetic construct
  misc_feature 1..45: note = Target Specific Oligonucleotide
  source 1..45: mol_type = other DNA
atatttgcctcgtaagatcccctgtaacgatagatttatacgacg

SEQ ID NO: 680 - DNA, 45 bp, synthetic construct
  misc_feature 1..45: note = Target Specific Oligonucleotide
  source 1..45: mol_type = other DNA
ctgaagcggtgttgtatcatagcgtaacgatagatttatacgacg

SEQ ID NO: 681 - DNA, 45 bp, synthetic construct
  misc_feature 1..45: note = Target Specific Oligonucleotide
  source 1..45: mol_type = other DNA
tagaataatggttcgtgaccttcgtaacgatagatttatacgacg

SEQ ID NO: 682 - DNA, 41 bp, synthetic construct
  misc_feature 1..41: note = Target Specific Oligonucleotide
  source 1..41: mol_type = other DNA
acctgaaggcgacgaggaggtaacgatagatttatacgacg

SEQ ID NO: 683 - DNA, 44 bp, synthetic construct
  misc_feature 1..44: note = Target Specific Oligonucleotide
  source 1..44: mol_type = other DNA
gcctggagtctatcgtacaatcgtaacgatagatttatacgacg

SEQ ID NO: 684 - DNA, 47 bp, synthetic construct
  misc_feature 1..47: note = Target Specific Oligonucleotide
  source 1..47: mol_type = other DNA
acatttaattagaccgtaacccttcgtaacgatagatttatacgacg

SEQ ID NO: 685 - DNA, 45 bp, synthetic construct
  misc_feature 1..45: note = Target Specific Oligonucleotide
  source 1..45: mol_type = other DNA
gctctggccatacttaacagatagtaacgatagatttatacgacg

SEQ ID NO: 686 - DNA, 46 bp, synthetic construct
  misc_feature 1..46: note = Target Specific Oligonucleotide
  source 1..46: mol_type = other DNA
cagcattgagtgataatgcaatctgtaacgatagatttatacgacg

SEQ ID NO: 687 - DNA, 44 bp, synthetic construct
  misc_feature 1..44: note = Target Specific Oligonucleotide
  source 1..44: mol_type = other DNA
atacacacccgctaccttactggtaacgatagatttatacgacg

SEQ ID NO: 688 - DNA, 44 bp, synthetic construct
  misc_feature 1..44: note = Target Specific Oligonucleotide
  source 1..44: mol_type = other DNA
acaaagacgagaactatggcaagtaacgatagatttatacgacg

SEQ ID NO: 689 - DNA, 44 bp, synthetic construct
  misc_feature 1..44: note = Target Specific Oligonucleotide
  source 1..44: mol_type = other DNA
aggcagacgctctaagtctaaagtaacgatagatttatacgacg

SEQ ID NO: 690 - DNA, 44 bp, synthetic construct
  misc_feature 1..44: note = Target Specific Oligonucleotide
  source 1..44: mol_type = other DNA
tctcttactcatcgtgaaaggcgtaacgatagatttatacgacg

SEQ ID NO: 691 - DNA, 42 bp, synthetic construct
  misc_feature 1..42: note = Target Specific Oligonucleotide
  source 1..42: mol_type = other DNA
gcctcaagttcgcattcagcgtaacgatagatttatacgacg

SEQ ID NO: 692 - DNA, 44 bp, synthetic construct
  misc_feature 1..44: note = Target Specific Oligonucleotide
  source 1..44: mol_type = other DNA
gaaatggaaacgttaacagccagtaacgatagatttatacgacg

SEQ ID NO: 693 - DNA, 43 bp, synthetic construct
  misc_feature 1..43: note = Target Specific Oligonucleotide
  source 1..43: mol_type = other DNA
cacctgcttcggaatctcagtgtaacgatagatttatacgacg

SEQ ID NO: 694 - DNA, 46 bp, synthetic construct
  misc_feature 1..46: note = Target Specific Oligonucleotide
  source 1..46: mol_type = other DNA
gacttgtgcttcgttaattaaacagtaacgatagatttatacgacg

SEQ ID NO: 695 - DNA, 46 bp, synthetic construct
  misc_feature 1..46: note = Target Specific Oligonucleotide
  source 1..46: mol_type = other DNA
gctgtaatatacgcccactttagcgtaacgatagatttatacgacg

SEQ ID NO: 696 - DNA, 44 bp, synthetic construct
  misc_feature 1..44: note = Target Specific Oligonucleotide
  source 1..44: mol_type = other DNA
ttccattgtgcggttgggattgtaacttataaaggatagacatc

SEQ ID NO: 697 - DNA, 45 bp, synthetic construct
  misc_feature 1..45: note = Target Specific Oligonucleotide
  source 1..45: mol_type = other DNA
ctccagtaatcgcttatggtccgtaacttataaaggatagacatc

SEQ ID NO: 698 - DNA, 45 bp, synthetic construct
  misc_feature 1..45: note = Target Specific Oligonucleotide
  source 1..45: mol_type = other DNA
cctcttctccgtcaagaagttcgtaacttataaaggatagacatc

SEQ ID NO: 699 - DNA, 45 bp, synthetic construct
  misc_feature 1..45: note = Target Specific Oligonucleotide
  source 1..45: mol_type = other DNA
tgattgagatggcggtatttgagtaacttataaaggatagacatc

SEQ ID NO: 700 - DNA, 45 bp, synthetic construct
  misc_feature 1..45: note = Target Specific Oligonucleotide
  source 1..45: mol_type = other DNA
gccaattataagtactcgggctgtaacttataaaggatagacatc

SEQ ID NO: 701 - DNA, 42 bp, synthetic construct
  misc_feature 1..42: note = Target Specific Oligonucleotide
  source 1..42: mol_type = other DNA
ccaggccagcgtatgttccgtaacttataaaggatagacatc

SEQ ID NO: 702 - DNA, 43 bp, synthetic construct
  misc_feature 1..43: note = Target Specific Oligonucleotide
  source 1..43: mol_type = other DNA
ctggggcaaccaacacttgtgtaacttataaaggatagacatc

SEQ ID NO: 703 - DNA, 43 bp, synthetic construct
  misc_feature 1..43: note = Target Specific Oligonucleotide
  source 1..43: mol_type = other DNA
ggctggacaataggctcccagtaacttataaaggatagacatc

SEQ ID NO: 704 - DNA, 44 bp, synthetic construct
  misc_feature 1..44: note = Target Specific Oligonucleotide
  source 1..44: mol_type = other DNA
aaagctatggcacgaaagccggtaacttataaaggatagacatc

SEQ ID NO: 705 - DNA, 44 bp, synthetic construct
  misc_feature 1..44: note = Target Specific Oligonucleotide
  source 1..44: mol_type = other DNA
gcagtccatctaaggtggactgtaacttataaaggatagacatc

SEQ ID NO: 706 - DNA, 46 bp, synthetic construct
  misc_feature 1..46: note = Target Specific Oligonucleotide
  source 1..46: mol_type = other DNA
tgtatatggagtaccctcttgctgtaacttataaaggatagacatc

SEQ ID NO: 707 - DNA, 44 bp, synthetic construct
  misc_feature 1..44: note = Target Specific Oligonucleotide
  source 1..44: mol_type = other DNA
ctggatcaccaaaacggcaatgtaacttataaaggatagacatc

SEQ ID NO: 708 - DNA, 45 bp, synthetic construct
  misc_feature 1..45: note = Target Specific Oligonucleotide
  source 1..45: mol_type = other DNA
ccaggataacctttacttgccagtaacttataaaggatagacatc

SEQ ID NO: 709 - DNA, 46 bp, synthetic construct
  misc_feature 1..46: note = Target Specific Oligonucleotide
  source 1..46: mol_type = other DNA
atttccagctgcgatatctactcgtaacttataaaggatagacatc

SEQ ID NO: 710 - DNA, 47 bp, synthetic construct
  misc_feature 1..47: note = Target Specific Oligonucleotide
  source 1..47: mol_type = other DNA
tttatagcagctagtcctttcttggtaacttataaaggatagacatc

SEQ ID NO: 711 - DNA, 46 bp, synthetic construct
  misc_feature 1..46: note = Target Specific Oligonucleotide
  source 1..46: mol_type = other DNA
tgatatgagattttcccggatgggtaacttataaaggatagacatc

SEQ ID NO: 712 - DNA, 43 bp, synthetic construct
  misc_feature 1..43: note = Target Specific Oligonucleotide
  source 1..43: mol_type = other DNA
tgacaacagttagggcggtggtaacttataaaggatagacatc

SEQ ID NO: 713 - DNA, 44 bp, synthetic construct
  misc_feature 1..44: note = Target Specific Oligonucleotide
  source 1..44: mol_type = other DNA
ttggttcccgagtactgtctagtaacttataaaggatagacatc

SEQ ID NO: 714 - DNA, 45 bp, synthetic construct
  misc_feature 1..45: note = Target Specific Oligonucleotide
  source 1..45: mol_type = other DNA
gtttagttccaacgccatctgtgtaacttataaaggatagacatc

SEQ ID NO: 715 - DNA, 45 bp, synthetic construct
  misc_feature 1..45: note = Target Specific Oligonucleotide
  source 1..45: mol_type = other DNA
aagagttgcgacagtatcacccgtaacttataaaggatagacatc

SEQ ID NO: 716 - DNA, 44 bp, synthetic construct
  misc_feature 1..44: note = Target Specific Oligonucleotide
  source 1..44: mol_type = other DNA
gctgctggatatccctcagaagtaacttataaaggatagacatc

SEQ ID NO: 717 - DNA, 46 bp, synthetic construct
  misc_feature 1..46: note = Target Specific Oligonucleotide
  source 1..46: mol_type = other DNA
tgcaagacagactatgtctatgggtaacttataaaggatagacatc

SEQ ID NO: 718 - DNA, 46 bp, synthetic construct
  misc_feature 1..46: note = Target Specific Oligonucleotide
  source 1..46: mol_type = other DNA
cttcaaggactaggtcaattgcagtaacttataaaggatagacatc

SEQ ID NO: 719 - DNA, 47 bp, synthetic construct
  misc_feature 1..47: note = Target Specific Oligonucleotide
  source 1..47: mol_type = other DNA
gatatgctgcactaactagtcttggtaacttataaaggatagacatc

SEQ ID NO: 720 - DNA, 40 bp, synthetic construct
  misc_feature 1..40: note = Target Specific Oligonucleotide
  source 1..40: mol_type = other DNA
atctcgagcaagacgttcagtgtaacggttatcgtggaaa

SEQ ID NO: 721 - DNA, 43 bp, synthetic construct
  misc_feature 1..43: note = Target Specific Oligonucleotide
  source 1..43: mol_type = other DNA
ctgtccataattagtccatgaggagtaacggttatcgtggaaa

SEQ ID NO: 722 - DNA, 43 bp, synthetic construct
  misc_feature 1..43: note = Target Specific Oligonucleotide
  source 1..43: mol_type = other DNA
atctttggattatactgcctgaccgtaacggttatcgtggaaa

SEQ ID NO: 723 - DNA, 39 bp, synthetic construct
  misc_feature 1..39: note = Target Specific Oligonucleotide
  source 1..39: mol_type = other DNA
cagcaagcttgcgaccttgagtaacggttatcgtggaaa

SEQ ID NO: 724 - DNA, 44 bp, synthetic construct
  misc_feature 1..44: note = Target Specific Oligonucleotide
  source 1..44: mol_type = other DNA
agttaaagttgagagatcatctccagtaacggttatcgtggaaa

SEQ ID NO: 725 - DNA, 44 bp, synthetic construct
  misc_feature 1..44: note = Target Specific Oligonucleotide
  source 1..44: mol_type = other DNA
aggaatggatctatcactatttctagtaacggttatcgtggaaa

SEQ ID NO: 726 - DNA, 44 bp, synthetic construct
  misc_feature 1..44: note = Target Specific Oligonucleotide
  source 1..44: mol_type = other DNA
cagcataatgattaggtatgcaaaagtaacggttatcgtggaaa

SEQ ID NO: 727 - DNA, 44 bp, synthetic construct
  misc_feature 1..44: note = Target Specific Oligonucleotide
  source 1..44: mol_type = other DNA
ttcagtctgataaaatctacagtcagtaacggttatcgtggaaa

SEQ ID NO: 728 - DNA, 39 bp, synthetic construct
  misc_feature 1..39: note = Target Specific Oligonucleotide
  source 1..39: mol_type = other DNA
tccaacacttcgtggggtccgtaacggttatcgtggaaa

SEQ ID NO: 729 - DNA, 39 bp, synthetic construct
  misc_feature 1..39: note = Target Specific Oligonucleotide
  source 1..39: mol_type = other DNA
agggctacaatgtgatggccgtaacggttatcgtggaaa

SEQ ID NO: 730 - DNA, 42 bp, synthetic construct
  misc_feature 1..42: note = Target Specific Oligonucleotide
  source 1..42: mol_type = other DNA
tcaaggtcataacctggttcatcgtaacggttatcgtggaaa

SEQ ID NO: 731 - DNA, 42 bp, synthetic construct
  misc_feature 1..42: note = Target Specific Oligonucleotide
  source 1..42: mol_type = other DNA
cctacaacaaacttgtctggaatgtaacggttatcgtggaaa

SEQ ID NO: 732 - DNA, 40 bp, synthetic construct
  misc_feature 1..40: note = Target Specific Oligonucleotide
  source 1..40: mol_type = other DNA
aaacaacaatccgcccaaagggtaacggttatcgtggaaa

SEQ ID NO: 733 - DNA, 41 bp, synthetic construct
  misc_feature 1..41: note = Target Specific Oligonucleotide
  source 1..41: mol_type = other DNA
aaagctctactaagcagatggcgtaacggttatcgtggaaa

SEQ ID NO: 734 - DNA, 41 bp, synthetic construct
  misc_feature 1..41: note = Target Specific Oligonucleotide
  source 1..41: mol_type = other DNA
actgctgacaaagattcactgggtaacggttatcgtggaaa

SEQ ID NO: 735 - DNA, 44 bp, synthetic construct
  misc_feature 1..44: note = Target Specific Oligonucleotide
  source 1..44: mol_type = other DNA
ctgatagtctataggctcatagtgcgtaacggttatcgtggaaa

SEQ ID NO: 736 - DNA, 40 bp, synthetic construct
  misc_feature 1..40: note = Target Specific Oligonucleotide
  source 1..40: mol_type = other DNA
atcactaatcacgacgccagggtaacggttatcgtggaaa

SEQ ID NO: 737 - DNA, 40 bp, synthetic construct
  misc_feature 1..40: note = Target Specific Oligonucleotide
  source 1..40: mol_type = other DNA
tggcgatgtcaataggactccgtaacggttatcgtggaaa

SEQ ID NO: 738 - DNA, 42 bp, synthetic construct
  misc_feature 1..42: note = Target Specific Oligonucleotide
  source 1..42: mol_type = other DNA
gaactctcatcttaggctttgtagtaacggttatcgtggaaa

SEQ ID NO: 739 - DNA, 44 bp, synthetic construct
  misc_feature 1..44: note = Target Specific Oligonucleotide
  source 1..44: mol_type = other DNA
tcaacattactgaaacactactaaagtaacggttatcgtggaaa

SEQ ID NO: 740 - DNA, 38 bp, synthetic construct
  misc_feature 1..38: note = Target Specific Oligonucleotide
  source 1..38: mol_type = other DNA
ggtcgccataacggagccggtaacggttatcgtggaaa

SEQ ID NO: 741 - DNA, 45 bp, synthetic construct
  misc_feature 1..45: note = Target Specific Oligonucleotide
  source 1..45: mol_type = other DNA
ttcttacagatatgagttcaatgtttgtaacggttatcgtggaaa

SEQ ID NO: 742 - DNA, 44 bp, synthetic construct
  misc_feature 1..44: note = Target Specific Oligonucleotide
  source 1..44: mol_type = other DNA
aggacagaacaaaacttcttagatggtaacggttatcgtggaaa

SEQ ID NO: 743 - DNA, 36 bp, synthetic construct
  misc_feature 1..36: note = Target Specific Oligonucleotide
  source 1..36: mol_type = other DNA
tcgcagccgcctgaagcgtaacggttatcgtggaaa

SEQ ID NO: 744 - DNA, 41 bp, synthetic construct
  misc_feature 1..41: note = Target Specific Oligonucleotide
  source 1..41: mol_type = other DNA
ggaatttggaaccggcttgcgtaacggtactttatctagct

SEQ ID NO: 745 - DNA, 46 bp, synthetic construct
  misc_feature 1..46: note = Target Specific Oligonucleotide
  source 1..46: mol_type = other DNA
agagtcttgagtataatcttggtaggtaacggtactttatctagct

SEQ ID NO: 746 - DNA, 46 bp, synthetic construct
  misc_feature 1..46: note = Target Specific Oligonucleotide
  source 1..46: mol_type = other DNA
attcaggaagcatacactaattcttgtaacggtactttatctagct

SEQ ID NO: 747 - DNA, 44 bp, synthetic construct
  misc_feature 1..44: note = Target Specific Oligonucleotide
  source 1..44: mol_type = other DNA
tccagaaataagcgaaaatagcagtaacggtactttatctagct

SEQ ID NO: 748 - DNA, 47 bp, synthetic construct
  misc_feature 1..47: note = Target Specific Oligonucleotide
  source 1..47: mol_type = other DNA
atcttttgaactactagctacaaaatgtaacggtactttatctagct

SEQ ID NO: 749 - DNA, 42 bp, synthetic construct
  misc_feature 1..42: note = Target Specific Oligonucleotide
  source 1..42: mol_type = other DNA
taacaagctcgtaatggcccagtaacggtactttatctagct

SEQ ID NO: 750 - DNA, 42 bp, synthetic construct
  misc_feature 1..42: note = Target Specific Oligonucleotide
  source 1..42: mol_type = other DNA
catccaaaccgtgtgaaagctgtaacggtactttatctagct

SEQ ID NO: 751 - DNA, 41 bp, synthetic construct
  misc_feature 1..41: note = Target Specific Oligonucleotide
  source 1..41: mol_type = other DNA
acaacgccatgtgcgatttggtaacggtactttatctagct

SEQ ID NO: 752 - DNA, 45 bp, synthetic construct
  misc_feature 1..45: note = Target Specific Oligonucleotide
  source 1..45: mol_type = other DNA
ctacctaccactaggtacctatcagtaacggtactttatctagct

SEQ ID NO: 753 - DNA, 43 bp, synthetic construct
  misc_feature 1..43: note = Target Specific Oligonucleotide
  source 1..43: mol_type = other DNA
aggacatttacgtgtacagcacgtaacggtactttatctagct

SEQ ID NO: 754 - DNA, 43 bp, synthetic construct
  misc_feature 1..43: note = Target Specific Oligonucleotide
  source 1..43: mol_type = other DNA
cgcctgaatacaacgagttaacgtaacggtactttatctagct

SEQ ID NO: 755 - DNA, 44 bp, synthetic construct
  misc_feature 1..44: note = Target Specific Oligonucleotide
  source 1..44: mol_type = other DNA
cttccagattgtcgccgttaattgtaacggtactttatctagct

SEQ ID NO: 756 - DNA, 44 bp, synthetic construct
  misc_feature 1..44: note = Target Specific Oligonucleotide
  source 1..44: mol_type = other DNA
cagatttacctaaaccaccaccagtaacggtactttatctagct

SEQ ID NO: 757 - DNA, 45 bp, synthetic construct
  misc_feature 1..45: note = Target Specific Oligonucleotide
  source 1..45: mol_type = other DNA
tttgagctgatctagtaggtcttcgtaacggtactttatctagct

SEQ ID NO: 758 - DNA, 41 bp, synthetic construct
  misc_feature 1..41: note = Target Specific Oligonucleotide
  source 1..41: mol_type = other DNA
tctgtagctcgatccctggagtaacggtactttatctagct

SEQ ID NO: 759 - DNA, 44 bp, synthetic construct
  misc_feature 1..44: note = Target Specific Oligonucleotide
  source 1..44: mol_type = other DNA
caactgtcattcggttaaactgagtaacggtactttatctagct

SEQ ID NO: 760 - DNA, 44 bp, synthetic construct
  misc_feature 1..44: note = Target Specific Oligonucleotide
  source 1..44: mol_type = other DNA
tttgcttccgttcatcattagtcgtaacggtactttatctagct

SEQ ID NO: 761 - DNA, 45 bp, synthetic construct
  misc_feature 1..45: note = Target Specific Oligonucleotide
  source 1..45: mol_type = other DNA
acttccaatagtctggaaatcacagtaacggtactttatctagct

SEQ ID NO: 762 - DNA, 45 bp, synthetic construct
  misc_feature 1..45: note = Target Specific Oligonucleotide
  source 1..45: mol_type = other DNA
tcatctgctggattaagctaatacgtaacggtactttatctagct

SEQ ID NO: 763 - DNA, 41 bp, synthetic construct
  misc_feature 1..41: note = Target Specific Oligonucleotide
  source 1..41: mol_type = other DNA
agataggctcccgtatgcccgtaacggtactttatctagct

SEQ ID NO: 764 - DNA, 41 bp, synthetic construct
  misc_feature 1..41: note = Target Specific Oligonucleotide
  source 1..41: mol_type = other DNA
gcttctcggctaccacagacgtaacggtactttatctagct

SEQ ID NO: 765 - DNA, 46 bp, synthetic construct
  misc_feature 1..46: note = Target Specific Oligonucleotide
  source 1..46: mol_type = other DNA
agacttactcttaagaccatacttcgtaacggtactttatctagct

SEQ ID NO: 766 - DNA, 42 bp, synthetic construct
  misc_feature 1..42: note = Target Specific Oligonucleotide
  source 1..42: mol_type = other DNA
gttgagaggtggcgtttttgagtaacggtactttatctagct

SEQ ID NO: 767 - DNA, 44 bp, synthetic construct
  misc_feature 1..44: note = Target Specific Oligonucleotide
  source 1..44: mol_type = other DNA
tcaatacgtttctgcaagagtttgtaacggtactttatctagct

SEQ ID NO: 768 - DNA, 41 bp, synthetic construct
  misc_feature 1..41: note = Target Specific Oligonucleotide
  source 1..41: mol_type = other DNA
ttatggcctctatagcatctcccgtaactccatctagaccg

SEQ ID NO: 769 - DNA, 40 bp, synthetic construct
  misc_feature 1..40: note = Target Specific Oligonucleotide
  source 1..40: mol_type = other DNA
cagtccgggcaactatcttattgtaactccatctagaccg

SEQ ID NO: 770 - DNA, 42 bp, synthetic construct
  misc_feature 1..42: note = Target Specific Oligonucleotide
  source 1..42: mol_type = other DNA
ttcaagatacgaataatcctccgagtaactccatctagaccg

SEQ ID NO: 771 - DNA, 40 bp, synthetic construct
  misc_feature 1..40: note = Target Specific Oligonucleotide
  source 1..40: mol_type = other DNA
gtaggcgcttagcttgattttcgtaactccatctagaccg

SEQ ID NO: 772 - DNA, 38 bp, synthetic construct
  misc_feature 1..38: note = Target Specific Oligonucleotide
  source 1..38: mol_type = other DNA
cttcaagtcgggctcttgcagtaactccatctagaccg

SEQ ID NO: 773 - DNA, 38 bp, synthetic construct
  misc_feature 1..38: note = Target Specific Oligonucleotide
  source 1..38: mol_type = other DNA
agaatgaggcacgcaaagctgtaactccatctagaccg

SEQ ID NO: 774 - DNA, 40 bp, synthetic construct
  misc_feature 1..40: note = Target Specific Oligonucleotide
  source 1..40: mol_type = other DNA
ctcagcatccgataggactttcgtaactccatctagaccg

SEQ ID NO: 775 - DNA, 40 bp, synthetic construct
  misc_feature 1..40: note = Target Specific Oligonucleotide
  source 1..40: mol_type = other DNA
aagtcactataacacccgtcctgtaactccatctagaccg

SEQ ID NO: 776 - DNA, 39 bp, synthetic construct
  misc_feature 1..39: note = Target Specific Oligonucleotide
  source 1..39: mol_type = other DNA
ttggcatggtagatcgtccaggtaactccatctagaccg

SEQ ID NO: 777 - DNA, 39 bp, synthetic construct
  misc_feature 1..39: note = Target Specific Oligonucleotide
  source 1..39: mol_type = other DNA
caccttccaacgactcaatccgtaactccatctagaccg

SEQ ID NO: 778 - DNA, 42 bp, synthetic construct
  misc_feature 1..42: note = Target Specific Oligonucleotide
  source 1..42: mol_type = other DNA
acaacctgtaagtgtagttctctggtaactccatctagaccg

SEQ ID NO: 779 - DNA, 43 bp, synthetic construct
  misc_feature 1..43: note = Target Specific Oligonucleotide
  source 1..43: mol_type = other DNA
aaatcagatactcgtctacagaatggtaactccatctagaccg

SEQ ID NO: 780 - DNA, 38 bp, synthetic construct
  misc_feature 1..38: note = Target Specific Oligonucleotide
  source 1..38: mol_type = other DNA
agcagcaagtcgcattttccgtaactccatctagaccg

SEQ ID NO: 781 - DNA, 39 bp, synthetic construct
  misc_feature 1..39: note = Target Specific Oligonucleotide
  source 1..39: mol_type = other DNA
actggcatggcggataaaaaggtaactccatctagaccg

SEQ ID NO: 782 - DNA, 38 bp, synthetic construct
  misc_feature 1..38: note = Target Specific Oligonucleotide
  source 1..38: mol_type = other DNA
cgggtgctctcgtgaacaaagtaactccatctagaccg

SEQ ID NO: 783 - DNA, 41 bp, synthetic construct
  misc_feature 1..41: note = Target Specific Oligonucleotide
  source 1..41: mol_type = other DNA
gatcgaagagtcgagataaggatgtaactccatctagaccg

SEQ ID NO: 784 - DNA, 41 bp, synthetic construct
  misc_feature 1..41: note = Target Specific Oligonucleotide
  source 1..41: mol_type = other DNA
ttgttgaagacgcttccataagtgtaactccatctagaccg

SEQ ID NO: 785 - DNA, 37 bp, synthetic construct
  misc_feature 1..37: note = Target Specific Oligonucleotide
  source 1..37: mol_type = other DNA
gtggtgacgcagaatcccggtaactccatctagaccg

SEQ ID NO: 786 - DNA, 45 bp, synthetic construct
  misc_feature 1..45: note = Target Specific Oligonucleotide
  source 1..45: mol_type = other DNA
cattataaagacgtaataatttagggtgtaactccatctagaccg

SEQ ID NO: 787 - DNA, 39 bp, synthetic construct
  misc_feature 1..39: note = Target Specific Oligonucleotide
  source 1..39: mol_type = other DNA
gctcccactcctagacatgttgtaactccatctagaccg

SEQ ID NO: 788 - DNA, 40 bp, synthetic construct
  misc_feature 1..40: note = Target Specific Oligonucleotide
  source 1..40: mol_type = other DNA
tttgttacacggttaaacagccgtaactccatctagaccg

SEQ ID NO: 789 - DNA, 42 bp, synthetic construct
  misc_feature 1..42: note = Target Specific Oligonucleotide
  source 1..42: mol_type = other DNA
gaggtaaataaacgtcgataggaagtaactccatctagaccg

SEQ ID NO: 790 - DNA, 40 bp, synthetic construct
  misc_feature 1..40: note = Target Specific Oligonucleotide
  source 1..40: mol_type = other DNA
ttaatgtggagtgcaagttggggtaactccatctagaccg

SEQ ID NO: 791 - DNA, 41 bp, synthetic construct
  misc_feature 1..41: note = Target Specific Oligonucleotide
  source 1..41: mol_type = other DNA
ccttgtatcttagcttgggatgtgtaactccatctagaccg

SEQ ID NO: 792 - DNA, 43 bp, synthetic construct
  misc_feature 1..43: note = Target Specific Oligonucleotide
  source 1..43: mol_type = other DNA
tcaatgatcagcggatacctattgtaactcagtagatatagcg

SEQ ID NO: 793 - DNA, 43 bp, synthetic construct
  misc_feature 1..43: note = Target Specific Oligonucleotide
  source 1..43: mol_type = other DNA
cgtgtgatcttacgatccttatagtaactcagtagatatagcg

SEQ ID NO: 794 - DNA, 43 bp, synthetic construct
  misc_feature 1..43: note = Target Specific Oligonucleotide
  source 1..43: mol_type = other DNA
tcatcctctacgagtctatcttggtaactcagtagatatagcg

SEQ ID NO: 795 - DNA, 40 bp, synthetic construct
  misc_feature 1..40: note = Target Specific Oligonucleotide
  source 1..40: mol_type = other DNA
tcaaagtaacccgcgtaggcgtaactcagtagatatagcg

SEQ ID NO: 796 - DNA, 41 bp, synthetic construct
  misc_feature 1..41: note = Target Specific Oligonucleotide
  source 1..41: mol_type = other DNA
ataggacgcatactgtcgcaagtaactcagtagatatagcg

SEQ ID NO: 797 - DNA, 41 bp, synthetic construct
  misc_feature 1..41: note = Target Specific Oligonucleotide
  source 1..41: mol_type = other DNA
ttatgtccgtcgaccttgcatgtaactcagtagatatagcg

SEQ ID NO: 798 - DNA, 42 bp, synthetic construct
  misc_feature 1..42: note = Target Specific Oligonucleotide
  source 1..42: mol_type = other DNA
ttccaaggaacggacttctagggtaactcagtagatatagcg

SEQ ID NO: 799 - DNA, 42 bp, synthetic construct
  misc_feature 1..42: note = Target Specific Oligonucleotide
  source 1..42: mol_type = other DNA
ataggtacgaacgctctcgatcgtaactcagtagatatagcg

SEQ ID NO: 800 - DNA, 42 bp, synthetic construct
  misc_feature 1..42: note = Target Specific Oligonucleotide
  source 1..42: mol_type = other DNA
tcttggatgatcgcatgaaaccgtaactcagtagatatagcg

SEQ ID NO: 801 - DNA, 42 bp, synthetic construct
  misc_feature 1..42: note = Target Specific Oligonucleotide
  source 1..42: mol_type = other DNA
acaacccaattaacgtgaagccgtaactcagtagatatagcg

SEQ ID NO: 802 - DNA, 41 bp, synthetic construct
  misc_feature 1..41: note = Target Specific Oligonucleotide
  source 1..41: mol_type = other DNA
atgcggtataacgcacgttccgtaactcagtagatatagcg

SEQ ID NO: 803 - DNA, 41 bp, synthetic construct
  misc_feature 1..41: note = Target Specific Oligonucleotide
  source 1..41: mol_type = other DNA
ctcaagtcacgacgcagttctgtaactcagtagatatagcg

SEQ ID NO: 804 - DNA, 40 bp, synthetic construct
  misc_feature 1..40: note = Target Specific Oligonucleotide
  source 1..40: mol_type = other DNA
gcaagtcacgaacgtgactcgtaactcagtagatatagcg

SEQ ID NO: 805 - DNA, 40 bp, synthetic construct
  misc_feature 1..40: note = Target Specific Oligonucleotide
  source 1..40: mol_type = other DNA
ttggcgttatactgcgccacgtaactcagtagatatagcg

SEQ ID NO: 806 - DNA, 40 bp, synthetic construct
  misc_feature 1..40: note = Target Specific Oligonucleotide
  source 1..40: mol_type = other DNA
agtggtgcgtatcgtaagcggtaactcagtagatatagcg

SEQ ID NO: 807 - DNA, 40 bp, synthetic construct
  misc_feature 1..40: note = Target Specific Oligonucleotide
  source 1..40: mol_type = other DNA
atccaggcgacgaaacgagagtaactcagtagatatagcg

SEQ ID NO: 808 - DNA, 40 bp, synthetic construct
  misc_feature 1..40: note = Target Specific Oligonucleotide
  source 1..40: mol_type = other DNA
ttggatgaatcggcaggctggtaactcagtagatatagcg

SEQ ID NO: 809 - DNA, 43 bp, synthetic construct
  misc_feature 1..43: note = Target Specific Oligonucleotide
  source 1..43: mol_type = other DNA
tcagtcaattcgtaatgtacaccgtaactcagtagatatagcg

SEQ ID NO: 810 - DNA, 41 bp, synthetic construct
  misc_feature 1..41: note = Target Specific Oligonucleotide
  source 1..41: mol_type = other DNA
catcgaagacgatccactggcgtaactcagtagatatagcg

SEQ ID NO: 811 - DNA, 39 bp, synthetic construct
  misc_feature 1..39: note = Target Specific Oligonucleotide
  source 1..39: mol_type = other DNA
cacggatcaagcggcacttgtaactcagtagatatagcg

SEQ ID NO: 812 - DNA, 39 bp, synthetic construct
  misc_feature 1..39: note = Target Specific Oligonucleotide
  source 1..39: mol_type = other DNA
tccaccgtatcgcaagctggtaactcagtagatatagcg

SEQ ID NO: 813 - DNA, 41 bp, synthetic construct
  misc_feature 1..41: note = Target Specific Oligonucleotide
  source 1..41: mol_type = other DNA
atgcaaagtttcgtagggcgagtaactcagtagatatagcg

SEQ ID NO: 814 - DNA, 38 bp, synthetic construct
  misc_feature 1..38: note = Target Specific Oligonucleotide
  source 1..38: mol_type = other DNA
gcgttggcacgacgacatgtaactcagtagatatagcg

SEQ ID NO: 815 - DNA, 38 bp, synthetic construct
  misc_feature 1..38: note = Target Specific Oligonucleotide
  source 1..38: mol_type = other DNA
ttgcagacggacgcccaagtaactcagtagatatagcg

SEQ ID NO: 816 - DNA, 40 bp, synthetic construct
  misc_feature 1..40: note = Target Specific Oligonucleotide
  source 1..40: mol_type = other DNA
agctgtatcgtcaaggcactcgtaaccttttgaaacgcta

SEQ ID NO: 817 - DNA, 39 bp, synthetic construct
  misc_feature 1..39: note = Target Specific Oligonucleotide
  source 1..39: mol_type = other DNA
ctcgctcccagtccgaaatggtaaccttttgaaacgcta

SEQ ID NO: 818 - DNA, 44 bp, synthetic construct
  misc_feature 1..44: note = Target Specific Oligonucleotide
  source 1..44: mol_type = other DNA
acttgtactagtatgccttaagaaagtaaccttttgaaacgcta

SEQ ID NO: 819 - DNA, 42 bp, synthetic construct
  misc_feature 1..42: note = Target Specific Oligonucleotide
  source 1..42: mol_type = other DNA
gtgaccatgactaatagcagtgggtaaccttttgaaacgcta

SEQ ID NO: 820 - DNA, 41 bp, synthetic construct
  misc_feature 1..41: note = Target Specific Oligonucleotide
  source 1..41: mol_type = other DNA
gctgtgtcgagaatatccaagagtaaccttttgaaacgcta

SEQ ID NO: 821 - DNA, 41 bp, synthetic construct
  misc_feature 1..41: note = Target Specific Oligonucleotide
  source 1..41: mol_type = other DNA
cctactgtcgctaatggattgggtaaccttttgaaacgcta

SEQ ID NO: 822 - DNA, 41 bp, synthetic construct
  misc_feature 1..41: note = Target Specific Oligonucleotide
  source 1..41: mol_type = other DNA
acactagcactacctaaggaccgtaaccttttgaaacgcta

SEQ ID NO: 823 - DNA, 41 bp, synthetic construct
  misc_feature 1..41: note = Target Specific Oligonucleotide
  source 1..41: mol_type = other DNA
gtctgtgaactagttcaggcacgtaaccttttgaaacgcta

SEQ ID NO: 824 - DNA, 40 bp, synthetic construct
  misc_feature 1..40: note = Target Specific Oligonucleotide
  source 1..40: mol_type = other DNA
ctaggtcagcgcaaccaaatggtaaccttttgaaacgcta

SEQ ID NO: 825 - DNA, 42 bp, synthetic construct
  misc_feature 1..42: note = Target Specific Oligonucleotide
  source 1..42: mol_type = other DNA
cctaataagctataactggcccagtaaccttttgaaacgcta

SEQ ID NO: 826 - DNA, 43 bp, synthetic construct
  misc_feature 1..43: note = Target Specific Oligonucleotide
  source 1..43: mol_type = other DNA
ctaaattcctatgcagtgtgactcgtaaccttttgaaacgcta

SEQ ID NO: 827 - DNA, 42 bp, synthetic construct
  misc_feature 1..42: note = Target Specific Oligonucleotide
  source 1..42: mol_type = other DNA
atcttgttgagctatccaaactggtaaccttttgaaacgcta

SEQ ID NO: 828 - DNA, 42 bp, synthetic construct
  misc_feature 1..42: note = Target Specific Oligonucleotide
  source 1..42: mol_type = other DNA
aaatacgcatcgtgttatctctggtaaccttttgaaacgcta

SEQ ID NO: 829 - DNA, 42 bp, synthetic construct
  misc_feature 1..42: note = Target Specific Oligonucleotide
  source 1..42: mol_type = other DNA
tgttaagagaactagccaaacctgtaaccttttgaaacgcta

SEQ ID NO: 830 - DNA, 43 bp, synthetic construct
  misc_feature 1..43: note = Target Specific Oligonucleotide
  source 1..43: mol_type = other DNA
cataaagtagcttgatcgaagagtgtaaccttttgaaacgcta

SEQ ID NO: 831 - DNA, 41 bp, synthetic construct
  misc_feature 1..41: note = Target Specific Oligonucleotide
  source 1..41: mol_type = other DNA
taagcaaacacgcctttacatagtaaccttttgaaacgcta

SEQ ID NO: 832 - DNA, 44 bp, synthetic construct
  misc_feature 1..44: note = Target Specific Oligonucleotide
  source 1..44: mol_type = other DNA
aaggccttagtaagatattacagacgtaaccttttgaaacgcta

SEQ ID NO: 833 - DNA, 45 bp, synthetic construct
  misc_feature 1..45: note = Target Specific Oligonucleotide
  source 1..45: mol_type = other DNA
acaatatacgtctgctatattcttccgtaaccttttgaaacgcta

SEQ ID NO: 834 - DNA, 41 bp, synthetic construct
  misc_feature 1..41: note = Target Specific Oligonucleotide
  source 1..41: mol_type = other DNA
tccactggatagggttctgtctgtaaccttttgaaacgcta

SEQ ID NO: 835 - DNA, 44 bp, synthetic construct
  misc_feature 1..44: note = Target Specific Oligonucleotide
  source 1..44: mol_type = other DNA
aagttatactatgaaagagcagtctgtaaccttttgaaacgcta

SEQ ID NO: 836 - DNA, 44 bp, synthetic construct
  misc_feature 1..44: note = Target Specific Oligonucleotide
  source 1..44: mol_type = other DNA
acatgttaatgcctaagtctatgtagtaaccttttgaaacgcta

SEQ ID NO: 837 - DNA, 40 bp, synthetic construct
  misc_feature 1..40: note = Target Specific Oligonucleotide
  source 1..40: mol_type = other DNA
gcctagaatgcctacttgggagtaaccttttgaaacgcta

SEQ ID NO: 838 - DNA, 41 bp, synthetic construct
  misc_feature 1..41: note = Target Specific Oligonucleotide
  source 1..41: mol_type = other DNA
tcagtggaatcgtagcaaaacagtaaccttttgaaacgcta

SEQ ID NO: 839 - DNA, 45 bp, synthetic construct
  misc_feature 1..45: note = Target Specific Oligonucleotide
  source 1..45: mol_type = other DNA
acaataattaggagtagtacagttcagtaaccttttgaaacgcta

SEQ ID NO: 840 - DNA, 42 bp, synthetic construct
  misc_feature 1..42: note = Target Specific Oligonucleotide
  source 1..42: mol_type = other DNA
ttccactcggataagatgctgagtaactcagacatgtagatt

SEQ ID NO: 841 - DNA, 42 bp, synthetic construct
  misc_feature 1..42: note = Target Specific Oligonucleotide
  source 1..42: mol_type = other DNA
aatactccacacgcaaatttccgtaactcagacatgtagatt

SEQ ID NO: 842 - DNA, 40 bp, synthetic construct
  misc_feature 1..40: note = Target Specific Oligonucleotide
  source 1..40: mol_type = other DNA
ggccagaccatcgctatctggtaactcagacatgtagatt

SEQ ID NO: 843 - DNA, 42 bp, synthetic construct
  misc_feature 1..42: note = Target Specific Oligonucleotide
  source 1..42: mol_type = other DNA
caccacactatgtcgaaaagtggtaactcagacatgtagatt

SEQ ID NO: 844 - DNA, 42 bp, synthetic construct
  misc_feature 1..42: note = Target Specific Oligonucleotide
  source 1..42: mol_type = other DNA
ccctttcttgcggagattctctgtaactcagacatgtagatt

SEQ ID NO: 845 - DNA, 40 bp, synthetic construct
  misc_feature 1..40: note = Target Specific Oligonucleotide
  source 1..40: mol_type = other DNA
acaaacacgcacctcaaagcgtaactcagacatgtagatt

SEQ ID NO: 846 - DNA, 42 bp, synthetic construct
  misc_feature 1..42: note = Target Specific Oligonucleotide
  source 1..42: mol_type = other DNA
cccagtagattaccactggagtgtaactcagacatgtagatt

SEQ ID NO: 847 - DNA, 39 bp, synthetic construct
  misc_feature 1..39: note = Target Specific Oligonucleotide
  source 1..39: mol_type = other DNA
gcagtgctcgcttagtgctgtaactcagacatgtagatt

SEQ ID NO: 848 - DNA, 40 bp, synthetic construct
  misc_feature 1..40: note = Target Specific Oligonucleotide
  source 1..40: mol_type = other DNA
ggctcgacgctaggatctgagtaactcagacatgtagatt

SEQ ID NO: 849 - DNA, 39 bp, synthetic construct
  misc_feature 1..39: note = Target Specific Oligonucleotide
  source 1..39: mol_type = other DNA
aggatgggcctccggttcagtaactcagacatgtagatt

SEQ ID NO: 850 - DNA, 40 bp, synthetic construct
  misc_feature 1..40: note = Target Specific Oligonucleotide
  source 1..40: mol_type = other DNA
ggaacatctcgaagcgctcagtaactcagacatgtagatt

SEQ ID NO: 851 - DNA, 40 bp, synthetic construct
  misc_feature 1..40: note = Target Specific Oligonucleotide
  source 1..40: mol_type = other DNA
agacggaaaccgtagctgccgtaactcagacatgtagatt

SEQ ID NO: 852 - DNA, 41 bp, synthetic construct
  misc_feature 1..41: note = Target Specific Oligonucleotide
  source 1..41: mol_type = other DNA
tgttcaatatcgtccggggacgtaactcagacatgtagatt

SEQ ID NO: 853 - DNA, 40 bp, synthetic construct
  misc_feature 1..40: note = Target Specific Oligonucleotide
  source 1..40: mol_type = other DNA
cattgcttgggacggcaagggtaactcagacatgtagatt

SEQ ID NO: 854 - DNA, 42 bp, synthetic construct
  misc_feature 1..42: note = Target Specific Oligonucleotide
  source 1..42: mol_type = other DNA
agtggagaatgtcagtctgagtgtaactcagacatgtagatt

SEQ ID NO: 855 - DNA, 43 bp, synthetic construct
  misc_feature 1..43: note = Target Specific Oligonucleotide
  source 1..43: mol_type = other DNA
agttttttatggcgggaggtagagtaactcagacatgtagatt

SEQ ID NO: 856 - DNA, 41 bp, synthetic construct
  misc_feature 1..41: note = Target Specific Oligonucleotide
  source 1..41: mol_type = other DNA
tagaaactaccaacccaccgagtaactcagacatgtagatt

SEQ ID NO: 857 - DNA, 42 bp, synthetic construct
  misc_feature 1..42: note = Target Specific Oligonucleotide
  source 1..42: mol_type = other DNA
ttccctggttagtacggtgaaggtaactcagacatgtagatt

SEQ ID NO: 858 - DNA, 42 bp, synthetic construct
  misc_feature 1..42: note = Target Specific Oligonucleotide
  source 1..42: mol_type = other DNA
taagctggtatgtcctactcccgtaactcagacatgtagatt

SEQ ID NO: 859 - DNA, 41 bp, synthetic construct
  misc_feature 1..41: note = Target Specific Oligonucleotide
  source 1..41: mol_type = other DNA
ctgacgcacacctattgcaaggtaactcagacatgtagatt

SEQ ID NO: 860 - DNA, 39 bp, synthetic construct
  misc_feature 1..39: note = Target Specific Oligonucleotide
  source 1..39: mol_type = other DNA
tcaccgtcgtggaaagcacgtaactcagacatgtagatt

SEQ ID NO: 861 - DNA, 44 bp, synthetic construct
  misc_feature 1..44: note = Target Specific Oligonucleotide
  source 1..44: mol_type = other DNA
gtaaactaacccttaactgcaagagtaactcagacatgtagatt

SEQ ID NO: 862 - DNA, 44 bp, synthetic construct
  misc_feature 1..44: note = Target Specific Oligonucleotide
  source 1..44: mol_type = other DNA
tttaggtactaaggttcaccaagagtaactcagacatgtagatt

SEQ ID NO: 863 - DNA, 46 bp, synthetic construct
  misc_feature 1..46: note = Target Specific Oligonucleotide
  source 1..46: mol_type = other DNA
tcatcatatacaagagatgaaatcctgtaactcagacatgtagatt

SEQ ID NO: 864 - DNA, 41 bp, synthetic construct
  misc_feature 1..41: note = Target Specific Oligonucleotide
  source 1..41: mol_type = other DNA
ccatggtgacgatcctcacggtaactaacataaattcgatg

SEQ ID NO: 865 - DNA, 42 bp, synthetic construct
  misc_feature 1..42: note = Target Specific Oligonucleotide
  source 1..42: mol_type = other DNA
gtgaacgtcttgggttgcttcgtaactaacataaattcgatg

SEQ ID NO: 866 - DNA, 39 bp, synthetic construct
  misc_feature 1..39: note = Target Specific Oligonucleotide
  source 1..39: mol_type = other DNA
ggcctggacggcgtaagagtaactaacataaattcgatg

SEQ ID NO: 867 - DNA, 40 bp, synthetic construct
  misc_feature 1..40: note = Target Specific Oligonucleotide
  source 1..40: mol_type = other DNA
gaagctcatgcgggaagcggtaactaacataaattcgatg

SEQ ID NO: 868 - DNA, 43 bp, synthetic construct
  misc_feature 1..43: note = Target Specific Oligonucleotide
  source 1..43: mol_type = other DNA
tgtcactgtacgagatgtttcggtaactaacataaattcgatg

SEQ ID NO: 869 - DNA, 43 bp, synthetic construct
  misc_feature 1..43: note = Target Specific Oligonucleotide
  source 1..43: mol_type = other DNA
ttgaagcttggtatgtcaggaggtaactaacataaattcgatg

SEQ ID NO: 870 - DNA, 42 bp, synthetic construct
  misc_feature 1..42: note = Target Specific Oligonucleotide
  source 1..42: mol_type = other DNA
ggttcccgatacatagctggagtaactaacataaattcgatg

SEQ ID NO: 871 - DNA, 44 bp, synthetic construct
  misc_feature 1..44: note = Target Specific Oligonucleotide
  source 1..44: mol_type = other DNA
atctttgaagtcgaaagacaggcgtaactaacataaattcgatg

SEQ ID NO: 872 - DNA, 44 bp, synthetic construct
  misc_feature 1..44: note = Target Specific Oligonucleotide
  source 1..44: mol_type = other DNA
gcttattaacacgaagctttgaggtaactaacataaattcgatg

SEQ ID NO: 873 - DNA, 42 bp, synthetic construct
  misc_feature 1..42: note = Target Specific Oligonucleotide
  source 1..42: mol_type = other DNA
tttcattccacgggaaggagagtaactaacataaattcgatg

SEQ ID NO: 874 - DNA, 41 bp, synthetic construct
  misc_feature 1..41: note = Target Specific Oligonucleotide
  source 1..41: mol_type = other DNA
tcagtgcttcgttggccattgtaactaacataaattcgatg

SEQ ID NO: 875 - DNA, 43 bp, synthetic construct
  misc_feature 1..43: note = Target Specific Oligonucleotide
  source 1..43: mol_type = other DNA
ctctgcgtaccaagcagtaattgtaactaacataaattcgatg

SEQ ID NO: 876 - DNA, 43 bp, synthetic construct
  misc_feature 1..43: note = Target Specific Oligonucleotide
  source 1..43: mol_type = other DNA
tttcaaggcctcgatttctgtcgtaactaacataaattcgatg

SEQ ID NO: 877 - DNA, 43 bp, synthetic construct
  misc_feature 1..43: note = Target Specific Oligonucleotide
  source 1..43: mol_type = other DNA
tagttcacaccgtacatctccagtaactaacataaattcgatg

SEQ ID NO: 878 - DNA, 42 bp, synthetic construct
  misc_feature 1..42: note = Target Specific Oligonucleotide
  source 1..42: mol_type = other DNA
cccacatttccggagtcatctgtaactaacataaattcgatg

SEQ ID NO: 879 - DNA, 45 bp, synthetic construct
  misc_feature 1..45: note = Target Specific Oligonucleotide
  source 1..45: mol_type = other DNA
ggtcctgagctatcttcagatattgtaactaacataaattcgatg

SEQ ID NO: 880 - DNA, 39 bp, synthetic construct
  misc_feature 1..39: note = Target Specific Oligonucleotide
  source 1..39: mol_type = other DNA
tcctccgtgcgaatcgctgtaactaacataaattcgatg

SEQ ID NO: 881 - DNA, 43 bp, synthetic construct
  misc_feature 1..43: note = Target Specific Oligonucleotide
  source 1..43: mol_type = other DNA
gccctttttattccggattgcagtaactaacataaattcgatg

SEQ ID NO: 882 - DNA, 40 bp, synthetic construct
  misc_feature 1..40: note = Target Specific Oligonucleotide
  source 1..40: mol_type = other DNA
gctgtctcctcagaccgcagtaactaacataaattcgatg

SEQ ID NO: 883 - DNA, 41 bp, synthetic construct
  misc_feature 1..41: note = Target Specific Oligonucleotide
  source 1..41: mol_type = other DNA
cacccctgtcggagttctcagtaactaacataaattcgatg

SEQ ID NO: 884 - DNA, 46 bp, synthetic construct
  misc_feature 1..46: note = Target Specific Oligonucleotide
  source 1..46: mol_type = other DNA
cagatttattaccctttttggaagcgtaactaacataaattcgatg

SEQ ID NO: 885 - DNA, 39 bp, synthetic construct
  misc_feature 1..39: note = Target Specific Oligonucleotide
  source 1..39: mol_type = other DNA
tcatgggcgggtacgtgggtaactaacataaattcgatg

SEQ ID NO: 886 - DNA, 42 bp, synthetic construct
  misc_feature 1..42: note = Target Specific Oligonucleotide
  source 1..42: mol_type = other DNA
caaaaatccccgcttgtgaacgtaactaacataaattcgatg

SEQ ID NO: 887 - DNA, 42 bp, synthetic construct
  misc_feature 1..42: note = Target Specific Oligonucleotide
  source 1..42: mol_type = other DNA
ctggggtcgtagtcaccatacgtaactaacataaattcgatg

SEQ ID NO: 888 - DNA, 39 bp, synthetic construct
  misc_feature 1..39: note = Target Specific Oligonucleotide
  source 1..39: mol_type = other DNA
cacacgataccggcaaagaaggtaacgtgacgattaccc

SEQ ID NO: 889 - DNA, 38 bp, synthetic construct
  misc_feature 1..38: note = Target Specific Oligonucleotide
  source 1..38: mol_type = other DNA
ggtgtcagtctccgacgtgagtaacgtgacgattaccc

SEQ ID NO: 890 - DNA, 39 bp, synthetic construct
  misc_feature 1..39: note = Target Specific Oligonucleotide
  source 1..39: mol_type = other DNA
gtgatggtgatcatctgggccgtaacgtgacgattaccc

SEQ ID NO: 891 - DNA, 36 bp, synthetic construct
  misc_feature 1..36: note = Target Specific Oligonucleotide
  source 1..36: mol_type = other DNA
ttggcctcgggaccaagcgtaacgtgacgattaccc

SEQ ID NO: 892 - DNA, 39 bp, synthetic construct
  misc_feature 1..39: note = Target Specific Oligonucleotide
  source 1..39: mol_type = other DNA
ctgtcatcttggtcaggtggtgtaacgtgacgattaccc

SEQ ID NO: 893 - DNA, 38 bp, synthetic construct
  misc_feature 1..38: note = Target Specific Oligonucleotide
  source 1..38: mol_type = other DNA
atcaccttgccgaaagtgccgtaacgtgacgattaccc

SEQ ID NO: 894 - DNA, 37 bp, synthetic construct
  misc_feature 1..37: note = Target Specific Oligonucleotide
  source 1..37: mol_type = other DNA
actgcacggccgtagtcatgtaacgtgacgattaccc

SEQ ID NO: 895 - DNA, 37 bp, synthetic construct
  misc_feature 1..37: note = Target Specific Oligonucleotide
  source 1..37: mol_type = other DNA
aggtactcaggtgtgccgcgtaacgtgacgattaccc

SEQ ID NO: 896 - DNA, 38 bp, synthetic construct
  misc_feature 1..38: note = Target Specific Oligonucleotide
  source 1..38: mol_type = other DNA
gaaggtgcgttcgatgacaggtaacgtgacgattaccc

SEQ ID NO: 897 - DNA, 39 bp, synthetic construct
  misc_feature 1..39: note = Target Specific Oligonucleotide
  source 1..39: mol_type = other DNA
ctcctcaggagtctccacatggtaacgtgacgattaccc

SEQ ID NO: 898 - DNA, 40 bp, synthetic construct
  misc_feature 1..40: note = Target Specific Oligonucleotide
  source 1..40: mol_type = other DNA
gtctggaaagagtacttcaggggtaacgtgacgattaccc

SEQ ID NO: 899 - DNA, 36 bp, synthetic construct
  misc_feature 1..36: note = Target Specific Oligonucleotide
  source 1..36: mol_type = other DNA
caggacgcggttctcggtgtaacgtgacgattaccc

SEQ ID NO: 900 - DNA, 38 bp, synthetic construct
  misc_feature 1..38: note = Target Specific Oligonucleotide
  source 1..38: mol_type = other DNA
atctcagcgccatagaagcggtaacgtgacgattaccc

SEQ ID NO: 901 - DNA, 37 bp, synthetic construct
  misc_feature 1..37: note = Target Specific Oligonucleotide
  source 1..37: mol_type = other DNA
tcgttcatggtcacgcggtgtaacgtgacgattaccc

SEQ ID NO: 902 - DNA, 39 bp, synthetic construct
  misc_feature 1..39: note = Target Specific Oligonucleotide
  source 1..39: mol_type = other DNA
cagcccgaagtctgtgatcttgtaacgtgacgattaccc

SEQ ID NO: 903 - DNA, 37 bp, synthetic construct
  misc_feature 1..37: note = Target Specific Oligonucleotide
  source 1..37: mol_type = other DNA
tcatggtggcaccgtccttgtaacgtgacgattaccc

SEQ ID NO: 904 - DNA, 38 bp, synthetic construct
  misc_feature 1..38: note = Target Specific Oligonucleotide
  source 1..38: mol_type = other DNA
ggtagtccagggctgacacagtaacgtgacgattaccc

SEQ ID NO: 905 - DNA, 41 bp, synthetic construct
  misc_feature 1..41: note = Target Specific Oligonucleotide
  source 1..41: mol_type = other DNA
ccatcattcttgaggaggaagtagtaacgtgacgattaccc

SEQ ID NO: 906 - DNA, 37 bp, synthetic construct
  misc_feature 1..37: note = Target Specific Oligonucleotide
  source 1..37: mol_type = other DNA
acaaagcagaggcggtcgtgtaacgtgacgattaccc

SEQ ID NO: 907 - DNA, 38 bp, synthetic construct
  misc_feature 1..38: note = Target Specific Oligonucleotide
  source 1..38: mol_type = other DNA
cggtcctcggagaacacacggtaacgtgacgattaccc

SEQ ID NO: 908 - DNA, 37 bp, synthetic construct
  misc_feature 1..37: note = Target Specific Oligonucleotide
  source 1..37: mol_type = other DNA
gagcctcacgttggtccacgtaacgtgacgattaccc

SEQ ID NO: 909 - DNA, 40 bp, synthetic construct
  misc_feature 1..40: note = Target Specific Oligonucleotide
  source 1..40: mol_type = other DNA
ctccttgtagccaatgaaggtggtaacgtgacgattaccc

SEQ ID NO: 910 - DNA, 38 bp, synthetic construct
  misc_feature 1..38: note = Target Specific Oligonucleotide
  source 1..38: mol_type = other DNA
ttcacaatagccacgtcgctgtaacgtgacgattaccc

SEQ ID NO: 911 - DNA, 37 bp, synthetic construct
  misc_feature 1..37: note = Target Specific Oligonucleotide
  source 1..37: mol_type = other DNA
ccctcgtttgtgcagccaagtaacgtgacgattaccc

SEQ ID NO: 912 - DNA, 43 bp, synthetic construct
  misc_feature 1..43: note = Target Specific Oligonucleotide
  source 1..43: mol_type = other DNA
agctatgtccgctatcttcatcggtaactctcttctattgcag

SEQ ID NO: 913 - DNA, 41 bp, synthetic construct
  misc_feature 1..41: note = Target Specific Oligonucleotide
  source 1..41: mol_type = other DNA
ttggcacatcgagacatggttgtaactctcttctattgcag

SEQ ID NO: 914 - DNA, 42 bp, synthetic construct
  misc_feature 1..42: note = Target Specific Oligonucleotide
  source 1..42: mol_type = other DNA
ccatcttggtcgtactttgtgagtaactctcttctattgcag

SEQ ID NO: 915 - DNA, 41 bp, synthetic construct
  misc_feature 1..41: note = Target Specific Oligonucleotide
  source 1..41: mol_type = other DNA
ccaactgagcatacgctaggagtaactctcttctattgcag

SEQ ID NO: 916 - DNA, 41 bp, synthetic construct
  misc_feature 1..41: note = Target Specific Oligonucleotide
  source 1..41: mol_type = other DNA
tcacgtactagccgttccatcgtaactctcttctattgcag

SEQ ID NO: 917 - DNA, 43 bp, synthetic construct
  misc_feature 1..43: note = Target Specific Oligonucleotide
  source 1..43: mol_type = other DNA
tttctccaagtcgtctctcatctgtaactctcttctattgcag

SEQ ID NO: 918 - DNA, 42 bp, synthetic construct
  misc_feature 1..42: note = Target Specific Oligonucleotide
  source 1..42: mol_type = other DNA
tcgtaagaaacgccactagaaagtaactctcttctattgcag

SEQ ID NO: 919 - DNA, 39 bp, synthetic construct
  misc_feature 1..39: note = Target Specific Oligonucleotide
  source 1..39: mol_type = other DNA
cactggcgtgcctaaaccagtaactctcttctattgcag

SEQ ID NO: 920 - DNA, 45 bp, synthetic construct
  misc_feature 1..45: note = Target Specific Oligonucleotide
  source 1..45: mol_type = other DNA
acatatcatacgtggacattagaacgtaactctcttctattgcag

SEQ ID NO: 921 - DNA, 40 bp, synthetic construct
  misc_feature 1..40: note = Target Specific Oligonucleotide
  source 1..40: mol_type = other DNA
gccgatggaatgctccatccgtaactctcttctattgcag

SEQ ID NO: 922 - DNA, 43 bp, synthetic construct
  misc_feature 1..43: note = Target Specific Oligonucleotide
  source 1..43: mol_type = other DNA
gattagcaatcggtcacaaagacgtaactctcttctattgcag

SEQ ID NO: 923 - DNA, 43 bp, synthetic construct
  misc_feature 1..43: note = Target Specific Oligonucleotide
  source 1..43: mol_type = other DNA
ggtgtagtagtacacattggagcgtaactctcttctattgcag

SEQ ID NO: 924 - DNA, 42 bp, synthetic construct
  misc_feature 1..42: note = Target Specific Oligonucleotide
  source 1..42: mol_type = other DNA
gatgaaacttcggttgtcagctgtaactctcttctattgcag

SEQ ID NO: 925 - DNA, 42 bp, synthetic construct
  misc_feature 1..42: note = Target Specific Oligonucleotide
  source 1..42: mol_type = other DNA
ttcttgaggctagcaacttgtggtaactctcttctattgcag

SEQ ID NO: 926 - DNA, 43 bp, synthetic construct
  misc_feature 1..43: note = Target Specific Oligonucleotide
  source 1..43: mol_type = other DNA
gtcccaaaactcgattagcttccgtaactctcttctattgcag

SEQ ID NO: 927 - DNA, 42 bp, synthetic construct
  misc_feature 1..42: note = Target Specific Oligonucleotide
  source 1..42: mol_type = other DNA
tattcgtggaacccctaacagcgtaactctcttctattgcag

SEQ ID NO: 928 - DNA, 43 bp, synthetic construct
  misc_feature 1..43: note = Target Specific Oligonucleotide
  source 1..43: mol_type = other DNA
caaagttgatatcgcccaaaatggtaactctcttctattgcag

SEQ ID NO: 929 - DNA, 42 bp, synthetic construct
  misc_feature 1..42: note = Target Specific Oligonucleotide
  source 1..42: mol_type = other DNA
tagatccaagcggttccatttcgtaactctcttctattgcag

SEQ ID NO: 930 - DNA, 42 bp, synthetic construct
  misc_feature 1..42: note = Target Specific Oligonucleotide
  source 1..42: mol_type = other DNA
tgaccagttcccgtaaaacactgtaactctcttctattgcag

SEQ ID NO: 931 - DNA, 41 bp, synthetic construct
  misc_feature 1..41: note = Target Specific Oligonucleotide
  source 1..41: mol_type = other DNA
tggcacatggtcgatacacatgtaactctcttctattgcag

SEQ ID NO: 932 - DNA, 41 bp, synthetic construct
  misc_feature 1..41: note = Target Specific Oligonucleotide
  source 1..41: mol_type = other DNA
tgacagcacaacgatcacaacgtaactctcttctattgcag

SEQ ID NO: 933 - DNA, 42 bp, synthetic construct
  misc_feature 1..42: note = Target Specific Oligonucleotide
  source 1..42: mol_type = other DNA
aagctcatgaactatgccagaggtaactctcttctattgcag

SEQ ID NO: 934 - DNA, 41 bp, synthetic construct
  misc_feature 1..41: note = Target Specific Oligonucleotide
  source 1..41: mol_type = other DNA
cgtcatacctgaccgagtactgtaactctcttctattgcag

SEQ ID NO: 935 - DNA, 43 bp, synthetic construct
  misc_feature 1..43: note = Target Specific Oligonucleotide
  source 1..43: mol_type = other DNA
tcttgattgtcgagaaagacttggtaactctcttctattgcag

SEQ ID NO: 936 - DNA, 42 bp, synthetic construct
  misc_feature 1..42: note = Target Specific Oligonucleotide
  source 1..42: mol_type = other DNA
tactgagccctatcggaaagtgtaactgatcttcatattacg

SEQ ID NO: 937 - DNA, 42 bp, synthetic construct
  misc_feature 1..42: note = Target Specific Oligonucleotide
  source 1..42: mol_type = other DNA
ccaaatcccttcgcagcaattgtaactgatcttcatattacg

SEQ ID NO: 938 - DNA, 42 bp, synthetic construct
  misc_feature 1..42: note = Target Specific Oligonucleotide
  source 1..42: mol_type = other DNA
gccatgatcaatcgcatcactgtaactgatcttcatattacg

SEQ ID NO: 939 - DNA, 43 bp, synthetic construct
  misc_feature 1..43: note = Target Specific Oligonucleotide
  source 1..43: mol_type = other DNA
atcatccagattcgaaccgtccgtaactgatcttcatattacg

SEQ ID NO: 940 - DNA, 43 bp, synthetic construct
  misc_feature 1..43: note = Target Specific Oligonucleotide
  source 1..43: mol_type = other DNA
ggactacattcgtgaagaagctgtaactgatcttcatattacg

SEQ ID NO: 941 - DNA, 42 bp, synthetic construct
  misc_feature 1..42: note = Target Specific Oligonucleotide
  source 1..42: mol_type = other DNA
ggagaggatccgtccaagaaagtaactgatcttcatattacg

SEQ ID NO: 942 - DNA, 42 bp, synthetic construct
  misc_feature 1..42: note = Target Specific Oligonucleotide
  source 1..42: mol_type = other DNA
gtcccagcattagaacctgtggtaactgatcttcatattacg

SEQ ID NO: 943 - DNA, 42 bp, synthetic construct
  misc_feature 1..42: note = Target Specific Oligonucleotide
  source 1..42: mol_type = other DNA
actctcagccagacctttagcgtaactgatcttcatattacg

SEQ ID NO: 944 - DNA, 44 bp, synthetic construct
  misc_feature 1..44: note = Target Specific Oligonucleotide
  source 1..44: mol_type = other DNA
catagtgtcatgtgcatgtagtcgtaactgatcttcatattacg

SEQ ID NO: 945 - DNA, 44 bp, synthetic construct
  misc_feature 1..44: note = Target Specific Oligonucleotide
  source 1..44: mol_type = other DNA
ccctgaccagcatattatctacagtaactgatcttcatattacg

SEQ ID NO: 946 - DNA, 41 bp, synthetic construct
  misc_feature 1..41: note = Target Specific Oligonucleotide
  source 1..41: mol_type = other DNA
cagcgcctgttacaagctctgtaactgatcttcatattacg

SEQ ID NO: 947 - DNA, 41 bp, synthetic construct
  misc_feature 1..41: note = Target Specific Oligonucleotide
  source 1..41: mol_type = other DNA
cgccagtcttaactgccatggtaactgatcttcatattacg

SEQ ID NO: 948 - DNA, 43 bp, synthetic construct
  misc_feature 1..43: note = Target Specific Oligonucleotide
  source 1..43: mol_type = other DNA
tcttcccaacaccgtaatctttgtaactgatcttcatattacg

SEQ ID NO: 949 - DNA, 43 bp, synthetic construct
  misc_feature 1..43: note = Target Specific Oligonucleotide
  source 1..43: mol_type = other DNA
gtgacttatgttcgggtctctcgtaactgatcttcatattacg

SEQ ID NO: 950 - DNA, 43 bp, synthetic construct
  misc_feature 1..43: note = Target Specific Oligonucleotide
  source 1..43: mol_type = other DNA
tgttaggttctagggccatagcgtaactgatcttcatattacg

SEQ ID NO: 951 - DNA, 42 bp, synthetic construct
  misc_feature 1..42: note = Target Specific Oligonucleotide
  source 1..42: mol_type = other DNA
ctggatagctttgggcaactcgtaactgatcttcatattacg

SEQ ID NO: 952 - DNA, 45 bp, synthetic construct
  misc_feature 1..45: note = Target Specific Oligonucleotide
  source 1..45: mol_type = other DNA
tgcagacagggtatgtatatttgggtaactgatcttcatattacg

SEQ ID NO: 953 - DNA, 44 bp, synthetic construct
  misc_feature 1..44: note = Target Specific Oligonucleotide
  source 1..44: mol_type = other DNA
aagaaaatatagctgagctccctgtaactgatcttcatattacg

SEQ ID NO: 954 - DNA, 40 bp, synthetic construct
  misc_feature 1..40: note = Target Specific Oligonucleotide
  source 1..40: mol_type = other DNA
gagaccggagaaacccggcgtaactgatcttcatattacg

SEQ ID NO: 955 - DNA, 40 bp, synthetic construct
  misc_feature 1..40: note = Target Specific Oligonucleotide
  source 1..40: mol_type = other DNA
ccgcaggagaccgaggtctgtaactgatcttcatattacg

SEQ ID NO: 956 - DNA, 42 bp, synthetic construct
  misc_feature 1..42: note = Target Specific Oligonucleotide
  source 1..42: mol_type = other DNA
aggctagggctcaagacaagggtaactgatcttcatattacg

SEQ ID NO: 957 - DNA, 38 bp, synthetic construct
  misc_feature 1..38: note = Target Specific Oligonucleotide
  source 1..38: mol_type = other DNA
gcgtccggtgtcgccatgtaactgatcttcatattacg

SEQ ID NO: 958 - DNA, 41 bp, synthetic construct
  misc_feature 1..41: note = Target Specific Oligonucleotide
  source 1..41: mol_type = other DNA
actctgggtcggcttgatccgtaactgatcttcatattacg

SEQ ID NO: 959 - DNA, 41 bp, synthetic construct
  misc_feature 1..41: note = Target Specific Oligonucleotide
  source 1..41: mol_type = other DNA
catgggaggatcgagcctgggtaactgatcttcatattacg

SEQ ID NO: 960 - DNA, 39 bp, synthetic construct
  misc_feature 1..39: note = Target Specific Oligonucleotide
  source 1..39: mol_type = other DNA
ggcactggaaacgattgacttgtaacggcaatacatcgt

SEQ ID NO: 961 - DNA, 38 bp, synthetic construct
  misc_feature 1..38: note = Target Specific Oligonucleotide
  source 1..38: mol_type = other DNA
tcaggcattcgttcacctgtgtaacggcaatacatcgt

SEQ ID NO: 962 - DNA, 40 bp, synthetic construct
  misc_feature 1..40: note = Target Specific Oligonucleotide
  source 1..40: mol_type = other DNA
actgtagcgattaatgccatccgtaacggcaatacatcgt

SEQ ID NO: 963 - DNA, 38 bp, synthetic construct
  misc_feature 1..38: note = Target Specific Oligonucleotide
  source 1..38: mol_type = other DNA
atagcctccattgcggttgggtaacggcaatacatcgt

SEQ ID NO: 964 - DNA, 40 bp, synthetic construct
  misc_feature 1..40: note = Target Specific Oligonucleotide
  source 1..40: mol_type = other DNA
cattcgggtgatcgatacacttgtaacggcaatacatcgt

SEQ ID NO: 965 - DNA, 39 bp, synthetic construct
  misc_feature 1..39: note = Target Specific Oligonucleotide
  source 1..39: mol_type = other DNA
gcacactcatcgagttgctccgtaacggcaatacatcgt

SEQ ID NO: 966 - DNA, 37 bp, synthetic construct
  misc_feature 1..37: note = Target Specific Oligonucleotide
  source 1..37: mol_type = other DNA
tcccgcttaatgcgcaggtgtaacggcaatacatcgt

SEQ ID NO: 967 - DNA, 37 bp, synthetic construct
  misc_feature 1..37: note = Target Specific Oligonucleotide
  source 1..37: mol_type = other DNA
ggtcccctgtatggcgtgagtaacggcaatacatcgt

SEQ ID NO: 968 - DNA, 38 bp, synthetic construct
  misc_feature 1..38: note = Target Specific Oligonucleotide
  source 1..38: mol_type = other DNA
aacacactggcggttgtcaagtaacggcaatacatcgt

SEQ ID NO: 969 - DNA, 39 bp, synthetic construct
  misc_feature 1..39: note = Target Specific Oligonucleotide
  source 1..39: mol_type = other DNA
cacacatcgacgaaggtttcagtaacggcaatacatcgt

SEQ ID NO: 970 - DNA, 39 bp, synthetic construct
  misc_feature 1..39: note = Target Specific Oligonucleotide
  source 1..39: mol_type = other DNA
ggctcttgcacgagttaacttgtaacggcaatacatcgt

SEQ ID NO: 971 - DNA, 39 bp, synthetic construct
  misc_feature 1..39: note = Target Specific Oligonucleotide
  source 1..39: mol_type = other DNA
atgtcaatggtacaccgctgagtaacggcaatacatcgt

SEQ ID NO: 972 - DNA, 38 bp, synthetic construct
  misc_feature 1..38: note = Target Specific Oligonucleotide
  source 1..38: mol_type = other DNA
gccatgcttacgctttcgttgtaacggcaatacatcgt

SEQ ID NO: 973 - DNA, 40 bp, synthetic construct
  misc_feature 1..40: note = Target Specific Oligonucleotide
  source 1..40: mol_type = other DNA
ggtataccagtaacacggaccagtaacggcaatacatcgt

SEQ ID NO: 974 - DNA, 40 bp, synthetic construct
  misc_feature 1..40: note = Target Specific Oligonucleotide
  source 1..40: mol_type = other DNA
gactctctcacgcataaacctggtaacggcaatacatcgt

SEQ ID NO: 975 - DNA, 40 bp, synthetic construct
  misc_feature 1..40: note = Target Specific Oligonucleotide
  source 1..40: mol_type = other DNA
aggagatgcgtaggtcaattcagtaacggcaatacatcgt

SEQ ID NO: 976 - DNA, 39 bp, synthetic construct
  misc_feature 1..39: note = Target Specific Oligonucleotide
  source 1..39: mol_type = other DNA
ccaacaggacgctagtgtagagtaacggcaatacatcgt

SEQ ID NO: 977 - DNA, 42 bp, synthetic construct
  misc_feature 1..42: note = Target Specific Oligonucleotide
  source 1..42: mol_type = other DNA
cagtaccaattaggttagcttctggtaacggcaatacatcgt

SEQ ID NO: 978 - DNA, 38 bp, synthetic construct
  misc_feature 1..38: note = Target Specific Oligonucleotide
  source 1..38: mol_type = other DNA
tccatggccgtcgatcaatggtaacggcaatacatcgt

SEQ ID NO: 979 - DNA, 40 bp, synthetic construct
  misc_feature 1..40: note = Target Specific Oligonucleotide
  source 1..40: mol_type = other DNA
tagtcaggaatatgcggtcattgtaacggcaatacatcgt

SEQ ID NO: 980 - DNA, 41 bp, synthetic construct
  misc_feature 1..41: note = Target Specific Oligonucleotide
  source 1..41: mol_type = other DNA
tatagcaactacttcgcatttccgtaacggcaatacatcgt

SEQ ID NO: 981 - DNA, 39 bp, synthetic construct
  misc_feature 1..39: note = Target Specific Oligonucleotide
  source 1..39: mol_type = other DNA
cgatggtgtcctacggatgtcgtaacggcaatacatcgt

SEQ ID NO: 982 - DNA, 44 bp, synthetic construct
  misc_feature 1..44: note = Target Specific Oligonucleotide
  source 1..44: mol_type = other DNA
gtaaccatagtaccacttattagtgggtaacggcaatacatcgt

SEQ ID NO: 983 - DNA, 37 bp, synthetic construct
  misc_feature 1..37: note = Target Specific Oligonucleotide
  source 1..37: mol_type = other DNA
cagcatcagcccgtgagtagtaacggcaatacatcgt

SEQ ID NO: 984 - DNA, 39 bp, synthetic construct
  misc_feature 1..39: note = Target Specific Oligonucleotide
  source 1..39: mol_type = other DNA
tcagatgctactggccgcttagacaagttaagatcggaa

SEQ ID NO: 985 - DNA, 41 bp, synthetic construct
  misc_feature 1..41: note = Target Specific Oligonucleotide
  source 1..41: mol_type = other DNA
cttgatcaaggcgctgaacaatagacaagttaagatcggaa

SEQ ID NO: 986 - DNA, 41 bp, synthetic construct
  misc_feature 1..41: note = Target Specific Oligonucleotide
  source 1..41: mol_type = other DNA
tcccttcgtatctcagcgagatagacaagttaagatcggaa

SEQ ID NO: 987 - DNA, 41 bp, synthetic construct
  misc_feature 1..41: note = Target Specific Oligonucleotide
  source 1..41: mol_type = other DNA
agactgttgactggcgtgatgtagacaagttaagatcggaa

SEQ ID NO: 988 - DNA, 41 bp, synthetic construct
  misc_feature 1..41: note = Target Specific Oligonucleotide
  source 1..41: mol_type = other DNA
ccaacatgctcgcatgagttctagacaagttaagatcggaa

SEQ ID NO: 989 - DNA, 41 bp, synthetic construct
  misc_feature 1..41: note = Target Specific Oligonucleotide
  source 1..41: mol_type = other DNA
ttccttcaccgaactgaggagtagacaagttaagatcggaa

SEQ ID NO: 990 - DNA, 40 bp, synthetic construct
  misc_feature 1..40: note = Target Specific Oligonucleotide
  source 1..40: mol_type = other DNA
agttccaacgagcggcttcatagacaagttaagatcggaa

SEQ ID NO: 991 - DNA, 40 bp, synthetic construct
  misc_feature 1..40: note = Target Specific Oligonucleotide
  source 1..40: mol_type = other DNA
ccatggctgacgagatctgatagacaagttaagatcggaa

SEQ ID NO: 992 - DNA, 40 bp, synthetic construct
  misc_feature 1..40: note = Target Specific Oligonucleotide
  source 1..40: mol_type = other DNA
tagcctaagacccggagctttagacaagttaagatcggaa

SEQ ID NO: 993 - DNA, 40 bp, synthetic construct
  misc_feature 1..40: note = Target Specific Oligonucleotide
  source 1..40: mol_type = other DNA
gcagatactcagcggcattgtagacaagttaagatcggaa

SEQ ID NO: 994 - DNA, 40 bp, synthetic construct
  misc_feature 1..40: note = Target Specific Oligonucleotide
  source 1..40: mol_type = other DNA
catagctcacgcagaacgtgtagacaagttaagatcggaa

SEQ ID NO: 995 - DNA, 39 bp, synthetic construct
  misc_feature 1..39: note = Target Specific Oligonucleotide
  source 1..39: mol_type = other DNA
gtcggccaccgttgaatgatagacaagttaagatcggaa

SEQ ID NO: 996 - DNA, 39 bp, synthetic construct
  misc_feature 1..39: note = Target Specific Oligonucleotide
  source 1..39: mol_type = other DNA
agaacggtcagcgagcgtatagacaagttaagatcggaa

SEQ ID NO: 997 - DNA, 44 bp, synthetic construct
  misc_feature 1..44: note = Target Specific Oligonucleotide
  source 1..44: mol_type = other DNA
gttatgcttagagtgttatctccatagacaagttaagatcggaa

SEQ ID NO: 998 - DNA, 40 bp, synthetic construct
  misc_feature 1..40: note = Target Specific Oligonucleotide
  source 1..40: mol_type = other DNA
tggtgatgtccgtgcgttcctagacaagttaagatcggaa

SEQ ID NO: 999 - DNA, 40 bp, synthetic construct
  misc_feature 1..40: note = Target Specific Oligonucleotide
  source 1..40: mol_type = other DNA
ggtccagaggatcgctctcttagacaagttaagatcggaa

SEQ ID NO: 1000 - DNA, 39 bp, synthetic construct
  misc_feature 1..39: note = Target Specific Oligonucleotide
  source 1..39: mol_type = other DNA
cgtggactgcaagtcccgatagacaagttaagatcggaa

SEQ ID NO: 1001 - DNA, 41 bp, synthetic construct
  misc_feature 1..41: note = Target Specific Oligonucleotide
  source 1..41: mol_type = other DNA
acaggtcaattcccgggtaagtagacaagttaagatcggaa

SEQ ID NO: 1002 - DNA, 41 bp, synthetic construct
  misc_feature 1..41: note = Target Specific Oligonucleotide
  source 1..41: mol_type = other DNA
ctgcaccaggttagggtgttttagacaagttaagatcggaa

SEQ ID NO: 1003 - DNA, 43 bp, synthetic construct
  misc_feature 1..43: note = Target Specific Oligonucleotide
  source 1..43: mol_type = other DNA
ggaaatggcatatacgtgggatttagacaagttaagatcggaa

SEQ ID NO: 1004 - DNA, 40 bp, synthetic construct
  misc_feature 1..40: note = Target Specific Oligonucleotide
  source 1..40: mol_type = other DNA
cttttgtctttgcgcccaggtagacaagttaagatcggaa

SEQ ID NO: 1005 - DNA, 38 bp, synthetic construct
  misc_feature 1..38: note = Target Specific Oligonucleotide
  source 1..38: mol_type = other DNA
tcctgccggttgcactcctagacaagttaagatcggaa

SEQ ID NO: 1006 - DNA, 43 bp, synthetic construct
  misc_feature 1..43: note = Target Specific Oligonucleotide
  source 1..43: mol_type = other DNA
atcatacagtgcaacgaaaaggttagacaagttaagatcggaa

SEQ ID NO: 1007 - DNA, 40 bp, synthetic construct
  misc_feature 1..40: note = Target Specific Oligonucleotide
  source 1..40: mol_type = other DNA
tcccacttgtcgtagttgggtagacaagttaagatcggaa

SEQ ID NO: 1008 - DNA, 39 bp, synthetic construct
  misc_feature 1..39: note = Target Specific Oligonucleotide
  source 1..39: mol_type = other DNA
aggtggtgttccgaacgagctagacagcccaattaaacg

SEQ ID NO: 1009 - DNA, 39 bp, synthetic construct
  misc_feature 1..39: note = Target Specific Oligonucleotide
  source 1..39: mol_type = other DNA
agatccgtgagcggaatgtatagacagcccaattaaacg

SEQ ID NO: 1010 - DNA, 41 bp, synthetic construct
  misc_feature 1..41: note = Target Specific Oligonucleotide
  source 1..41: mol_type = other DNA
ctcactcgctttagtggactcctagacagcccaattaaacg

SEQ ID NO: 1011 - DNA, 37 bp, synthetic construct
  misc_feature 1..37: note = Target Specific Oligonucleotide
  source 1..37: mol_type = other DNA
gccaaagaccgtggcgagtagacagcccaattaaacg

SEQ ID NO: 1012 - DNA, 41 bp, synthetic construct
  misc_feature 1..41: note = Target Specific Oligonucleotide
  source 1..41: mol_type = other DNA
agagttcttggtcgttggatcctagacagcccaattaaacg

SEQ ID NO: 1013 - DNA, 39 bp, synthetic construct
  misc_feature 1..39: note = Target Specific Oligonucleotide
  source 1..39: mol_type = other DNA
ctcctttgcaaccgggtctgtagacagcccaattaaacg

SEQ ID NO: 1014 - DNA, 41 bp, synthetic construct
  misc_feature 1..41: note = Target Specific Oligonucleotide
  source 1..41: mol_type = other DNA
cttgttattgacgtcgaaggcttagacagcccaattaaacg

SEQ ID NO: 1015 - DNA, 40 bp, synthetic construct
  misc_feature 1..40: note = Target Specific Oligonucleotide
  source 1..40: mol_type = other DNA
caggaacgtgtaactcttgcctagacagcccaattaaacg

SEQ ID NO: 1016 - DNA, 40 bp, synthetic construct
  misc_feature 1..40: note = Target Specific Oligonucleotide
  source 1..40: mol_type = other DNA
ggagtttcacacacgagttggtagacagcccaattaaacg

SEQ ID NO: 1017 - DNA, 39 bp, synthetic construct
  misc_feature 1..39: note = Target Specific Oligonucleotide
  source 1..39: mol_type = other DNA
aggttcccgctctacggatgtagacagcccaattaaacg

SEQ ID NO: 1018 - DNA, 38 bp, synthetic construct
  misc_feature 1..38: note = Target Specific Oligonucleotide
  source 1..38: mol_type = other DNA
accgtcatggactgccgtctagacagcccaattaaacg

SEQ ID NO: 1019 - DNA, 40 bp, synthetic construct
  misc_feature 1..40: note = Target Specific Oligonucleotide
  source 1..40: mol_type = other DNA
tgggacctttagacttcggtgtagacagcccaattaaacg

SEQ ID NO: 1020 - DNA, 37 bp, synthetic construct
  misc_feature 1..37: note = Target Specific Oligonucleotide
  source 1..37: mol_type = other DNA
gtccctgtagacgcgcgttagacagcccaattaaacg

SEQ ID NO: 1021 - DNA, 38 bp, synthetic construct
  misc_feature 1..38: note = Target Specific Oligonucleotide
  source 1..38: mol_type = other DNA
ccagagagggcgcatcttgtagacagcccaattaaacg

SEQ ID NO: 1022 - DNA, 40 bp, synthetic construct
  misc_feature 1..40: note = Target Specific Oligonucleotide
  source 1..40: mol_type = other DNA
tccactctgcacgctcatagttagacagcccaattaaacg

SEQ ID NO: 1023 - DNA, 40 bp, synthetic construct
  misc_feature 1..40: note = Target Specific Oligonucleotide
  source 1..40: mol_type = other DNA
gtgaaactcgacgttcacgtatagacagcccaattaaacg

SEQ ID NO: 1024 - DNA, 40 bp, synthetic construct
  misc_feature 1..40: note = Target Specific Oligonucleotide
  source 1..40: mol_type = other DNA
acacctctttgtcgttgaccttagacagcccaattaaacg

SEQ ID NO: 1025 - DNA, 39 bp, synthetic construct
  misc_feature 1..39: note = Target Specific Oligonucleotide
  source 1..39: mol_type = other DNA
actgttgcgagttctgcgagtagacagcccaattaaacg

SEQ ID NO: 1026 - DNA, 39 bp, synthetic construct
  misc_feature 1..39: note = Target Specific Oligonucleotide
  source 1..39: mol_type = other DNA
tggcaactccgtagttgtcctagacagcccaattaaacg

SEQ ID NO: 1027 - DNA, 38 bp, synthetic construct
  misc_feature 1..38: note = Target Specific Oligonucleotide
  source 1..38: mol_type = other DNA
tcctcggacgctaagctcatagacagcccaattaaacg

SEQ ID NO: 1028 - DNA, 38 bp, synthetic construct
  misc_feature 1..38: note = Target Specific Oligonucleotide
  source 1..38: mol_type = other DNA
ttacccggagaaccctcgctagacagcccaattaaacg

SEQ ID NO: 1029 - DNA, 38 bp, synthetic construct
  misc_feature 1..38: note = Target Specific Oligonucleotide
  source 1..38: mol_type = other DNA
tcccggaacatgcggtaggtagacagcccaattaaacg

SEQ ID NO: 1030 - DNA, 38 bp, synthetic construct
  misc_feature 1..38: note = Target Specific Oligonucleotide
  source 1..38: mol_type = other DNA
tgctcgatgtcgcccactgtagacagcccaattaaacg

SEQ ID NO: 1031 - DNA, 37 bp, synthetic construct
  misc_feature 1..37: note = Target Specific Oligonucleotide
  source 1..37: mol_type = other DNA
tgccggtgccgcttatggtagacagcccaattaaacg

SEQ ID NO: 1032 - DNA, 45 bp, synthetic construct
  misc_feature 1..45: note = Target Specific Oligonucleotide
  source 1..45: mol_type = other DNA
ccaaagtctgctagcttgatggtagacggtataatacttatttcc

SEQ ID NO: 1033 - DNA, 48 bp, synthetic construct
  misc_feature 1..48: note = Target Specific Oligonucleotide
  source 1..48: mol_type = other DNA
tattaggatggttaagctccttaagtagacggtataatacttatttcc

SEQ ID NO: 1034 - DNA, 45 bp, synthetic construct
  misc_feature 1..45: note = Target Specific Oligonucleotide
  source 1..45: mol_type = other DNA
catgggtgtaagtacgaacaggtagacggtataatacttatttcc

SEQ ID NO: 1035 - DNA, 43 bp, synthetic construct
  misc_feature 1..43: note = Target Specific Oligonucleotide
  source 1..43: mol_type = other DNA
ggcagaaagctaggccctggtagacggtataatacttatttcc

SEQ ID NO: 1036 - DNA, 43 bp, synthetic construct
  misc_feature 1..43: note = Target Specific Oligonucleotide
  source 1..43: mol_type = other DNA
gtacacaactccgtacgtgctagacggtataatacttatttcc

SEQ ID NO: 1037 - DNA, 45 bp, synthetic construct
  misc_feature 1..45: note = Target Specific Oligonucleotide
  source 1..45: mol_type = other DNA
atggaacgcagtatacctctcgtagacggtataatacttatttcc

SEQ ID NO: 1038 - DNA, 46 bp, synthetic construct
  misc_feature 1..46: note = Target Specific Oligonucleotide
  source 1..46: mol_type = other DNA
cttggcttgtaatcaggcatagatagacggtataatacttatttcc

SEQ ID NO: 1039 - DNA, 41 bp, synthetic construct
  misc_feature 1..41: note = Target Specific Oligonucleotide
  source 1..41: mol_type = other DNA
ctccatgaagcgccagcgtagacggtataatacttatttcc

SEQ ID NO: 1040 - DNA, 43 bp, synthetic construct
  misc_feature 1..43: note = Target Specific Oligonucleotide
  source 1..43: mol_type = other DNA
ccgcttgttagggtcgtagttagacggtataatacttatttcc

SEQ ID NO: 1041 - DNA, 42 bp, synthetic construct
  misc_feature 1..42: note = Target Specific Oligonucleotide
  source 1..42: mol_type = other DNA
ctcggtggaggacccgatgtagacggtataatacttatttcc

SEQ ID NO: 1042 - DNA, 44 bp, synthetic construct
  misc_feature 1..44: note = Target Specific Oligonucleotide
  source 1..44: mol_type = other DNA
ttactaaaatcttgccgggcctagacggtataatacttatttcc

SEQ ID NO: 1043 - DNA, 44 bp, synthetic construct
  misc_feature 1..44: note = Target Specific Oligonucleotide
  source 1..44: mol_type = other DNA
gcagcatttgcgataacaagctagacggtataatacttatttcc

SEQ ID NO: 1044 - DNA, 45 bp, synthetic construct
  misc_feature 1..45: note = Target Specific Oligonucleotide
  source 1..45: mol_type = other DNA
agaaggctatcagagtcgaagatagacggtataatacttatttcc

SEQ ID NO: 1045 - DNA, 43 bp, synthetic construct
  misc_feature 1..43: note = Target Specific Oligonucleotide
  source 1..43: mol_type = other DNA
ttcaggagctcggtaccacatagacggtataatacttatttcc

SEQ ID NO: 1046 - DNA, 44 bp, synthetic construct
  misc_feature 1..44: note = Target Specific Oligonucleotide
  source 1..44: mol_type = other DNA
ccccagagtccgaaagatccgtagacggtataatacttatttcc

SEQ ID NO: 1047 - DNA, 42 bp, synthetic construct
  misc_feature 1..42: note = Target Specific Oligonucleotide
  source 1..42: mol_type = other DNA
acagggcccagtaccgtggtagacggtataatacttatttcc

SEQ ID NO: 1048 - DNA, 46 bp, synthetic construct
  misc_feature 1..46: note = Target Specific Oligonucleotide
  source 1..46: mol_type = other DNA
tcagcaataactaagagaaggggtagacggtataatacttatttcc

SEQ ID NO: 1049 - DNA, 46 bp, synthetic construct
  misc_feature 1..46: note = Target Specific Oligonucleotide
  source 1..46: mol_type = other DNA
gtacggcaaatctaacgtgtaggtagacggtataatacttatttcc

SEQ ID NO: 1050 - DNA, 44 bp, synthetic construct
  misc_feature 1..44: note = Target Specific Oligonucleotide
  source 1..44: mol_type = other DNA
tgaaacaatgttgccgcctcctagacggtataatacttatttcc

SEQ ID NO: 1051 - DNA, 47 bp, synthetic construct
  misc_feature 1..47: note = Target Specific Oligonucleotide
  source 1..47: mol_type = other DNA
taataattatggggcattcagagatagacggtataatacttatttcc

SEQ ID NO: 1052 - DNA, 43 bp, synthetic construct
  misc_feature 1..43: note = Target Specific Oligonucleotide
  source 1..43: mol_type = other DNA
tctcacctgcctcataaggctagacggtataatacttatttcc

SEQ ID NO: 1053 - DNA, 47 bp, synthetic construct
  misc_feature 1..47: note = Target Specific Oligonucleotide
  source 1..47: mol_type = other DNA
atggtgaagcaatagattggaatgtagacggtataatacttatttcc

SEQ ID NO: 1054 - DNA, 49 bp, synthetic construct
  misc_feature 1..49: note = Target Specific Oligonucleotide
  source 1..49: mol_type = other DNA
cactaactagggtataaactgattggtagacggtataatacttatttcc

SEQ ID NO: 1055 - DNA, 44 bp, synthetic construct
  misc_feature 1..44: note = Target Specific Oligonucleotide
  source 1..44: mol_type = other DNA
agaagggacaaacaaggggactagacggtataatacttatttcc

SEQ ID NO: 1056 - DNA, 43 bp, synthetic construct
  misc_feature 1..43: note = Target Specific Oligonucleotide
  source 1..43: mol_type = other DNA
agtccgatactcaagcgcaaatagacaataaattctaacgagc

SEQ ID NO: 1057 - DNA, 42 bp, synthetic construct
  misc_feature 1..42: note = Target Specific Oligonucleotide
  source 1..42: mol_type = other DNA
cttgttgcgctcacggtatgtagacaataaattctaacgagc

SEQ ID NO: 1058 - DNA, 43 bp, synthetic construct
  misc_feature 1..43: note = Target Specific Oligonucleotide
  source 1..43: mol_type = other DNA
ccagagttgtagacgagcagctagacaataaattctaacgagc

SEQ ID NO: 1059 - DNA, 41 bp, synthetic construct
  misc_feature 1..41: note = Target Specific Oligonucleotide
  source 1..41: mol_type = other DNA
ttagcatggcaacacggcgtagacaataaattctaacgagc

SEQ ID NO: 1060 - DNA, 41 bp, synthetic construct
  misc_feature 1..41: note = Target Specific Oligonucleotide
  source 1..41: mol_type = other DNA
atgatgcgcacttcacgggtagacaataaattctaacgagc

SEQ ID NO: 1061 - DNA, 42 bp, synthetic construct
  misc_feature 1..42: note = Target Specific Oligonucleotide
  source 1..42: mol_type = other DNA
cccaccgctaggagggtagctagacaataaattctaacgagc

SEQ ID NO: 1062 - DNA, 42 bp, synthetic construct
  misc_feature 1..42: note = Target Specific Oligonucleotide
  source 1..42: mol_type = other DNA
gcagtgtggttaggccgatgtagacaataaattctaacgagc

SEQ ID NO: 1063 - DNA, 41 bp, synthetic construct
  misc_feature 1..41: note = Target Specific Oligonucleotide
  source 1..41: mol_type = other DNA
gacacggcggtgcactatctagacaataaattctaacgagc

SEQ ID NO: 1064 - DNA, 40 bp, synthetic construct
  misc_feature 1..40: note = Target Specific Oligonucleotide
  source 1..40: mol_type = other DNA
gcccaaagcggcttgcagtagacaataaattctaacgagc

SEQ ID NO: 1065 - DNA, 44 bp, synthetic construct
  misc_feature 1..44: note = Target Specific Oligonucleotide
  source 1..44: mol_type = other DNA
atacacacacggctaatactgctagacaataaattctaacgagc

SEQ ID NO: 1066 - DNA, 43 bp, synthetic construct
  misc_feature 1..43: note = Target Specific Oligonucleotide
  source 1..43: mol_type = other DNA
gtgaccgtgtacaccaacaactagacaataaattctaacgagc

SEQ ID NO: 1067 - DNA, 46 bp, synthetic construct
  misc_feature 1..46: note = Target Specific Oligonucleotide
  source 1..46: mol_type = other DNA
ctcttcatactagcatgactgtcatagacaataaattctaacgagc

SEQ ID NO: 1068 - DNA, 42 bp, synthetic construct
  misc_feature 1..42: note = Target Specific Oligonucleotide
  source 1..42: mol_type = other DNA
agaaggctttcgtcgttccctagacaataaattctaacgagc

SEQ ID NO: 1069 - DNA, 42 bp, synthetic construct
  misc_feature 1..42: note = Target Specific Oligonucleotide
  source 1..42: mol_type = other DNA
tttgctccgtgcgttcaaggtagacaataaattctaacgagc

SEQ ID NO: 1070 - DNA, 42 bp, synthetic construct
  misc_feature 1..42: note = Target Specific Oligonucleotide
  source 1..42: mol_type = other DNA
tccagtgcatccgtcacatctagacaataaattctaacgagc

SEQ ID NO: 1071 - DNA, 41 bp, synthetic construct
  misc_feature 1..41: note = Target Specific Oligonucleotide
  source 1..41: mol_type = other DNA
gatgaacagcggtgcgttgtagacaataaattctaacgagc

SEQ ID NO: 1072 - DNA, 42 bp, synthetic construct
  misc_feature 1..42: note = Target Specific Oligonucleotide
  source 1..42: mol_type = other DNA
cactgataccggctctccgttagacaataaattctaacgagc

SEQ ID NO: 1073 - DNA, 42 bp, synthetic construct
  misc_feature 1..42: note = Target Specific Oligonucleotide
  source 1..42: mol_type = other DNA
gaacgcttgcgttccctagctagacaataaattctaacgagc

SEQ ID NO: 1074 - DNA, 45 bp, synthetic construct
  misc_feature 1..45: note = Target Specific Oligonucleotide
  source 1..45: mol_type = other DNA
cagatagatttccgggatacagctagacaataaattctaacgagc

SEQ ID NO: 1075 - DNA, 40 bp, synthetic construct
  misc_feature 1..40: note = Target Specific Oligonucleotide
  source 1..40: mol_type = other DNA
tctgctgcggcgattgtgtagacaataaattctaacgagc

SEQ ID NO: 1076 - DNA, 41 bp, synthetic construct
  misc_feature 1..41: note = Target Specific Oligonucleotide
  source 1..41: mol_type = other DNA
ctcggtgatcatgcgtccctagacaataaattctaacgagc

SEQ ID NO: 1077 - DNA, 43 bp, synthetic construct
  misc_feature 1..43: note = Target Specific Oligonucleotide
  source 1..43: mol_type = other DNA
tccaatttcagggcgttctcgtagacaataaattctaacgagc

SEQ ID NO: 1078 - DNA, 40 bp, synthetic construct
  misc_feature 1..40: note = Target Specific Oligonucleotide
  source 1..40: mol_type = other DNA
tgcgcaggtcgacgtcagtagacaataaattctaacgagc

SEQ ID NO: 1079 - DNA, 43 bp, synthetic construct
  misc_feature 1..43: note = Target Specific Oligonucleotide
  source 1..43: mol_type = other DNA
gtccaggtagtatccggagggtagacaataaattctaacgagc

SEQ ID NO: 1080 - DNA, 40 bp, synthetic construct
  misc_feature 1..40: note = Target Specific Oligonucleotide
  source 1..40: mol_type = other DNA
attcccgacagactcatcgctagacgataaattgcgaacg

SEQ ID NO: 1081 - DNA, 40 bp, synthetic construct
  misc_feature 1..40: note = Target Specific Oligonucleotide
  source 1..40: mol_type = other DNA
cagttacatgttcgcgcacgtagacgataaattgcgaacg

SEQ ID NO: 1082 - DNA, 39 bp, synthetic construct
  misc_feature 1..39: note = Target Specific Oligonucleotide
  source 1..39: mol_type = other DNA
acgagcactagggtcgtcgtagacgataaattgcgaacg

SEQ ID NO: 1083 - DNA, 46 bp, synthetic construct
  misc_feature 1..46: note = Target Specific Oligonucleotide
  source 1..46: mol_type = other DNA
ttttgataatcgaatgaaaaagtcactagacgataaattgcgaacg

SEQ ID NO: 1084 - DNA, 43 bp, synthetic construct
  misc_feature 1..43: note = Target Specific Oligonucleotide
  source 1..43: mol_type = other DNA
ctgaatactgctagacgagaagctagacgataaattgcgaacg

SEQ ID NO: 1085 - DNA, 43 bp, synthetic construct
  misc_feature 1..43: note = Target Specific Oligonucleotide
  source 1..43: mol_type = other DNA
ctccttggaacgaactaatcctgtagacgataaattgcgaacg

SEQ ID NO: 1086 - DNA, 42 bp, synthetic construct
  misc_feature 1..42: note = Target Specific Oligonucleotide
  source 1..42: mol_type = other DNA
ttcaggcggttaattggtaccctagacgataaattgcgaacg

SEQ ID NO: 1087 - DNA, 42 bp, synthetic construct
  misc_feature 1..42: note = Target Specific Oligonucleotide
  source 1..42: mol_type = other DNA
gttaggaatggcgaacagacagtagacgataaattgcgaacg

SEQ ID NO: 1088 - DNA, 42 bp, synthetic construct
  misc_feature 1..42: note = Target Specific Oligonucleotide
  source 1..42: mol_type = other DNA
gatggtcttaaagcgtgaagcctagacgataaattgcgaacg

SEQ ID NO: 1089 - DNA, 42 bp, synthetic construct
  misc_feature 1..42: note = Target Specific Oligonucleotide
  source 1..42: mol_type = other DNA
ctgtgcctagccctaacttatatagacgataaattgcgaacg

SEQ ID NO: 1090 - DNA, 42 bp, synthetic construct
  misc_feature 1..42: note = Target Specific Oligonucleotide
  source 1..42: mol_type = other DNA
agagaccacccataacaagagttagacgataaattgcgaacg

SEQ ID NO: 1091 - DNA, 43 bp, synthetic construct
  misc_feature 1..43: note = Target Specific Oligonucleotide
  source 1..43: mol_type = other DNA
ttggagcaccgatatagatatggtagacgataaattgcgaacg

SEQ ID NO: 1092 - DNA, 41 bp, synthetic construct
  misc_feature 1..41: note = Target Specific Oligonucleotide
  source 1..41: mol_type = other DNA
tcaaagacgcaaacgtccaaatagacgataaattgcgaacg

SEQ ID NO: 1093 - DNA, 42 bp, synthetic construct
  misc_feature 1..42: note = Target Specific Oligonucleotide
  source 1..42: mol_type = other DNA
gagagctgattaggtgctcaggtagacgataaattgcgaacg

SEQ ID NO: 1094 - DNA, 40 bp, synthetic construct
  misc_feature 1..40: note = Target Specific Oligonucleotide
  source 1..40: mol_type = other DNA
tgcttgtacgcggagaaggttagacgataaattgcgaacg

SEQ ID NO: 1095 - DNA, 40 bp, synthetic construct
  misc_feature 1..40: note = Target Specific Oligonucleotide
  source 1..40: mol_type = other DNA
ctgcagacacgtgtatgtgctagacgataaattgcgaacg

SEQ ID NO: 1096 - DNA, 40 bp, synthetic construct
  misc_feature 1..40: note = Target Specific Oligonucleotide
  source 1..40: mol_type = other DNA
catccaatgcaagtccggtgtagacgataaattgcgaacg

SEQ ID NO: 1097 - DNA, 42 bp, synthetic construct
  misc_feature 1..42: note = Target Specific Oligonucleotide
  source 1..42: mol_type = other DNA
accaagtgataacgtgttcagatagacgataaattgcgaacg

SEQ ID NO: 1098 - DNA, 42 bp, synthetic construct
  misc_feature 1..42: note = Target Specific Oligonucleotide
  source 1..42: mol_type = other DNA
aatctgagctttgcgaaatggttagacgataaattgcgaacg

SEQ ID NO: 1099 - DNA, 44 bp, synthetic construct
  misc_feature 1..44: note = Target Specific Oligonucleotide
  source 1..44: mol_type = other DNA
caaagcccacataacactatcttttagacgataaattgcgaacg

SEQ ID NO: 1100 - DNA, 44 bp, synthetic construct
  misc_feature 1..44: note = Target Specific Oligonucleotide
  source 1..44: mol_type = other DNA
tgtactggactatagacaccaatttagacgataaattgcgaacg

SEQ ID NO: 1101 - DNA, 45 bp, synthetic construct
  misc_feature 1..45: note = Target Specific Oligonucleotide
  source 1..45: mol_type = other DNA
atcaatataccacgttagtagtcactagacgataaattgcgaacg

SEQ ID NO: 1102 - DNA, 42 bp, synthetic construct
  misc_feature 1..42: note = Target Specific Oligonucleotide
  source 1..42: mol_type = other DNA
tgtattttaacgccaagcaggctagacgataaattgcgaacg

SEQ ID NO: 1103 - DNA, 42 bp, synthetic construct
  misc_feature 1..42: note = Target Specific Oligonucleotide
  source 1..42: mol_type = other DNA
cactgaaaacacggttatgtcctagacgataaattgcgaacg

SEQ ID NO: 1104 - DNA, 40 bp, synthetic construct
  misc_feature 1..40: note = Target Specific Oligonucleotide
  source 1..40: mol_type = other DNA
cgagacagatcggagtagctgtagacgaccgaatttacgg

SEQ ID NO: 1105 - DNA, 41 bp, synthetic construct
  misc_feature 1..41: note = Target Specific Oligonucleotide
  source 1..41: mol_type = other DNA
tctctcaatgacgggacaaagatagacgaccgaatttacgg

SEQ ID NO: 1106 - DNA, 41 bp, synthetic construct
  misc_feature 1..41: note = Target Specific Oligonucleotide
  source 1..41: mol_type = other DNA
cagatgtggtgtatggtccacatagacgaccgaatttacgg

SEQ ID NO: 1107 - DNA, 40 bp, synthetic construct
  misc_feature 1..40: note = Target Specific Oligonucleotide
  source 1..40: mol_type = other DNA
ttctcctcggttacttcgtgatagacgaccgaatttacgg

SEQ ID NO: 1108 - DNA, 39 bp, synthetic construct
  misc_feature 1..39: note = Target Specific Oligonucleotide
  source 1..39: mol_type = other DNA
tgtgctaagccgaaggaggttagacgaccgaatttacgg

SEQ ID NO: 1109 - DNA, 44 bp, synthetic construct
  misc_feature 1..44: note = Target Specific Oligonucleotide
  source 1..44: mol_type = other DNA
gtaaacatagaacgactcatagtcatagacgaccgaatttacgg

SEQ ID NO: 1110 - DNA, 43 bp, synthetic construct
  misc_feature 1..43: note = Target Specific Oligonucleotide
  source 1..43: mol_type = other DNA
ttccacttcaattagctcttgaattagacgaccgaatttacgg

SEQ ID NO: 1111 - DNA, 40 bp, synthetic construct
  misc_feature 1..40: note = Target Specific Oligonucleotide
  source 1..40: mol_type = other DNA
ctggggtagagatagctcgcttagacgaccgaatttacgg

SEQ ID NO: 1112 - DNA, 41 bp, synthetic construct
  misc_feature 1..41: note = Target Specific Oligonucleotide
  source 1..41: mol_type = other DNA
gctataagctcgcctaccattctagacgaccgaatttacgg

SEQ ID NO: 1113 - DNA, 39 bp, synthetic construct
  misc_feature 1..39: note = Target Specific Oligonucleotide
  source 1..39: mol_type = other DNA
ttcttggggcgggtagagtgtagacgaccgaatttacgg

SEQ ID NO: 1114 - DNA, 40 bp, synthetic construct
  misc_feature 1..40: note = Target Specific Oligonucleotide
  source 1..40: mol_type = other DNA
aagacctcagaccgtcagaagtagacgaccgaatttacgg

SEQ ID NO: 1115 - DNA, 39 bp, synthetic construct
  misc_feature 1..39: note = Target Specific Oligonucleotide
  source 1..39: mol_type = other DNA
tcagatcctgacgacctgcatagacgaccgaatttacgg

SEQ ID NO: 1116 - DNA, 40 bp, synthetic construct
  misc_feature 1..40: note = Target Specific Oligonucleotide
  source 1..40: mol_type = other DNA
gcagaaatctcgagaggacgctagacgaccgaatttacgg

SEQ ID NO: 1117 - DNA, 39 bp, synthetic construct
  misc_feature 1..39: note = Target Specific Oligonucleotide
  source 1..39: mol_type = other DNA
ctcgcagtctaggccgaagatagacgaccgaatttacgg

SEQ ID NO: 1118 - DNA, 39 bp, synthetic construct
  misc_feature 1..39: note = Target Specific Oligonucleotide
  source 1..39: mol_type = other DNA
ccggtctccctcgagaatcatagacgaccgaatttacgg

SEQ ID NO: 1119 - DNA, 39 bp, synthetic construct
  misc_feature 1..39: note = Target Specific Oligonucleotide
  source 1..39: mol_type = other DNA
tcctggtgagaacgtcctgctagacgaccgaatttacgg

SEQ ID NO: 1120 - DNA, 38 bp, synthetic construct
  misc_feature 1..38: note = Target Specific Oligonucleotide
  source 1..38: mol_type = other DNA
tgccctttggtaaggcgcctagacgaccgaatttacgg

SEQ ID NO: 1121 - DNA, 38 bp, synthetic construct
  misc_feature 1..38: note = Target Specific Oligonucleotide
  source 1..38: mol_type = other DNA
cacagacacagccgaggactagacgaccgaatttacgg

SEQ ID NO: 1122 - DNA, 39 bp, synthetic construct
  misc_feature 1..39: note = Target Specific Oligonucleotide
  source 1..39: mol_type = other DNA
tcccaaaagctaccgctgagtagacgaccgaatttacgg

SEQ ID NO: 1123 - DNA, 39 bp, synthetic construct
  misc_feature 1..39: note = Target Specific Oligonucleotide
  source 1..39: mol_type = other DNA
taaggtagagtcgggcgaagtagacgaccgaatttacgg

SEQ ID NO: 1124 - DNA, 38 bp, synthetic construct
  misc_feature 1..38: note = Target Specific Oligonucleotide
  source 1..38: mol_type = other DNA
gagtggccggaaaaacccgtagacgaccgaatttacgg

SEQ ID NO: 1125 - DNA, 37 bp, synthetic construct
  misc_feature 1..37: note = Target Specific Oligonucleotide
  source 1..37: mol_type = other DNA
atgcggacatggtcgccctagacgaccgaatttacgg

SEQ ID NO: 1126 - DNA, 37 bp, synthetic construct
  misc_feature 1..37: note = Target Specific Oligonucleotide
  source 1..37: mol_type = other DNA
ggctcccaaacgccctgatagacgaccgaatttacgg

SEQ ID NO: 1127 - DNA, 40 bp, synthetic construct
  misc_feature 1..40: note = Target Specific Oligonucleotide
  source 1..40: mol_type = other DNA
tccatttcaccgtcagaaaggtagacgaccgaatttacgg

SEQ ID NO: 1128 - DNA, 43 bp, synthetic construct
  misc_feature 1..43: note = Target Specific Oligonucleotide
  source 1..43: mol_type = other DNA
ttgcctagacagcaccgtaatgtagacacgaatattccaattc

SEQ ID NO: 1129 - DNA, 40 bp, synthetic construct
  misc_feature 1..40: note = Target Specific Oligonucleotide
  source 1..40: mol_type = other DNA
ttcccagagcgtggttccatagacacgaatattccaattc

SEQ ID NO: 1130 - DNA, 42 bp, synthetic construct
  misc_feature 1..42: note = Target Specific Oligonucleotide
  source 1..42: mol_type = other DNA
taaggacatcgcttttccggttagacacgaatattccaattc

SEQ ID NO: 1131 - DNA, 40 bp, synthetic construct
  misc_feature 1..40: note = Target Specific Oligonucleotide
  source 1..40: mol_type = other DNA
ttctccacgtcgcggacactagacacgaatattccaattc

SEQ ID NO: 1132 - DNA, 46 bp, synthetic construct
  misc_feature 1..46: note = Target Specific Oligonucleotide
  source 1..46: mol_type = other DNA
gttctcatgatagaggttccttaagtagacacgaatattccaattc

SEQ ID NO: 1133 - DNA, 38 bp, synthetic construct
  misc_feature 1..38: note = Target Specific Oligonucleotide
  source 1..38: mol_type = other DNA
ggccacgtcggtgcagatagacacgaatattccaattc

SEQ ID NO: 1134 - DNA, 41 bp, synthetic construct
  misc_feature 1..41: note = Target Specific Oligonucleotide
  source 1..41: mol_type = other DNA
gttgtggacgatcaacggggtagacacgaatattccaattc

SEQ ID NO: 1135 - DNA, 42 bp, synthetic construct
  misc_feature 1..42: note = Target Specific Oligonucleotide
  source 1..42: mol_type = other DNA
tgtccccttcggggtcatacctagacacgaatattccaattc

SEQ ID NO: 1136 - DNA, 44 bp, synthetic construct
  misc_feature 1..44: note = Target Specific Oligonucleotide
  source 1..44: mol_type = other DNA
aatagaacctcatccggtagtggtagacacgaatattccaattc

SEQ ID NO: 1137 - DNA, 42 bp, synthetic construct
  misc_feature 1..42: note = Target Specific Oligonucleotide
  source 1..42: mol_type = other DNA
cagtgcagcgtatgtggtatttagacacgaatattccaattc

SEQ ID NO: 1138 - DNA, 42 bp, synthetic construct
  misc_feature 1..42: note = Target Specific Oligonucleotide
  source 1..42: mol_type = other DNA
aaagcttgtccgattggatggtagacacgaatattccaattc

SEQ ID NO: 1139 - DNA, 42 bp, synthetic construct
  misc_feature 1..42: note = Target Specific Oligonucleotide
  source 1..42: mol_type = other DNA
aacccttgtagatcggtggtatagacacgaatattccaattc

SEQ ID NO: 1140 - DNA, 42 bp, synthetic construct
  misc_feature 1..42: note = Target Specific Oligonucleotide
  source 1..42: mol_type = other DNA
agtagagaacgtttccaccgttagacacgaatattccaattc

SEQ ID NO: 1141 - DNA, 42 bp, synthetic construct
  misc_feature 1..42: note = Target Specific Oligonucleotide
  source 1..42: mol_type = other DNA
actgctcattgtcgttggttctagacacgaatattccaattc

SEQ ID NO: 1142 - DNA, 41 bp, synthetic construct
  misc_feature 1..41: note = Target Specific Oligonucleotide
  source 1..41: mol_type = other DNA
aacgtattgccgagaacccatagacacgaatattccaattc

SEQ ID NO: 1143 - DNA, 43 bp, synthetic construct
  misc_feature 1..43: note = Target Specific Oligonucleotide
  source 1..43: mol_type = other DNA
acaaagtgcatatagagtggcctagacacgaatattccaattc

SEQ ID NO: 1144 - DNA, 42 bp, synthetic construct
  misc_feature 1..42: note = Target Specific Oligonucleotide
  source 1..42: mol_type = other DNA
agccatccctagacactcgtttagacacgaatattccaattc

SEQ ID NO: 1145 - DNA, 42 bp, synthetic construct
  misc_feature 1..42: note = Target Specific Oligonucleotide
  source 1..42: mol_type = other DNA
gcgatttggagcataccagagtagacacgaatattccaattc

SEQ ID NO: 1146 - DNA, 42 bp, synthetic construct
  misc_feature 1..42: note = Target Specific Oligonucleotide
  source 1..42: mol_type = other DNA
aggatccgatcgaaactcagctagacacgaatattccaattc

SEQ ID NO: 1147 - DNA, 44 bp, synthetic construct
  misc_feature 1..44: note = Target Specific Oligonucleotide
  source 1..44: mol_type = other DNA
ctggaatatatcgcttgtagctgtagacacgaatattccaattc

SEQ ID NO: 1148 - DNA, 41 bp, synthetic construct
  misc_feature 1..41: note = Target Specific Oligonucleotide
  source 1..41: mol_type = other DNA
cttcaggtcatgcgtggacatagacacgaatattccaattc

SEQ ID NO: 1149 - DNA, 41 bp, synthetic construct
  misc_feature 1..41: note = Target Specific Oligonucleotide
  source 1..41: mol_type = other DNA
agagaagcgcacttccgtgttagacacgaatattccaattc

SEQ ID NO: 1150 - DNA, 41 bp, synthetic construct
  misc_feature 1..41: note = Target Specific Oligonucleotide
  source 1..41: mol_type = other DNA
tgatgttatccgtgcgcaggtagacacgaatattccaattc

SEQ ID NO: 1151 - DNA, 44 bp, synthetic construct
  misc_feature 1..44: note = Target Specific Oligonucleotide
  source 1..44: mol_type = other DNA
aaagaccgtaatcgttcacattgtagacacgaatattccaattc

SEQ ID NO: 1152 - DNA, 42 bp, synthetic construct
  misc_feature 1..42: note = Target Specific Oligonucleotide
  source 1..42: mol_type = other DNA
agagatttgtctacgtagagccgtagactcagaaatctcacg

SEQ ID NO: 1153 - DNA, 41 bp, synthetic construct
  misc_feature 1..41: note = Target Specific Oligonucleotide
  source 1..41: mol_type = other DNA
tcctttgcatacggtctcttcgtagactcagaaatctcacg

SEQ ID NO: 1154 - DNA, 43 bp, synthetic construct
  misc_feature 1..43: note = Target Specific Oligonucleotide
  source 1..43: mol_type = other DNA
aagtttgtttacatccgatgttactagactcagaaatctcacg

SEQ ID NO: 1155 - DNA, 39 bp, synthetic construct
  misc_feature 1..39: note = Target Specific Oligonucleotide
  source 1..39: mol_type = other DNA
ttcccatacagaacgtggcctagactcagaaatctcacg

SEQ ID NO: 1156 - DNA, 40 bp, synthetic construct
  misc_feature 1..40: note = Target Specific Oligonucleotide
  source 1..40: mol_type = other DNA
aacagatcaacggcaaaagcctagactcagaaatctcacg

SEQ ID NO: 1157 - DNA, 42 bp, synthetic construct
  misc_feature 1..42: note = Target Specific Oligonucleotide
  source 1..42: mol_type = other DNA
catccttgggtagtaggatgaactagactcagaaatctcacg

SEQ ID NO: 1158 - DNA, 40 bp, synthetic construct
  misc_feature 1..40: note = Target Specific Oligonucleotide
  source 1..40: mol_type = other DNA
gaatttgctagttgcagggcatagactcagaaatctcacg

SEQ ID NO: 1159 - DNA, 45 bp, synthetic construct
  misc_feature 1..45: note = Target Specific Oligonucleotide
  source 1..45: mol_type = other DNA
tgatcttacaattgatactgtcaatgtagactcagaaatctcacg

SEQ ID NO: 1160 - DNA, 38 bp, synthetic construct
  misc_feature 1..38: note = Target Specific Oligonucleotide
  source 1..38: mol_type = other DNA
tggatgtggaggcgaccgttagactcagaaatctcacg

SEQ ID NO: 1161 - DNA, 42 bp, synthetic construct
  misc_feature 1..42: note = Target Specific Oligonucleotide
  source 1..42: mol_type = other DNA
acttctggacctatagtagcctgtagactcagaaatctcacg

SEQ ID NO: 1162 - DNA, 42 bp, synthetic construct
  misc_feature 1..42: note = Target Specific Oligonucleotide
  source 1..42: mol_type = other DNA
caacacggatagcttatccaacctagactcagaaatctcacg

SEQ ID NO: 1163 - DNA, 40 bp, synthetic construct
  misc_feature 1..40: note = Target Specific Oligonucleotide
  source 1..40: mol_type = other DNA
tgtcaaggtaacgtgagcacttagactcagaaatctcacg

SEQ ID NO: 1164 - DNA, 41 bp, synthetic construct
  misc_feature 1..41: note = Target Specific Oligonucleotide
  source 1..41: mol_type = other DNA
tttacagaaatcggcatccacatagactcagaaatctcacg

SEQ ID NO: 1165 - DNA, 44 bp, synthetic construct
  misc_feature 1..44: note = Target Specific Oligonucleotide
  source 1..44: mol_type = other DNA
cagctactagaggtctataattccttagactcagaaatctcacg

SEQ ID NO: 1166 - DNA, 40 bp, synthetic construct
  misc_feature 1..40: note = Target Specific Oligonucleotide
  source 1..40: mol_type = other DNA
ctggcacctctatggaatccctagactcagaaatctcacg

SEQ ID NO: 1167 - DNA, 43 bp, synthetic construct
  misc_feature 1..43: note = Target Specific Oligonucleotide
  source 1..43: mol_type = other DNA
attctgatatcggctattgtgagatagactcagaaatctcacg

SEQ ID NO: 1168 - DNA, 39 bp, synthetic construct
  misc_feature 1..39: note = Target Specific Oligonucleotide
  source 1..39: mol_type = other DNA
gccaccagtctagtgccaactagactcagaaatctcacg

SEQ ID NO: 1169 - DNA, 40 bp, synthetic construct
  misc_feature 1..40: note = Target Specific Oligonucleotide
  source 1..40: mol_type = other DNA
caactaccccggacaatttgatagactcagaaatctcacg

SEQ ID NO: 1170 - DNA, 41 bp, synthetic construct
  misc_feature 1..41: note = Target Specific Oligonucleotide
  source 1..41: mol_type = other DNA
aggacttaacatgggctatgcctagactcagaaatctcacg

SEQ ID NO: 1171 - DNA, 41 bp, synthetic construct
  misc_feature 1..41: note = Target Specific Oligonucleotide
  source 1..41: mol_type = other DNA
ctctgcgaacgaagtgtttttgtagactcagaaatctcacg

SEQ ID NO: 1172 - DNA, 43 bp, synthetic construct
  misc_feature 1..43: note = Target Specific Oligonucleotide
  source 1..43: mol_type = other DNA
tgcagcatattaaggtgttgattttagactcagaaatctcacg

SEQ ID NO: 1173 - DNA, 44 bp, synthetic construct
  misc_feature 1..44: note = Target Specific Oligonucleotide
  source 1..44: mol_type = other DNA
aagtaactaaacccatagactgaaatagactcagaaatctcacg

SEQ ID NO: 1174 - DNA, 40 bp, synthetic construct
  misc_feature 1..40: note = Target Specific Oligonucleotide
  source 1..40: mol_type = other DNA
ctgccagggcttaacataaggtagactcagaaatctcacg

SEQ ID NO: 1175 - DNA, 42 bp, synthetic construct
  misc_feature 1..42: note = Target Specific Oligonucleotide
  source 1..42: mol_type = other DNA
ataacatttgatagggccactcctagactcagaaatctcacg

SEQ ID NO: 1176 - DNA, 41 bp, synthetic construct
  misc_feature 1..41: note = Target Specific Oligonucleotide
  source 1..41: mol_type = other DNA
ctgaagataatcgcagacaccctagacgtctcccgtatttt

SEQ ID NO: 1177 - DNA, 41 bp, synthetic construct
  misc_feature 1..41: note = Target Specific Oligonucleotide
  source 1..41: mol_type = other DNA
tgactgaacacgtgttctgttctagacgtctcccgtatttt

SEQ ID NO: 1178 - DNA, 39 bp, synthetic construct
  misc_feature 1..39: note = Target Specific Oligonucleotide
  source 1..39: mol_type = other DNA
tttcagccatacccggatggtagacgtctcccgtatttt

SEQ ID NO: 1179 - DNA, 41 bp, synthetic construct
  misc_feature 1..41: note = Target Specific Oligonucleotide
  source 1..41: mol_type = other DNA
tgagaagagtaaccgctgaatatagacgtctcccgtatttt

SEQ ID NO: 1180 - DNA, 38 bp, synthetic construct
  misc_feature 1..38: note = Target Specific Oligonucleotide
  source 1..38: mol_type = other DNA
acactgcaattccgccgtctagacgtctcccgtatttt

SEQ ID NO: 1181 - DNA, 38 bp, synthetic construct
  misc_feature 1..38: note = Target Specific Oligonucleotide
  source 1..38: mol_type = other DNA
ttgtcgtggccggtactgatagacgtctcccgtatttt

SEQ ID NO: 1182 - DNA, 39 bp, synthetic construct
  misc_feature 1..39: note = Target Specific Oligonucleotide
  source 1..39: mol_type = other DNA
ggcaggcgacactaccttcatagacgtctcccgtatttt

SEQ ID NO: 1183 - DNA, 40 bp, synthetic construct
  misc_feature 1..40: note = Target Specific Oligonucleotide
  source 1..40: mol_type = other DNA
ctgggccagtattgaccactgtagacgtctcccgtatttt

SEQ ID NO: 1184 - DNA, 40 bp, synthetic construct
  misc_feature 1..40: note = Target Specific Oligonucleotide
  source 1..40: mol_type = other DNA
tctcagatcagtgcggagaaatagacgtctcccgtatttt

SEQ ID NO: 1185 - DNA, 39 bp, synthetic construct
  misc_feature 1..39: note = Target Specific Oligonucleotide
  source 1..39: mol_type = other DNA
gcccacaaatatccgtctgctagacgtctcccgtatttt

SEQ ID NO: 1186 - DNA, 36 bp, synthetic construct
  misc_feature 1..36: note = Target Specific Oligonucleotide
  source 1..36: mol_type = other DNA
ccatgttcgcgcggcgatagacgtctcccgtatttt

SEQ ID NO: 1187 - DNA, 42 bp, synthetic construct
  misc_feature 1..42: note = Target Specific Oligonucleotide
  source 1..42: mol_type = other DNA
atgaaaggtcgctagagaagtcctagacgtctcccgtatttt

SEQ ID NO: 1188 - DNA, 40 bp, synthetic construct
  misc_feature 1..40: note = Target Specific Oligonucleotide
  source 1..40: mol_type = other DNA
aatcacaaacccggacttggatagacgtctcccgtatttt

SEQ ID NO: 1189 - DNA, 40 bp, synthetic construct
  misc_feature 1..40: note = Target Specific Oligonucleotide
  source 1..40: mol_type = other DNA
actagcctgtcacgtttgtcttagacgtctcccgtatttt

SEQ ID NO: 1190 - DNA, 41 bp, synthetic construct
  misc_feature 1..41: note = Target Specific Oligonucleotide
  source 1..41: mol_type = other DNA
atctttacaatgcggtcggtcctagacgtctcccgtatttt

SEQ ID NO: 1191 - DNA, 41 bp, synthetic construct
  misc_feature 1..41: note = Target Specific Oligonucleotide
  source 1..41: mol_type = other DNA
tgtcctggtatgatcccaacactagacgtctcccgtatttt

SEQ ID NO: 1192 - DNA, 41 bp, synthetic construct
  misc_feature 1..41: note = Target Specific Oligonucleotide
  source 1..41: mol_type = other DNA
ctgatcatttcgcatcacagtgtagacgtctcccgtatttt

SEQ ID NO: 1193 - DNA, 41 bp, synthetic construct
  misc_feature 1..41: note = Target Specific Oligonucleotide
  source 1..41: mol_type = other DNA
ctttccttacgtgtgtcacacatagacgtctcccgtatttt

SEQ ID NO: 1194 - DNA, 42 bp, synthetic construct
  misc_feature 1..42: note = Target Specific Oligonucleotide
  source 1..42: mol_type = other DNA
tagtttgatcgcctccttaaacctagacgtctcccgtatttt

SEQ ID NO: 1195 - DNA, 42 bp, synthetic construct
  misc_feature 1..42: note = Target Specific Oligonucleotide
  source 1..42: mol_type = other DNA
aaatccctaggatagcacctttgtagacgtctcccgtatttt

SEQ ID NO: 1196 - DNA, 42 bp, synthetic construct
  misc_feature 1..42: note = Target Specific Oligonucleotide
  source 1..42: mol_type = other DNA
cttacacttacgatgagcagatgtagacgtctcccgtatttt

SEQ ID NO: 1197 - DNA, 43 bp, synthetic construct
  misc_feature 1..43: note = Target Specific Oligonucleotide
  source 1..43: mol_type = other DNA
catttgctacctagcatatgagagtagacgtctcccgtatttt

SEQ ID NO: 1198 - DNA, 41 bp, synthetic construct
  misc_feature 1..41: note = Target Specific Oligonucleotide
  source 1..41: mol_type = other DNA
acagatgtcaaacggtcacatttagacgtctcccgtatttt

SEQ ID NO: 1199 - DNA, 43 bp, synthetic construct
  misc_feature 1..43: note = Target Specific Oligonucleotide
  source 1..43: mol_type = other DNA
ttctaagatacgtcactccaattctagacgtctcccgtatttt

SEQ ID NO: 1200 - DNA, 42 bp, synthetic construct
  misc_feature 1..42: note = Target Specific Oligonucleotide
  source 1..42: mol_type = other DNA
acagtccgtcgattctttacaatagactctataaatcagcgt

SEQ ID NO: 1201 - DNA, 41 bp, synthetic construct
  misc_feature 1..41: note = Target Specific Oligonucleotide
  source 1..41: mol_type = other DNA
tctccacgtaaatcccgatcttagactctataaatcagcgt

SEQ ID NO: 1202 - DNA, 40 bp, synthetic construct
  misc_feature 1..40: note = Target Specific Oligonucleotide
  source 1..40: mol_type = other DNA
cacgtgctgaaccaaccacttagactctataaatcagcgt

SEQ ID NO: 1203 - DNA, 39 bp, synthetic construct
  misc_feature 1..39: note = Target Specific Oligonucleotide
  source 1..39: mol_type = other DNA
tcttgccgaagttggccgttagactctataaatcagcgt

SEQ ID NO: 1204 - DNA, 41 bp, synthetic construct
  misc_feature 1..41: note = Target Specific Oligonucleotide
  source 1..41: mol_type = other DNA
agcagcagttggatctacagttagactctataaatcagcgt

SEQ ID NO: 1205 - DNA, 43 bp, synthetic construct
  misc_feature 1..43: note = Target Specific Oligonucleotide
  source 1..43: mol_type = other DNA
gtctagcagtaaacctgtaaacctagactctataaatcagcgt

SEQ ID NO: 1206 - DNA, 42 bp, synthetic construct
  misc_feature 1..42: note = Target Specific Oligonucleotide
  source 1..42: mol_type = other DNA
gtgcattattggacgaacatcatagactctataaatcagcgt

SEQ ID NO: 1207 - DNA, 45 bp, synthetic construct
  misc_feature 1..45: note = Target Specific Oligonucleotide
  source 1..45: mol_type = other DNA
gctgttgttaatggtctataatccatagactctataaatcagcgt

SEQ ID NO: 1208 - DNA, 45 bp, synthetic construct
  misc_feature 1..45: note = Target Specific Oligonucleotide
  source 1..45: mol_type = other DNA
cacacatatcctatgttcatcaatatagactctataaatcagcgt

SEQ ID NO: 1209 - DNA, 43 bp, synthetic construct
  misc_feature 1..43: note = Target Specific Oligonucleotide
  source 1..43: mol_type = other DNA
ttgagtggtcgttttcttacacatagactctataaatcagcgt

SEQ ID NO: 1210 - DNA, 45 bp, synthetic construct
  misc_feature 1..45: note = Target Specific Oligonucleotide
  source 1..45: mol_type = other DNA
cataatcaaaacgaaatgtttggtttagactctataaatcagcgt

SEQ ID NO: 1211 - DNA, 41 bp, synthetic construct
  misc_feature 1..41: note = Target Specific Oligonucleotide
  source 1..41: mol_type = other DNA
tttgtagcatcaacgtcctggtagactctataaatcagcgt

SEQ ID NO: 1212 - DNA, 41 bp, synthetic construct
  misc_feature 1..41: note = Target Specific Oligonucleotide
  source 1..41: mol_type = other DNA
caacacatttgacctcccgtttagactctataaatcagcgt

SEQ ID NO: 1213 - DNA, 43 bp, synthetic construct
  misc_feature 1..43: note = Target Specific Oligonucleotide
  source 1..43: mol_type = other DNA
ctaaccaccgaatagattcctggtagactctataaatcagcgt

SEQ ID NO: 1214 - DNA, 41 bp, synthetic construct
  misc_feature 1..41: note = Target Specific Oligonucleotide
  source 1..41: mol_type = other DNA
gcagatgtttgaccggatgtttagactctataaatcagcgt

SEQ ID NO: 1215 - DNA, 42 bp, synthetic construct
  misc_feature 1..42: note = Target Specific Oligonucleotide
  source 1..42: mol_type = other DNA
tgcttgatgtattcggccatcgtagactctataaatcagcgt

SEQ ID NO: 1216 - DNA, 42 bp, synthetic construct
  misc_feature 1..42: note = Target Specific Oligonucleotide
  source 1..42: mol_type = other DNA
tgagtttacttgcacggaaaggtagactctataaatcagcgt

SEQ ID NO: 1217 - DNA, 42 bp, synthetic construct
  misc_feature 1..42: note = Target Specific Oligonucleotide
  source 1..42: mol_type = other DNA
cactttatcccgcagttcagtttagactctataaatcagcgt

SEQ ID NO: 1218 - DNA, 41 bp, synthetic construct
  misc_feature 1..41: note = Target Specific Oligonucleotide
  source 1..41: mol_type = other DNA
gcaaagcatgtagccattccctagactctataaatcagcgt

SEQ ID NO: 1219 - DNA, 43 bp, synthetic construct
  misc_feature 1..43: note = Target Specific Oligonucleotide
  source 1..43: mol_type = other DNA
aaagacatctcgagctgctaatgtagactctataaatcagcgt

SEQ ID NO: 1220 - DNA, 43 bp, synthetic construct
  misc_feature 1..43: note = Target Specific Oligonucleotide
  source 1..43: mol_type = other DNA
gtcaccacccatagtatgagttttagactctataaatcagcgt

SEQ ID NO: 1221 - DNA, 43 bp, synthetic construct
  misc_feature 1..43: note = Target Specific Oligonucleotide
  source 1..43: mol_type = other DNA
tttctacctaaggctctgatgcatagactctataaatcagcgt

SEQ ID NO: 1222 - DNA, 41 bp, synthetic construct
  misc_feature 1..41: note = Target Specific Oligonucleotide
  source 1..41: mol_type = other DNA
tagagctgcacggaaagattttagactctataaatcagcgt

SEQ ID NO: 1223 - DNA, 43 bp, synthetic construct
  misc_feature 1..43: note = Target Specific Oligonucleotide
  source 1..43: mol_type = other DNA
gtgtagcatatgaatcgacatcatagactctataaatcagcgt

SEQ ID NO: 1224 - DNA, 43 bp, synthetic construct
  misc_feature 1..43: note = Target Specific Oligonucleotide
  source 1..43: mol_type = other DNA
tagccttgtatggcagaatcgtagacctaaattactgtgaatg

SEQ ID NO: 1225 - DNA, 45 bp, synthetic construct
  misc_feature 1..45: note = Target Specific Oligonucleotide
  source 1..45: mol_type = other DNA
tgaattgccctgggtataactcctagacctaaattactgtgaatg

SEQ ID NO: 1226 - DNA, 42 bp, synthetic construct
  misc_feature 1..42: note = Target Specific Oligonucleotide
  source 1..42: mol_type = other DNA
ttcctgttgcaacgtgagggtagacctaaattactgtgaatg

SEQ ID NO: 1227 - DNA, 46 bp, synthetic construct
  misc_feature 1..46: note = Target Specific Oligonucleotide
  source 1..46: mol_type = other DNA
atgaaagacacgatcaaaattgaatagacctaaattactgtgaatg

SEQ ID NO: 1228 - DNA, 43 bp, synthetic construct
  misc_feature 1..43: note = Target Specific Oligonucleotide
  source 1..43: mol_type = other DNA
gtcagcacgaagtgagttcaatagacctaaattactgtgaatg

SEQ ID NO: 1229 - DNA, 44 bp, synthetic construct
  misc_feature 1..44: note = Target Specific Oligonucleotide
  source 1..44: mol_type = other DNA
cagtttgcagcgtagaatctcttagacctaaattactgtgaatg

SEQ ID NO: 1230 - DNA, 45 bp, synthetic construct
  misc_feature 1..45: note = Target Specific Oligonucleotide
  source 1..45: mol_type = other DNA
gtgcagataatacgtgtctttgctagacctaaattactgtgaatg

SEQ ID NO: 1231 - DNA, 45 bp, synthetic construct
  misc_feature 1..45: note = Target Specific Oligonucleotide
  source 1..45: mol_type = other DNA
tccaatgagtatgacaagtccattagacctaaattactgtgaatg

SEQ ID NO: 1232 - DNA, 45 bp, synthetic construct
  misc_feature 1..45: note = Target Specific Oligonucleotide
  source 1..45: mol_type = other DNA
tgtaagaaagctcggtcttcaaatagacctaaattactgtgaatg

SEQ ID NO: 1233 - DNA, 46 bp, synthetic construct
  misc_feature 1..46: note = Target Specific Oligonucleotide
  source 1..46: mol_type = other DNA
ccatgtaagatacacgtaagagaatagacctaaattactgtgaatg

SEQ ID NO: 1234 - DNA, 46 bp, synthetic construct
  misc_feature 1..46: note = Target Specific Oligonucleotide
  source 1..46: mol_type = other DNA
gactcaagagcatttcgtaattgttagacctaaattactgtgaatg

SEQ ID NO: 1235 - DNA, 46 bp, synthetic construct
  misc_feature 1..46: note = Target Specific Oligonucleotide
  source 1..46: mol_type = other DNA
atgtcattagtcacctcaagttcttagacctaaattactgtgaatg

SEQ ID NO: 1236 - DNA, 39 bp, synthetic construct
  misc_feature 1..39: note = Target Specific Oligonucleotide
  source 1..39: mol_type = other DNA
ccgcactcgcacgcagatagacctaaattactgtgaatg

SEQ ID NO: 1237 - DNA, 47 bp, synthetic construct
  misc_feature 1..47: note = Target Specific Oligonucleotide
  source 1..47: mol_type = other DNA
tgttaacagtatcgtgaatatcacttagacctaaattactgtgaatg

SEQ ID NO: 1238 - DNA, 45 bp, synthetic construct
  misc_feature 1..45: note = Target Specific Oligonucleotide
  source 1..45: mol_type = other DNA
actttgccatctataagggaggttagacctaaattactgtgaatg

SEQ ID NO: 1239 - DNA, 48 bp, synthetic construct
  misc_feature 1..48: note = Target Specific Oligonucleotide
  source 1..48: mol_type = other DNA
cttgctatctcgataatttatgaaactagacctaaattactgtgaatg

SEQ ID NO: 1240 - DNA, 48 bp, synthetic construct
  misc_feature 1..48: note = Target Specific Oligonucleotide
  source 1..48: mol_type = other DNA
ttaatgtatcaagagtgttactgaaatagacctaaattactgtgaatg

SEQ ID NO: 1241 - DNA, 46 bp, synthetic construct
  misc_feature 1..46: note = Target Specific Oligonucleotide
  source 1..46: mol_type = other DNA
tcaaatgggataccttaacagatctagacctaaattactgtgaatg

SEQ ID NO: 1242 - DNA, 50 bp, synthetic construct
  misc_feature 1..50: note = Target Specific Oligonucleotide
  source 1..50: mol_type = other DNA
ataaataagcttcgatttatattacagctagacctaaattactgtgaatg

SEQ ID NO: 1243 - DNA, 45 bp, synthetic construct
  misc_feature 1..45: note = Target Specific Oligonucleotide
  source 1..45: mol_type = other DNA
cttgtgtggtaggattaacctggtagacctaaattactgtgaatg

SEQ ID NO: 1244 - DNA, 43 bp, synthetic construct
  misc_feature 1..43: note = Target Specific Oligonucleotide
  source 1..43: mol_type = other DNA
gttggtcaactcgagcatctttagacctaaattactgtgaatg

SEQ ID NO: 1245 - DNA, 44 bp, synthetic construct
  misc_feature 1..44: note = Target Specific Oligonucleotide
  source 1..44: mol_type = other DNA
tttggtttgtaggcctgtagcttagacctaaattactgtgaatg

SEQ ID NO: 1246 - DNA, 45 bp, synthetic construct
  misc_feature 1..45: note = Target Specific Oligonucleotide
  source 1..45: mol_type = other DNA
cattaggcagtaactcttgttcctagacctaaattactgtgaatg

SEQ ID NO: 1247 - DNA, 45 bp, synthetic construct
  misc_feature 1..45: note = Target Specific Oligonucleotide
  source 1..45: mol_type = other DNA
cttctcagatacgcttcttcttatagacctaaattactgtgaatg

SEQ ID NO: 1248 - DNA, 31 bp, synthetic construct
  misc_feature 1..31: note = Decoding Oligonucleotide
  source 1..31: mol_type = other DNA
ggctaatttattaccttctagctataacgat

SEQ ID NO: 1249 - DNA, 31 bp, synthetic construct
  misc_feature 1..31: note = Decoding Oligonucleotide
  source 1..31: mol_type = other DNA
accttaagaatggaattctagctataacgat

SEQ ID NO: 1250 - DNA, 30 bp, synthetic construct
  misc_feature 1..30: note = Decoding Oligonucleotide
  source 1..30: mol_type = other DNA
acttaacttgagtggtctagctataacgat

SEQ ID NO: 1251 - DNA, 29 bp, synthetic construct
  misc_feature 1..29: note = Decoding Oligonucleotide
  source 1..29: mol_type = other DNA
ggctaatttattacctcggtctagatgga

SEQ ID NO: 1252 - DNA, 29 bp, synthetic construct
  misc_feature 1..29: note = Decoding Oligonucleotide
  source 1..29: mol_type = other DNA
accttaagaatggaatcggtctagatgga

SEQ ID NO: 1253 - DNA, 28 bp, synthetic construct
  misc_feature 1..28: note = Decoding Oligonucleotide
  source 1..28: mol_type = other DNA
acttaacttgagtggcggtctagatgga

SEQ ID NO: 1254 - DNA, 32 bp, synthetic construct
  misc_feature 1..32: note = Decoding Oligonucleotide
  source 1..32: mol_type = other DNA
ggctaatttattacctgttaactccgtagtaa

SEQ ID NO: 1255 - DNA, 32 bp, synthetic construct
  misc_feature 1..32: note = Decoding Oligonucleotide
  source 1..32: mol_type = other DNA
accttaagaatggaatgttaactccgtagtaa

SEQ ID NO: 1256 - DNA, 31 bp, synthetic construct
  misc_feature 1..31: note = Decoding Oligonucleotide
  source 1..31: mol_type = other DNA
acttaacttgagtgggttaactccgtagtaa

SEQ ID NO: 1257 - DNA, 31 bp, synthetic construct
  misc_feature 1..31: note = Decoding Oligonucleotide
  source 1..31: mol_type = other DNA
ggctaatttattacctcgctatatctactga

SEQ ID NO: 1258 - DNA, 31 bp, synthetic construct
  misc_feature 1..31: note = Decoding Oligonucleotide
  source 1..31: mol_type = other DNA
accttaagaatggaatcgctatatctactga

SEQ ID NO: 1259 - DNA, 30 bp, synthetic construct
  misc_feature 1..30: note = Decoding Oligonucleotide
  source 1..30: mol_type = other DNA
acttaacttgagtggcgctatatctactga

SEQ ID NO: 1260 - DNA, 30 bp, synthetic construct
  misc_feature 1..30: note = Decoding Oligonucleotide
  source 1..30: mol_type = other DNA
ggctaatttattacctgtttccgtttcgaa

SEQ ID NO: 1261 - DNA, 30 bp, synthetic construct
  misc_feature 1..30: note = Decoding Oligonucleotide
  source 1..30: mol_type = other DNA
accttaagaatggaatgtttccgtttcgaa

SEQ ID NO: 1262 - DNA, 29 bp, synthetic construct
  misc_feature 1..29: note = Decoding Oligonucleotide
  source 1..29: mol_type = other DNA
acttaacttgagtgggtttccgtttcgaa

SEQ ID NO: 1263 - DNA, 30 bp, synthetic construct
  misc_feature 1..30: note = Decoding Oligonucleotide
  source 1..30: mol_type = other DNA
ggctaatttattaccttagcgtttcaaaag

SEQ ID NO: 1264 - DNA, 30 bp, synthetic construct
  misc_feature 1..30: note = Decoding Oligonucleotide
  source 1..30: mol_type = other DNA
accttaagaatggaattagcgtttcaaaag

SEQ ID NO: 1265 - DNA, 29 bp, synthetic construct
  misc_feature 1..29: note = Decoding Oligonucleotide
  source 1..29: mol_type = other DNA
acttaacttgagtggtagcgtttcaaaag

SEQ ID NO: 1266 - DNA, 28 bp, synthetic construct
  misc_feature 1..28: note = Decoding Oligonucleotide
  source 1..28: mol_type = other DNA
ggctaatttattaccttgatccgacgag

SEQ ID NO: 1267 - DNA, 28 bp, synthetic construct
  misc_feature 1..28: note = Decoding Oligonucleotide
  source 1..28: mol_type = other DNA
accttaagaatggaattgatccgacgag

SEQ ID NO: 1268 - DNA, 27 bp, synthetic construct
  misc_feature 1..27: note = Decoding Oligonucleotide
  source 1..27: mol_type = other DNA
acttaacttgagtggtgatccgacgag

SEQ ID NO: 1269 - DNA, 31 bp, synthetic construct
  misc_feature 1..31: note = Decoding Oligonucleotide
  source 1..31: mol_type = other DNA
ggctaatttattacctaatctacatgtctga

SEQ ID NO: 1270 - DNA, 31 bp, synthetic construct
  misc_feature 1..31: note = Decoding Oligonucleotide
  source 1..31: mol_type = other DNA
accttaagaatggaataatctacatgtctga

SEQ ID NO: 1271 - DNA, 30 bp, synthetic construct
  misc_feature 1..30: note = Decoding Oligonucleotide
  source 1..30: mol_type = other DNA
acttaacttgagtggaatctacatgtctga

SEQ ID NO: 1272 - DNA, 30 bp, synthetic construct
  misc_feature 1..30: note = Decoding Oligonucleotide
  source 1..30: mol_type = other DNA
ggctaatttattacctggacgttgaataca

SEQ ID NO: 1273 - DNA, 30 bp, synthetic construct
  misc_feature 1..30: note = Decoding Oligonucleotide
  source 1..30: mol_type = other DNA
accttaagaatggaatggacgttgaataca

SEQ ID NO: 1274 - DNA, 29 bp, synthetic construct
  misc_feature 1..29: note = Decoding Oligonucleotide
  source 1..29: mol_type = other DNA
acttaacttgagtggggacgttgaataca

SEQ ID NO: 1275 - DNA, 31 bp, synthetic construct
  misc_feature 1..31: note = Decoding Oligonucleotide
  source 1..31: mol_type = other DNA
ggctaatttattaccttaaagtgacaattca

SEQ ID NO: 1276 - DNA, 31 bp, synthetic construct
  misc_feature 1..31: note = Decoding Oligonucleotide
  source 1..31: mol_type = other DNA
accttaagaatggaattaaagtgacaattca

SEQ ID NO: 1277 - DNA, 30 bp, synthetic construct
  misc_feature 1..30: note = Decoding Oligonucleotide
  source 1..30: mol_type = other DNA
acttaacttgagtggtaaagtgacaattca

SEQ ID NO: 1278 - DNA, 32 bp, synthetic construct
  misc_feature 1..32: note = Decoding Oligonucleotide
  source 1..32: mol_type = other DNA
ggctaatttattacctgattcccataattagg

SEQ ID NO: 1279 - DNA, 32 bp, synthetic construct
  misc_feature 1..32: note = Decoding Oligonucleotide
  source 1..32: mol_type = other DNA
accttaagaatggaatgattcccataattagg

SEQ ID NO: 1280 - DNA, 31 bp, synthetic construct
  misc_feature 1..31: note = Decoding Oligonucleotide
  source 1..31: mol_type = other DNA
acttaacttgagtgggattcccataattagg

SEQ ID NO: 1281 - DNA, 33 bp, synthetic construct
  misc_feature 1..33: note = Decoding Oligonucleotide
  source 1..33: mol_type = other DNA
ggctaatttattacctcgtcgtataaatctatc

SEQ ID NO: 1282 - DNA, 33 bp, synthetic construct
  misc_feature 1..33: note = Decoding Oligonucleotide
  source 1..33: mol_type = other DNA
accttaagaatggaatcgtcgtataaatctatc

SEQ ID NO: 1283 - DNA, 32 bp, synthetic construct
  misc_feature 1..32: note = Decoding Oligonucleotide
  source 1..32: mol_type = other DNA
acttaacttgagtggcgtcgtataaatctatc

SEQ ID NO: 1284 - DNA, 34 bp, synthetic construct
  misc_feature 1..34: note = Decoding Oligonucleotide
  source 1..34: mol_type = other DNA
ggctaatttattacctgatgtctatcctttataa

SEQ ID NO: 1285 - DNA, 34 bp, synthetic construct
  misc_feature 1..34: note = Decoding Oligonucleotide
  source 1..34: mol_type = other DNA
accttaagaatggaatgatgtctatcctttataa

SEQ ID NO: 1286 - DNA, 33 bp, synthetic construct
  misc_feature 1..33: note = Decoding Oligonucleotide
  source 1..33: mol_type = other DNA
acttaacttgagtgggatgtctatcctttataa

SEQ ID NO: 1287 - DNA, 30 bp, synthetic construct
  misc_feature 1..30: note = Decoding Oligonucleotide
  source 1..30: mol_type = other DNA
ggctaatttattaccttttccacgataacc

SEQ ID NO: 1288 - DNA, 30 bp, synthetic construct
  misc_feature 1..30: note = Decoding Oligonucleotide
  source 1..30: mol_type = other DNA
accttaagaatggaattttccacgataacc

SEQ ID NO: 1289 - DNA, 29 bp, synthetic construct
  misc_feature 1..29: note = Decoding Oligonucleotide
  source 1..29: mol_type = other DNA
acttaacttgagtggtttccacgataacc

SEQ ID NO: 1290 - DNA, 31 bp, synthetic construct
  misc_feature 1..31: note = Decoding Oligonucleotide
  source 1..31: mol_type = other DNA
ggctaatttattaccttagtaccgaataagg

SEQ ID NO: 1291 - DNA, 31 bp, synthetic construct
  misc_feature 1..31: note = Decoding Oligonucleotide
  source 1..31: mol_type = other DNA
accttaagaatggaattagtaccgaataagg

SEQ ID NO: 1292 - DNA, 30 bp, synthetic construct
  misc_feature 1..30: note = Decoding Oligonucleotide
  source 1..30: mol_type = other DNA
acttaacttgagtggtagtaccgaataagg

SEQ ID NO: 1293 - DNA, 32 bp, synthetic construct
  misc_feature 1..32: note = Decoding Oligonucleotide
  source 1..32: mol_type = other DNA
ggctaatttattaccttaaagttgtaaaatcc

SEQ ID NO: 1294 - DNA, 32 bp, synthetic construct
  misc_feature 1..32: note = Decoding Oligonucleotide
  source 1..32: mol_type = other DNA
accttaagaatggaattaaagttgtaaaatcc

SEQ ID NO: 1295 - DNA, 31 bp, synthetic construct
  misc_feature 1..31: note = Decoding Oligonucleotide
  source 1..31: mol_type = other DNA
acttaacttgagtggtaaagttgtaaaatcc

SEQ ID NO: 1296 - DNA, 32 bp, synthetic construct
  misc_feature 1..32: note = Decoding Oligonucleotide
  source 1..32: mol_type = other DNA
ggctaatttattacctggataagtcggtaatc

SEQ ID NO: 1297 - DNA, 32 bp, synthetic construct
  misc_feature 1..32: note = Decoding Oligonucleotide
  source 1..32: mol_type = other DNA
accttaagaatggaatggataagtcggtaatc

SEQ ID NO: 1298 - DNA, 31 bp, synthetic construct
  misc_feature 1..31: note = Decoding Oligonucleotide
  source 1..31: mol_type = other DNA
acttaacttgagtggggataagtcggtaatc

SEQ ID NO: 1299 - DNA, 33 bp, synthetic construct
  misc_feature 1..33: note = Decoding Oligonucleotide
  source 1..33: mol_type = other DNA
ggctaatttattacctggtatttcactctaatt

SEQ ID NO: 1300 - DNA, 33 bp, synthetic construct
  misc_feature 1..33: note = Decoding Oligonucleotide
  source 1..33: mol_type = other DNA
accttaagaatggaatggtatttcactctaatt

SEQ ID NO: 1301 - DNA, 32 bp, synthetic construct
  misc_feature 1..32: note = Decoding Oligonucleotide
  source 1..32: mol_type = other DNA
acttaacttgagtggggtatttcactctaatt

SEQ ID NO: 1302 - DNA, 32 bp, synthetic construct
  misc_feature 1..32: note = Decoding Oligonucleotide
  source 1..32: mol_type = other DNA
ggctaatttattacctagctagataaagtacc

SEQ ID NO: 1303 - DNA, 32 bp, synthetic construct
  misc_feature 1..32: note = Decoding Oligonucleotide
  source 1..32: mol_type = other DNA
accttaagaatggaatagctagataaagtacc

SEQ ID NO: 1304 - DNA, 31 bp, synthetic construct
  misc_feature 1..31: note = Decoding Oligonucleotide
  source 1..31: mol_type = other DNA
acttaacttgagtggagctagataaagtacc

SEQ ID NO: 1305 - DNA, 32 bp, synthetic construct
  misc_feature 1..32: note = Decoding Oligonucleotide
  source 1..32: mol_type = other DNA
ggctaatttattacctccgaaactacgattat

SEQ ID NO: 1306 - DNA, 32 bp, synthetic construct
  misc_feature 1..32: note = Decoding Oligonucleotide
  source 1..32: mol_type = other DNA
accttaagaatggaatccgaaactacgattat

SEQ ID NO: 1307 - DNA, 31 bp, synthetic construct
  misc_feature 1..31: note = Decoding Oligonucleotide
  source 1..31: mol_type = other DNA
acttaacttgagtggccgaaactacgattat

SEQ ID NO: 1308 - DNA, 32 bp, synthetic construct
  misc_feature 1..32: note = Decoding Oligonucleotide
  source 1..32: mol_type = other DNA
ggctaatttattacctcatcgaatttatgtta

SEQ ID NO: 1309 - DNA, 32 bp, synthetic construct
  misc_feature 1..32: note = Decoding Oligonucleotide
  source 1..32: mol_type = other DNA
accttaagaatggaatcatcgaatttatgtta

SEQ ID NO: 1310 - DNA, 31 bp, synthetic construct
  misc_feature 1..31: note = Decoding Oligonucleotide
  source 1..31: mol_type = other DNA
acttaacttgagtggcatcgaatttatgtta

SEQ ID NO: 1311 - DNA, 30 bp, synthetic construct
  misc_feature 1..30: note = Decoding Oligonucleotide
  source 1..30: mol_type = other DNA
ggctaatttattacctccgtaattctaccg

SEQ ID NO: 1312 - DNA, 30 bp, synthetic construct
  misc_feature 1..30: note = Decoding Oligonucleotide
  source 1..30: mol_type = other DNA
accttaagaatggaatccgtaattctaccg

SEQ ID NO: 1313 - DNA, 29 bp, synthetic construct
  misc_feature 1..29: note = Decoding Oligonucleotide
  source 1..29: mol_type = other DNA
acttaacttgagtggccgtaattctaccg

SEQ ID NO: 1314 - DNA, 29 bp, synthetic construct
  misc_feature 1..29: note = Decoding Oligonucleotide
  source 1..29: mol_type = other DNA
ggctaatttattacctgggtaatcgtcac

SEQ ID NO: 1315 - DNA, 29 bp, synthetic construct
  misc_feature 1..29: note = Decoding Oligonucleotide
  source 1..29: mol_type = other DNA
accttaagaatggaatgggtaatcgtcac

SEQ ID NO: 1316 - DNA, 28 bp, synthetic construct
  misc_feature 1..28: note = Decoding Oligonucleotide
  source 1..28: mol_type = other DNA
acttaacttgagtgggggtaatcgtcac

SEQ ID NO: 1317 - DNA, 31 bp, synthetic construct
  misc_feature 1..31: note = Decoding Oligonucleotide
  source 1..31: mol_type = other DNA
ggctaatttattacctagtgttatgagaagc

SEQ ID NO: 1318 - DNA, 31 bp, synthetic construct
  misc_feature 1..31: note = Decoding Oligonucleotide
  source 1..31: mol_type = other DNA
accttaagaatggaatagtgttatgagaagc

SEQ ID NO: 1319 - DNA, 30 bp, synthetic construct
  misc_feature 1..30: note = Decoding Oligonucleotide
  source 1..30: mol_type = other DNA
acttaacttgagtggagtgttatgagaagc

SEQ ID NO: 1320 - DNA, 31 bp, synthetic construct
  misc_feature 1..31: note = Decoding Oligonucleotide
  source 1..31: mol_type = other DNA
ggctaatttattacctctgcaatagaagaga

SEQ ID NO: 1321 - DNA, 31 bp, synthetic construct
  misc_feature 1..31: note = Decoding Oligonucleotide
  source 1..31: mol_type = other DNA
accttaagaatggaatctgcaatagaagaga

SEQ ID NO: 1322 - DNA, 30 bp, synthetic construct
  misc_feature 1..30: note = Decoding Oligonucleotide
  source 1..30: mol_type = other DNA
acttaacttgagtggctgcaatagaagaga

SEQ ID NO: 1323 - DNA, 30 bp, synthetic construct
  misc_feature 1..30: note = Decoding Oligonucleotide
  source 1..30: mol_type = other DNA
ggctaatttattacctgatttttcagcgac

SEQ ID NO: 1324 - DNA, 30 bp, synthetic construct
  misc_feature 1..30: note = Decoding Oligonucleotide
  source 1..30: mol_type = other DNA
accttaagaatggaatgatttttcagcgac

SEQ ID NO: 1325 - DNA, 29 bp, synthetic construct
  misc_feature 1..29: note = Decoding Oligonucleotide
  source 1..29: mol_type = other DNA
acttaacttgagtgggatttttcagcgac

SEQ ID NO: 1326 - DNA, 32 bp, synthetic construct
  misc_feature 1..32: note = Decoding Oligonucleotide
  source 1..32: mol_type = other DNA
ggctaatttattacctcgtaatatgaagatca

SEQ ID NO: 1327 - DNA, 32 bp, synthetic construct
  misc_feature 1..32: note = Decoding Oligonucleotide
  source 1..32: mol_type = other DNA
accttaagaatggaatcgtaatatgaagatca

SEQ ID NO: 1328 - DNA, 31 bp, synthetic construct
  misc_feature 1..31: note = Decoding Oligonucleotide
  source 1..31: mol_type = other DNA
acttaacttgagtggcgtaatatgaagatca

SEQ ID NO: 1329 - DNA, 32 bp, synthetic construct
  misc_feature 1..32: note = Decoding Oligonucleotide
  source 1..32: mol_type = other DNA
ggctaatttattacctgaattggaatattcgt

SEQ ID NO: 1330 - DNA, 32 bp, synthetic construct
  misc_feature 1..32: note = Decoding Oligonucleotide
  source 1..32: mol_type = other DNA
accttaagaatggaatgaattggaatattcgt

SEQ ID NO: 1331 - DNA, 31 bp, synthetic construct
  misc_feature 1..31: note = Decoding Oligonucleotide
  source 1..31: mol_type = other DNA
acttaacttgagtgggaattggaatattcgt

SEQ ID NO: 1332 - DNA, 33 bp, synthetic construct
  misc_feature 1..33: note = Decoding Oligonucleotide
  source 1..33: mol_type = other DNA
ggctaatttattacctggtattaatcttgaaga

SEQ ID NO: 1333 - DNA, 33 bp, synthetic construct
  misc_feature 1..33: note = Decoding Oligonucleotide
  source 1..33: mol_type = other DNA
accttaagaatggaatggtattaatcttgaaga

SEQ ID NO: 1334 - DNA, 32 bp, synthetic construct
  misc_feature 1..32: note = Decoding Oligonucleotide
  source 1..32: mol_type = other DNA
acttaacttgagtggggtattaatcttgaaga

SEQ ID NO: 1335 - DNA, 29 bp, synthetic construct
  misc_feature 1..29: note = Decoding Oligonucleotide
  source 1..29: mol_type = other DNA
ggctaatttattacctacgatgtattgcc

SEQ ID NO: 1336 - DNA, 29 bp, synthetic construct
  misc_feature 1..29: note = Decoding Oligonucleotide
  source 1..29: mol_type = other DNA
accttaagaatggaatacgatgtattgcc

SEQ ID NO: 1337 - DNA, 28 bp, synthetic construct
  misc_feature 1..28: note = Decoding Oligonucleotide
  source 1..28: mol_type = other DNA
acttaacttgagtggacgatgtattgcc

SEQ ID NO: 1338 - DNA, 32 bp, synthetic construct
  misc_feature 1..32: note = Decoding Oligonucleotide
  source 1..32: mol_type = other DNA
ggctaatttattacctcatatacaaacggtac

SEQ ID NO: 1339 - DNA, 32 bp, synthetic construct
  misc_feature 1..32: note = Decoding Oligonucleotide
  source 1..32: mol_type = other DNA
accttaagaatggaatcatatacaaacggtac

SEQ ID NO: 1340 - DNA, 31 bp, synthetic construct
  misc_feature 1..31: note = Decoding Oligonucleotide
  source 1..31: mol_type = other DNA
acttaacttgagtggcatatacaaacggtac

SEQ ID NO: 1341 - DNA, 31 bp, synthetic construct
  misc_feature 1..31: note = Decoding Oligonucleotide
  source 1..31: mol_type = other DNA
ggctaatttattacctttccgatcttaactt

SEQ ID NO: 1342 - DNA, 31 bp, synthetic construct
  misc_feature 1..31: note = Decoding Oligonucleotide
  source 1..31: mol_type = other DNA
accttaagaatggaatttccgatcttaactt

SEQ ID NO: 1343 - DNA, 30 bp, synthetic construct
  misc_feature 1..30: note = Decoding Oligonucleotide
  source 1..30: mol_type = other DNA
acttaacttgagtggttccgatcttaactt

SEQ ID NO: 1344 - DNA, 31 bp, synthetic construct
  misc_feature 1..31: note = Decoding Oligonucleotide
  source 1..31: mol_type = other DNA
ggctaatttattacctgtgttaattcgcatc

SEQ ID NO: 1345 - DNA, 31 bp, synthetic construct
  misc_feature 1..31: note = Decoding Oligonucleotide
  source 1..31: mol_type = other DNA
accttaagaatggaatgtgttaattcgcatc

SEQ ID NO: 1346 - DNA, 30 bp, synthetic construct
  misc_feature 1..30: note = Decoding Oligonucleotide
  source 1..30: mol_type = other DNA
acttaacttgagtgggtgttaattcgcatc

SEQ ID NO: 1347 - DNA, 30 bp, synthetic construct
  misc_feature 1..30: note = Decoding Oligonucleotide
  source 1..30: mol_type = other DNA
ggctaatttattacctcgtttaattgggct

SEQ ID NO: 1348 - DNA, 30 bp, synthetic construct
  misc_feature 1..30: note = Decoding Oligonucleotide
  source 1..30: mol_type = other DNA
accttaagaatggaatcgtttaattgggct

SEQ ID NO: 1349 - DNA, 29 bp, synthetic construct
  misc_feature 1..29: note = Decoding Oligonucleotide
  source 1..29: mol_type = other DNA
acttaacttgagtggcgtttaattgggct

SEQ ID NO: 1350 - DNA, 31 bp, synthetic construct
  misc_feature 1..31: note = Decoding Oligonucleotide
  source 1..31: mol_type = other DNA
ggctaatttattacctcaacacttataacgg

SEQ ID NO: 1351 - DNA, 31 bp, synthetic construct
  misc_feature 1..31: note = Decoding Oligonucleotide
  source 1..31: mol_type = other DNA
accttaagaatggaatcaacacttataacgg

SEQ ID NO: 1352 - DNA, 30 bp, synthetic construct
  misc_feature 1..30: note = Decoding Oligonucleotide
  source 1..30: mol_type = other DNA
acttaacttgagtggcaacacttataacgg

SEQ ID NO: 1353 - DNA, 34 bp, synthetic construct
  misc_feature 1..34: note = Decoding Oligonucleotide
  source 1..34: mol_type = other DNA
ggctaatttattacctggaaataagtattatacc

SEQ ID NO: 1354 - DNA, 34 bp, synthetic construct
  misc_feature 1..34: note = Decoding Oligonucleotide
  source 1..34: mol_type = other DNA
accttaagaatggaatggaaataagtattatacc

SEQ ID NO: 1355 - DNA, 33 bp, synthetic construct
  misc_feature 1..33: note = Decoding Oligonucleotide
  source 1..33: mol_type = other DNA
acttaacttgagtggggaaataagtattatacc

SEQ ID NO: 1356 - DNA, 29 bp, synthetic construct
  misc_feature 1..29: note = Decoding Oligonucleotide
  source 1..29: mol_type = other DNA
ggctaatttattacctcttaacgacggtt

SEQ ID NO: 1357 - DNA, 29 bp, synthetic construct
  misc_feature 1..29: note = Decoding Oligonucleotide
  source 1..29: mol_type = other DNA
accttaagaatggaatcttaacgacggtt

SEQ ID NO: 1358 - DNA, 28 bp, synthetic construct
  misc_feature 1..28: note = Decoding Oligonucleotide
  source 1..28: mol_type = other DNA
acttaacttgagtggcttaacgacggtt

SEQ ID NO: 1359 - DNA, 33 bp, synthetic construct
  misc_feature 1..33: note = Decoding Oligonucleotide
  source 1..33: mol_type = other DNA
ggctaatttattacctgctcgttagaatttatt

SEQ ID NO: 1360 - DNA, 33 bp, synthetic construct
  misc_feature 1..33: note = Decoding Oligonucleotide
  source 1..33: mol_type = other DNA
accttaagaatggaatgctcgttagaatttatt

SEQ ID NO: 1361 - DNA, 32 bp, synthetic construct
  misc_feature 1..32: note = Decoding Oligonucleotide
  source 1..32: mol_type = other DNA
acttaacttgagtgggctcgttagaatttatt

SEQ ID NO: 1362 - DNA, 32 bp, synthetic construct
  misc_feature 1..32: note = Decoding Oligonucleotide
  source 1..32: mol_type = other DNA
ggctaatttattaccttctttatcatcattga

SEQ ID NO: 1363 - DNA, 32 bp, synthetic construct
  misc_feature 1..32: note = Decoding Oligonucleotide
  source 1..32: mol_type = other DNA
accttaagaatggaattctttatcatcattga

SEQ ID NO: 1364 - DNA, 31 bp, synthetic construct
  misc_feature 1..31: note = Decoding Oligonucleotide
  source 1..31: mol_type = other DNA
acttaacttgagtggtctttatcatcattga

SEQ ID NO: 1365 - DNA, 31 bp, synthetic construct
  misc_feature 1..31: note = Decoding Oligonucleotide
  source 1..31: mol_type = other DNA
ggctaatttattacctcgttcgcaatttatc

SEQ ID NO: 1366 - DNA, 31 bp, synthetic construct
  misc_feature 1..31: note = Decoding Oligonucleotide
  source 1..31: mol_type = other DNA
accttaagaatggaatcgttcgcaatttatc

SEQ ID NO: 1367 - DNA, 30 bp, synthetic construct
  misc_feature 1..30: note = Decoding Oligonucleotide
  source 1..30: mol_type = other DNA
acttaacttgagtggcgttcgcaatttatc

SEQ ID NO: 1368 - DNA, 31 bp, synthetic construct
  misc_feature 1..31: note = Decoding Oligonucleotide
  source 1..31: mol_type = other DNA
ggctaatttattacctcgatcttttgttgta

SEQ ID NO: 1369 - DNA, 31 bp, synthetic construct
  misc_feature 1..31: note = Decoding Oligonucleotide
  source 1..31: mol_type = other DNA
accttaagaatggaatcgatcttttgttgta

SEQ ID NO: 1370 - DNA, 30 bp, synthetic construct
  misc_feature 1..30: note = Decoding Oligonucleotide
  source 1..30: mol_type = other DNA
acttaacttgagtggcgatcttttgttgta

SEQ ID NO: 1371 - DNA, 32 bp, synthetic construct
  misc_feature 1..32: note = Decoding Oligonucleotide
  source 1..32: mol_type = other DNA
ggctaatttattacctaccactcaatataaga

SEQ ID NO: 1372 - DNA, 32 bp, synthetic construct
  misc_feature 1..32: note = Decoding Oligonucleotide
  source 1..32: mol_type = other DNA
accttaagaatggaataccactcaatataaga

SEQ ID NO: 1373 - DNA, 31 bp, synthetic construct
  misc_feature 1..31: note = Decoding Oligonucleotide
  source 1..31: mol_type = other DNA
acttaacttgagtggaccactcaatataaga

SEQ ID NO: 1374 - DNA, 30 bp, synthetic construct
  misc_feature 1..30: note = Decoding Oligonucleotide
  source 1..30: mol_type = other DNA
ggctaatttattacctccgtaaattcggtc

SEQ ID NO: 1375 - DNA, 30 bp, synthetic construct
  misc_feature 1..30: note = Decoding Oligonucleotide
  source 1..30: mol_type = other DNA
accttaagaatggaatccgtaaattcggtc

SEQ ID NO: 1376 - DNA, 29 bp, synthetic construct
  misc_feature 1..29: note = Decoding Oligonucleotide
  source 1..29: mol_type = other DNA
acttaacttgagtggccgtaaattcggtc

SEQ ID NO: 1377 - DNA, 30 bp, synthetic construct
  misc_feature 1..30: note = Decoding Oligonucleotide
  source 1..30: mol_type = other DNA
ggctaatttattacctcgtgagatttctga

SEQ ID NO: 1378 - DNA, 30 bp, synthetic construct
  misc_feature 1..30: note = Decoding Oligonucleotide
  source 1..30: mol_type = other DNA
accttaagaatggaatcgtgagatttctga

SEQ ID NO: 1379 - DNA, 29 bp, synthetic construct
  misc_feature 1..29: note = Decoding Oligonucleotide
  source 1..29: mol_type = other DNA
acttaacttgagtggcgtgagatttctga

SEQ ID NO: 1380 - DNA, 31 bp, synthetic construct
  misc_feature 1..31: note = Decoding Oligonucleotide
  source 1..31: mol_type = other DNA
ggctaatttattacctcgaagcatagaatag

SEQ ID NO: 1381 - DNA, 31 bp, synthetic construct
  misc_feature 1..31: note = Decoding Oligonucleotide
  source 1..31: mol_type = other DNA
accttaagaatggaatcgaagcatagaatag

SEQ ID NO: 1382 - DNA, 30 bp, synthetic construct
  misc_feature 1..30: note = Decoding Oligonucleotide
  source 1..30: mol_type = other DNA
acttaacttgagtggcgaagcatagaatag

SEQ ID NO: 1383 - DNA, 30 bp, synthetic construct
  misc_feature 1..30: note = Decoding Oligonucleotide
  source 1..30: mol_type = other DNA
ggctaatttattacctaaaatacgggagac

SEQ ID NO: 1384 - DNA, 30 bp, synthetic construct
  misc_feature 1..30: note = Decoding Oligonucleotide
  source 1..30: mol_type = other DNA
accttaagaatggaataaaatacgggagac

SEQ ID NO: 1385 - DNA, 29 bp, synthetic construct
  misc_feature 1..29: note = Decoding Oligonucleotide
  source 1..29: mol_type = other DNA
acttaacttgagtggaaaatacgggagac

SEQ ID NO: 1386 - DNA, 31 bp, synthetic construct
  misc_feature 1..31: note = Decoding Oligonucleotide
  source 1..31: mol_type = other DNA
ggctaatttattacctcgcatggtataacat

SEQ ID NO: 1387 - DNA, 31 bp, synthetic construct
  misc_feature 1..31: note = Decoding Oligonucleotide
  source 1..31: mol_type = other DNA
accttaagaatggaatcgcatggtataacat

SEQ ID NO: 1388 - DNA, 30 bp, synthetic construct
  misc_feature 1..30: note = Decoding Oligonucleotide
  source 1..30: mol_type = other DNA
acttaacttgagtggcgcatggtataacat

SEQ ID NO: 1389 - DNA, 31 bp, synthetic construct
  misc_feature 1..31: note = Decoding Oligonucleotide
  source 1..31: mol_type = other DNA
ggctaatttattacctacgctgatttataga

SEQ ID NO: 1390 - DNA, 31 bp, synthetic construct
  misc_feature 1..31: note = Decoding Oligonucleotide
  source 1..31: mol_type = other DNA
accttaagaatggaatacgctgatttataga

SEQ ID NO: 1391 - DNA, 30 bp, synthetic construct
  misc_feature 1..30: note = Decoding Oligonucleotide
  source 1..30: mol_type = other DNA
acttaacttgagtggacgctgatttataga

SEQ ID NO: 1392 - DNA, 33 bp, synthetic construct
  misc_feature 1..33: note = Decoding Oligonucleotide
  source 1..33: mol_type = other DNA
ggctaatttattacctcgtagggtaaatagatt

SEQ ID NO: 1393 - DNA, 33 bp, synthetic construct
  misc_feature 1..33: note = Decoding Oligonucleotide
  source 1..33: mol_type = other DNA
accttaagaatggaatcgtagggtaaatagatt

SEQ ID NO: 1394 - DNA, 32 bp, synthetic construct
  misc_feature 1..32: note = Decoding Oligonucleotide
  source 1..32: mol_type = other DNA
acttaacttgagtggcgtagggtaaatagatt

SEQ ID NO: 1395 - DNA, 33 bp, synthetic construct
  misc_feature 1..33: note = Decoding Oligonucleotide
  source 1..33: mol_type = other DNA
ggctaatttattacctcattcacagtaatttag

SEQ ID NO: 1396 - DNA, 33 bp, synthetic construct
  misc_feature 1..33: note = Decoding Oligonucleotide
  source 1..33: mol_type = other DNA
accttaagaatggaatcattcacagtaatttag

SEQ ID NO: 1397 - DNA, 32 bp, synthetic construct
  misc_feature 1..32: note = Decoding Oligonucleotide
  source 1..32: mol_type = other DNA
acttaacttgagtggcattcacagtaatttag

SEQ ID NO: 1398 - DNA, 15 bp, synthetic construct
  misc_feature 1..15: note = Signal Oligonucleotide
  source 1..15: mol_type = other DNA
ccactcaagttaagt

SEQ ID NO: 1399 - DNA, 16 bp, synthetic construct
  misc_feature 1..16: note = Signal Oligonucleotide
  source 1..16: mol_type = other DNA
attccattcttaaggt

SEQ ID NO: 1400 - DNA, 16 bp, synthetic construct
  misc_feature 1..16: note = Signal Oligonucleotide
  source 1..16: mol_type = other DNA
aggtaataaattagcc